(19) United States
(12) Patent Application Publication
(10) Pub. No.: US 2025/0049682 A1
(43) Pub. Date: Feb. 13, 2025

(54) METHODS FOR ACTIVATION AND PREPARATION OF KERATIN FIBER COATINGS, PREFERABLY COLOR COATINGS

(71) Applicant: Wella Germany GMBH, Darmstadt (DE)

(72) Inventors: Mathias Kurt HERRLEIN, Kronberg (DE); Graham Neil MCKELVEY, Glashütten (DE); Matija CRNE, Wiesbaden (DE); Simon Paul GODFREY, Oberursel (DE); Corinne Violette MOHR, Lorsch (DE); Ingo WEBER, Basel (CH); Swapna PINAKATTU, Mainz (DE); Tatjana SCHAEFER, Butzbach (DE); Patrick Alexander KIEFER, Darmstadt (DE); Petra Barbara BRAUN, Münster (DE); Andrej GROSS, Darmstadt (DE); Felix HERKNER, Eppstein (DE); Axel MEYER, Frankfurt am Main (DE); Carl Uwe Oswald Ludwig SCHMIDT, Reinheim (DE); Michael A. BROOK, Ancaster (CA); Claus SCHREINER, Osterholz-Scharmbeck (DE); Timothy Robert CLARK, Obermichelbach (DE); Juergen Karl Anton SCHATZ, Adelsdorf (DE); Galina GROSS, Darmstadt (DE); Heiko BAUKNECHT, Bremen (DE)

(73) Assignee: Wella Germany GMBH, Darmstadt (DE)

(21) Appl. No.: 18/574,989

(22) PCT Filed: Dec. 22, 2021
(86) PCT No.: PCT/EP2021/087400

§ 371 Date: Dec. 28, 2023

(30) Foreign Application Priority Data
Jun. 29, 2021 (WO) PCT/EP2021/067925
Jun. 29, 2021 (WO) PCT/EP2021/067926
Jun. 29, 2021 (WO) PCT/EP2021/067927
Jun. 29, 2021 (WO) PCT/EP2021/067928

(51) Int. Cl. A61K 8/58; A61Q 5/10

(52) U.S. Cl. CPC A61K 8/585; A61Q 5/10; A61K 2800/43; A61K 2800/54; A61K 2800/884; A61K 2800/95

(57) ABSTRACT

A coating, preferably a color coating, method for keratin fibers is described which includes three steps: activation, pretreatment and binding. The activation step includes one or both of a Praeparatur procedure and a Fundamenta procedure to produce modified fibers. The pretreatment step applies a pretreatment composition of at least a PTH alkoxysilane with PTH as a thiol, a protected thiol or a group complementarily reactive with thiol to the modified fibers to form pretreated fibers. The binding step applies a film forming composition to the pretreated fibers to form the color coating.

### BACKGROUND

The recent two decades have seen significant effort directed toward development of external hair coloring technology that can be characterized as permanent but avoids bleach and penetration of dye precursors into the keratin fiber cortex. This technology in the main involves production of coloration of hair strands without involving the cortex. Hair coloration can be achieved by coating the surfaces of keratin fibers with a cosmetically acceptable polymer film containing pigment particles or with multi-layer polar attractive films containing coloration dyes instead of pigments. Numerous difficulties have plagued non-cortex hair coloration, however. Water soluble polymer films are easily removed by shampoo. Water insoluble and multi-layer films seem to have better remanence but they also typically survive only two or three shampoo washings. Film inflexibility, film irregularity, film thickness, inability to distribute color appropriately throughout hair tresses, film weakness and film dissolution through solvation, polar and/or ionic interaction with shampoo, organic liquids and other hair dressing components deliver undesirable tactile responses and physical displacement of the polymer film. These difficulties lead to flaking, hair strand breakage, artificial appearance, and irregular, undesirable color removal.

Hair coloring technology has made progress over the past five years in solving these issues. Better remanence and development of elasticity of the color coating on the hair tresses have been achieved by various technologies. Additional improvements include development of techniques for combinations of various pigments and pigment distribution designed to mimic at least to some extent natural highlights of hair.

These kinds of coatings and preferably color coatings span the range from organic, silicone and organosilicone compositions to biological protein derivative compositions. Mixtures and layers of coatings and preferably color coatings are typical in this respect. Suitability and compatibility of such mixtures and layers and their interactions with hair and scalp are still problematic, however. These coatings and especially color coatings still exhibit flaking, roughness, stiffness, irregular films, lack of flexibility, rough texture, touch and feel, and lack of remanence. Inner-coating connections can lead to coatings and especially color coatings that exhibit stiffness, flaking, thick texture/feel and extreme difficulty in attempts to remove and/or replace the coating with another coating. Rapid setting and lack of flowability of the compositions for application can lead to patchy coverage instead of contiguous coating formation.

When such coatings and especially color coatings have been combined with keratin fibers such as hair, esthetically pleasing coloration with long remanence has proved impossible to achieve at present due to the intrinsic qualities of anagenic hair. To date, experimentation to develop keratin fiber coloration such as hair coloration has focused on the use of hair tresses or swatches. These tresses are formed of natural hair but are detached from source (a person) and are usually preprocessed to deliver ease of use for experimental purposes. Such tresses do not enable experimentation and exploitation of the issues and problems intrinsic with anagenic hair, i.e., hair growing from the scalp of a person. Anagenic hair differs from hair tresses because of hair root, mid-length and tip color differences of anagenic hair, keratin structural differences among and between root, mid and end portions of a living hair and continuous sebum secretions extending from the root to the end portion of each strand of anagenic hair. Individual chemical compositions differences from person to person include sebum compositional components, anagenic hair strand dimension, individualized topographic character of strand surfaces and differences in the fatty acid or F layer. Person to person tertiary hair characteristics also differ including the variation of curl and color of differing regions of anagenic hair on the scalp and scalp skin issues. Additional differences of anagenic hair relative to hair tresses include but are not limited to lack of cleanliness of anagenic hair, presence of pre-existing hair treatments including but not limited to hair styling formulations, permanent oxidative dye applications, permanent wave and/or curl treatment, application of oils and smoothing compositions and conditioning treatments typically applied to anagenic hair. Still other difficulties involve regulatory requirements and the need to avoid tissue damaging and/or environmental attack by sunlight, UV, wind, rain and airborne chemicals, degrading chemicals in water and in hair care and hair dye compositions, as well as sweat and sebum.

Yet even with the recognition of the distinguishing properties of anagenic hair, long lasting remanence coupled with softness, low keratin damage and freedom from toxic and/or irritating chemicals has been an elusive goal. These color coatings continue to deliver stiff hair, hair strand clumping, undesirable lack of fluffiness and irregular coloration, not to mention toxicity, high skin irritation and regulatory prohibition of ingredients.

Achievement of a truly successful hair coating and coloring technology solving these problems focuses on development of long lasting hair strand color of anagenic hair that exhibits softness, fluffiness and the feel and appearance of untreated anagenic hair while at the same time avoiding hair strand damage typically associated with oxidative permanent dye techniques. While current hair strand coatings with pigments provide a starting point, connection of this technology to anagenic hair has yet to be achieved because of these intrinsic problems and issues. This technology has failed to demonstrate success in terms of remanence, natural color mimic and approximation of the anagenic hair qualities when transferred from tresses to anagenic hair. In particular, a signal goal of this technology has been and remains development of hair color like that achieved by permanent oxidative dye technology without the severe damage to anagenic hair.

### SUMMARY OF THE INVENTION

These and other difficulties are addressed by aspects of the present invention. The present invention meets these objectives through the design and application of methods to enable development of hair coating technology including hair restoration technology and hair styling technology and preferably the development of hair coloring technology directed toward surface coating and preferably coating coloration of keratinous fibers such as, but not limited to, hair tresses, hair tresses designed to mimic anagenic hair, anagenic hair, eyebrow hair and eyelash hair and more preferably anagenic hair on the scalp of a person. The design and application of the methods of the invention feature but are not limited to several embodiments including, initiation of activation processes to prepare keratin fiber surfaces for chemically and or physically interactive acceptance of coatings and preferably color coatings provided by a pretreatment composition and a film forming composition. Further embodiments are directed to adjustment of components of each of the methods and control of parameters, conditions and additives for the methods. These multiple design features enable ready methods for activation of keratin fibers and application of the compositions to be dressed onto activated, modified keratin fibers, preferably anagenic hair.

These multiple design features enable achievement of contiguous coating formation under processing parameters that enable unhurried dressing but rapid coating formation when desired. These multiple design features enable robust remanence, long wear-fastness, pleasing texture qualities, uniform color distribution and/or varied color distribution as well as establishing triggers for coating and color coating removal.

These multiple design features also enable solution of “downstream problems” that can otherwise be associated with anagenic hair. Such downstream problems include but are not limited to development of strong but flexible interconnection between and among coating, pigment and hair strand surfaces, sebum and F layer effects upon such coating interconnections, root idiosyncrasies affecting interaction between coating and preferably color coating and keratin fiber surfaces and the effect of incomplete or ineffective removal of grubbiness, dirtiness, foulness of anagenic hair. In addition, the multiple design features of color coatings benefit the color arrangement, distribution, and maintenance of the color of anagenic hair as it is assailed by environmental factors including but not limited to UV rays, shampoo, brushing, combing, rinsing, rain, wind, coverings by scarves and hats, rubbing and drying with towels and hair dryers, hair conditioners, styling hair sprays, hot iron curling and other environmental and hair care factors. At the same time, these multiple design features according to the invention provide tactile, visual and sound sensations at least approximately similar to untreated anagenic hair.

Aspects of the present invention include but are not limited to embodiments of methods for obtaining coating and preferably color coating of keratin fibers, preferably anagenic hair, as well as embodiments of the color composition and the components thereof. These aspects additionally include qualities of the coatings and preferably color coatings that deliver the above-described characteristics for hair coloration on keratin fibers.

The embodiments of these methods are directed to an activating step, a pretreatment step and a binder step. The activating step comprises contact of the keratin fibers with either or both of a Praeparatur procedure and a Fundamenta procedure to form modified keratin fibers. The Praeparatur procedure comprises at least a cleaning of the keratin fibers. The Fundamenta procedure comprises at least a chemical disruption of the keratin protein and or the bound lipids at the surfaces and possibly the subsurface of the keratin fibers. The pretreatment step is practiced simultaneous with or sequentially with the activating step and comprises application of a pretreatment composition to the keratin fibers. The pretreatment composition comprises at least a PTH alkoxysilane compound including embodiments such as a PTH organo-alkoxysilane and/or a PTH organo multidimethylsiloxanyl alkoxysilane and/or their disulfides and tetrasulfides wherein PTH is a symbol representing functional groups such as a thiol (—SH or mercaptan), a protected thiol, hydroxyl, and a complementary group that can react with a thiol. The binder step comprises application of a film forming composition to the modified keratin fibers precoated with pretreatment composition. The film forming composition comprises organic, silicone or organosilicone binders having binder functional groups. The binder may be a unitary organic, silicone or organosilicone polymer with a single binder functional group. Alternatively, the binder may be a dual polymer binder comprising first and second organic, silicone or organosilicone polymers which have different binder functional groups. When the binder is unitary, it will have a single binder functional group which may be self-reactive or may be interactive with a component of the pretreatment composition. When the binder is a dual polymer binder, it comprises first and second polymeric components which differ in structure and their different binder functional groups comprise at least complementary pairs including a) alkenoyloxy and amine, b) alkenoyloxy and thiol, and c) carboxyl and carbodiimide.

A first aspect of the invention directed to achievement of the coating, preferably the color coating on keratin fibers, especially on anagenic hair, concerns embodiments for activation of the surfaces and optional sub-surfaces of the keratin fibers. These embodiments are achieved through practice of Praeparatur procedures and Fundamenta procedures. The Praeparatur procedure deep cleans the keratin fiber surfaces and the Fundamenta procedure disrupts, alters and/or chemically changes the keratin protein and or bound lipids at the surface and possibly subsurface of the keratin fibers to produce modified keratin fibers including but not limited to chemically modified keratin fibers.

Embodiments of the Praeparatur technique include but are not limited to mild agitation with an aqueous surfactant composition to strong interaction with an aqueous or aqueous organic medium containing an anionic surfactant and/or organic solvent cleaning and/or optional rinsing with aqueous media optionally having pH adjustment. Additional procedures include optional mechanical agitation with such aqueous media and combing, brushing, vibrating, ultrasound and similar chafing and/or scrubbing of the surfaces of keratin fibers.

Embodiments of the Fundamenta method involve F layer removal, restructuring and disruption of the hair strand surfaces including but are not limited to one or more of a chemical restructuring with an acidic or basic oxidative agent such as persulfate, ozone or a peroxide such as benzoyl peroxide or hydrogen peroxide, a reductive chemical restructuring with a reducing agent, restructuring with a non-thermal equilibrium plasma treatment; or a chemical restructuring with a phase transfer tenside such as a fatty-alkyl trimethyl ammonium halide.

The activating step sets the stage for interaction of a pretreatment composition comprising at least a PTH alkoxysilane compound as described above with the keratin fiber surfaces and optional sub-surfaces. The activating techniques of Praeparatur and Fundamenta may be practiced prior to practice of the pretreatment step with the PTH alkoxysilane compound or may be combined with the application of the pretreatment composition.

A second aspect of the invention thus concerns the embodiments of the pretreatment step. The pretreatment step comprises addition of the pretreatment composition to the modified keratin fibers. Embodiments of the pretreatment composition comprise at least the PTH alkoxysilane compound which includes one or more of the PTH organo-alkoxysilane and/or the PTH organo multidimethylsiloxanyl alkoxysilane. Each of these two PTH alkoxysilane compounds has at least one PTH group, and at least one alkoxysilane group. The PTH group may be a thiol or protected thiol or a thiol complementarily reactive group. More specifically, the PTH group includes R³S— in which R³ comprises hydrogen or a sulfur protecting group. In addition, the PTH group can be a thiol reactive group such as OHC—, H₂C═CR¹⁰—CO₂— and HO—, wherein R¹⁰ may be hydrogen or a C1-C6, preferably C1 alkyl. The PTH alkoxysilane compound with PTH as SH may also be configured as multisulfide form of the thiol groups (e.g., disulfide and tetrasulfide). Preferably the pretreatment composition comprises at least the PTH organo-alkoxysilane with PTH as thiol and/or the PTH organo multidimethylsiloxanyl alkoxysilane with PTH as thiol, and more preferably the pretreatment composition comprises at least the thiolorgano-alkoxysilane.

Embodiments of the pretreatment composition may also comprise in addition an aminoorgano-alkoxysilane and/or an organo PTH compound having one or more PTH groups.

A third aspect of the invention is directed to embodiments of the binder step. The binder step comprises application of embodiments of the film forming composition. The film forming composition comprises any one of four embodiments of a binder polymer having binder functional groups. In two of these embodiments, the binder polymer may be a unitary organic, silicone or organosilicone component with a single binder functional group. In two other embodiments, the binder polymer may be a first organic, silicone or organosilicone component and a second organic, silicone or organosilicone component wherein the first and second components have complementary binder functional groups.

In a first embodiment of the film forming composition, the binder polymer is unitary and may comprise an in situ self-cross linkable organic polymer binder having two or more pendant and/or terminal alkoxysilane groups, preferably at least terminal alkoxysilane groups.

In a second embodiment of the film forming composition, the binder polymer is unitary and may comprise an organic polymer of one or more monomeric units selected from an olefinic carboxylate ester unit, an olefinic carboxamide unit, a carbon-hydrogen olefinic unit, an ester monomeric unit, an amide monomeric unit, a urethane monomeric unit, a urea monomeric unit and any combination thereof. In this second embodiment, the organic polymer further comprises at least one and preferably at least two pendant and/or terminal binder functional monogroups comprising a carboxylic acid group. Optionally, the organic polymer may also be substituted by pendant organoalkoxysilane groups. For this second embodiment, the pretreatment composition may also comprise the aminoorgano alkoxysilane in addition to the PTH alkoxysilane compound. The aminoorgano alkoxysilane delivers amino groups to the condensed pretreatment layer. These amino groups are believed to enable electrostatic interaction with the carboxyl groups of this film forming composition.

In a third embodiment of the film forming composition, binder polymer is a dual binder and comprises first and second polymer components which are different. The first component may comprise an organic, silicon or oganosilicon polymer having at least one pendant and/or terminal first binder functional group. The second component of this third embodiment may comprise a small molecule, a prepolymer or polymer having at least one pendant and/or terminal second binder functional group. The first and second binder functional groups of this third embodiment comprise a complementary pair respectively of an alkenoyloxy group and an amine and/or an alkenoyloxy group and a thiol, also known as Michael addition groups. Although it is not a limitation, the thiol and the hydroxyl groups of the PTH alkoxysilane compound and the optional amine groups of the aminoorganoalkoxysilane of the pretreatment composition are thought also to interact with the alkenoyloxy group of the first component of the film forming composition.

In a fourth embodiment of the film forming composition, the binder polymer is a dual binder and comprises first and second components which are different. The first component may comprise an organic, silicon or oganosilicone polymer having at least one pendant and/or terminal first binder functional group. The second component of this fourth embodiment may comprise a small molecule, a prepolymer or polymer having at least one pendant and/or terminal second binder functional group. The first and second binder functional groups of this fourth embodiment comprise a complementary pair respectively of a carboxylic acid group and a carbodiimide group. Although it is not a limitation, the thiol or hydroxyl groups of the PTH alkoxysilane compound and the optional amine groups of the aminoorganoalkoxysilane of the pretreatment composition are thought also to interact with the carbodiimide and/or intermediates formed from the carboxylic acid and carbodiimide.

Additionally, in preferred versions of the second, third and fourth embodiments of the film forming composition, as well as generally for the first embodiment, the binder polymer as a unitary polymer (second embodiment) or as first and second components (third and fourth embodiments) may also optionally and preferably comprise alkoxysilyl groups as well as the binder functional groups. The first embodiment already includes alkoxysilyl groups as the primary binder functional group. The alkoxysilyl groups of these embodiments of the film forming composition enable supplemental interconnection of the polymers of these film forming compositions with the alkoxysilyl groups of the pretreatment composition. The supplemental interconnection adds to the silicon-oxygen-silicon connection of the first embodiment, the carboxy-amine electrostatic interaction of the second embodiment, the Michael addition connection of the third embodiment and the carbodiimide—carboxyl connection of the fourth embodiment.

Accordingly, an additional aspect of the function of the PTH alkoxysilane compounds of the pretreatment composition is their interaction with the film forming composition. The film forming composition also preferably possess alkoxysilyl groups. Together, these alkoxysilyl groups hydrolyze and interact to form silicon-oxygen-silicone linkages. These linkages tie together the pretreatment and film forming compositions as a coating and preferably a color coating.

The action of the Praeparatur and/or Fundamenta procedures upon the keratin fibers to produce modified keratin fibers coupled with application of the pretreatment composition to the modified keratin fibers is believed to result in a chemical interaction of the PTH alkoxysilane compounds with the keratin protein at anagenic hair surfaces and optional subsurfaces. The Fundamenta procedure is thought to function through chemical interaction to produce modified protein moieties on the keratin fiber surfaces and optional subsurfaces such as but not limited to protein molecules having one or more of thiol/mercapto groups, oxidized sulfur groups, carboxyl groups and hydroxyl groups. From a chemical perspective, the PTH alkoxysilane compounds of the pretreatment composition are adapted to react with these modified keratin protein groups to form such adducts as disulfide groups, thioester groups, β-thio ethylcarboxyl adducts resulting from addition of thiol to an α, β unsaturated carboxyl group, as well as sulfonyl and sulfate ester groups resulting from hydroxyl addition to partially oxidized sulfur groups, and especially disulfide adducts. This interaction is thought to be a basis for anchoring the PTH alkoxysilane compound and the resulting self-condensed PTH alkoxysilane oligomers to the surfaces and possible sub-surfaces of the keratin fibers. In addition, the thiol and disulfide versions of the PTH alkoxysilane compounds themselves also can function as a reducing/recombination agent relative to the di-cysteine disulfide groups of keratin proteins to form disulfide linkages with the keratin surface and subsurface cysteine groups.

The results of the sequential or simultaneous practice of the Praeparatur/Fundamenta techniques with the pretreatment step form a pretreatment silicone polymer network intimately adhering to the deep cleaned and chemically modified topographic surfaces of keratin fibers. The final stage of this coating method and preferably this coloration method is set by introduction of the film forming composition practiced according to the binder step. Following formation of the pre-coating of the pretreatment composition on the modified keratin fibers, the film forming composition is applied to form a combination of the pretreatment composition and the film forming composition on the modified keratin fibers. This combination composition is an uncured combination of its components. The combination composition may be cured to provide the coating and preferably the color coating on the modified keratin fibers. The adherence and chemical interaction among and between the molecules of the pretreatment network and the network formed from film forming composition produce a highly remanent coating and preferably a highly remanent color coating on anagenic hair.

Embodiments of the film forming composition and pretreatment composition may be applied separately or together to the keratin fibers and cured (e.g., interbonded) according to methods of the invention to produce the coating and preferably the color coating of an interconnected, overlapping and/or intermixed composite film interconnected with the keratin fibers such as surfaces of anagenic hair strands. The coating and preferably the colored coating on the keratin fibers, preferably on anagenic hair, displays desirable characteristics including but not limited to remanence, wash-fastness, resistance to environmental attack. For keratin fibers, preferably anagenic hair, the coating and preferably the colored coating delivers elastomeric flexibility to enable free movement of the coated keratin fibers, pleasing texture characteristics similar to uncoated hair, tensile strength to resist flaking and breakage, and for the color coating, color mimicking of appropriate shades for roots, mid-length and tips of keratin fibers.

According to embodiments methods of the invention, embodiments of the pretreatment composition are applied to keratin fibers simultaneous with and/or in sequence following a Fundamenta procedure, especially an acidic oxidation process, a reduction process or a combination of a reduction process followed by an acidic oxidation process. In some instances, the pretreatment composition may be applied and processed at least partially to facilitate condensation-cure of some of the alkoxysilyl groups thereof before application of the film forming composition. In other instances, the pretreatment composition may be applied and film forming composition immediately applied thereafter followed by processing to cure the binder functional groups and optional alkoxyl silyl groups of the film forming composition together with the PTH groups and alkoxysilyl groups of the pretreatment composition. In still other instances, the pretreatment composition and film forming composition may be combined together and applied as a mixture to the keratin fibers. Use of a catalyst with the film forming composition may provide advantageous condensation rate of the pretreatment composition and the film forming composition irrespective of whether they are applied separately with intermediate curing, applied rapidly in sequence or pre-combined and applied as a mixture. Although it is not a limitation of the invention, it is thought that irrespective of the order of addition, the pretreatment composition distributes preferentially to the keratin fiber surfaces so as to enable its interaction with features of the keratin protein at the surfaces of the keratin fibers.

Aspects of the coating and preferably the color coating embodiments of the invention (cured coating) include at least in part a three-dimensional network of the coating and preferably the color coating formed through the mutual chemical interaction features of the composition and preferably the color composition components comprising the film forming composition and pretreatment composition and the modified keratin fibers. It is thought that the chemical interaction of the modified keratin fibers and the PTH alkoxysilane to form disulfide bonds and other entanglements enable intimate networking interaction with keratin fiber surfaces and enable networking interaction with the components of the film forming composition. The demonstrated result of this method is the experimental showing that initial residual sebum coating of anagenic hair and subsequent sebum secretion onto anagenic hair do not at least in part remove the coating and preferably the color coating from the anagenic hair surfaces. In other words, the remanence of the coating/color coating resulting from practice of the methods of the invention is longer lasting in its coverage of hair strands extending to the hair root sections than is the remanence of a coating/color coating not produced according to the steps of the method of the invention. These results demonstrate the binding and bonding interaction by and between the keratin fiber surfaces and the cured coating of pretreatment and film forming compositions.

The methods of the invention involving parameters, conditions and techniques for forming the coating and preferably the color coating on keratin fibers are directed to the combination of the activating and pretreatment steps along with the binder step to form the coating and preferably color coating. Aspects of these methods call for application of Praeparatur and/or Fundamenta procedures to keratin fibers, preferably anagenic hair simultaneous with or in sequence with application of the PTH alkoxysilane of the pretreatment composition. The methods of the invention further involve parameters, conditions and techniques for application of the pretreatment composition to modified keratin fibers, preferably modified anagenic hair before, or simultaneous with, or mixed with, or in combination with the application of film forming composition.

According to the invention, the desirable characteristics of the coating and preferably the color coatings on keratin fibers, preferably anagenic hair, may be demonstrated by tests of the coloration on hair tresses prepared from unbleached natural white human hair (hereinafter untreated hair tresses), bleached natural white human hair (hereinafter treated hair tresses) and untreated hair tresses specially prepared with synthetic sebum so as to mimic anagenic hair (hereinafter mimic hair tresses). Following formation of the coating and preferably the color coating on the mimic hair tresses, the coating mimic hair tresses are recoated with synthetic sebum and shampooed to represent closely the sebum secretion process on anagenic hair of a human scalp. The sebum recoating and shampooing are multiply repeated to assess remanence. Additionally, the damage conditions of the keratin fibers prepared according to the methods of the invention can be assessed. The results of these tests demonstrate significant remanence and lack of fading as well as minimal damage when embodiments of the invention are practiced.

For efficiency of experimental practice, the above described mimic hair tress has been developed to come as close as possible to the behavior of anagenic hair, especially the root segments of anagenic hair. Through use of the mimic hair tress, it has been surprisingly discovered that Praeparatur and Fundamenta procedures and the PTH alkoxysilane compound pretreatment network coupled with the film forming composition preserve remanence for coating and preferably the color coatings on mimic hair whereas those coatings and preferably those color coatings on mimic hair prepared without application of the Praeparatur and/or Fundamenta techniques and the PTH alkoxysilane pretreatment composition demonstrate significant to almost full fading during the multiple sebum—shampoo applications designed to examine remanence in real life conditions.

### BRIEF DESCRIPTION OF THE FIGURES

FIGS. 1A and 1B illustrate versions of the color coating prepared with three different experimental processes S4-S6 of Table 11. The salon model hair was washed 15 times over a period of many days after the color coating treatments.

FIG. 1A is a color photograph of the salon model's hair coloration with three different treatments S4, S5 and S6. The remanent differences among S4, S5 and S6 are shown by the extent of coloration up to the hair roots. S6 coloration extends closest to the roots while S5 and S4 colorations are further away from the roots. Color remanence at the hair roots is more difficult to achieve than at the tips because of anagenic hair and scalp bioactivity.

FIG. 1B is a grey scale (black and white) photograph of the color photograph of FIG. 1A. The black and white photograph does not illustrate the differences of S4, S5 and S6 treatments shown by the color photograph of FIG. 1A.

FIG. 2A illustrates a wavelength measurement (Wlm) photograph of the salon model hair of FIG. 1A. This measurement shows the degree of red color appearing on the hair. While the Wlm photograph is a grey scale photograph, it illustrates the differences of S4, S5 and S6 treatments shown by FIG. 1A.

FIG. 2B is the same Wlm photograph as FIG. 2A but is marked to show the extent of red coloration at the root region of the hair.

FIGS. 3A and 3B show the other side of the same salon model's hair after 15 wash cycles. This other hair side was prepared three different experimental processes S1-S3 of Table 11.

FIG. 3A is a color photograph showing the results of the three different processes. The differences in red color extending to the hair roots is clearly seen.

FIG. 3B shows a grey scale photograph of the colored hair of FIG. 3A. The color differences of FIG. 3A are not shown.

FIGS. 4A and 4B present Wlm photographs of the hair of FIG. 3A.

FIG. 4A shows the unmarked color extend differences.

FIG. 4B shows the marked limit of the coloration.

FIGS. 5A and 5B compare the Wlm photographs of FIGS. 2B and 4B

FIG. 5A reproduces FIG. 2B.

FIG. 5B reproduces FIG. 4B.

### DEFINITIONS

Unless defined otherwise, all technical and scientific terms used herein have the same meaning as commonly understood by a person of ordinary skill in the art.

As used in the specification and the appended statements, the singular forms “a,” “an” and “the” include plural referents unless the context clearly dictates otherwise

The term “may” in the context of this application means “is permitted to” or “is able to” and is a synonym for the term “can.” The term “may” as used herein does not mean possibility or chance.

The term and/or in the context of this application means one or the other or both. For example, an aqueous solution of A and/or B means an aqueous solution of A alone, an aqueous solution of B alone and an aqueous solution of a combination of A and B.

The molecular weight of a polymer or oligomer used according to the invention may be measured by a weight average molecular weight, and the distribution of molecules of different molecular weights of a polymer used according to the invention is determined by its polydispersity. Molecular weight is expressed as daltons (Da), kiloDaltons (KDa) and megaDaltons, which is million daltons or (MDa). The acronym Mw stands for weight average molecular weight, Mn is the number average molecular weight of a given polymer. Polydispersity is a unit-less number and indicates the breadth of the distribution of the polymer molecular weights and is defined as the Mw/Mn.

The term “about” is understood to mean±10 percent of the recited number, numbers or range of numbers.

The term “about 0 wt %” is understood to mean that no substance, compound or material to which zero (0) refers is present, up to a negligible but detectable amount is present, assuming that the detectability can be determined on a parts per million basis.

Where features or aspects of the invention are described in terms of Markush groups, those skilled in the art will recognize that the invention is also thereby described in terms of any individual member or subgroup of members of the Markush group. For example, if X is described as selected from the group consisting of methyl, ethyl or propyl, claims for X being methyl and claims for X being ethyl and X being propyl are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4. Similarly, values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range.

Keratin fibers means any natural material containing keratin protein including hair, eyebrows, and eyelashes. Natural keratin fibers include those from mammals and/or on mammals including human, primate, ruminant, camelid, equine, rodent and neovison including but not limited to cow, sheep, deer, goat, buffalo, lama, alpaca, camel, guanaco, vicuna, horse, antelope, moose, elk, rat, mouse, beaver, rabbit, mink, monkey, ape and similar species. Natural keratin material may include hair and fur. Keratin fibers include scalp hair, eyebrow hair and eyelash hair. Keratin fibers may be removed from their source such as hair cut from the scalp of a living person or may mimic anagenic hair when treated with sebum. As used herein keratin fibers includes cut hair and anagenic hair. For experimental purposes, keratin fibers are formed into tresses. A tress is a shock of keratin fibers e.g., hair, held in a clamp at one end and free at the other end. The hair on the head of the average person weighs about 100 g. A tress is formed with about 1 gram of hair or about 1/100 the weight of the hair on the head of a person. Typical commercial hair products for application to hair weigh about 100 to 120 g which translates into about 1 g of product per gram of a person's hair. This relationship establishes the amount of experimental product to be applied to a tress of hair, 1 gm of experimental product per tress weighing about 1 g.

As used herein “anagenic hair” means hair strands that are in direct connection with a hair follicle which is in either the anagen, catagen or telogen state. Anagenic hair is present in one of these states on a scalp of a person, a human. The follicle of anagenic hair produces long chain fatty acids, so-called F-layer, which form a water resistant coating on the cuticle of the hair shaft. Joining the hair follicle channel is a sebaceous gland that secretes sebum onto the hair shaft and onto the scalp. As a strand of hair grows from the follicle and extends from the scalp, sebum produced at the follicle spreads out from the follicle and continues to coat the strand. Sebum is removed at least in part from strand ends by shampooing but is replenished by this continued production. Hair cut from a living person is no longer anagenic hair.

As used herein, the terms “covalent, coordinate, electrostatic, ionic, dipolar and entanglement or entwining interactions” mean a chemical relationship between two atoms or two groups of atoms. The interaction includes a covalent bond between the atoms such as the covalent bond between the two carbons of ethane. The interaction includes a coordinate bond between two or more atoms such as the coordinate bond between oxygen and sulfur of the sulfate anion (SO₄⁻²) or a complex of zinc and EDTA. The interaction includes an electrostatic or ionic interaction between two charged atoms or particles such as the interaction between sodium and chloride of salt or between ammonium and acetate of ammonium acetate. Dipolar interaction includes hydrogen bonding such as the interaction between water and the hydroxyl of methyl alcohol. The interaction includes entanglement or entwining which is lipophilic interaction or mechanical/physical twisting together such as is present in the molecules of polyethylene.

Adherence as used herein generally refers to an arrangement in which a substance formed of a polymer, oligomer or small molecule exhibits a connective aspect with another material such as another polymer, oligomer, small molecule, keratin protein, through such forces as covalent bonding, hydrogen bonding, coordinate interaction, electrostatic interaction, dipolar interaction, small force interaction, dispersion force at least as a result of entropy, molecular entanglement, mechanical interaction as may be exhibited on a molecular level by a molecular chain wrapping around irregular terrain features of a surface. Adherence in this context may be, but not necessarily, shown by the inability of the adhered material to be removed from the substance without exertion of any force.

Entanglement as used herein generally refers to an arrangement in which a chain crosses an arbitrary plane 3 times. The chain is then entangled. If the chain is shorter and crossed only two times, it can be pulled in the middle and both ends will release without being bound. With three crossings, if the chain is pulled at one point, it will trap another polymer chain at a different place.

As used herein, the term “transfer resistance” or rub off resistance generally refers to the quality exhibited by colored coatings that are not readily removed by contact with another material, such as, for example, an item of clothing or the skin. Transfer resistance can be evaluated by any method known in the art for evaluating such transfer. For example, transfer resistance of a colored coating can be evaluated by the amount transferred from a wearer to any other substrate after the expiration of a certain amount of time following application of the colored coating to the hair. The amount of colored coating transferred to the substrate can then be evaluated and compared. For example, a colored coating can be transfer resistant if a majority is left on the wearer's hair. Preferably little or no colored coating is transferred to the substrate from the hair.

As used herein, the term “modified keratin fibers, upon application” generally means that the keratin protein at least at the surfaces of the fibers has an altered state. An altered state includes one or more of cysteine disulfide cleavage, amide group cleavage, ester group cleavage, sulfone production, ester formation, thioester formation, and similar chemical changes to keratin protein. The state of the modified keratin fibers can be assessed for example using ATR FT-TR for oxidative damage as described later or through tensile testing methods known to those skilled in the art for assessing fiber strength for example using equipment such as those designed and sold by Dia-Stron™.

As used herein, the term “converting” means causing covalently co-reactive pairs of components of a composition such as but not limited to the binder and linker of the film forming composition to react together chemically to produce the reacted form such as, for example a chain-extended and/or cross linked polymer functioning as coating or film. Converting is accomplished by the application of an activity designed to cause the covalent bonding of the reactive groups or pairs of the co-reactive components. Activities enabling conversion include but are not limited to drying, heating, curing as in causing the curing/reacting together the co-reactive components, allowing the co-reactive components to combine or mix at standard conditions without further intervention, addition of a catalyst, changing pH of the composition and any other activity that is capable of influencing the reactivity and/or rate of the reaction of the co-reactive components.

The term “remanence” means preservation of an original property of a substance, such as color, attached or connected to or upon a substrate when the substance is subjected to a process that could but not necessarily will remove it from the substrate. An example of remanence is the ability of a color coating on a substrate, such as paint on wood, to withstand environmental factors and washing factors that could remove the color coating such as paint from the substrate such as wood. A cosmetic example of remanence is the ability of a coating such as a hair styling composition to resist removal by water such as rain, or by rinsing. The extent of remanence may be measured by the ability of the substance to maintain its original size, color, intensity, hue and any other original characterization upon being subjected to multiple times of a procedure that could remove the substance. An example of the extent of remanence for a cosmetic coating is shown by the ability of a color coating on anagenic hair to maintain its original color intensity hue and shade and to avoid fading while being treated with a commercial shampoo preparation. The number of shampoos needed to begin fading and/or loss of original properties measures the extent of remanence for this example. A test for remanence is described in the Examples section under the title “full root simulation color remanence test.”

“Aliphatic substituent, group or component” refers to any organic group that is non-aromatic. Included are acyclic and cyclic organic compounds composed of carbon, hydrogen and optionally of oxygen, nitrogen, sulfur and other heteroatoms. This term encompasses all of the following organic groups except the following defined aromatic and heteroaromatic groups. Examples of such groups include but are not limited to alkyl, alkenyl, alkynyl, corresponding groups with heteroatoms, cyclic analogs, heterocyclic analogs, branched, dendritic, star or fullerene-like and linear versions and such groups optionally substituted with functional groups, as these groups and others meeting this definition of “aliphatic” are defined below.

“Aromatic substituent, group or component” refers to any and all aromatic groups including but not limited to aryl, aralkyl, heteroalkylaryl, heteroalkylheteroaryl and heteroaryl groups. The term “aromatic” is general in that it encompasses all compounds containing aryl groups optionally substituted with functional groups (all carbon aromatic groups) and all compounds containing heteroaryl groups optionally substituted with functional groups (carbon-heteroatom aromatic groups), as these groups and others meeting this definition of “aromatic” are defined below.

As used herein, the term “optionally” means that the corresponding substituent or thing may or may not be present. It includes both possibilities.

“Alkyl” refers to a straight or branched, dendritic, star or fullerene-like or cyclic hydrocarbon chain group consisting solely of carbon and hydrogen atoms, unless otherwise specifically described as having additional heteroatoms or heterogroups. The alkyl group contains no unsaturation, having from one to twenty four carbon atoms (e.g., C1-C24 alkyl). Whenever it appears herein, a numerical range such as for example but not limited to “1 to 24” refers to each integer in the given range; e.g., “1 to 24 carbon atoms” means that the alkyl group may consist of 1 carbon atom, 2 carbon atoms, 3 carbon atoms, etc., up to and including 24 carbon atoms, although the present definition also covers the occurrence of the term “alkyl” where no numerical range is designated. In some embodiments, it is a C₁-C₄ alkyl group. In other instances, it is a C₁-C₆ alkyl group and in still other instances it is a C1-C24 alkyl group. Typical alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl, decyl, and the like. The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), 3-methylhexyl, 2-methylhexyl, and the like.

Alkylenyl” refers to a straight or branched, dendritic or star divalent hydrocarbon chain consisting solely of carbon and hydrogen atoms, unless otherwise specifically described as having additional heteroatoms or heterogroups. The alkylenyl group contains no unsaturation and has a dangling valence bond at either end of the chain for bonding to two other moieties. The alkylenyl group may have a carbon number range of 1 to 24 carbon atoms unless otherwise specified. In all cases the general and specific numerical range of carbon atoms includes each integer in the range. An example of a divalent C4 hydrocarbon chain designated as an alkylenyl group is as follows: —CH₂—CH₂—CH₂—CH₂—; the dashes (-) indicate valence bonds to other atoms or moieties not shown. This example of an alkylenyl group is butylenyl.

“Cycloalkyl” is a subcategory of “alkyl” and refers to a monocyclic or polycyclic group that contains only carbon and hydrogen, and may be saturated, or partially unsaturated. Cycloalkyl includes one or more rings, such as two or three or four rings either linked in tandem or through alkyl group or fused. Cycloalkyl groups include groups having from 3 to 24 ring atoms (i.e., C₃-C₂₄ cycloalkyl). Whenever it appears herein, a numerical range such as but not limited to “3 to 24” refers to each integer in the given range; e.g., “3 to 24 carbon atoms” means that the cycloalkyl group may consist of 3 carbon atoms, etc., up to and including 24 carbon atoms. In some embodiments, it is a C₃-C₈ cycloalkyl group. In some embodiments, it is a C₃-C₅cycloalkyl group. Pursuant to the definition of alkylenyl, a cycloalkyenyl group is a monocyclic or polycyclic group with two dangling valences for bonding to two other moieties. Illustrative examples of cycloalkyl groups include but are not limited to the following moieties: cyclopropyl, cyclobutyl, cyclopentyl, cyclopentenyl, cyclohexyl, cyclohexenyl, cycloseptyl, cyclooctyl, cyclononyl, cyclodecyl, norbornyl, and the like.

“Alkoxy” refers to the group —O-alkyl, including from 1 to 24 carbon atoms of a straight, branched, dendritic, star or cyclic configuration and combinations thereof attached to the parent structure through an oxygen. Examples include methoxy, ethoxy, propoxy, isopropoxy, cyclopropyloxy, cyclohexyloxy and the like. “Lower alkoxy” refers to alkoxy groups containing one to six carbons. In some embodiments, alkyl is an alkyl group which encompasses both linear, branched, dendritic, star or fullerene-like chain alkyls of multiple carbon atoms. Without further definition of the number or carbon atoms present, as used herein, the term “alkoxy” such as an alkoxysilyl group means a C₁-C₆, preferably C₁-C₄, more preferably C₁-C₂ alkoxy such as methoxy and ethoxy.

The terms alkoxysilane and alkoxysilyl are synonymous terms and mean a group of the formula

—Si(R′)₃₋ₜ(OR)ₜ

wherein R′ is a C1-C3 alkyl group, preferably methyl or ethyl, R is an alkyl group of 1 to 6, preferably 1 or 3, more preferably 1 or 2 carbons, e.g., methyl or ethyl, t is an integer of 1, 2 or 3. Preferably the alkoxysilane has three OR groups. Furthermore, depending upon the construction of the compound substituted with the alkoxysilane group, the alkoxysilane group may also have one of the OR groups as OH. A compound with this arrangement can be the result of hydrolysis of an Si-OR bond in which R is alkyl. Thus, the term alkoxysilane means that the silicon atom is bound to one, two or three alkoxy groups and in some instances of compounds with alkoxysilane groups, an alkoxy group may be incidentally be a hydroxy group. The dangling valence of the silicon atom of the alkoxysilane is bound either to an organic group or to a dialkylsiloxanyl group such that the silicon atom joins either a carbon or an oxygen depending upon the identity of the moiety to which the alkoxysilyl group is bound, such as but not limited to an organic compound, a siloxane compound, an organosiloxane compound, an organic polymer backbone, a silicone polymer backbone or an organosilicone backbone. Also, because the hydrolysis intermediate of each alkoxy of the alkoxysilyl group is hydroxy group as in hydroxysilyl, the hydroxysilyl/hydroxysilane group is included in this definition as discussed above. Furthermore, one of the alkoxys of the alkoxysilyl group may hydrolyze and the resulting hydroxysilyl may condense with another hydroxysilyl group derived from another alkoxysilyl group to form an Si—O—Si bond. Because there are three alkoxy groups on this moiety, the formation of a silicon-oxygen-silicon bond may occur as many as three times for a single alkoxysilyl (trialkoxysilyl) group. Irrespective of whether the alkoxysilyl group is pendant or terminal on a molecule such as a small molecule, oligomer or polymer, this multiple Si—O—Si bonding arrangement for a single alkoxysilyl group means that the molecule with the single alkoxysilyl group may undergo multiple condensations. The molecule with an alkoxysilyl may be chain extended with another molecule with an alkoxysilyl to produce a linear chain extended molecule. This linear chain extended molecule contains additional Si-OR functions at this Si—O-Si chain extension. These additional Si—OR functions can again condense with a corresponding Si—OR function of another linear chain extended molecule. The result is a cross-link at the intermediate section of these molecules bearing the Si—O-Si link. These additional Si—OR's of separate chain extended molecules can therefor condense to cross link the separate chain extended molecules.

“Amino” or “amine” refers to an —N(R^{a})₂ group, where each R^{a} is independently hydrogen or an alkyl group of 1 to 3 carbons, eg, methyl, ethyl or propyl.

“Aryl” is a subcategory of aromatic and refers to a conjugated pi ring or multiple rings with six to twenty two ring atoms. The aryl group has at least one ring having a conjugated pi electron system which is carbocyclic (e.g., phenyl, fluorenyl, naphthyl and anthracenyl). Included are partially saturated aryl rings such as tetrahydro naphthyl.

“Heteroalkyl” “heteroalkenyl” and “heteroalkynyl” include optionally substituted alkyl, alkenyl and alkynyl groups and which have one or more skeletal chain atoms selected from an atom other than carbon, e.g., oxygen, nitrogen, sulfur, phosphorus or combinations thereof. A numerical range may be given, e.g. C₁-C₂₄ heteroalkyl which refers to the chain length in total, which in this example may be as long as 24 atoms long. For example, a —CH₂OCH₂CH₃ group is referred to as a “C₄” heteroalkyl, which includes the heteroatom center in the atom chain length description. Connection to the rest of the molecule may be through either a heteroatom or a carbon in the heteroalkyl chain.

“Heteroaryl” or heteroaromatic refers to a 5, 6 or 10-membered aromatic group (e.g., C₅-C₁₃ heteroaryl) that includes one or more ring heteroatoms selected from nitrogen, oxygen and sulfur, and which may be a monocyclic, bicyclic, tricyclic or tetracyclic ring system or a conjugated ring system such as cyclopentadienyl optionally with a bridging atom providing conjugation such as pyrrole or thiophene. Whenever it appears herein, a numerical range refers to each integer in the given range. An N-containing “heteroaromatic” or “heteroaryl” moiety refers to an aromatic group in which at least one of the skeletal atoms of the ring is a nitrogen atom. The polycyclic heteroaryl group may be monocyclic or non-monocyclic. The heteroatom(s) in the heteroaryl group is optionally oxidized. One or more nitrogen atoms, if present, are optionally quaternized. The heteroaryl is attached to the rest of the molecule through any atom of the ring(s). Examples of heteroaryls include, but are not limited to pyrrolyl, furanyl, thiophenyl, imidazolyl, pyranyl, pyridinyl, pyrimidinyl, benzimidazole, benzothiophenyl, quinolinyl, quinazolinyl, and similar heteroaryl compounds of 6 to 12 carbons and 1, 2 or 3 heteroatoms including any combination of nitrogen, oxygen and sulfur.

“Heterocyclic” refers to any monocyclic or polycyclic moiety comprising at least one heteroatom selected from nitrogen, oxygen and sulfur. As used herein, heterocyclyl moieties can be a partially saturated aromatic ring or a saturated monocyclic or polycyclic ring wherein the ring may be formed of 3 to 8 atoms.

The term “polymer” or “Poly” means any or more of an organic, silicone or organosilicone compound formed from multiple monomeric units such as two or more. The units may be identical or may be a combination of units of differing identities. The number of units present may range from at least 2 to compounds having very large number of units. Typical weight average molecular weights of a polymer may range from less than one hundred Da to a million or more Da.

Compounds and groups including a polymer, an alkyl, an alkylenyl, a carbon or silicone chain, a carbon or silicon backbone, an aliphatic group of multiple carbons, hetero forms of any of the foregoing compounds and groups, as well as groups including aromatic, heteroaromatic cycloalkyl heterocycloalkyl or hetero forms thereof bearing any of these foregoing compounds and groups may have a structural configuration of linear, branched, star or dendritic which is a sub-category of branched. A preferred configuration is branched or linear and a more preferred configuration is linear. Use of any of these terms without indicating a particular configuration incorporates all of these configurations and means that linear and/or branched is preferred and linear is most preferred.

The terms “In situ linking” and “in situ linkable” and “cross linkable” mean the potential at a future time to form covalent bonds to provide interactions and/or connections between molecules. The terms “in situ linked” and “cross linked” mean that in the present state, covalent bonds have already occurred.

“in situ” is a Latin phase meaning in its original place. In the context of this invention, it means an activity such a cross linking that takes place on the hair.

The average reactive functional group equivalent weight as used herein means for a reactive functional group of a complementary pair, the ratio of the weight average molecular weight of the polymer, oligomer or small molecule containing the reactive functional group to the average number of occurrences of that reactive functional group in the polymer, oligomer or small molecule. If the Mw of a polymer is 1KDa and the average number of occurrences of the reactive functional group in the polymer is 2, the Mw for the reactive functional group equivalent weight is (1 KDa)/2 or 500 Da.

Coefficient of thermal expansion refers to the fractional increase in length of a species per Celsius increase in temperature at a constant pressure with a starting temperature of 25° C.

Zeta potential relating to pigment microparticles means the electrokinetic potential of extremely small particles suspended in colloidal dispersions. It is caused by the net electrical charge at the particle interface with the suspending fluid. It is an indicator of the stability of a colloidal dispersion. The magnitude indicates the degree of electrostatic repulsion between adjacent similar charged particles in a dispersion. At zero or minimal + or − potential, rapid coagulation can occur. At a + or − zeta potential above about 40 mV, good colloidal stability is maintained. Zeta potential can be measured using approaches known to those skilled in the art. For example a Zetasizer Nano Z from Malvern Panalytical Ltd, Malvern U.K. may be used to assess the zeta potential of the components.

Microfibril length as used herein general refers to a distribution of lengths for any given microfibril, and the fiber length refers to the average fiber length, assessed over a minimum of 10 fibers chosen randomly from a sample of the microfibrils. The length refers to the end to end distance along the major axis of the material and is not a measure of the cross sectional width.

Hansen Solubility Parameters constitute a technique for characterizing solubility, dispersion, diffusion, chromatography and related topics for a particular material. The material such as a solvent or solute can be characterized by three parameters δD for Dispersion (van der Waals), δP for Polarity (related to dipole moment) and δH for hydrogen bonding. See “Hansen Solubility Parameters—A User's Handbook”, CRC Press, Boca Raton, 2007, ISBN-10: 0849372488.

Hydrogen bonding refers to a weak bond between two molecules resulting from an electrostatic attraction between a proton in one molecule and an electronegative atom in the other. Ionic bonding refers to a type of chemical bonding that involves the electrostatic attraction between oppositely charged ions.

Young's modulus, or the Young modulus, is a mechanical property that measures the stiffness (e.g., stretchiness) of a solid material. It defines the relationship between stress (force per unit area) and strain (proportional deformation) in a material in the linear Hookean elastic regime of a uniaxial deformation. In other words, the ability of a material to withstand changes in length when under lengthwise tension or compression.

The term Tg or glass transition temperature refers to the temperature range through which a material, such as but not limited to a polymer, transitions from amorphous solid-like or glass-like properties at a lower temperature to viscous or rubber-like properties at a higher temperature. The transition is not a phase transition such as solid to liquid. Embodiments of the color coatings on mimic hair, treated hair and untreated hair typically will exhibit a Tg range well below ambient temperature so that the films produced will exhibit flexible, elastomeric, smooth physical properties.

The term ultimate compression refers to the amount of compression a given material can experience under a specific test method before failure occurs and the material breaks.

A nanoemulsion is a liquid/liquid, liquid/solid, liquid/gas or gas/gas composition in which there is at least one discontinuous phase dispersed in a continuous phase and in which the average particle or micellar diameter of the discontinuous phase is in the range 10 nm-300 nm.

The term “sebum” is an oily, waxy substance produced by the sebaceous glands of the human body. It coats, moisturizes, and protects skin and hair. Sebum is primarily composed of triglycerides (≈41%), wax esters (≈26%), squalene (≈12%), and free fatty acids (≈16%). The sebum used to form mimic hair is Hautfett nach BEY, sold by Wfk-Testgewebe GmbH which comprises 18.0% free fatty acids, 32.8% beef tallow, 3.6% triglycerides, 18.3% wool fat, 3.7% cholesterol, 12.0% hydrocarbons, 11.6% cutina.

The term “surface energy” quantifies the disruption of intermolecular bonds that occurs when a surface is created. The surface energy may be defined as the excess energy at the surface of a material compared to the bulk, or it is the work required to build an area of a particular surface. Perhaps the most widely used definition of surface energy, historically, is that of Zisman (“Relation of Equilibrium Contact Angle to Liquid and Solid Constitution”, W. A. Zisman, ACS Advances in Chemistry Series #43, 1961, pp. 1-51.). Zisman defines the surface energy of a solid to be equal to the surface tension of the highest surface tension liquid (real or imaginary) that will completely wet the solid, with a contact angle of 0°. This comes from the widely observed tendency of contact angle to decrease as liquid surface tension decreases on the same solid sample. A Zisman plot is created for a test surface using a series of different probe liquids with known surface tensions, with the known surface tensions plotted on the x axis, and the cosine of the resulting contact angle with the test surface plotted on the y axis. The highest surface tension when the cosine of the contact angle reaches 1 is determined to be the surface energy of the test substrate. The Owens/Wendt theory (Owens, D. K.; Wendt, R. C.; Jour. of Applied Polymer Science, 13, 1741, (1969)) is a further development to measure the surface energy of a test substrate. It considers the surface energy being comprised of two components—a dispersive component and a polar component. The dispersive component accounts for van der Waals and other non-site specific interactions that a surface is capable of having with applied liquids. The polar component theoretically accounts for dipole-dipole, dipole-induced dipole, hydrogen bonding, and other interactions which a surface is capable of having with applied liquids. Owens and Wendt developed a two parameter model for describing surface interactions, as opposed to the one parameter model of Zisman. The units of surface energy are mN m¹.

The terms “priming”, “deep cleaning” and “chemical modification” refer to the substantial to essentially complete removal of sebum and F-layer substances from the surfaces of anagenic hair, removal of synthetic sebum and F layer substances on mimic hair tresses and chemical disruption/breakage of chemical bonds of keratin protein at the surfaces and sub surfaces of keratin fibers. The chemical bonds include at least cysteine-cysteine disulfide bonds, protein chain and side chain amide bonds and side chain ester bonds. Also included are oxidation and reduction of groups such as thiol, amine, hydroxyl and similar amino acid functional groups. The Praeparatur and Fundamenta techniques accomplish the priming, deep cleaning and chemical modification of keratin fiber surfaces. Practice of these priming, deep cleaning and chemical modification techniques may accomplish adjustment of the keratin fiber surfaces so as to expose variable surface topography and gain an intimate interaction with chemically active agents such as but not limited to thiols.

### DETAILED DESCRIPTION

The present invention is directed to methods and compositions for development of coatings and preferably color coatings on keratin fibers, particularly anagenic hair and especially anagenic hair on the scalp of a human. These methods and compositions may also be applied to keratin fibers relating to all sources such as hair tresses, animal hair and similar keratin fibers. The qualities and properties of the aspects of the activating, pretreatment and binder steps of the method contribute to, enhance and promote qualities of the resulting coating and preferably color coating so that the coated keratin fibers and especially coated mimic tresses and anagenic hair demonstrate significant remanence while also demonstrating a performance similar to that of young, uncoated, vibrant, attractive hair of the scalp.

Embodiments of the methods are directed to the three steps of activation, pretreatment, and binding. Embodiments of the activation step are directed to either or both of the Praeparatur and Fundamenta procedures. Embodiments of the Praeparatur procedure are directed to cleaning the keratin fibers with a cleaning composition comprising a surfactant and other optional compounds which are capable of solubilizing, dispersing and/or lifting dirt, grime, grease and other untoward contaminants. Embodiments of the Fundamenta procedure are directed to one or more processes including an acidic oxidation, a basic oxidation, a plasma treatment, a PETT treatment, a reduction or any combination thereof. Practice of the activating step is believed to remove sebum from keratin fibers, especially anagenic hair and to disrupt chemically the surfaces and sub-surfaces of the keratin fibers to form modified keratin fibers. It is believed that the disruption breaks chemical bonds of the keratin protein at the fiber surfaces and sub-surfaces. The disruption is believed to produce thiol groups, sulfone groups and other bondable groups at the fibers surfaces and sub-surfaces. The order of practice of these two procedures of the activating step at least in part affects the properties of the coating and preferably color coating on the keratin fibers.

A first order of practice of the Praeparatur and Fundamenta procedures combines a Praeparatur procedure with each of the processes of the Fundamenta procedure and with several combinations of the processes of the Fundamenta procedure. A second order of practice constitutes each of the processes of the Fundamenta procedure alone and combinations of some of the processes of the Fundamenta procedure without the Praeparatur procedure. The first order of practice combines a Praeparatur procedure which preferably involves treatment with a mild or moderate or strong surfactant composition with each of the acidic oxidation, basic oxidation, plasma treatment, PETT treatment and reduction. Additionally, the first order of practice may combine a Praeparatur procedure with a reduction followed by acidic oxidation. In this first order of practice, the Praeparatur procedure is practiced first followed by optional rinsing. Each of the processes of the Fundamenta procedure may sequentially follow this Praeparatur procedure or may be initiated during an intermediate stage of the practice of the Praeparatur procedure or the Praeparatur procedure and a process of the Fundamenta procedure may be practiced simultaneously.

The second order of practice involves the Fundamenta procedure alone. Each of the processes of the Fundamenta procedure may be practiced alone, i.e., without the Praeparatur procedure. These include acidic oxidation, basic oxidation, plasma treatment, PETT treatment and reduction. The first and last procedures may be combined as reduction followed by acidic oxidation.

Embodiments of the pretreatment step are directed to application of a pretreatment composition to the modified keratin fibers. Embodiments of the pretreatment composition are directed at least to include a PTH alkoxysilane compound including a PTH organo-alkoxysilane and/or a PTH organo multidimethylsiloxanyl alkoxysilane, (together PTH alkoxysilane compound or compounds, and PTH is defined in the Summary as well as herein below) as well as the protected thiol derivatives of the PTH alkoxysilane compounds with PTH as thiol and the disulfide dimers and the tetrasulfide dimers of the PTH alkoxysilane compounds with PTH as thiol. Additional embodiments of the pretreatment composition add an aminorgano-alkoxysilane and/or a PTH organic compound to the composition containing the PTH alkoxysilane compounds. Preferably, the pretreatment composition comprises at least the PTH organo-alkoxysilane with PTH as thiol. The pretreatment step is practiced immediately following the activation step which may or may not include a rinse step prior to initiation of the pretreatment step. Alternatively, the pretreatment step may be practiced simultaneously or in an overlapping manner with at least some of the embodiments of the activating step.

Embodiments of the binder step are directed to application of the film forming composition. The film forming composition may comprise a binder polymer composition comprising a unitary organic, silicone or organosilicone polymer with a binder functional group or dual polymers comprising a first organic, silicone or organosilicone component with a first binder functional group and a second organic, silicone or organosilicone component with a second binder functional group. The first and second components differ at least because of the identities of first and second binder functional groups. When the binder polymer comprises a single organic, silicone or organosilicone polymer binder the binder functional group may be either a) alkoxysilane, or b) carboxylic acid. When the binder polymer comprises dual polymers, the first and second functional binder groups are different and the first and second components are different. In this dual binder polymer situation, the first and second binder functional groups form complementary pairs including: a) alkenoyloxy and amine, b) alkenoyloxy and mercapto, c) carboxylic acid and carbodiimide.

To accomplish the above described qualities of the coating and preferably the color coating on keratin fibers, an aspect of the method at least in part involves the dual activity of the activating step and application of the PTH alkoxysilane compound to the surfaces of keratin fibers. Although it is not a limitation of the invention, the activating step likely disrupts the keratin protein and keratin-lipid conjugates such as 18-methyleicosanoic acid thioester at the surfaces and sub-surfaces of keratin fibers to provide thiol/mercapto groups, sulfide groups, sulfoxyl groups and intermediates of these groups. The PTH alkoxysilane compounds with PTH as thiol or disulfide or tetrasulfide dimers may also contribute to the disruption. The disruption is believed to include at least scission and/or breakage and/or rearrangement of keratin protein bonds such as but not limited to amide bonds, ester bonds thioester bonds and disulfide bonds present in keratin protein. In combination with the PTH alkoxysilane compounds, as well as the corresponding protected versions, disulfides and tetrasulfides of the pretreatment composition, the reformation, rearrangement and recoupling of the disrupted keratin protein bonds with the PTH alkoxysilane compound by coupling the PTH groups of the PTH alkoxysilane compounds and the thiols and other groups of the disrupted keratin protein molecules. It is believed that the PTH group including the —SH group and/or the aldehyde group and/or the α,β unsaturated carboxyl group and/or the hydroxyl group and/or the protected sulfur group of the PTH alkoxysilane compound interacts with these keratin protein groups to form chemical linkages. The chemical linkage of the PTH alkoxysilane compound and the keratin protein at the surfaces and sub-surfaces of the keratin fibers likely results at least in formation of disulfide, thioester, ester, Michael adduct and sulfone ester bonds. This linkage is believed to enable development, at least in part, of the strong remanence and resistance to sebum displacement of the coating and preferably color coating. The rearrangement and recoupling enables a chemically interconnected pre-coating of the pretreatment composition to the keratin fibers. The alkoxysilane group of the pretreatment composition also hydrolyzes and condenses to form silicon-oxygen-silicon bonds resulting in siloxane polymer formation. Addition of the film forming composition of the binder step, completes the three steps of the method and forms keratin fibers interconnected to the composite film of pretreatment composition and film forming composition. The first or second binder groups from the complementary pairs are chosen not only to react together as a reactive complementary pair but also so that they are capable of interacting with the functional groups of the PTH alkoxysilane including: a) alkenoyloxy and amine, b) alkenoyloxy and mercapto, c) carboxylic acid and carbodiimide, d) mercapto and carbodiimide, e) mercapto and aldehyde, f) double bond and mercapto in the optional presence of a radical initiator. The combination of the linked PTH alkoxysilane with the film forming composition provides chemical linkages from the keratin fiber surfaces through the pretreatment composition to the film forming composition. Because of the chemical linkages, displacement of the coating and preferably color coating by means such as physical, surfactants, sebum or other non-specific activities is minimized.

Damage of anagenic hair by oxidative dye processes is an oxidative issue sought to be avoided by pigment coloration of keratin fiber surfaces. Over at least the past fifteen years patents and scientific publications directed to surface coloration of keratin fibers state that avoidance of this damage is an important aspect of surface coloration. Oxidative damage is most severe when basic, strong oxidation is practiced. The base renders the cuticle porous to oxidants and small molecules which penetrate to the cortex where they do their work. While not as powerful, techniques such as weak base oxidation, plasma treatment, PETT treatment, reduction and acidic oxidation also damage the keratin fibers. FTIR and tactile examination of keratin fibers enable assessment of this damage by processes of the Fundamenta procedure and enable development of a hierarchy of activating/pretreatment (PTH alkoxysilane compounds) steps ranging from significant damage to minimal damage. Coordination of this hierarchy with remanence delivers a system for the combination of the activating and pretreatment steps that ranks the combinations from best to adequate.

Tables I and II coordinate the various activation processes, the PTH alkoxysilane compound pretreatment step and the binder step with a resultant combination for hair damage and remanence. The performance expectations of combinations of steps are ranked as green, yellow and red meaning superior, good and adequate respectively for the resulting combination. For these Tables, the activation processes include Praeparatur procedure (surfactant shampoo), and Fundamenta procedure including acid oxidation, base oxidation, plasma treatment, PETT treatment and reduction as well as a combination of reduction followed by acidic oxidation. The Praeparatur procedure may be conducted simultaneous with a particular Fundamenta procedure (indicated by no space between these two activities) or may be conducted sequentially with a particular Fundamenta procedure (indicated by a space between these two activities). The pretreatment includes application at least of the PTH alkoxysilane compound with PTH as thiol (OSSI for organosulfur silane). The Binder is the film forming composition and includes a polyolefin with pendant/terminal carboxylic acid groups (EAA), a single organic polymer with pendant/terminal alkoxysilane groups (Winnie), two silicone polymers with the complementary functional binder pair alkenoyloxy and amine (Michael), and an organic polymer and oligomer with the complementary functional binder pair carboxylic acid and carbodiimide (CDI). Table II provides the results for the Binder EAA because the pretreatment composition provides an aminoorgano-alkoxysilane included with the PTH alkoxysilane compound. The aminoorgano-alkoxysilane provides the amine group for electrostatic interaction with the carboxylic acid of EAA.

The experimental results of these combinations as provided by Tables I and II show that a Praeparatur procedure combined with acidic oxidation provides the best combination of results (all green) while the Praeparatur procedure combined with basic oxidation provides the lowest rated combination. The Fundamenta procedures of PETT, plasma and reduction all provide adequate results but show significant hair damage by the tactile test. The combination of reduction and acidic oxidation also provides adequate results but shows some hair damage by the FTIR test.

TABLE I

| | | | | | Performance Expectations | | | |
|---|---|---|---|---|---|---|---|---|
| | | | | | | Hair | Hair | Back to |
| Initiating Step | | | Pretreatment | | Sebum | Damage - | Damage - | original |
| Praeparatur | Fundementa | | Step | Binder Step | Test | FT-IR | Feel | color |
| Shampoo | acidic oxidation | | OSSI | Binder | Green | Green | Green | Green |
| Shampoo | acidic oxidation | | OSSI with Binder | | Green | Green | Green | Green |
| Shampoo with acidic oxidation | | | OSSI | Binder | Green | Green | Green | Green |
| Shampoo with acidic oxidation | | | OSSI with Binder | | Green | Green | Green | Green |
| Shampoo | basic oxidation | | OSSI | Binder | Green | Red | Yellow | Red |
| Shampoo | basic oxidation | | OSSI with Binder | | | Red | Yellow | Red |
| Shampoo with basic oxidation | | | OSSI | Binder | Green | Red | Yellow | Red |
| Shampoo with basic oxidation | | | OSSI with Binder | | | Red | Yellow | Red |
| Shampoo | CTAB | | OSSI | Binder | Green | Green | Yellow | Green |
| Shampoo | CTAB | | OSSI with Binder | | | Green | Yellow | Green |
| | CTAB | | OSSI | Binder | Green | Green | Yellow | Green |
| | CTAB | | OSSI with Binder | | | Green | Yellow | Green |
| Shampoo | Plasma | | OSSI | Binder | Green | Green | Yellow | Green |
| Shampoo | Plasma | | OSSI with Binder | | | Green | Yellow | Green |
| Shampoo | Reduction | | OSSI | Binder | Green | Green | Yellow | Yellow |
| Shampoo | Reduction | | OSSI with Binder | | | Green | Yellow | Yellow |
| Shampoo with reduction | | | OSSI | Binder | Green | Green | Yellow | Yellow |
| Shampoo with reduction | | | OSSI with Binder | | | Green | Yellow | Yellow |
| Shampoo | reduction | acidic oxid | OSSI | Binder | Green | Yellow | Green | Green |

| | | | | | | |
|---|---|---|---|---|---|---|
| Shampoo | reduction | acidic oxid | OSSI with Binder | Yellow | Green | Green |

| | | | | | | | |
|---|---|---|---|---|---|---|---|
| Shampoo with reduction | acidic oxid | OSSI | Binder | Green | Yellow | Green | Green |

| | | | | | |
|---|---|---|---|---|---|
| Shampoo with reduction | acidic oxid | OSSI with Binder | Yellow | Green | Green |

TABLE II

| | | | | | Performance Expectations | | | |
|---|---|---|---|---|---|---|---|---|
| | | | | | | Hair | Hair | Back to |
| Initiating Step | | | Pretreatment | | Sebum | Damage - | Damage - | original |
| Praeparatur | Fundementa | | Step | Binder Step | Test | FT-IR | Feel | color |
| Shampoo | acidic oxidation | | OSSI + Amino | EAA Binder | Green | Green | Green | Green |
| Shampoo with acidic oxidation | | | OSSI + Amino | EAA Binder | Green | Green | Green | Green |
| Shampoo | basic oxidation | | OSSI + Amino | EAA Binder | Green | Red | Yellow | Red |
| Shampoo with basic oxidation | | | OSSI + Amino | EAA Binder | Green | Red | Yellow | Red |
| Shampoo | CTAB | | OSSI + Amino | EAA Binder | Green | Green | Yellow | Green |
| | CTAB | | OSSI + Amino | EAA Binder | Green | Green | Yellow | Green |
| Shampoo | Plasma | | OSSI + Amino | EAA Binder | Green | Green | Yellow | Green |
| Shampoo | Reduction | | OSSI + Amino | EAA Binder | Green | Green | Yellow | Yellow |
| Shampoo with | reduction | | OSSI + Amino | EAA Binder | Green | Green | Yellow | Yellow |
| Shampoo | reduction | acidic oxid | OSSI + Amino | EAA Binder | Green | Yellow | Green | Green |

| | | | | | | | |
|---|---|---|---|---|---|---|---|
| Shampoo with reduction | acidic oxid | OSSI + Amino | EAA Binder | Green | Yellow | Green | Green |

The results presented by these tables demonstrate the superior remanence achieved by the method of the invention. When compared to a coating method that does not include a pretreatment of PTH alkoxysilanes and/or derivatives thereof, especially with PTH as thiol, in combination with a film forming composition having alkoxysilane groups and Fundamenta procedure that is strong enough to disrupt the keratin fibers, the method of the invention evinces the covalent linkage/coupling of the coating to the keratin fibers. The Figures associated with the Experimental section Examples titled “Salon Text Example” show that coating method procedures without the PTH alkoxysilane (experiments S1, S2, S3 using a non-thiol alkoxysilane, MEMO which is trimethoxysilylpropyl(meth)acrylate ester) produce coatings of anagenic hair (salon model hair) that are removed by shampooing while the coatings according to the method of the invention (experiments S4, S5, S6 with mercaptopropyl trimethoxysilane) are not removed. The anagenic hair on the head of the salon model is living, growing hair. Because of this, sebum and other substances are continuously secreted onto the hair roots. This flow of natural oils, sebum, fatty acids and the like undermines the coating produced without the PTH alkoxysilane. The result is that shampooing readily removes this coating. In contrast, the coating produced according to the method of the invention is not removed by shampooing. The comparison of these results shows that the flow of natural oils, sebum, fatty acids and the like cannot undermine the coating made according to the method of the invention. This result is evidence that bonding occurs between the coating and the keratin fibers, e.g., the anagenic hair of the salon model. This evidence provides a demonstration of the above described reasons for the bonding, namely the formation of disulfide links and other covalent links between the PTH alkoxysilanes with PTH as thiol and the keratin proteins of the surfaces of the anagenic hair.

A further demonstration of the covalent linkage between the coating and the keratin protein may be shown by the Model Example 9 set forth below in the Examples section. In this Model, a membrane carrying cysteine or di-cysteine disulfide can be used as a simplified characterization of keratin fibers. A method modeled after the method of the invention may be tested with samples of this membrane to determine whether a thiol compound would bind to the membrane sample. A comparison between the thiol compound test and a similar test with a compound like the thiol compound but without the thiol group would provide test information that may show the binding. Alternatively, a color composition with an in situ linking binder polymer can be applied to such membrane samples to determine which sample would deliver better color remanence.

### Practice of the Activating Step of Praeparatur and Fundamenta Procedures

Embodiments of the activating step according to the invention are directed to cleaning, removal of sebum and disruption of the surfaces and sub-surfaces of keratin fibers, especially anagenic hair, especially anagenic hair on the scalp of a human. The activating step includes two procedures: Praeparatur and Fundamenta. The Praeparatur procedure cleans the keratin fibers to remove dirt, grime, grease and similar untoward substances on keratin fibers such as anagenic hair. The Fundamenta procedure continues the cleaning process though removal of sebum and engages in chemical disruption of the surfaces and sub-surfaces of the keratin fibers.

### Praeparatur Procedure

Substantially complete initial removal of sebum coating the surfaces of anagenic hair delivers a cleaned hair strand surface exposing the microscopic topographic variability provided by keratin protein at this surface. To obtain such keratin fiber cleaning, a Praeparatur procedure is applied. The Praeparatur procedure may be any cleaning operation that removes sebum from the surfaces of keratin fibers. Exemplary Praeparatur procedures include use of one or more applications of a non-conditioning surfactant or substantially non-conditioning surfactant which is free of conditioning actives or substantially free of conditioning additives such as silicones, e.g., amodimethicone, or cetrimonium chloride and polymers such as the polyquaternium versions of cellulose and guar gum derivatives. This procedure calls for one or more applications of the surfactant in an aqueous or aqueous-alcoholic medium with optional agents for ionicity and pH control and organic liquids and solvents for solubilization, dispersion and lifting of dirt, grease and grime in which the kinds and concentrations of components are adjusted to achieve the desired cleaning effect. The procedure involves use of a mild to moderate aqueous composition of an anionic, non-ionic, amphoteric or zwitterionic surfactant at a concentration beginning at about 2 wt % and escalating to about 30 wt %, preferably up to about 25 wt %, more preferably up to about 10 wt % to about 25 wt % relative to the total weight of the composition. The surfactant composition may also include agents for adjustment of viscosity and ionicity and optional adjustment of pH from acidic to neutral to basic. The surfactant composition may begin with a mild surfactant such as a non-ionic or its mixture with other surfactants and may escalate to higher concentrations of anionic surfactant. A preferable surfactant is an anionic surfactant displaying amphiphilic properties such as an alkali metal salt of a C8-C16 alkyl carboxylate, phosphate, sulfonate, sulfate in which the strength of amphiphilic character increases from carboxylate to sulfate. The initial nonionic surfactant used may be followed by a stronger anionic surfactant and then by a solubilizing anionic surfactant having either a PEG group such as PEG-2 to PEG-20, preferably PEG-2 to PEG-5 for increased hydrophicity or a PPG group such as PPG-2 to PPG-5 for increased lipophilicity inserted between the anionic head and the alkyl lipophilic tail of the anionic surfactant. Yet stronger solubilizing media may be formulated by increasing the ionic strength and adjusting the pH. Ionicity builders such as alkali metal sulfates, carbonates, phosphates, nitrates and/or xylene sulfonate may be added. The nature of the medium may also be adjusted to provide organic solvents that are capable of solubilizing oils and sebum. Included are C2 to C8 alcohols, preferably isopropanol, isobutanol and neohexanol as well as acetone, methyl ethyl ketone and other similar organic solvents. This escalating cleaning treatment is designed to escalate in mild stepwise fashion so as to avoid overchallenge of the hair.

This escalating cleaning treatment may be coupled with mechanical agitation such as by a fine tooth comb and/or by a sound vibration such as with an ultrasound device operating at least at 20K Hertz. The mechanical and/or sound vibration can agitate the anagenic hair strands to loosen coatings of sebum, natural oils and secreted sweat and minerals. The ultrasound device may be designed as a fine tooth comb, the teeth of which vibrate to produce the ultrasound. Alternatively, the ultrasound device may be a hand-held generator held in combination with a fine tooth comb which is run through the anagenic hair under the above described cleaning conditions.

### Fundamenta Procedure

The application of the Fundamenta procedure accomplishes disruption of the cleaned surfaces and the sub-surfaces of the keratin fibers such as anagenic hair. The Fundamenta procedure may be applied subsequent to application of the Praeparatur procedure or applied without prior application of the Praeparatur procedure. The Fundamenta procedure structurally disrupts the surface topography and chemical make-up of the surfaces of keratin fibers and removes the F layer coating on the keratin fibers if present. Exemplary activities include use of one or more of an acidic oxidation, a basic oxidation, reduction, a cold plasma discharge, and/or an alkali phase transfer tenside (PETT) such as a multi-alkyl ammonium halide, examples of which are C26-C20 alkyl trimethyl ammonium chloride (CTAC) or bromide (CTAB) such as choline halide, cetyl trimethyl ammonium halide or stearyl trimethyl ammonium halide.

The acidic oxidation may be accomplished is accomplished by exposing anagenic hair or mimic tresses to a dilute acidic oxidizer solution. The oxidizer solution may be formulated with an oxidizing agent at a 0.1 to 6 percent, preferably 0.5 to 3 percent by weight of active oxidizer (calculated by accounting for the concentration of the oxidizer in solution as provided by the supplier) in an aqueous medium at pH from 2 to 5. The oxidizing agent may be hydrogen peroxide. Because persulfates are more powerful oxidizers, they are disfavored for the acidic oxidation process. Oxidizers are typically supplied by commercial sources as acidic solutions. However, the pH may be adjusted if needed with a mineral acid such as hydrochloric acid or sulfuric acid or an organic acid such as acetic acid. The oxidizer solution is applied to a mimic tress or anagenic hair and massaged through the hair strands either by hand or by brush for a period of from 10 seconds to about 5 minutes, preferably about 10 seconds to about 1 to 2 minutes. Thereafter, the tress or anagenic hair, which is substantially saturated with oxidizer solution, may optionally be briefly rinsed with water to remove excess oxidizer solution but not rinsed to the extent to remove all of the oxidizer solution. The presence of some concentration oxidizer is thought to be needed to accomplish coupling of the reactive group(s), e.g., thiol, aldehyde and the like, especially thiol, of the PTH alkoxysilane compound with the sulfur moieties of the disrupted keratin protein and form disulfide and other coupling bonds.

The basic oxidizer treatment may be accomplished by exposing anagenic hair or mimic tresses to a dilute oxidizer solution. The oxidizer solution may be formulated as an aqueous solution of a persulfate, hypochlorite, peroxide or ozone typically at a concentration of from about 0.5 wt % to about 10 wt %, preferably about 0.5 wt % to about 5 wt %, more preferably about 0.5 wt % to about 3 wt %. The pH of the oxidizer solution can be elevated to a basic pH of 9 to 10.5 by addition of ammonia or MEA or sodium silicate or metasilicate. The oxidizer solution is applied to a mimic tress or anagenic hair and massaged through the hair strands either by hand or by brush for a period of from 10 seconds to about 5 minutes, preferably about 10 seconds to about 1 to 2 minutes. Thereafter the tress or anagenic hair, which is substantially saturated with oxidizer solution, is repeatedly rinsed with water to remove the oxidizer solution.

The reduction treatment may be accomplished by exposing anagenic hair or mimic tresses to an aqueous solution or emulsion of from 1 to 30 percent, preferably 2 to 25 percent thioglycolic acid at basic pH such as pH 8.5-9.5. Contact is maintained for approximately 5 to 15 minutes, preferably about 10 minutes and the hair or tress is then rinsed with water and dried to the touch. A commercial perm step one product such as Wella Creatine (N) Perm Emulsion available from Wella Professionals may also be used as a reducing agent.

The cold plasma treatment may be accomplished by passing partially ionized gas over anagenic hair or mimic tresses. Cold plasma is a non-equilibrium atmospheric plasma of a gas such as air or oxygen and/or nitrogen having an effective gas temperature approximating ambient temperature while the electron temperature may be much higher. The gas is passed between dielectric coated electrodes at a high AC voltage potential difference or through an RF field. The electromagnetic field dislodges some electrons from the gas atoms to produce a cascade of ionization processes which lead to the cold plasma stream. An example is an ozone generator which passes air through a high voltage spark discharge. Cold plasma generators are commercial devices designed for production of ambient temperature (cold) plasma. The plasma is transported through a flexible tube to a nozzle. The nozzle through which the plasma stream flows may be passed over the keratin fibers to accomplish plasma treatment. A typical treatment of a mimic hair tress involve passing the nozzle with flowing plasma over the keratin fibers for approximately 1 to 5 minutes, preferably about 1 to about 3 minutes.

The alkali phase transfer tenside treatment is accomplished by washing anagenic hair and/or mimic tresses with an aqueous solution of a phase transfer tenside with an alkaline base or a nucleophile such as an alkoxide. A phase transfer tenside (PETT) generically is a C2-C20 multi-alkyl ammonium halide such as choline or preferably a C12-C20 alkyl trimethyl ammonium chloride or bromide, more preferably cetyl (C16) and/or stearyl (C18) trimethyl ammonium bromide (CTAB). The PETT may be formulated as a 0.1 wt % to 25 wt % aqueous solution. An alkali or thiol aqueous solution (basic alkali pH >10, basic thiol pH>7) of the PETT may be applied to a mimic tress or anagenic hair and massaged through the hair strands either by hand or by brush for a period of 5 to 30 minutes, preferably 5 to 15 minutes to obtain PETT treatment. Thereafter, the tress or anagenic hair, which is substantially saturated with aqueous, basic PETT, is repeatedly rinsed with shampoo in acidic medium to remove the PETT solution.

Use of acidic and/or nucleophilic agents with any of the Fundamenta treatment may facilitate the removal of the 18-methyleicosanoic acid (F-layer acid) esterified and/or thioesterified to the keratin fiber surfaces through interaction with hydroxyl or cysteine or mercapto groups of keratin protein. This action will hydrolyze the F-layer bonds and enable dissolution of the resulting free F-layer acid. An example of such a medium is thioglycolic acid or thioglycolate or an alkyl or aromatic thiol such as hexyl thiol or thiophenol in acetone or aqueous acetone.

Any combination of the Praeparatur procedure and any one of the processes of the Fundamenta procedure may be practiced according to the invention. These combinations include Praeparatur with acidic oxidation, Praeparatur with basic oxidation, Praeparatur with reduction and Praeparatur with reduction followed by acidic oxidation. Each of these combinations can be practiced sequentially or simultaneously. These combination further include Praeparatur with Plasma treatment and Praeparatur with PETT which can be practiced sequentially.

Alternatively, each of the processes of the Fundamenta procedure can be practiced alone without the Praeparatur procedure. These include individual practice of each of acidic oxidation, of basic oxidation, reduction, plasma treatment, PETT treatment alone. In addition reduction followed by acidic oxidation may be practiced without the Praeparatur procedure.

### Pre-Treatment Composition

The significant remanence, wear-fastness and resistance to environmental attack of the coating and preferably color coating on keratin fibers according to aspects of the invention may be developed through interaction between and among any one or more of the components of the film forming composition, the pre-treatment composition and the modified keratin fibers, preferably anagenic hair. The interactions are complex and involve cooperation of binder and pretreatment small molecule components and the disrupted keratin protein at the surfaces and sub-surfaces of the keratin fibers, preferably anagenic hair, through rearranged covalent bonding, hydrogen bonding, dipolar interaction, and molecular entwining (entangling).

The amount of PTH as thiol or thiol derivative (herein after thiol for this paragraph) per unit mass of the pre-treatment composition will determine the reactivity of the thiol group toward keratin protein thiols and potentially reactive groups of the film forming composition. The amount per unit mass is a more accurate determinant of reaction ability than is the overall thiol concentration, because the concentration of a thiol becomes very high during drying step, but the drying does not affect the value of thiol per unit mass. This thiol per unit mass can be expressed as Functional Equivalent Molecular Weight (FEMw) where the total molecular weight of the molecule or polymer is divided by the number of thiol or protected thiol functional groups.

Embodiments of the pre-treatment composition may comprise PTH alkoxysilane compounds as pretreat molecules defined above that incorporate PTH groups as well as alkoxysilyl groups. In addition to the PTH group as thiol, the PTH group includes the sulfur protected derivatives, as well as complementary reactive groups that will combine with thiol of the keratin protein. The pretreat molecules of the pre-treatment composition preferably have a weight average molecular weight of from about 100 Da to about 40 KDa, preferably from about 100 Da to about 5 KDa, more preferably from about 100 Da to about 3 KDa, especially more preferably from about 100 Da to about 2 KDa. Optionally, the pretreatment composition may also incorporate aminoorgano alkoxysilane and thiolorganic compounds.

Embodiments of the PTH alkoxysilane compounds of the pretreatment composition comprise an organic core of 1 to 10 carbons, or from 1 to 100 repeat organic monomeric units, preferably from 1 to 50 repeat organic monomeric units, more preferably from 1 to 10 repeat organic monomeric units, wherein the monomeric units may be olefinic, ester, amide, urethane, urea, ether units and any combination thereof. Alternatively the pretreatment composition may comprise silicone cores of from about 1 to about 100 dimethylsiloxanyl units, preferably about 1 to 50 dimethylsiloxanyl units, more preferably about 1 to about 20 dimethylsiloxanyl units, especially more preferably about 1 to about 10 dimethylsiloxanyl units, most preferably about 1 to about 5 dimethylsiloxanyl units. Alternatively, the silicone core may be 1 to 3 silicone units, more preferably 1 or 2 silicone units in addition to the alkoxysilane group. The organic and silicone core molecule embodiments of the pre-treatment composition also comprise one or more PTH groups and one or more alkoxysilyl groups. The number of PTH groups and alkoxy silyl groups present in the pretreat molecule will depend upon the kinds of inter molecular connections desired for the pre-treatment composition. In particular, the pretreat molecule comprises at least one PTH group for chemical interaction with modified keratin protein and is believed to form chemical bonds such as disulfide, sulfone ester, thioester and similar functional groups with the modified keratin protein. In particular, the pretreat molecule also comprises at least one alkoxysilyl group. The PTH and alkoxysilyl group containing molecule can also be formed in-situ out of compatible PTH and alkoxysilyl precursors, some examples of which are: a) multi-mercapto molecule and epoxy-alkoxysilyl molecule, b) multi-mercapto molecule and alkenoyloxy-alkoxysilyl molecule, c) b) multi-mercapto molecule and alkene-alkoxysilyl molecule, d) mercapto-amino molecule and aldehyde-alkoxysilyl molecule. The alkoxysilyl group provides covalent silicon-oxygen-silicon or silicon-oxygen-carbon bond formation with the binder and with the PTH alkoxysilane compounds themselves to form extended silicone polymer moieties. Embodiments of the PTH alkoxysilane compound include the PTH organo alkoxysilane and the PTH organo multidimethylsiloxanyl alkoxysilane as described above and by Formulas IIIA and IIIB described below respectively.

In addition to the PTH alkoxysilane compounds themselves, dimers with disulfide and tetrasulfide are included. The dimers with disulfide are formed from the PTH alkoxysilane compounds with PTH as thiol. They comprise two thiolorgano-alkoxysilanes or two thiolorgano multidimethylsiloxanyl alkoxysilanes joined together at their thiol groups to form a bis[organo-alkoxysilanyl]disulfide or a bis[organo-multidimethylsiloxanyl alkoxysilane]disulfide respectively. The dimers with tetrasulfide comprise two thiolorgano-alkoxysilanes or two thiolorgano multidimethylsiloxanyl alkoxysilanes joined together at their thiol groups by combination with sulfur as S2 to form a bis[organo-alkoxysilanyl]tetrasulfide or bis[organo-multidimethylsiloxanyl alkoxysilane]tetrasulfide.

Additionally included as optional components for the pretreatment composition are an optional thiol organic component of Formula V and an optional aminorgano alkoxysilane component of Formula VI, both described below.

The components of the pre-treatment composition comprise at least one compound of the group of PTH alkoxysilane compounds including the PTH organo-alkoxysilane, the PTH organo multidimethylsiloxanyl alkoxysilane and/or the cyclic thiol alkoxysilane.

These PTH alkoxysilane compounds are structurally characterized by Formulas IIIA, IIIB and IV and include PTH compounds in which the PTH group comprises the free thiol group, sulfide dimers and tetramers, groups complementarily reactive with thiol and the sulfur protected analogs.

(PTH—(CH₂)ₖ—(Y)ₗ)_{d}-(ORG)ₘ-SiR¹₃₋ₙ(OR)ₙ Formula IIIA

PTH—(CH₂)ₖ—(SiMe₂O)ₒ—SiR¹₃₋ₙ(OR)ₙ Formula IIIB

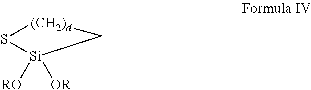

Formulas IIIA, IIIB and IV are characterized by the designators k, l, d, m, n, o and by the substituent groups PTH, Y, and ORG. In order of appearance in Formulas IIIA, IIIB and IV, the designators are as follows.
1) Designator k is an integer of 1 to 20, preferably 1 to 10, more preferably 1 to 6.
2) Designator 1 is zero or 1.
3) Designator d is an integer of 1, 2 or 3.
4) Designator m is zero or an integer of 1 to 6.
5) Designator n is an integer of 1 to 3.
6) Designator o is an integer of from 1 to 20.

The substituent group PTH defines the thiol moiety and may comprise R³S—, as well as chemical groups that react with thiol including OHC—, H₂C═CR¹⁰—CO₂— or HO—. The group R³ of PTH defines whether the thiol moiety is a free thiol (—SH) or a protected thiol. Accordingly, the substituent R³ may comprise hydrogen, cyano, alkanoyl of 2 to 10 carbons, a phenyl group, a heteroaromatic group, a phenylalkyl group or a heteroaromatic alkyl group in which heteroaromatic group is pyridyl, pyrimidinyl, pyrrolyl or thiophenyl and the alkyl group is a C1-C4 alkyl group, and R¹⁰ may be hydrogen or methyl.

The group R defines the alkyl character of the alkoxy group. Accordingly, R may comprise a C1-C4 alkyl, preferably C1-C3 alkyl, more preferably methyl or ethyl.

The group Y defines how the PTH—(CH₂)ₖ moiety is connected to the remainder of Formula IIIA. Group Y may be absent (l is zero) or may be present (l is 1). When present, Y may comprise —COO—, —OOC— (carboxyl, oxycarbonyl), ether oxygen, ether thiol, —NH—, —NMe— —HNCO— or —CONH—.

The group ORG is an organic connecting group joining the left and right halves of Formula IIIA. As a connecting group, ORG may be divalent or multivalent. The divalent configuration connects the left (PTH—(CH₂)ₖ—(Y)ₗ)_{d} moiety with the right moiety. The multivalency connects multiple left (PTH—(CH₂)ₖ—(Y)ₗ)_{d} moieties with a single alkoxysilane group, the right side group. Accordingly Group ORD may comprise two different configurations.

In a first configuration, Group ORG comprises a divalent organic group including alkyldithioalkyl, alkyldiazoalkyl, alkylurethanylalkyl, alkylureidoalkyl, alkylcarboxylalkyl, alkylamidoalkyl, alkylesteralkyl or alkyl in which each alkyl group independently in each instance is a C1-C20 linear or branched alkyl group, preferably a linear C1-C6 alkyl group, more preferably a linear C1-C3 alkyl group such that ORG connects the left (PTH—(CH₂)ₖ—(Y)ₗ)_{d} section and the right —SiR¹₃₋ₙ(OR²)ₙ section of Formula IIIA,

In a second configuration, Group ORG comprises a multivalent C1-C20 alkylenyl group of the following formula with f as zero or an integer of 1-19:

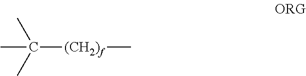

In this second configuration, the moiety of Formula IIIA comprising

(PTH—(CH₂)ₖ—(Y)ₗ)_{d}—(ORG)ₘ

becomes Formula A

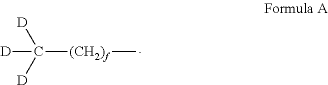

Formula A has two or three (PTH—(CH₂)ₖ—(Y)ₗ)_{d} sections connected as D's to the ORG dangling multiple valences. When two of the (PTH—(CH₂)ₖ—(Y)ₗ)_{d} sections are connected to the dangling multiple valences of ORG, the third D of Formula A may be hydrogen or C1-C6 alkyl, preferably C1-C3 alkyl, more preferably methyl. The dangling valence of (CH₂)_{f}— of Formula A is bound to the right —SiR¹₃₋ₙ(OR²)n section of Formula IIIA.

In addition to the PTH alkoxysilane compound of Formula IIIA or Formula IIIB these compounds can be at least partially precondensed to be a polycondensate of the PTH alkoxysilane of Formula IIIA and/or Formula IIIB with PTH as thiol or protected thiol wherein the PTH alkoxysilane of Formula IIIA and/or Formula IIIB is at least partially polycondensed with itself or together and/or an alkylalkoxysilane of Formula B wherein R⁸ is a linear or branched alkyl group of 1 to 10 carbons:

R⁸—SiR¹₃₋ₙ(OR)ₙ Formula B

to produce a linear or branched oligomeric silicone polycondensate having a linear or branched silicone chain of a combination of M, D and T groups wherein the polycondensate has pendant alkoxy groups, pendant thiolalkyl groups and pendant alkyl groups, and the polycondensate has an M_{w} from 350 to 3500 Da and a functional equivalent M_{w} (FEM_{w}) of the thiol and or protected thiol group from 70 to 900 and a FEM_{w} for the alkoxy groups from 50 to 900.

Preferably a precondensed PTH alkoxysilane compound is Formula IIIA is partially precondensed alone to form a linear or branched oligomeric silicone polycondensate.

An optional PTH organic compound having no alkoxysilyl groups may be included as a supplemental sulfur compound in the pretreatment composition along with the PTH organic alkoxysilane. The optional PTH organic compound provides a multiplicity of links by and between the sulfur groups of the proteins of the modified keratin fibers and at least the film forming composition. The optional PTH organic compound comprises Formula V

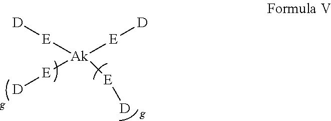

For Formula V, D is (PTH—(CH₂)ₖ—(Y)ₗ) as defined above and the variables of this D group are independent for each instance of D. Each of the designators g is independently zero or 1. The group E may be a bond or a C1-C6 alkylenyl group. The group Ak may be a carbon atom Ak0 or the structures Ak1, Ak2, Ak3, Ak4 depicted as follows. The dangling valences of the central carbon of Ak0, Ak1, Ak2 and Ak3 are bonded to E-D and the CH₂ valence is bonded to D. All dangling valences of Ak4 are bound to E-D.

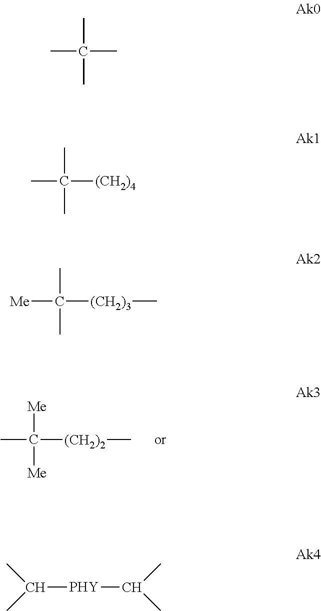

For Formula V, the Ak2 and Ak3 formulas with methyl bound to a central carbon have one or two of the g designators of Formula V respectively as zero and have methyl in place of the zeroed out E-D group. PHY of Ak4 is an oligomer of 2 to 10 units of a C3-C8 α,ω hydroxyalkanoic acid ester having a —O—(CH₂)ₕ—O— at its carboxy terminus and a —(CH₂)ᵢ—O— group at its hydroxyl terminus in which the —O—(CH₂)ₕ—O— and —(CH₂)ᵢ—O— groups are bonded respectively to the CH groups and the designator h is an integer of from 2 to 4 and the designator i is an integer of from 1 to 3.

For all of the foregoing compounds with sulfur, the preferred form of PTH is —SH.

The optional aminoorgano-alkoxysilane may be included as a supplemental amine compound in the pretreatment composition. The aminoorgano-alkoxysilane provides additional electrostatic interaction especially with the film forming composition as the olefin polymer with carboxyl groups. The aminoorgano-alkoxysilane compound comprises Formula VI

[H₂N—(CH₂)ₘ—(NH—R¹⁴)ₙ]ₐ—[ROₜMe₃₋ₜSi—O]_{b}—(—SiMe₂-O)ₚ—[(—SiMe₂₋ᵣ[(CH₂)_{m′}—NH₂]ᵣ—O]ₛ-[A]_{c}-(—SiMe₂-O)ᵤ—(SiMe₃₋ₜOR₁) Formula VI

For Formula VI, R¹⁴ is a C1-C6 alkylenyl group and R may be methyl or ethyl.

The designators m, n, a, t, b, p, r, m′, s, c, and u indicate the presence or absence of the associated groups and if the associated group is present the corresponding designator indicates how many of that group are present. These designators are as follows.

Designators m and m′ may be an integer of 1 to 6 preferably 1 to 3.

Designators b, r, s, c, may be zero or 1.

Designator n may be zero or an integer of 1-6, preferably zero or 1-3.

Designator a may be zero or an integer of 1-3.

Designator t may be 1 to 3.

Designators p and u may be zero or an integer of 1 to 12.

The groups in brackets, i.e. the groups with designator a and b and the group with designator s are terminal and pendant groups respectively so that the group with designator b would be a terminal group if b is 1 and a is zero and the group with designator a would be a terminal group if a is 1 and b is zero. In these instances of terminal groups, if b is 1, a must be zero and s and r must be 1 to provide an aminoorgano-alkoxysilane with the pendant amine group —CH₂)_{m′}—NH₂. Alternatively, in these instances of terminal groups, if a is 1, 2 or 3, b must be zero.

The remaining substituent Group A may be any one of three alternative moieties. These are as follows.
1) Group A may be a divalent group including dithio, diazo, urethanyl, ureido, carboxyl, amido, ester, or aminoethyloxycarbonyl, or a C1-C20 alkylenyl group connecting the left and right sections of the aminoorgano-alkoxysiloxane compound.
2) Group A may be a multivalent C1-C20 alkylenyl group connecting two or three left sections and one right section of the aminoorganoalkoxysiloxane compound when a is 2 or 3 and b, p and s are zero.
3) Group A may be a linear or branched polyethylene imine moiety of from 2 to 2000 ethylene imine units in which case, b, p, s and u are all zero and optionally the group —(SiMe₃₋ₜORₜ) may be replaced by —NH₂.
4) Group A may be a terminal group selected from C2-C8 alkylenyl(meth)acrylate or —(CH₂)ₙ—O—CH₂—CHOHCH₂—O₂C(R)═CH₂ wherein R is H or CH₃ and n is an integer of 2 to 8.

A preferred version of the PTH organo-alkoxysilane of Formula IIIA comprises Formula OSSI in which k is an integer of 1 to 20, preferably 1-6, the multi (CH₂) chain may be linear or branched, n is an integer of 1 to 3, preferably 3, R¹ is methyl and R² is methyl or ethyl.

HS—(CH₂)ₖ—SiR¹₃₋ₙ(OR²)ₙ Formula OSSI

A more preferred version of Formula OSSI designates n as 3 and R² as methyl (Me) or ethyl (Et) so as to provide the following embodiment of the thiolorgano-alkoxysilane.

HS—(CH₂)ₖ—Si(OMe)₃ or HS—(CH₂)ₖ—Si(OEt)₃

A preferred version of the aminoorgano-alkoxysilane of Formula V comprises Formula OASI in which m is an integer of 1 to 6, n is zero or an integer of 1 to 3, p and u are each independently zero or an integer of 1 to 3, c is zero or 1, each instance of R¹⁴ is independently ethyl, propyl, butyl or isobutyl, A is C1-C6 alkylenyl, R³ is methyl and R⁴ is methyl or ethyl.

H₂N—(CH₂)ₘ—(NH—R¹⁴—)ₙ—(SiMe₂O)ₚ-A_{c}-(—SiMe₂-O]ᵤ—SiR³₃₋ₜOR⁴ₜ Formula OASI

A more preferred version of Formula OASI designates, p, u and c as zero, m as 2, 3 or 4 (butyl or isobutyl), n as 1 or 2, each instance of R¹⁴ independently as ethyl, propyl, butyl or isobutyl, t as 3 and R⁴ as methyl (ME) or ethyl (Et) so as to provide at least the following embodiments of the aminoorganoalkoxysilane:

H₂N—(CH₂)ₘ—Si(OR⁴)₃ or H₂N—(CH₂)ₘ—(NH—R¹⁴)ₙ—NH—R¹⁴—Si(OR⁴)₃ or H₂N—(CH₂)ₘ—NH—R¹⁴—Si(OR)₃.

Exemplary embodiments of the thiolorgano alkoxysilane component of the pre-treatment composition include but are not limited to 3-mercaptopropyltriethoxysilane, 2-mercaptoethyltriethoxysilane, 4-mercaptobutyltriethoxysilane, 1-mercaptomethyltriethoxysilane bis[3-(triethoxylsilyl)propyl]disulfide, 2,2-diethoxy-1-thio-2silacyclopentane as well as the trimethoxysilane and trimethoxysilyl versions thereof.

Table III provides a listing of appropriate thiolorgano alkoxysilane compounds as pretreat molecules of the pre-treatment composition.

TABLE III
THIOLORGANO ALKOXYSILANE PRETREATMENT
SMALL MOLECULES

| Chemical Name | CAS No. |
|---|---|
| 2,2-DIMETHOXY-1-THIA-2-SILACYCLOPENTANE | 26903-85-5 |
| 3-MERCAPTOPROPYLTRIETHOXYSILANE | 14814-09-6 |
| 3-MERCAPTOPROPYLTRIMETHOXYSILANE | 4420-74-0 |
| 1-MERCAPTOUNDECYLTRIMETHOXYSILANE | 877593-17-4 |
| S-(OCTANOYL)MERCAPTOPROPYLTRIETHOXYSILANE | 220727-26-4 |
| 3-(2-PYRIDYLETHYL)THIOPROPYLTRIMETHOXYSILANE | 198567-47-4 |
| 3-THIOCYANATOPROPYLTRIETHOXYSILANE, | 34708-08-2 |
| 2-(3-TRIMETHOXYSILYLPROPYLTHIO)THIOPHENE | 1364140-50-0 |
| (MERCAPTOMETHYL)METHYLDIETHOXYSILANE | 55161-63-2 |
| 3-AMINOPROPYLTRIETHOXYSILANE | 919-30-2 |
| 3-AMINOPROPYLTRIMETHOXYSILANE | 13822-56-5 |
| 3-AMINOPROPYLTRIS(METHOXYETHOXYETHOXY)SILANE | 87994-64-7 |
| 11-AMINOUNDECYLTRIETHOXYSILANE | 116821-45-5 |
| 3-MERCAPTOPROPYLMETHYLDIMETHOXYSILANE | 31001-77-1 |
| BIS[m-(2-TRIETHOXYSILYLETHYL)TOLYL]POLYSULFIDE | 67873-85-2 |
| BIS[3-(TRIETHOXYSILYL)PROPYL]DISULFIDE | 56706-10-6 |
| BIS(TRIETHOXYSILYL)-4,5-DITHIOOCTANE | |
| BIS[3-(TRIETHOXYSILYL)PROPYL]TETRASULFIDE | 40372-72-3 |
| N,N′-BIS[3-(TRIETHOXYSILYL)PROPYL]THIOUREA | 69952-89-2 |
| TRIMETHOXYSILYLPROPYL THIOGLYCOLATE | |
| TRIETHOXYSILYLPROPYL THIOGLYCOLATE | |
| TRIMETHOXYSILYLBUTYL THIOGLYCOLATE | |
| TRIMETHOXYSILYLPROPYL THIOPROPIONATE | |
| TRIMETHOXYSILYLBUTYL THIOPROPIONATE | |
| (TRIMETHOXYSILYL)METHANETHIOL | 30817-94-8 |
| BIS[(TRIMETHOXYSILYL)METHYL]DISULFIDE | |
| BIS[(TRIMETHOXYSILYL)METHYL]TETRASULFIDE | |

Additional preferred thioorganoalkoxysilanes, preferred aminoorgano-alkoxysilanes and other preferred alkoxysilanes of the pretreatment composition include:
a) trimethoxysilyl propyldiethylene triamine (SCA);
b) trimethoxysilyl propyl (meth)acrylate ester (MEMO);
c) aminopropyl triethoxysilane (APTES);
d) tetraethoxy silane (TEOS);
e) 3-mercaptopropylsilyltriol;
f) 3-mercaptopropyltrimethoxysilane;
g) 3-mercaptopropyltriethoxysilane;
h) 3-thioglycydyloxypropyltrimethoxysilane;
i) 3-thioglycoloyloxypropyltriethoxysilane.

### The Binder Step for Forming a Coating and Preferably Color Coating on Keratin Fibers

Embodiments of the method for forming the coating and preferably color coating on keratin fibers involve as a final step, the binder step. The binder step calls for application of the film forming composition onto the modified keratin fibers having the precoating of pretreatment composition. The film forming composition may comprise any one of four different kinds of polymer compositions. The first two embodiments of the film forming composition are based on a unitary binder polymer construction. The first embodiment comprises a unitary binder polymer with a monofunctional binder group comprising an alkoxysilane group. The second embodiment comprises a unitary binder polymer with a monofunctional binder group comprising a carboxylic acid or carboxylic acid salt group.

The third and fourth embodiments of the film forming composition are based upon dual in situ reactive polymer constructions. The third embodiment comprises a pair of binder polymers in which the first component binder polymer has a functional binder group comprising an alkenoyloxy group and the second component binder polymer has a functional binder group comprising an amine or thiol group. These two functional binder groups constitute a complementary pair or reactive groups, commonly known as a Michael addition pair. The fourth embodiment comprises a pair of binder polymers in which the first component binder polymer has a functional binder group comprising a carboxylic acid group and the second component binder polymer has a functional binder group comprising a carbodiimide group. These two functional binder groups constitute a complementary pair of reactive groups.

### The Single Polymer Alkoxysilane Film Forming Composition

In the first embodiment of the film forming composition, the binder polymer is unitary and comprises an organic polymer binder having two or more functional binder groups which are in situ crosslinkable and are both an alkoxysilyl group. The film forming composition further may comprise a substance that functions as a catalyst in relation to this in situ cross linkable polymer. More specifically, the organic polymer binder comprises an in situ cross linkable self covalently reactive organic polymer having two or more alkoxysilyl pendant and/or terminal groups, preferably at least terminal alkoxysilyl groups. The organic polymer binder may comprise a polymer or copolymer of ester, amide, urethane, urea, ether and/or olefinic monomeric units or any combination thereof. The binder polymer may be a random or block copolymer and may have a linear or branched, preferably a linear configuration.

In particular, the self-reactive organic polymer binder comprises Formula IA

X₃Si—R¹-Ct-[Poly]_{y}-Ct-R¹—Si—X₃ Formula IA

In Formula IA, X may be alkoxy of 1 to 3 carbons, preferably methoxy or ethoxy. The group R¹ is a C1 to C8 linear or branched alkylenyl group. The group Ct is a connector group which joins or connects X₃Si—R¹— to Poly.

Group Ct comprises Formula II.

—U¹—R²—U²— Formula II

For Formula II, U¹ and U² are each independently a urea or urethane group. The group U¹ is covalently bonded to R¹ and the group U² is covalently bonded to Poly. The group R² is a C2 to C12 linear or branched alkylenyl group, a C6-C16 alkylcycloalkyl group which may include one or multiple cycloalkyl rings linked in tandem or linked by alkyl groups, a C6-C12 aromatic group which may include one or multiple aromatic groups or a C6-C14 alkyl aromatic group which may include one or multiple aromatic groups linked in tandem or linked by alkyl groups. Embodiments of the group R2 are derived from common diisocyanates. For example, hexamethylene diisocyanate (1,6-hexane diisocyanate) produces an alkylenyl group. Isophorone diisocyanate produces an alkyl cycloalkyl group. Toluene diisocyanate produces an alkyl aromatic group. Methylene bis(cyclohexane isocyanate) also produces an alkylcycloalkyl group. Methylene diphenylisocyanate produces an alkyl aromatic group. Formula II is produced by combination of an R²-diisocyanate and the corresponding hydroxyl or amine from X₃SiR¹-G and G-Poly in which each G independently is an amine or hydroxy group.

The group Poly is the primary binder polymer backbone providing flexibility, tensile strength and film formation for the coating and preferably color coating. Preferably, Poly is an organic backbone of monomeric units such as but not limited to ester, amide, urethane, urea, olefin units and at least with terminal alkoxysilyl groups (i.e., trialkoxysilyl groups as defined in the Definitions section). The backbone of Poly may be of any configuration, which preferably is a linear or branched configuration, and the linear configuration is the most preferred configuration. In a branched configuration of Poly, the optional pendant alkoxysilyl groups may be the termini of the branches. Because of the multiple condensation ability of each alkoxysilyl group of the binder polymer, their condensation to form Si—O—Si bonds produces an in situ crosslinked binder polymer forming a three dimensional network. All configurations of Poly, and preferably the linear configuration of Poly, produce this binder polymer network with Si—O—Si connections which extend the backbone and interconnect separate backbones as cross links due to the multiple times an alkoxysilyl group can condense with other alkoxysilyl groups. Although it is not a limitation of the invention, it is believed that the in situ crosslinking occurs as well with the pretreatment alkoxysilyl small molecules to establish the three dimensional network throughout the combination of binder polymer and small molecules.

The group Poly may have any structural configuration as described in the Definitions section, and preferably has a linear backbone configuration and may be formed of monomeric units of an ester, urethane, urea, amide or polyol (ether) group or any combination thereof. The designator y indicates the extent of Poly and is an integer designating the number of monomeric units of Poly forming the backbone. Accordingly, y is an integer of from about 2 up to about 1 million, preferably up to about 300,000, more preferably up to about 250,000, most preferably up to about 200,000.

When the monomeric unit of Poly is an ester, the ester monomeric unit may be formed of a C2-C10 linear or branched alkane diol or a C8-C20 aromatic diol, and a C3 to C10 linear or branched alkanodioic acid or a C8-C10 aromatic dicarboxylic acid or formed of a C3-C10 hydroxy alkanoic acid or a C8-C10 aromatic hydroxy carboxylic acid.

When the monomeric unit of Poly is a urethane, the urethane monomeric unit is formed of a C2-C10 alkanodiol and an R³ diisocyanate wherein R³ is as described below.

When the monomeric unit of Poly is a urea, the urea monomeric unit is formed of a C2-C10 linear or branched alkanodiamine and an R³ diisocyanate.

When the monomeric unit of Poly is an amide, the amide monomeric unit is formed of a C2-C10 alkanodiamine and a C3 to C10 alkanodioic acid or C8-C10 aromatic dicarboxylic acid.

When the monomeric unit of Poly is a polyol, the polyol monomeric unit is a formed of ethylene oxide (linear) or propylene oxide (branched).

A preferred ester monomer of Poly is formed of glycol, 1,4-butanediol or 1,6-hexanediol and malonic acid, succinic acid, glutaric acid, adipic acid, phthalic acid, terephthalic acid or any combination thereof or alternatively, the Poly is formed from a hydroxy acid such as glycolic or lactic acid, o-hydroxy propanoic acid, o-hydroxybutanoic acid or p-hydroxybenzoic acid. An especially preferred ester monomer of Poly is formed of glycol(dihydroxy ethane) or 1,6-hexanediol and succinic acid, adipic acid or phthalic or terephthalic acid.

A preferred urethane monomer of Poly is formed of glycol, 1,4-butanediol or 1,6-hexanediol and isophorone diisocyanate, methylene bis(phenylisocyanate), toluene diisocyanate or 1,6-hexane diisocyanate. An especially preferred urethane monomer of Poly is formed of glycol or 1,6-hexanediol and isophorone diisocyanate or toluene diisocyanate.

A preferred urea monomer of Poly is formed of 1,3-propanediamine, 1,4-butanediamine or 1,6-hexanediamine and isophorone diisocyanate, methylene bis(phenylisocyanate), toluene diisocyanate or 1,6-hexane diisocyanate. An especially preferred urethane monomer of Poly is formed of 1,3-propanediamine or 1,6-hexanediamine and isophorone diisocyanate or toluene diisocyanate.

A preferred amide monomer of Poly is formed of 1,3-propanediamine, 1,4-butanediamine or 1,6-hexanediamine and malonic acid, succinic acid, glutaric acid, adipic acid, phthalic acid, terephthalic acid or any combination thereof. An especially preferred amide monomer of Poly is formed of 1,3-propanediamine or 1,6-hexanediamine and succinic acid, adipic acid or phthalic or terephthalic acid.

A preferred polyol of Poly is a PEG-200 to PEG-2000.

The Poly group may be any combination of ester, urethane, urea, amide and/or polyol block or random arrangements. For example:
a) a combination of polyester and polyurethane blocks may be formed from a diol and blocks of dicarboxylic acids and diisocyanates;
b) a combination of polyester and polyurea blocks may be formed from their respective reactants and the joinder between blocks may be formed as a urethane connection by reacting a polyester block terminating with a hydroxyl and a polyurea block terminating with an isocyanate;
c) a combination of polyester and polyamide blocks may be formed from a dicarboxylic acid and blocks of diols and diamines;
d) a combination of polyester and polyol blocks may be formed from a polyol and blocks of diol and dicarboxylic acid;
e) a combination of polyurethane and polyurea blocks may be formed from their respective reactants and the joinder between blocks may be formed as a urethane and/or urea connections;
f) a combination of a polyurethane and polyamide blocks may be formed from their respective reactants and the joinder between blocks may be formed as a urea connector by reacting a polyamide block terminating with an amine and a polyurethane block terminating with an isocyanate;
g) a combination of polyurethane and polyol blocks may be formed from a polyol and blocks of diol and diisocyanate;
h) a combination of polyurea and polyamide blocks may be formed from their respective reactants and the joinder between blocks may be formed as a urea connector by reacting a polyamide block terminating with an amine and a polyurea block terminating with an isocyanate;
i) a combination of polyurea and polyol blocks may be formed from a polyol and blocks of diamine and diisocyanate and the joinder between blocks may be formed as a urethane connector by reacting a polyol block terminating with a hydroxy and a polyurea block terminating with an isocyanate;
j) a combination of polyamide and polyol blocks may be formed from a polyol and blocks of diamine and dicarboxylic acid and the joinder between blocks may be formed as an ester connector by reacting a polyol block terminating with a hydroxy and a polyester block terminating with a carboxylic acid.

Provisos apply to the choice of the U² group. U¹ will always be urea when the trialkoxysilyl alkylenyl-G starting material is a trialkoxysilylalkylenylamine. Alternatively, U¹ will always be urethane when the trialkoxy alkylenyl-G starting material is an trialkoxysilylalkylenylalcohol (OH). In the following provisos, U¹ is always urea resulting from the preferred trialkoxysilylalkylenylamine starting material.
a) When Poly ends with an ester monomeric unit, U² is a urethane group and U¹ is a urea group.
b) When Poly ends with a urethane monomeric unit, U² is a urethane group and U¹ is a urea group.
c) When Poly ends with a urea group, U² and U¹ are both urea groups.
d) When Poly ends with an amide monomeric unit, U² and U¹ are both urea groups.
e) When Poly ends with polyol monomeric unit, U² is a urethane and U¹ is a urea group.

Like the R² group, the R³ group may be a C2 to C12 linear or branched alkylenyl group, a C6-C16 alkylcycloalkyl group or a C6-C14 aromatic group. R² and R³ are both the organic groups for the diisocyanate reactant forming the urethane and urea groups. Preferably, R² and R³ may each independently be methylene bisphenyl (as in methylene bis(phenylisocyanate), toluenylenyl (as in toluene diisocyanate), hexanylenyl (as in hexamethylene diisocyanate), naphthalenyl (as in naphthalene diisocyanate), methylene bis cyclohexylenyl (as in methylene bis (cyclohexylisocyanate) which is hydrogenated methylene bis (phenylisocyanate)) and isophoronylenyl (as in isophorone diisocyanate).

In each of the organic binder polymers of Formula IA, Poly may optionally contain one or more trifunctional groups such as a triol or triamine which function to provide pendant alkoxysilyl groups for the organic binder polymer.

The third hydroxyl or amine of the trifunctional group constitutes a link to a pendant SiX₃ through the same Ct-R¹ group of Formula IA. This version of the organic binder polymer comprises Formula IB in which Z is trifunctional group linked through Ct-R¹ to the third SiX₃:

X₃Si—R¹-Ct-[(Poly)ₓ-(Z)_{z}-(Poly)ₐ]_{y}-Ct-R¹—Si—X₃ Formula IB

For Formula IB, the Z group is derived from a triol or triamine starting material of the formula III

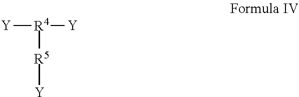

in which the Y groups are hydroxyl or amine or when Poly is an ester, the Z group alternatively may be a tricarboxylic acid. Except for the tricarboxylic acid embodiment, Formula IV is a homolog starting material of the diol or diamine starting material for the ester, urethane, urea, amide or polyol monomeric unit in which the —R⁵—Y branch group of Formula IV has the same configuration as the organic moiety of the diol or diamine. For example, if the Poly is a polyurethane or polyurea constructed from a propane diol, the triol compound would be 2-hydroxymethyl-1,3-propane diol, also known as trihydroxymethyl methane. As stated above for Z, the third Y group, the pendant Y group of the triol or triamine (hydroxyl or NH₂) is bonded through Ct to R¹—SiX₃ so that the pendant Y of the triol or triamine starting material of Formula IV becomes part of a urethane or urea group just as shown for Ct of Formula I. The resulting full Formula IB in which Z is has the structure ═R⁴—R⁵-Ct-R¹—SiX₃ is:

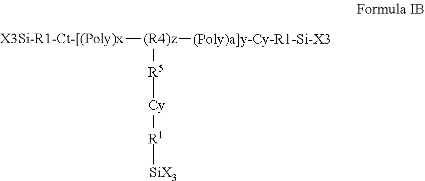

For Formula IB, the designator z indicates the number of pendant alkoxysilyl groups present in the binder polymer. For binder polymers having multiple pendant alkoxysilyl groups, Z of Formula IB (i.e., ═R⁴—R⁵-Ct-R¹—SiX₃) is randomly distributed throughout the binder polymer backbone. Accordingly, designator z is an integer from 1 to 1 thousand and specifies the number of trifunctional groups present in Formula I′. Preferably, z is an integer from 1 to 100, more preferably from 1 to 10, especially more preferably from 1 to 5 and most preferably from 1 to 3. The sum of the integer designators x, z and a equals y so that the weight average molecular weight of the binder polymer of Formula I′ is the same as the weight average weight of the binder polymer of Formula I.

Versions of the binder polymer may be all of Formula IA or all of Formula IB which is a having terminal and pendant alkoxysilyl groups. The binder polymer as well may be a mixture of Formula IA and Formula IB. For a mixture, the ratio of Formula IA to Formula IB may range from may be in a range of 100:1 to 1:100, preferably 50:1 to 10:9 or 25:1 to 2:1 or 20:1 to 10:1.

The weight average molecular weight of Formula IA and Formula IB may range from about 1 KDa to about 1MDa, preferably from about 1 KDa to about 500 KDa, more preferably from about 1 KDa to 300 KDa, especially more preferably from at least about 2 KDa up to about 250 KDa, most preferably from at least about 2KDa up to about 150 to about 200 KDa. The designator y of Formula IA is chosen to provide an average molecular weight in this range. Similarly, the sum of the designators x, z and a of Formula IB is chosen to equal the choice for y and the average molecular weight in this range.

The choice of the ratio of z relative to the two terminal alkoxysilyl groups for Formula IB may preferably range from 1:2 to 100:2 more preferably from 1:2 or 2:2 to 20:2, most preferably at least 1:2 up to 5:2 or 10:2. The presence of the pendant alkoxysilyl group provides additional crosslinking among the binder polymer molecules and with the pretreatment small molecule. Although it is not a limitation of the invention, it is believed that the additional crosslinking is capable of delivering a significant remanent coating and preferably color coating on keratin fibers.

Preferred embodiments of the organic binder polymer of Formula IA (with terminal alkoxysilyl groups alone) provide:
a) Poly as a polyurethane constructed of a C4-C6 alkane diol, preferably hexane diol and isophorone diisocyanate, toluene diisocyanate or methylene bis (phenylisocyanate), or
b) Poly as a polyethylene glycol or polypropylene glycol or
c) Poly as a polyester constructed of a C2-C6 alkane diol, preferably ethylene glycol and succinic acid, adipic acid or any form of phthalic acid, preferably terphthalic acid.
   The Ct group is formed from a C1-C4 alkane diisocyanate. The R¹—SiX₃ group is formed from an ω-amino propyl or isobutyl triethoxysilane or the trimethoxysilane homolog.

A preferred embodiment of the organic binder polymer of Formula IB has Poly, Ct and R¹—SiX₃ as described above for the preferred polyurethane and polyester organic binder polymers of Formula IA of the foregoing subparagraphs a and c except that from about 0.1 wt % to about 5 wt %, preferably from about 0.5 wt % to about 3 wt % of the C4-C6 alkane diol is replaced by 3-(3-hydroxyprop-1-yl)-1,6-hexanediol so that the ratio of pendant alkoxysilyl to terminal alkoxysilyl groups of preferred Formula IB is from 1:2 to 5:2, preferably 1:2 to 3:2.

An especially preferred embodiment of the binder polymer of Formula IA comprises Formula V which is a linear polyester with terminal alkoxysilyl groups:

(RO)₃Si—(CH₂)_{c}—NHCONH—R¹⁰—NHCOO—[—(CH₂)ₑ—O—CO—(R²⁰)—COO—]_{g}—(CH₂)ₑ—OCONH—R¹⁰—NHCONH—(CH₂)_{c}Si(OR)₃ Formula V

wherein c is an integer of 3-6, preferably 3, e is an integer of 2 to 8, preferably ethylene, butane or hexane diol, more preferably ethylene, R²⁰ is divalent benzenenyl (i.e., the divalent benzene residue of any benzene dicarboxylic acid including phthalic, isophthalic and terephthalic acids) or (CH₂)_{f} wherein f is an integer of 4 to 8, preferably R²⁰ is a terephthalic acid residue or a succinic or adipic acid residue, more preferably a terephthalic acid residue, g is an integer of 10 to 300,000, R¹⁰ is a C4 to C8 alkylenyl group, preferably hexylenyl and R is methyl or ethyl.

The preferred weight average molecular weight of preferred versions of Formulas IA, IB and V may be in the range of about 5KDa to about 200 KDa, preferably about 5 KDa to about 50 KDa to about 100 KDa.

Versions of the Poly monomer displaying flexible alkylenyl groups and stiff aromatic groups, and block combinations for Poly can enable development of hard and soft domains within the film formed of the binder polymer and pretreatment components. The flexibility of long alkylenyl group and the rigidity of the aromatic groups, as well as the hydrogen bonding between intermolecular carboxyl groups with ester, amine, urethane and/or urethan groups act in part to promote soft and hard domains. The presence of hard and soft domains at least in part contributes to the tensile strength and flexibility to the coating and preferably color coating.

### The Catalyst

The film forming composition further may comprise a catalyst to manage the rate of alkoxysilyl condensation to form Si—O-Si networks. As a base line procedure, contact of the binder polymer with water is sufficient to carry out the condensation. However, water hydrolysis and condensation of alkoxysilyl groups under neutral conditions is extremely slow. See for example the discussion of alkoxysilane condensation in A Issa and A Luyt, Polymers, 2019, 11, 537 et seq. Use of a catalyst to change the pH of the hydrolysis/condensation medium speeds the condensation and an acid medium is preferred. Lewis acid agents such as organosulfate, organophosphate, organozirconium, organo aluminum, organozinc, boron halides, organoboron, mineral acids such as hydrochloric, sulfuric and nitric acids and organic acids such as acetic, oxalic and trifluoroacetic acids are useful for increasing the rate of hydrolysis/condensation. Ammonia and organoamine compounds also are useful especially for the second phase of the process, condensation. Choice of a catalyst may be managed by consideration of the cosmetically acceptable and pharmaceutically acceptable nature of the catalyst. For this reason, an excellent catalyst for this purpose, organotin compounds are not acceptable because of their toxicity. A combination of an acidic agent such as an organophosphate, or organoboronate agent followed by a basic wash with dilute ammonia or an organic amine affects an efficient, rapid condensation of the alkoxysilyl groups of the binder polymer and its combination with the pretreatment composition. Preferable acidic catalysts in this regard include bis(2-ethylhexyl) phosphate ester, bis(acetylacetonate), bis(2-ethylhexyl) sulfate ester, methyl sulfate ester, tri(pentafluorophenyl) boron or mono or di acetoboronate.

### The Single Polyolefin Carboxylic Acid Film Forming Composition

In a second embodiment of the film forming composition, the binder polymer may comprise a unitary organic binder polymer of one or more monomeric units selected from an olefinic carboxylate ester unit, an olefinic carboxamide unit, a carbon-hydrogen olefinic unit, an ester monomeric unit, an amide monomeric unit, a urethane monomeric unit, a urea monomeric unit. The organic binder polymer further comprises at least one pendant and/or terminal binder functional monogroup and preferably at least two binder functional monogroups comprising a pendant and/or terminal carboxylic acid or sulfonic acid group, preferably a carboxylic acid group. For this second embodiment, the pretreatment composition preferably comprises an aminoorganoalkoxysilane in addition to the thiol alkoxysilane compounds. The aminoorganoalkoxysilane delivers amino groups to the condensed pretreatment layer. These amino groups enable electrostatic interaction with the carboxyl groups of the film forming composition. The organic polymer with carboxylic acid or sulfonic acid groups is preferably linear or branched, more preferably linear.

### Binder Polymer

Embodiments of the acid binder polymer with carboxylic acid groups comprise repeating units of a hydrophobic monomer or a hydrophilic monomer or a combination thereof, preferably a combination of the hydrophobic monomer and the hydrophilic monomer in addition to the monomeric units of olefinic carboxylic acid monomer or olefinic sulfonic acid monomer, preferably the olefinic carboxylic acid monomer.

The hydrophobic monomer of this organic polymer embodiment may be selected from one or more of an olefinic carboxylate ester monomer or an olefinic carboxamide monomer, an olefinic sulfonamide monomer, an olefin monomer or any combination thereof.

The olefinic carboxylate ester comprises an ester of an olefinic carboxylic acid and at least one saturated linear or branched C1 to C24 primary or secondary alcohol or a C4 to C24 cyclic or alkylcyclic alcohol.

The olefinic carboxamide monomer comprises an amide of an olefinic carboxylic acid and at least one linear or branched C1 to C24 primary amine.

The olefinic sulfonamide monomer comprises an amide of an olefinic sulfonic acid and at least one linear or branched C1 to C24 primary amine or a cyclic or alkylcyclic C4 to C24 alcohol.

The olefin monomer of the hydrophobic segment of the organic polymer embodiment has the formula:

H₂C═CHR

wherein R is selected from hydrogen, linear or branched alkyl of one to twenty four carbons, unsubstituted phenyl or phenyl substituted by one or more linear or branched alkyl of 1 to twenty four carbons, carboxylic ester of a linear or branched C1 to C214 alkanol, carboxamide of a linear or branched C1 to C24 primary amine; or R is selected from —CR²═CHR¹ wherein R¹ is hydrogen, methyl, ethyl or phenyl and R² is hydrogen or methyl.

The hydrophilic olefinic monomer of this embodiment of the organic polymer may be selected from:
(i) a hydroxyl ester of an olefinic carboxylic acid and a linear or branched alkyl diol of 2 to 24 carbons or a cyclic alkyl diol of 5 to 24 carbons;
(ii) an aminoalkyl ester of an olefinic carboxylic acid and a linear or branched C2-C24 aminoalkyl alcohol or a cyclic C5-C24 aminoalkyl alcohol; (ii) a mercaptoalkyl ester of an olefinic carboxylic acid, and a linear or branched C2-C23 mercaptoalkyl alcohol or a cyclic C5-C24 mercaptoalkyl alcohol;
(iii) a styrene in which the phenyl is substituted by carboxylic ester with methanol, carboxamide of ammonia, sulfonamide, sulfinamide;
(iv) vinyl alcohol;
(v) a polar olefinic compound of the formula H₂C═CHC₆H₄R wherein R is selected from selected from hydroxy, sulfonic acid, sulfinic acid, carboxylic acid, or a polyester polyol group having terminal and/or pendant hydroxyl groups;
(vi) vinylalkylenyltrialkoxysilane in which the alkylenyl group is propylenyl or butylenyl and alkoxy is methoxy or ethoxy so as to provide a pendant alkyltrialkoxysilane group for the binder polymer;
or,
(vii) any combination of (i) to (vi).

The olefinic carboxylic acid of this embodiment of the organic polymer is an alkenoic acid of 3 to 24 carbons or alkendioic acid of 4 to 24 carbons or partially hydrolyzed polyacrylonitile or any combination thereof.

Additional embodiments of the organic polymer may include polymers of olefinic carboxylic acids such as (meth)acrylic acid, crotonic acid, pentadienoic acid (butadienyl carboxylic acid) optionally combined with olefinic acid esters and amides and neutral olefinic monomers. The organic polymer may include units of olefinic carboxylic acid monomers including (meth)acrylic acid, maleic acid, fumaric acid, itaconic acid, crotonic acid, pentenoic acid pentadienoic acid, isoprenoic acid, partially hydrolyzed polyacrylonitile and optional olefinic acid monomer derivatives that are homologs of these olefinic carboxylic acid monomers.

The organic polymer of this second embodiment of the film forming composition may include units of the foregoing olefinic carboxylic acid monomers and in addition may include one or more monomeric units of esters of olefinic carboxylic acid monomers wherein the esterifying alcohol is a linear, branched or cyclic alkyl monoalcohol or diol of 1 to 12 carbons for the linear alkyl group (2 to 12 carbons for the diol), 3 to 12 carbons for the branched alkyl group and 3 to 12 carbons for the cyclic alkyl group, amides of said olefinic carboxylic acid monomers. N-alkyl amides of the olefinic carboxylic acid monomers wherein the alkyl group is a linear, branched or cyclic alkyl group as described for the monoalcohol, N-aminoalkyl amides of the olefinic carboxylic acid monomers wherein the amidating amine is a linear, branched or cyclic alkyl diamine with 2 to 12 carbons in the linear alkyl group, 3 to 12 carbons in the branched alkyl group and 3 to 12 carbons in the cyclic alkyl group. Neutral olefinic monomers including those of the formula: HR¹C═CHR² or HR¹C═CH—CR³═CHR⁴ wherein R¹, R², R3 and R⁴ are each independently selected from hydrogen, linear alkyl of 1 to 6 carbons, branched alkyl of 3 to 6 carbons, cyclic alkyl of 3 to 10 carbons, phenyl, phenyl substituted by methyl, ethyl, OH, CONH₂, COOH, —(CH₂)ₙCOOH, NO₂, CN, SO₃H, SONH₂, pyridyl, O₂CR′ wherein R′ is alkyl of 1 to 3 carbons, vinyl and alkyl vinyl having 1 to 3 carbons in the alkyl group.

Preferred embodiments of the hydrophilic monomer of the organic polymer include olefinic carboxylic acids and sulfonic acids selected from one or more of (meth)acrylic acid, crotonic acid, pentenoic acid, hexenoic acid, maleic acid, fumaric acid, glutaconic acid, itaconic acid, citraconic acid, mesaconic acid, vinyl sulfonic acid or any combination thereof. More preferred olefinic carboxylic acids include (meth)acrylic acid, crotonic acid, vinyl sulfonic acid, maleic acid, fumaric acid and itaconic acid. Most preferred olefinic carboxylic acids include (meth)acrylic acid, crotonic acid, maleic acid and itaconic acid. Especially preferred olefinic carboxylic acids include (meth)acrylic acid and crotonic acid.

Additional preferred embodiments of the hydrophilic monomer of the organic polymer include the preferred hydroxyalkyl esters of the foregoing preferred acids esterified with a C2-C6 diol including ethylene diol, propylene diol, butylene diol, pentylene diol or cyclohexane diol aminoethanol, aminopropanol and aminobutanol. Especially preferred hydroxyalkyl esters include the more preferred olefinic carboxylic acids esterified with any of these C2-C6 diols. More preferred hydroxyalkyl esters include the most preferred olefinic carboxylic acids with ethylene diol, propylene diol or butylene diol.

Additional preferred embodiments of the hydrophilic monomer of the organic polymer include the aminoalkyl esters of the preferred olefinic carboxylic and sulfonic acids esterified with a C2 C4 amino alcohol including amino ethanol, amino propanol and aminobutanol. More preferred aminoalkyl esters include the more preferred olefinic carboxylic acids esterified with amino ethanol or amino propanol.

Additional preferred embodiments of the hydrophilic monomer of the organic polymer include the mercapto alky esters of the preferred olefinic carboxylic and sulfonic acids. The preferred mercapto alcohols for these esters include mercaptoethanol, mercaptopropanol and mercaptobutanol. More preferred mercaptoalkyl esters include the more preferred olefinic carboxylic acids esterified with mercaptoethanol.

Additional preferred embodiments of the hydrophilic monomer of the organic polymer include polar olefinic monomers selected from p-hydroxystyrene, styrene-p-carboxylic acid, o,p-dihydroxystyrene, styrene-p-sulfonic acid and any combination thereof.

Preferred embodiments of the hydrophobic monomer of the organic polymer include the alkyl esters wherein the preferred olefinic carboxylic and sulfonic acids are esterified with a C1 to C8 alcohol including methanol, ethanol, propanol, isopropanol, butanol, isobutanol, pentanol, isopentanol, hexanol, isohexanol, ethylhexanol, cyclohexyl alcohol. More preferred alkyl esters include the more preferred olefinic carboxylic acids esterified with ethanol, propanol, butanol, ethylhexanol or cyclohexyl alcohol. Most preferred alkyl esters include the most preferred olefinic carboxylic acids esterified with ethanol, butanol, ethylhexanol or cyclohexyl alcohol.

Additional preferred embodiments of the hydrophobic monomer of the organic polymer include non-polar olefin monomers selected from ethene, styrene, methylstyrene, ethylstyrene, propylstyrene, butadiene, 1-phenylbutadiene, isoprene or any combination thereof.

Additional organic polymer embodiments may comprise one or more monomer unit(s) comprising one or more functional group(s) selected from the group consisting of sulfate, sulfonate, carboxylate, phosphate, phosphonate groups and mixtures thereof as substitutes for the olefinic carboxylic acids of the hydrophilic monomer of the organic polymer. These monomer units may be combined with the other hydrophilic monomers and with the hydrophobic monomers described above to form additional embodiments of the organic polymer. The functional group(s) may preferably be selected from the group consisting of sulfate, sulfonate, carboxylate groups and mixtures thereof. Additionally, anionic polymers of such monomeric units may be combined with the organic polymer embodiments described above to form a mixture of anionic polymer and organic polymer.

Preferred combinations of the recited species of the hydrophilic monomer and the hydrophobic monomer of the foregoing preferences include any combination of the recited preferred non-polar olefinic monomers, the recited preferred polar olefinic monomers, the recited preferred alkyl esters, the recited preferred hydroxyalkyl esters, the recited preferred aminoalkyl esters, the recited preferred mercapto alkyl esters and the preferred olefinic carboxylic and sulfonic acids. The choice of any combination of these species means selection of the first species of the preferred list of olefinic carboxylic and sulfonic acids, selection of the first species of the preferred list of hydroxy alkyl esters, selection of the first species of the preferred list of amino alkyl esters, selection of the first species of the preferred list of mercapto alkyl esters, selection of the first species of the preferred list of preferred polar olefinic monomers and selection of the first species of the preferred list of non-polar olefinic monomers and combining any two of the selections, any three of the selections, any four of the selections, any five of the selections or combining all six of the selections according to the parameters indicating the amounts of hydrophilic monomer and hydrophobic monomer are to be present in the organic polymer. The choice may also be made in a similar fashion by choosing any species from any preferred list and combining it with any species of any other list or multiple lists to provide all combinations of selections.

An especially preferred organic polymer of this second embodiment of the film forming composition comprises repeating units of at least one olefinic acid monomeric unit and at least one non-acid olefinic monomeric unit selected from any one or more of units a), b) c) and d) and any combination thereof. These non-acid olefinic monomeric units include a) an olefinic carboxylate ester monomer unit, b) an olefinic carboxamide monomer unit, c) a hydrophilic olefinic monomer unit, and d) a lipophilic olefin monomer unit.

For this embodiment of the preferred organic polymer, the olefinic acid monomeric unit is selected from (meth)acrylic acid, crotonic acid, maleic acid, fumaric acid, itaconic acid, gluconic acid, a C5-C10 ethenoic acid or any combination thereof.

For this embodiment of the preferred organic polymer, the olefinic carboxylate ester monomeric unit of group a) is selected from a C1-C30 linear or branched alkyl ester of any of the olefinic acid monomeric units or any combination thereof.

For this embodiment of the preferred organic polymer, the olefinic carboxamide monomeric unit of group b) is selected from an —NH₂, —NR¹H or —NR¹R² amide of any of the olefinic acid monomeric units or any combination thereof wherein R¹ and R² are each independently selected from a C1-C6 linear or branched alkyl.

For this embodiment of the preferred organic polymer, the hydrophilic olefinic monomer of group c) is selected from a hydroxy alkyl ester of the olefinic carboxylic acid monomeric unit and a linear or branched C2-C24 alkyl diol or is an aminoalkyl ester of the olefinic carboxylic acid monomeric unit and a linear or branched amino C2-C24 alkyl alcohol or any combination thereof.

For this embodiment of the preferred organic polymer, the lipophilic olefin monomer unit of group d) is selected from an olefin compound of the Formula OL2

R³HC═CHR⁴ Formula OL2

For this lipophilic olefin monomer unit of Formula OL2, the group R³ is selected from hydrogen, linear or branched alkyl of one to six carbons, unsubstituted phenyl or phenyl substituted by a linear or branched alkyl of 1 to six carbons.

For this lipophilic olefin monomer unit of Formula OL2, R⁴ is selected from hydrogen, linear or branched alkyl of one to six carbons, unsubstituted phenyl or phenyl substituted by a linear or branched alkyl of 1 to six carbons.

Alternatively R⁴ of Formula OL2 is an ethenyl group of the Formula OL2′

—CH═CHR⁵ Formula OL2′

For Formula OL2′, R⁵ is selected from hydrogen, linear or branched alkyl of one to six carbons, unsubstituted phenyl or phenyl substituted by a linear or branched alkyl of 1 to six carbons, methyl or ethyl carboxylate, carboxamide or hydroxyl.

A more especially preferred organic polymer of this second embodiment of the film forming composition comprises repeating units of at least one olefinic acid monomeric unit selected from (meth)acrylic acid, crotonic acid, maleic acid or fumaric acid and the lipophilic olefin monomer unit of Formula OL2 in which R³ and R⁴ are both hydrogen, i.e., Formula OL2 is ethene also known as ethylene. An even more especially preferred organic polymer of this second embodiment comprises repeating units of (meth)acrylic acid and ethylene with the acrylic acid version of the (meth)acrylic acid being preferred.

The organic polymer embodiments generally may have an acid value ranging from zero or 0.01 to about 700, preferably about 1 to about 500, more preferably 2 to 250, most preferably 7-90 with typical acid numbers below approximately 100. Typical hydroxyl content may average approximately 1 to 20 wt % or may be approximately 5-10 wt %. The organic polymer may have a weight average molecular weight in the range of about 2 KDa to about 2 MDa, preferably about 2 KDa to about 100 KDa, more preferably about 2 KDa to about 25 KDa. The organic polymer may have a glass transition temperature of from about −125° C. to about −40° C.

The organic polymer may be constructed with random distribution of the different monomer units along the polymer backbone or may be block copolymers which has blocks of single monomer units or may be a graft copolymer which has one monomer unit forming the polymer backbone and a different monomer unit forming polymeric side chains. The different constructions of polymer provide differing polymer to polymer binding properties and different macromolecular characteristics. The block copolymer can provide regions of hard and soft polymer characteristics. A block copolymer can display crystalline regions and amorphous regions that can enable development of water soluble and water resistant regions. Blocks of differing electronic and lipophilic character can impart an open repulsive character to the polymer so that tightly fit inter-structures are minimized. A grafted polymer or segmented polymer are capable of intertwined conformation and compact molecular dimension so as to enable tightly fitted inter-structures.

### The Dual Polymer Michael Film Forming Composition

The third embodiment of the film forming composition provides that the binder polymer is a dual binder polymer comprising first and second components of the film forming composition. These first and second components are different. The first component may be an organosilicone binder polymer having at least one pendant and/or terminal first binder functional group. The second component of this third embodiment may be a silicone binder polymer having at least one pendant and/or terminal second binder functional group. The first and second binder functional groups of this third embodiment comprise a complementary pair respectively of an alkenoyloxy group and an amine or an alkenoyloxy group and a thiol, also known as Michael addition groups.

More specifically, the first component of this third embodiment comprises a binder organosilicone polymer and a the second component comprises linker polymer that are adapted to combine in situ to crosslink through an Aza-Michael addition. The binder polymer comprises a silicone polymer having pendant and/or terminal α,β-unsaturated alkenoyloxy groups. The linker polymer comprises a silicone polymer having pendant and/or terminal organoamine groups and optional pendant and/or alkoxysilyl groups. Preferably, the binder and linker polymers are linear and/or branched, more preferably linear.

The binder first component and linker second component of this embodiment of the film forming composition are separately maintained until immediately prior to use. The film forming composition is prepared for use for application to keratin fibers by combining and mixing these binder and linker components in media according to the proportional quantities described below. The pigment/color bodies with dispersant may also be combined as described below to form the film forming composition with pigment/color bodies.

### First Component of Binder Polymer

The binder polymer may be a silicone polymer with at least two pendant and/or terminal α,β unsaturated alkenoyloxy groups, and preferably at least two of the α,β unsaturated alkenoyloxy groups are terminal groups. The binder polymer may have a linear or branched, preferably a linear configuration.

In particular, embodiments of the binder polymer of the film forming composition comprise a polydimethylsiloxane-type polymer having at least two or at least three α,β unsaturated alkenoyloxy groups attached to siloxane units of the polymer. The α,β-unsaturated alkenoyloxy groups comprise the Formula EOY:

R¹R²C═CR³COO—R⁴— Formula EOY

In Formula EOY, each of R¹ and R² may independently be hydrogen or a C1-C6 linear or branched alkyl group. Preferably at least one of R¹ and R² is hydrogen. The group R³ may be hydrogen or methyl. The group R⁴ is a part of the connector group that joins Formula EOY to silicon of a siloxane unit of the polydimethylsiloxane-type silicone polymer. The group R⁴ may be a C1-C12 linear alkylenyl group, a linear C3-C12 cycloalkylalkyl or cycloalkyl group, a linear C6-C20 arylalkyl group or C6 to C20 aryl group wherein R⁴ may be optionally substituted in chain by one or more of an ether oxygen, thioether sulfur and/or amine groups or pendantly by hydroxyl groups. The group R⁴ is bonded directly with a silicon atom of a siloxane unit of the dimethylsilxane-type silicone polymer. A preferred embodiment of Formula EOY is H₂C═CR³COO—R⁴-, and a more preferred embodiment is H₂C═CHCOO—R⁴—.

Embodiments of the binder polymer of the film forming composition may be linear and/or branched, preferably linear and comprise a silicone polymer constructed of D and M siloxane monomeric units. Branched forms may include T units (MeSiO₃) in the backbone which form branch junctions for branch chains carrying D and M units, however, linear forms are preferred. The binder silicone polymer comprises Formula I:

(X_{z})SiMe_{3-z}O-(Me₂SiO)ₓ—(XSiMeO)_{y}—(X_{z})SiOMe_{3-z} Formula I

For Formula I, two or more of the D and/or M units are modified with X which is the α,β-unsaturated alkenoyloxy group of Formula EOY. Each of the siloxane units Me₂SiO and Si(X)MeO comprise monomeric siloxane D units with Me being methyl. The terminal units (X_{z})SiMe_{3-z} comprise monomeric siloxane M units. The designator z is zero or 1 so that the terminal units may have a single Formula EOY group or may be a trimethylsiloxane unit. The designator x primarily determines the molecular size of the silicone binder polymer and may range from about 2 to 200,000, preferably from about 5 to about 50,000, more preferably from about 5 to about 1,000. The designator y primarily determines the number of Formula EOY groups in the binder and may range from 0 to about 100, preferably about 2 to about 25, more preferably about 2 to about 20. The sum of designators y and z must at least be 1 and preferably 2 so that Formula I has at least one, preferably at least 2 Formula EOY groups. The designator x primarily determines the length of the linear silicone polymer and may integer range from about 3 to about 200,000, preferably up to about 500, more preferably up to about 200 with exemplary integer sums of up to about 100. The multiple monomeric units of Me₂SiO and (X)SiMeO are randomly distributed in Formula I.

Preferred embodiments of Formula I are those with designator y as zero and z as 1. These embodiments provide binders with terminal Formula EOY only. Additional preferred embodiments are those with designator x as at least 5, designator y as 1 to 5 and z as 1. These embodiments provide binders with terminal Formula EOY's and from 1 to 5 pendant Formula EOY's. Yet other especially preferred embodiments are those with designator x as at least 10, designator y as 2 to 6 and z as zero. These embodiment provide binders with 2 to 6 pendant Formula EOY's and no Formula EOY's as termini. Instead, the binder is terminated with Me₃SiO groups.

Another preferred embodiment of the foregoing preferred embodiments of Formula I has Formula EOY as H₂C═CR³COO—R⁴—. More preferred of these especially preferred embodiments of Formula I (Formula EOY with designations of R¹ and R² as hydrogen) are those in which R⁴ is a linear C2-C8 alkylenyl group and more especially preferably is the R⁴ group as —CH₂CHOH—CH₂—O—(CH₂)ₙ— wherein n is an integer of 1 to 6. Most especially preferably for all of these embodiments of Formula I has Formula EOY as

H₂C═CR³COO—CH₂CHOH—CH₂—O—(CH₂)₃—.

An especially preferred embodiment of the binder polymer of Formula I is Formula IV:

H₂C═CHCOOCH₂CHOH—CH₂—O—(CH₂)_{c}—SiMe₂O—(SiMe₂O)ₘ-[MeSiO—(—(CH₂)_{c}—O—CH₂—CHOH—CH₂OOC—CH═CH₂)]_{g}-(Me₂SiO)ₚ—OSiMe₂-(CH₂)_{c}—O—CH₂—CHOHCH₂OOCCH═CH₂ Formula IV

This especially preferred embodiment of the binder provides Formula EOY as terminal groups and as pendant groups of the polydimethylsiloxane-type polymer. In this embodiment, designator c determines the length of the alkylenyloxo group connecting the α,β-unsaturated alkenoyloxy group to silicon of the polymer backbone. Designator c may be an integer of 1 to 6, preferably 3. The designators m and p establish the size or length of the linear silicone polymer and separate the pendant α,β-unsaturated alkenoyloxy groups from the terminal α,β-unsaturated alkenoyloxy groups. Designator m and p may each independently range from about 5 to about 100. Designator g establishes how many pendant α,β-unsaturated alkenoyloxy groups are present in this embodiment of the binder. Designator g may be zero which provides a Formula IV embodiment with no pendant α,β-unsaturated alkenoyloxy groups but with termini each with an α,β-unsaturated alkenoyloxy group. Designator g may alternatively be an integer of from 1 to about 10. In this embodiment, the —O—(CH₂)_{c}-moiety connects Formula EOY to the silicone backbone as a carbon to silicon bond. To accomplish the joinder, a route through an alkenyl moiety may be followed. An alkenyloxoalkyl bromide may be combined with a silicon halide using an alkyl lithium or Grignard reagent to provide an Si-alkyloxoalkene moiety. The olefin bond of the alkene group may be epoxidized and the epoxy group combined with the α,β-unsaturated alkenoic acid such as acrylic acid to form Formula EOY.

Preferred embodiments of Formula IV include those with g as an integer of from 1 to 5 and c and c′ each as an integer of 1 to 3 and each of m and p as 10 to 50. This embodiment provides a binder with Formula EOY as the termini and as 1 to 5 pendant groups. Another preferred embodiment of Formula IV provides g and p as zero, and c and c′ as an integer of 1 to 3. This embodiment provides a binder with Formula EOY as termini only.

### Second Component Linker Polymer

The embodiments of the second component linker polymer of the film forming composition may be linear and/or branched, preferably linear and comprise a polydimethylsiloxane-type silicone polymer comprising a combination of M1 units, D units and M2 units as Formula V:

M1-(D)_{d}-M2 Formula V

The M1 and M2 units constitute the termini of the silicone polymer as indicated. The D units form the backbone of the silicone polymer as indicated. Branched forms may include T units (MeSiO₃) in the backbone which form branch junctions for branch chains carrying D and M units, however, linear forms are preferred. The M1 and M2 units are selected from Me₃SiO units, A-SiMe₂O units in which A is an organoamine group and —SiOR₃ units (trialkoxysilyl units) in which R is ethyl or methyl. The D units are selected from SiMe₂O units and A-SiMeO units. For the D units, the designator d indicates the length of the linear silicone polymer and may range from 3 to 30,000, preferably 3 to 25,000, more preferably 2,000 to 10,000.

For the D and M units, the A moiety, an organoamine group, comprises Formula OA:

H₂N—(R¹⁰—NH)ᵣ—R¹¹— Formula OA

The group R¹⁰ may be a linear or branched C1-C10 alkylenyl group or a linear or branched C6-C14 alkylarylenyl group, preferably a linear C2-C4 alkylenyl group, more preferably an ethylenyl group. The group R¹¹ may be a linear or branched C1-C10 alkylenyl group or a linear or branched C6-C14 alkylarylenyl group, preferably a linear C2-C5 alkylenyl group, more preferably a propylenyl or iso-butyl group. The designator r may be zero or an integer of 1 to 3. The group R¹¹ is bonded to silicon of a siloxane unit and is also bonded to H₂N— when designator r is zero.

A first embodiment of the linker may have both M1 and M2 units as A-SiMe₂O units. A second embodiment of the linker may have M1 as an A-SiMe₂O unit and M2 as an —SiOR₃ unit. For these first and second embodiments D may have a multiple number of SiMe₂O units. For these first and second embodiments, D may alternatively have 1 to 10 A-SiMeO units and a multiple number of SiMe₂O units. A third embodiment of the linker may have both of M1 and M2 as —SiOR₃ units and D may have 1 to 10 A-SiMeO units and a multiple number of SiMe₂G units. A fourth embodiment of the linker may have both of M1 and M2 as Me₃SiO units and D may have 1 to 10 A-SiMeO units and a multiple number of SiMe₂G units.

Preferred embodiments of the linker as M₁-(D)_{d}-M₂ may be selected to provide at least 2 pendant and/or terminal D, M1 and M2 units with Formula OA groups and no trialkoxysilyl groups. Preferred embodiments may be also selected to provide at least one D unit and one of the M1 and M2 units with Formula OA groups and the other of the M1 and M2 units as a trialkoxysilyl group. Preferred embodiments may also be selected to provide at least 2 D units with Formula OA groups and the M1 and M2 units both as trialkoxysilyl groups. More preferred versions of these preceding preferred embodiments may also be selected to provide additional D units with from 2 to 6 Formula OA groups. Especially preferred versions of these preceding preferred and more preferred embodiments may be selected to provide Formula OA groups only in D units and trialkoxysilyl groups as both of the M1 and M2 units.

Formula V as the preferably linear polydimethylsiloxane-type polymeric linker may be expanded to show the monomeric units possible. Accordingly, the linker is formed from the following list of monomeric units with the M and D designations shown below the list:

| (Me₃SiO) | (Si(OR)₃ | (A-SiMe₂O) | (SiMe₂O)ₒ | (SiMeO-A)ₚ |
|---|---|---|---|---|
| M-T1 | M-T2 | M-T3 | D-B1 | D-B2 |

The first three units form termini (M-T1-M-T2-M-T3) for the linker. The last two units form the backbone of the linker (D-B1-D-B2) with the majority of the backbone units being the dimethylsiloxane unit, D-B1. The designator o for the dimethylsiloxane unit is an integer of from 2 to 100. The designator p for the siloxane backbone groups carrying Formula OA may be zero or an integer of from 1 to 10.

The symbol A stands for Formula OA described above. M-T3 and D-B2 carry the amine group Formula OA. Unit M-T2 is the trialkylsilyl group. As a terminus, silicon of the M-T2 unit is bound to oxygen of the adjacent D-B1 unit of the backbone of the dimethylsiloxane-type polymer forming the linker polymer.

As described above, embodiments of the linker may be ordered according to the identity of the group as the termini. In all of these embodiments the silicone backbone primarily is the D-B1 unit. The number of D-B1 units in the backbone is calculated to provide the weight average molecular weight range of the linker as described below. These embodiments include but are not limited to:
A) Both termini as M-T1 (trimethylsiloxane) in which case the backbone will carry at least one unit of the amine Formula OA as D-B2, and preferably two or three D-B2 units.
B) One terminus as M-T1 and the other terminus as M-T2 (alkoxysilyl). In this case, the backbone will carry at least one of the amine Formula OA as unit D-B2 and preferably two or three D-B2 units.
C) One terminus as M-T1 and the other as M-T3 (M unit carrying organoamine Formula OA) in which case the backbone will carry at least one amine Formula OA as unit D-B2 and preferably two or three D-B2 units.
D) One terminus as M-T2 (alkoxysilyl) and the other as M-T3 (M unit carrying organoamine Formula OA) in which case the backbone will carry at least one amine Formula OA as unit D-B2 and preferably two or three D-B2 units.
E) Both termini as M-T2 (alkoxysilyl) in which case the backbone will carry at least one amine Formula IA as unit D-B2 and preferably two or three D-B2 units.
F) Both termini as M-T3 (M unit carrying organoamine Formula OA) in which case the backbone may have no D-B2 units.
G) Both termini as M-T3 in which case the backbone may carry at least one or two D-B2 units.

Embodiments of the binder polymer of the film forming composition may have their designators chosen to indicate a number of siloxane units providing a weight average molecular weight for the binder in a range of about 0.5 KDa to about 10 KDa, preferably about 0.5 KDa to about 5 KDa, more preferably about 1 KDa to about 5 KDa, most preferably about 1 KDa to about 3 KDa., especially about 1KDa to about 2 KDa.

Embodiments of the linker polymer of the film forming composition may have the number of their monomeric units chosen to provide a weight average molecular weight for the linker in a range of about 5KDa to about 50 KDa, preferably about 5 KDa to about 30 KDa, more preferably about 5 KDa to about 20 KDa, most preferably about 8 KDa to about 20 KDa, especially about 10 KDa to about 20 KDa. The weight average molecular weight of the linker will primarily be provided by the number of dimethylsiloxane units present in backbone of the polydimethylsiloxane-type silicone linker.

The binder polymer and linker polymer molar concentrations in the film forming composition deliver a ratio of Michael to aza groups. In some embodiments of the film forming composition, the binder provides a number of α,β unsaturated alkenoyloxy groups (Michael groups) equal to the number of organoamine groups (aza groups) of the linker. In preferred embodiments of the film forming composition, the binder provides an excess number of Michael groups relative to the number of aza groups of the linker. This ratio enables Michael-aza addition of the binder with the amine groups of the small molecule of the pretreatment composition. In more preferred embodiments of the film forming composition, the binder provides at least 2 to 8 Michael groups per 2 to 6 aza groups of the linker.

### The Dual Polymer Carboxyl-Carbodiimide Film Forming Composition

The fourth embodiment of the film forming composition comprises a dual binder polymer comprising first and second binder components that are different. The first component may be an organic, silicon or oganosilicon binder polymer having at least one pendant and/or terminal first binder functional group. The second component of this fourth embodiment may be a small molecule, a prepolymer or polymer having at least one pendant and/or terminal second binder functional group. The first and second binder functional groups of this fourth embodiment comprise a complementary pair respectively of a carboxylic acid group and a carbodiimide group.

The first and second components of this fourth embodiment of the film forming composition are adapted to combine in situ to crosslink through a carboxylic acid—carbodiimide (acid-CDI) addition. The first component binder polymer comprises an olefinic polymer, silicone polymer or olefinic-silicone block copolymer having at least 2 pendant and/or terminal carboxylic acid groups. The binder is preferably linear or branched, more preferably linear. The second linker polymer comprises an alkylenyl, aromatic or alkylenyl aromatic polymer having multiple in chain segments of carbodiimide; or a polymer of ester, urethane or urea monomeric residues having pendant alkylenyl single carbodiimide groups. The linker is preferably linear or branched, more preferably linear.

The binder polymer and linker polymer components of the film forming composition are separately maintained until immediately prior to use. The film forming composition is prepared for use for application to keratin fibers by combining and mixing the binder and linker components in media according to the proportional quantities described below. The pigment/color bodies with dispersant may also be combined as described below to form the film forming composition with pigment/color bodies.

### First Component Binder Polymer

The first component binder polymer may be a homopolymer, a copolymer, a terpolymer or a multiple block polymer having at least two carboxylic acid groups. Moreover, the construction of the binder polymer may be an organic polymer, a silicone polymer or organosilicone polymer, each of which is configured to have a linear and/or branched configuration, preferably a linear configuration.

In particular, embodiments of the first component binder polymer of the film forming composition comprise an olefinic, silicone or organosilicone polymer of Formula I having at least two carboxylic acid groups.

MUE-(MU1)ₓ-(MUX)_{y}-(MU2)_{z}-(MU3)ₐ-(MU3X)_{b}-MUE Formula I

The symbols MUE, MU1, MUX, MU2, MU3 and MU3X stand for monomeric units of the carboxylic acid polymer. The binder polymer of Formula I may be linear or branched, preferably linear. The monomeric units MU1, MUX (X for acid) and MU2 respectively are hydrophobic, acid and hydrophilic olefinic monomeric units. MU3 and MU3X respectively are siloxane units with the X siloxane unit bearing a pendant alkanoic acid group. MUE (E for end) is the termination unit of the polymer and may be any of the olefinic monomeric units or the siloxane unit. An olefinic polymer comprises either or both of MU1 and MU2 combined with MUX and the termini of this polymer (MUE) may be any of these three former monomeric units. If hydrophilic and hydrophobic units are present in the olefinic polymer, these olefinic monomeric units may be randomly distributed throughout the olefin polymer or may form blocks of hydrophilic and hydrophobic units with the carboxylic acid units preferably being within the hydrophilic blocks. A silicone polymer comprises a combination of MU3 and MU3X with its termini being MU3. The carboxylic acid units may be randomly distributed throughout the silicone polymer. An organosilicone polymer comprises blocks of the olefinic polymer and the silicone polymer. The olefinic polymer blocks may have the monomeric units arranged as in the olefinic polymer. The acid containing units may be MUX or MU3X and preferably are MUX. The binder comprising the olefinic, silicone or organosilicone polymer formed of the foregoing monomeric units may linear or branched preferably be linear.

In particular, these monomeric units may be linear or branched, preferably linear and are as follows.
a) MU1 is a hydrophobic olefinic monomeric unit comprising a C2-C10 alkene residue, a C4-C12 alkadiene residue and/or a C6-C10 aromatic/alkylaromatic vinyl residue.
b) MU2 is a hydrophilic olefinic monomeric unit comprising a vinyl C2-C16 alkanoic ester residue, a C1-C14 alkyl or hydroxyalkyl C2-C14 alkenoic ester residue, a C2-C10 alkenoic amide residue or N—C1-C4 alkyl substituted version of the amide residue.
c) MUX is an acidic olefinic monomeric unit comprising a C3-C10 alkenoic acid residue or a C4-C10 alkadienoic acid residue.
d) MU3 is a dimethylsiloxane monomeric unit.
e) MU3X is a monomethylsiloxane monomeric unit bound to an alkanoic acid of at least 4 carbons with one of the alkyl carbons of the alkanoic acid optionally having a hydroxy group.
f) MUE is a single terminal monomeric unit of MU1, MU2 or MUX when the polymer is an olefinic polymer or an organosilicone polymer.
g) MUE is a single terminal monomeric unit of MU3 with an additional methyl, i.e., a trimethylsiloxane unit when the polymer is a silicone polymer.

The designators x, y, z, a and b indicate the number of the corresponding monomeric units present in the corresponding polymer. Irrespective of the kind of polymer, its molecular size is the sum of x, y, z, a and b which may be an integer of from about 3 up to about 1 million, preferably up to about 300,000, more preferably up to about 250,000, most preferably up to about 200,000. Each of the designators x, y, z, a and b independently indicates the number of corresponding monomeric units forming the linear polymeric backbone. Each of x, z and a may be zero or an integer of from 1 up to about 100,000. Designators y and b indicate the number of acid units present in the polymer with y indicating the number of olefinic carboxylic units and b indicating the number of siloxane carboxylic acid units. Designators y and b may each independently be zero or an integer of 1 to 100, preferably 1 to 50, more preferably 1 to 20 provided that at least two carboxylic acid groups are present. Additionally, when the polymer is a silicone polymer b is zero and y is an integer. When the polymer is an olefin polymer, b is an integer and y is zero. When the polymer is an organosilicone polymer one of b and y may be zero and the other an integer or both may be an integer.

Preferred forms of Formula I include:

Formula I in which the designators x and z are each at least 10, designator y is at least 3, designators a and b are both zero and terminal MUE is MUX. This is the olefinic polymer.

Formula I in which each of designators x, z and a are 10 to 100, designator y is 1 to 50, designator b is zero, terminal MUE is MUX. This is the organosilicone block copolymer with olefinic unit carboxylic acid groups.

Formula I in which designators x, y and z are zero, designator a is at least 20, preferably at least 40, designator b is 1 to 50 and terminal MUE is MU3X or as MU3. This is the silicone polymer with termini as either dimethylsiloxane bearing an alkylalkanoic acid group or a trimethylsiloxane unit.

Formula I in which each of designators x, z and a are 10 to 100, each of designators y and b independently is 1 to 50, terminal MUE is MUX or MU3. This is the organosilicone polymer with olefinic and siloxane units bearing the carboxylic acid.

A preferred binder polymer comprises an olefinic or organosilicone polymer with three or more pendant and/or terminal carboxylic acid groups, a weight average molecular weight of from about 0.5 KDa to about 10 KDa, preferably about 0.5 KDa to about 5 KDa; and at least one or more pendant groups selected from an alkyl alkylenylcarboxyate ester group, an alkyl group, an alkylenyloxycarbonylalkyl group and a hydroxalkyl group.

A preferred binder polymer also comprises a silicone polymer with three or more pendant C4-C6 alkanoic acid groups and a weight average molecular weight of from about 0.5 KDa to about 10 KDa, preferably about 0.5 KDa to about 5 KDa.

Another preferred binder polymer of Formula I comprises an olefin polymer of from 3 to 10, preferably 3 to 5 carboxylic acid groups, a weight average molecular weight of from about 0.5 KDa to about 10 KDa, preferably about 0.5 KDa to about 5 KDa; and in which MU1 is butene, pentene, hexene, styrene or any combination thereof, MUX is (meth)acrylic acid, crotonic acid, pentenoic acid, hexenoic acid, fumaric acid, maleic acid, itaconic acid glutaconic acid, citraconic acid or mesaconic acid, preferably (meth)acrylic acid, maleic acid, fumaric acid or crotonic acid; MU2 is vinyl acetate, vinyl propanate, vinyl butanate, C1-C3 alkyl or hydroxyalkyl (meth)acrylate, C1-C3 alkyl or hydroxyalkyl crotonate, C1-C3 alkyl or hydroxyalkyl pentanoate, C1-C3 dialkyl or di-(hydroxyalkyl) fumarate, C1-C3 maleate or the corresponding primary amides or C1-C3 alkyl secondary amides or any combination thereof.

Another preferred binder polymer of Formula I comprises a silicone polymer of from 3 to 10, preferably 3 to 5 carboxylic acid groups; the weight average molecular weight of from about 0.5 KDa to about 10 KDa, preferably about 0.5 KDa to about 5 KDa; and in which MU3X is MeSiO —(CH₂)ₙ—CHOH—(CH₂)₂—COOH with n as an integer of from 1 to 6, preferably 2 or 3.

Another preferred binder polymer of Formula I comprises an olefin polymer of from 3 to 10, preferably 3 to 5 carboxylic acid groups, a weight average molecular weight of from about 0.5 KDa to about 10 KDa, preferably about 0.5 KDa to about 5 and in which MU1 is hexene or styrene, MUX is (meth)acrylic acid or crotonic acid, MU2 is vinyl acetate, vinyl C8-C12 isoalkanoate, methyl, ethyl or isopropyl (meth)acrylate or the corresponding hydroxymethyl, hydroxyethyl or hydroxyisopropyl analogs, methyl, ethyl or isopropyl crotonate or the corresponding hydroxymethyl, hydroxyethyl or hydroxyisopropyl analogs.

Another preferred binder polymer of Formula I is an organosilicone block copolymer with carboxylic acid groups in the olefin block. The designators of this preferred binder include designator x as zero meaning no hydrophobic olefinic units, designator b as zero meaning no acid groups pendant to siloxane units, designator a as at least 10 meaning at least 10 dimethylsiloxane units, designator z as at least 10 meaning at least 10 hydrophilic olefinic units, designatory y as 1 to 50 meaning 1 to 50 carboxylic acid olefinic units and MUE is MUX meaning terminal olefinic carboxylic acid units.

Another preferred binder polymer of Formula I is an olefinic polymer comprising at least monomeric units of alkyl (meth)acrylate and/or crotonate, and (meth)acrylic acid and/or crotonic acid. The acid number of this polymer is from about 50 to about 600 preferably about 100 to about 400.

A more preferred binder polymer of Formula I is an olefinic polymer in which the acid monomeric unit is (meth)acrylic acid and/or crotonic acid at about 0.3% to about 75% by weight; the hydrophilic unit is hydroxyethyl or hydroxypropyl (meth)acrylate and/or crotonate at about 0% to about 20% by weight; the hydrophobic monomer is methyl or ethyl (meth)acrylate and/or crotonate at about 5% to about 20% by weight, wherein all weights are relative to the total weight of the polymer.

Exemplary olefinic polymers as the binder polymer include organic copolymers such as acrylic acid/ethyl acrylate/N-tert-butylacrylamide terpolymers such as the product sold under the name Ultrahold 8 and that sold under the name Ultrahold Strong by the company BASF; (meth)acrylic acid/tert-butyl (meth)acrylate and/or isobutyl (meth)acrylate/C1-C4 alkyl (meth)acrylate copolymers such as the acrylic acid/tert-butyl acrylate/ethyl acrylate terpolymer sold by the company BASF under the name Luvimer 100P; (meth)acrylic acid/ethyl acrylate/methyl methacrylate terpolymers and tetrapolymers such as the ethyl acrylate/methyl methacrylate/acrylic acid/methacrylic acid copolymer such as the product sold under the name Amerhold DR-25 by the company Amerchol; methyl methacrylate/butyl or ethyl acrylate/hydroxyethyl or 2-hydroxypropyl acrylate or methacrylate/(meth)acrylic acid tetrapolymers such as the methyl methacrylate/butyl acrylate/hydroxyethyl methacrylate/methacrylic acid tetrapolymers sold by the company Rohm & Haas under the name Acudyne 255.

Additional examples of organic polymers as binder polymer include copolymers of acrylic acid and of C1-C4 alkyl methacrylate and terpolymers of vinylpyrrolidone, of acrylic acid and of C1-C20 alkyl, for example lauryl, methacrylate, such as that sold by the company ISP under the name Acrylidone M and the copolymer of methacrylic acid and of ethyl acrylate sold under the name Luvimer MAEX by the company BASF.

Exemplary silicone polymers bearing pendant carboxylic acid groups as the binder polymer include dual-end carboxy silicones such as X-22-162C from Shin Etsu and Silform INX (INCI name: Bis-Carboxydecyl Dimethicone) from Momentive; single-end carboxy silicone such as X-22-3710 from Shin Etsu. and other carboxy silicones such as Grandsil PCA such as in Grandsil SiW-PCA-10 (INCI name: Dimethicone (and) PCA Dimethicone (and) Butylene Glycol (and) Decyl Glucoside from Grant Industries.

Exemplary organosilicone polymers as the binder polymer include multi-block carboxysilicone polymer (tradename Belsil® P1101) having INCI name: Crotonic Acid/Vinyl C8-12 Isoalkyl Esters/VA/Bis-Vinyldimethicone Crosspolymer and a similar organosilicone polymer having the technical name of Crotonic Acid/Vinyl C8-12 Isoalkyl Esters/VA/divinyldimethicone Crosspolymer from Wacker Chemie AG.

Additional exemplary silicone and organosilicone polymer functioning as the binder polymer include name HUILE M 642 by the company Wacker, under the names SLM 23 000/1 and SLM 23 000/2 by the company Wacker, under the name 176-12057 by the company General Electric, under the name FZ 3703 by the company OSI and under the name BY 16 880 by the company Toray Silicone as well as Noveon under the name Ultrasil® CA-1 Silicone (Dimethicone PEG-7 Phthalate) and Ultrasil® CA-2 Silicone (Dimethicone PEG-7 Succinate).

### Second Component Linker Polymer

Embodiments of the second component linker polymer of the film forming composition comprise an organic polymer of Formula II which is a polymer with in-chain carbodiimide groups and may be linear or branched, preferably linear. Alternatively, the linker may comprise an organic polymer of Formula X which is a polymer with pendant single carbodiimide groups and may have a linear or branched backbone, preferably a linear backbone.

Z-(L-N═C═N—)ₚ—Z Formula II

(Poly)_{q}-(K)ₛ-(Poly)ᵣ Formula X

For Formula II, p is an integer of at least 2. In many instances, L may be the organic group of an organic diisocyanate which is converted to the polycarbodiimide of Formula II. In other instances, L may be an oligomeric or polymeric moiety terminated by an isocyanate group which is converted to a carbodiimide by combination with another isocyanate group or converted to a urethanyl group by reaction with Z. This formational understanding shows that L may be an organic linker group comprising a saturated aliphatic divalent radical, an aromatic divalent radical or an alkylaromatic divalent radical or a polymer or oligomeric divalent radical with repeating olefinic, carbonate, ester, ether, amide, urethane or urea linkages. Preferably, L is a saturated aliphatic divalent radical, an aromatic divalent radical or an alkylaromatic divalent radical.

For Formula X, each Poly is an organic polymer segment of amide, urea, ester, olefinic, imine monomeric residues. Poly may be based upon a C3 to C6 alkane diamine and a C4-C10 alkane dicarboxylic acid or C4-C10 alkane diisocyanate, or an ester monomeric residue based upon a C3-C6 alkane diol and a C4-C10 alkane dicarboxylic acid and the designators q and r each being an integer of at least 2. Group K provides the pendant carbodiimide group and s is an integer of at least 2. When s is 2 or greater, the resulting multiple K groups are randomly distributed along the Poly backbone including at the termini. Group K comprises Formula XI

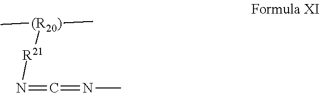

For Formula XI, R²⁰ is a C3 to C6 alkylenyl residue and R²¹ is a C3-C6 alkylenyl residue.

For Formulas II and XI, Z may be a non-reactive or reactive terminal group of the polycarbodiimide. As a reactive terminal group, Z may be an —(CH₂)ₙ—Si(OR)₃ in which R is methyl or ethyl and n is an integer of 3 to 6. As a non-reactive terminal group, Z may be a saturated aliphatic monovalent radical, an aromatic monovalent radical or an alkylaromatic monovalent radical.

A preferred linker polymer is Formula II in which L is a saturated aliphatic divalent radical selected from linear or branched or cyclic alkylenyl of 2 to 20 carbons, an aromatic divalent radical selected from benzene or diphenyl, or an alkylaromatic divalent radical selected from p-dimethylenylphenyl or methylenyldiphenyl.

Another preferred linker polymer is Formula II in which L is a saturated alkylenyl divalent radical of 2 to 6 carbons.

Another preferred linker polymer is Formula II in which L is a residue of toluene, diphenylmethane, phenyl, dicyclohexyl methane, methyl-3,5,5,-trimethylcyclohexane, hexane, cyclohexane, norbornane. These L residues are derived from the corresponding diisocyanate compounds.

A more preferred linker polymer is Formula II in which L is dicyclohexylmethane, methyl-3,5,5-trimethylcyclohexane (isophorone) or hexane.

For Formula II and Formula X, a preferred a nonreactive group for Z is a saturated aliphatic monovalent radical selected from linear or branched or cyclic alkyl of 2 to 20 carbons, an aromatic monovalent radical selected from benzene or diphenyl, or an alkylaromatic monovalent radical selected from p-dimethylenylphenyl or methylenyldiphenyl.

A preferred linker polymer as Formula X provides Poly is a polyamide or polyurea and the number of pendant carbodiimide K groups designated by being from 2 to 50, preferably 2 to 10, more preferably 2 to 5.

A further preferred linker polymer is Formula X, R²⁰ and R²¹ are each butylenyl or hexylenyl, and Z is butyl or hexyl.

A preferred nonreactive group of Z for Formulas II and X is butane or hexane.

The molecular size of a linker polymer of Formula II and of Formula X is determined by the number of carbodiimide groups and the size of L for Formula II and Poly for Formula X. For both of Formulas II and X, the preferred number of carbodiimide groups designated by p and k respectively is from 2 to 100, preferably from 2 to 50, more preferably from 2 to 10, most preferably 2 to 5. The foregoing preferred L groups (non-polymeric L groups for Formula II) provide the molecular size for these preferred versions of Formula II. For Formula X, the preferred Poly is polyamide formed of hexane diamine and adipic acid with the pendant K groups formed from 3-aminopropyl-1,6-hexane diamine. Based upon factors such as but not limited to the number of L groups, the size of Poly and the number of carbodiimide groups the weight average molecular weight of the linker may range from 0.5KDa to about 500 KDa, preferably about 0.5 KDa to about 400 KDa, more preferably about 0.5 KDa to about 10 KDa, most preferably about 0.5 KDa to about 3KDa to 5 KDa.

The binder polymer and linker polymer molar concentrations and their relative level of functional groups in the film forming composition deliver a ratio of carboxylic acid to carbodiimide groups. In some embodiments of the film forming composition, the binder provides a number of carboxylic acid groups equal to the number of carbodiimide of the linker. In preferred embodiments of the film forming composition, the linker provides an excess number of carbodiimide groups relative to the number of carboxylic acid groups of the binder. This ratio enables carbodiimide addition of the linker with the amine groups of the small molecule of the pretreatment composition. In more preferred embodiments of the film forming composition, the ratio of linker carbodiimide groups to binder carboxylic acid groups may range from about 50:1 to 1.2:1, preferably about 30:1 to 2:1, more preferably about 25:1 to 2.5:1, especially more preferably about 20:1 to about 3:1, most preferably about 20:1 to about 10:1.

### Medium

When applied to keratin fibers, the media of the film forming composition and pre-treatment composition embodiments of the invention may be an organic compound that is capable of being intimately mixed or preferably forming a solution with a minor amount of water. The preferred media comprise embodiments of alcoholic solvents such as an alkyl alcohol of 1 to 6 carbons with no intentionally added water. Included are methanol, ethanol, propanol, isopropanol n-butanol, isobutanol, pentanol, neopentanol, isopentanol and n-hexanol. Preferred organic alcohols include ethanol, n-propanol, isopropanol, ethylene glycol, propylene glycol, n-butanol and isobutanol and pentanol. The alcoholic solvent may be intentionally combined with a minor amount of water in amounts up to about 10 weight percent, preferably up to about 5 weight percent, more preferably up to about 2 or 3 weight percent and most preferably less than about 1 weight percent relative to the total weight of the media. It is recognized that alcohol solvents absorb water from the atmosphere so that an alcoholic solvent with no intentionally added water may contain a slight amount of water. Although it is not a limitation of the invention, it is believed that the presence of water molecules facilitate the condensation of the alkoxysilyl groups to silyloxysilyl groups of the film forming and pretreatment compositions.

Additionally, the medium for the pretreatment composition may include a minor amount of acetic acid, such as from about 0.1 wt % to about 2 wt %, preferably from about 0.1 wt % to about 1 wt %, more preferably from about 0.1 wt % to about 0.5 wt % relative to the total weight of the medium. Alternatively, a 90%-95% alcoholic-aqueous medium with ethanol and acetic acid may be used for the pretreatment composition. The presence of acetic acid facilitates hydrolysis of the alkoxysilyl groups to hydroxysilyl groups and renders the small molecule of the pretreatment composition more soluble in water. When the minor amount of acetic acid is present in the aqueous-alcoholic pretreatment composition, the lifetime of the pretreatment composition is on the order of on the order of a few hours. Consequently, this option for application of the pretreatment composition is typically conducted in small batches which are mixed and immediately used. The medium for the pretreatment composition may also include a balance among the amounts of alcohol, water and acetic acid present relative to the identity of the small molecule present. In some instances, the acid and/or water concentrations may be greater than in others. Determination of appropriate and/or optimum ratios of concentrations for the individual pretreat components, the choice and amount of medium and the presence and amount of acid or alkali are within the ordinary experimental ability and technique of the laboratory technician. Guidelines include a pretreat component concentration providing a viscosity enabling a free flowing liquid that will not readily run off keratin fibers when applied, no intentionally included water or a very minimal amount if needed to promote condensation,

The pretreatment composition, the first and second components or unitary film forming component (collapsed first and second components having the same functional binder group) and other reactive or catalytic components of the film forming composition are maintained separately until use. Packaging each in separate containers serves this purpose. Each of the components of the film forming composition, and the pretreatment composition may be maintained in a medium that does not interact with the reactive groups. Suitable media are non-aqueous organic solvents such as but not limited to the alcohols mentioned above, preferably isopropanol and isobutanol or liquid hydrocarbon or silicone solvents. The media for separately maintaining these components should not include water or agents that would hydrolyze or otherwise react with functional binder groups. Typically, the pretreatment and film forming components may be formulated as ready to use concentrations or may be concentrates which are to be diluted with appropriate media to prepare them for use or may be ready to use concentrations for application to keratin fibers.

When the film forming composition and the pretreatment composition are prepared for application to keratin fibers, they may be formulated with a single phase alcohol or alcohol medium as described above or may be formulated as a two phase aqueous medium with water or water-alcohol as the continuous phase and a water or water-alcohol immiscible organic liquid as the discontinuous phase. The continuous phase may carry water soluble constituents while the discontinuous phase may carry constituents such as those of the film forming composition and the PTH alkoxysilane of the pretreatment composition that would react with water. The discontinuous, non-aqueous phase will tend to isolate such compounds from degradation by water. Preferably, in situations when water is to be part of a medium but one or more of the components of the film forming composition and pretreatment composition are sensitive to water, the film forming composition and pretreatment composition are maintained in a non-aqueous environment until they are ready for dressing on keratin fibers. At the application stage, single phase or a two phase medium may be prepared as appropriate.

The polarity and protonic character of the medium are important for control of the several reactions that occur when the components of the film forming composition and pretreatment composition are combined. These reactions include the alkoxysilyl group condensation and the complementary pair of first and second functional binder groups including the Michael complementary pair and the carboxylic acid-carbodiimide pair. Preferably, the medium for application of the film forming composition and the pre-treatment composition is polar and can support the condensation and addition reactions. For both of the film forming composition and pretreatment composition, isopropanol or isobutanol with a minor amount of water as described above is appropriate. The application media may be combined with the separately stored concentrates of binder, catalyst and small molecule and the media of the stored concentrates preferably will be at least partially to substantially miscible with the application media.

The medium may be independently present in each of the film forming composition and the pretreatment composition in an amount ranging from about 0.1% to about 99% by weight, such as from about 1% to about 98% by weight, for example ranging from 50% to 95% by weight relative to the total weight of which of the film forming composition and pretreatment composition is under consideration. The concentrations for the components of the pretreatment composition and the film forming composition are discussed in the following sections.

### Viscosity, Compositional Constituent Concentrations

The viscosities of the film forming composition and pretreatment composition function to hold them in place on the keratin fibers while the coating and preferably color coating is formed. The viscosity substantially avoids free translational flow of these compositions. Free translation flow would cause the compositions to rapidly run and drip off the surfaces of the hair strands. Nevertheless, the viscosity is not so high that it will not undergo self-leveling to substantially uniformly coat the keratin fibers. Appropriate viscosity of the compositions is the result of the interaction of the various constituents of the film forming and pretreatment compositions, their concentrations, the pigment microparticles, and as appropriate, an optional viscosity control agent, an optional suspending agent and an optional thickening agent. Nevertheless, a viscosity approximating a flowable liquid such as but not limited to ethanol or isopropanol is appropriate when applied with an appropriate application device.

Generally, the viscosity of the film forming and pretreatment compositions may range from about 0.001 to about 2000 Pa s⁻¹. Viscosity measurements are carried out on a controlled stress rheometer e.g. Using an AR2000 type manufactured by TA Instruments, or equivalent instrument. A 6 cm flat acrylic cross hatched parallel plate geometry (TA item 518600.901) and a stainless steel cross hatched base plate (TA item 570011.001) are used. The rheometer is prepared for flow measurements as per standard manufacturer procedure. The parallel plate geometry gap is set to 1000 microns. The flow procedure is programmed to the rheometer with the following conditions: continuous stress ramp 0.1-300 Pa over 2 minutes at 25° C., including 250 measurement points in linear mode. The product is loaded into the geometry as per standard procedure and the measurement commences at 5 min after the mixture preparation. Shear stress value at 10 sec⁻¹ shear rate is obtained from the shear stress vs. shear rate curve, and the corresponding viscosity is calculated by dividing the obtained shear stress by 10.

The concentration of the first and second components of the film forming composition of the concentration of the unitary component of the film forming composition and the concentration of the PTH alkoxysilane constituent in the pretreatment composition may each independently range from about 0.1% to about 90%, preferably about 1% to about 40%, more preferably about 2% to about 30%, most preferably about 2% to about 15% and especially most preferably about 1% to about 10% by weight relative to the total weight of the composition. As discussed above, the viscosity is managed so that the film forming and pretreatment compositions will not readily run off the surfaces of strands of hair yet will level and flow relatively freely to substantially coat those surfaces. Development of appropriate viscosity in part by management of the concentrations of the constituents of the film forming and pretreatment compositions can be experimentally determined by routine methods such as formulation of several samples of differing concentrations of constituents in these compositions, coating those samples on a hair tress and observing the flow, spread and leveling of the composition on the hair strands.

The film forming and pretreatment compositions can be applied simultaneously, sequentially or in pre-combined form to keratin fibers such as a hair tress using the coloring procedure described herein afterwards. The top of the hair strand, where it is glued together is fixed such that the hair is aligned vertically downwards. After a 5 minute dwell time it is observed if any and how much product has dripped from the hair tress. The results obtained from the several samples can be plotted against flow time and leveling time to determine an appropriate concentration or range of concentrations of the constituents of the film forming and pretreatment compositions.

The extent of in situ linking between and among the reactive constituents of the film forming and pretreatment compositions may be controlled by manipulation of ratios, amounts present and concentrations as well as by physical means as described above so that the mechanical and chemical properties of the coating and preferably color coating as described herein are preserved. These properties include ability to adhere to hair strands, ability to maintain flexibility and free flowing character of the hair, ability to provide remanence, avoidance of stickiness and avoidance of clumping.

The glass transition temperatures of the polymer(s) produced by the film forming composition and the molecules of the pretreatment composition in part contribute to the flexibility, strength, hardness and similar qualities of the coating and preferably color coating on the keratin fiber surfaces. The glass transition temperatures of these embodiments preferably are well below ordinary minimum environmental temperatures such as −100° C. to +100° C. The glass transition temperature or T_{g} determines the solid-solid transition of the polymer from a hard glassy material to a soft rubbery material. For the purposes of the coating and preferably color coating on keratin fibers, the soft, rubbery, elastic state is the state to be achieved. This is an undesirable result. The coating should be soft, flexible, elastic and unnoticeable to touch and sight yet should not flake, break-up or otherwise release from the keratin fiber, and especially from anagenic hair, when stroked by a hand or brushed with a brush. The Tg of a coating and preferably color coating can be measured using ASTM D7426—08 (2008).

### Solute Content

Embodiments of the film forming composition and pretreatment composition are combinations of a medium which contains liquids and solids and so that these compositions generally comprise solvent and solute. The solvent is the medium which is volatile at ambient conditions; in other words, the medium is a liquid and functions as a solvent and/or a liquid in which solutes are dissolved and/or dispersed. The solute or solutes comprise all components and substances except the medium such as at least the liquid, gel and solid components of these compositions that remain after the medium is removed. Included as solute at least are the polymers, oligomers, pretreat compounds, associated catalyst/promotor materials, if any, and the pigment microparticles and colorants of these compositions. The optional components include the plasticizer, dispersing agent, surface treatment agent, cross linking agent and other materials which may be added to the medium. These optional components, if any, are included in the solute content as long as they remain with the polymers, oligomers, pretreat compounds, reaction materials and pigment microparticles following application and setting of the color composition and pretreatment composition as a coating on strands of human hair. This includes all substances of the pretreatment and film forming compositions that ordinarily would be regarded as liquids, gels and/or solids because they would remain in the coating on strands of hair.

The solute content of the film forming composition and pretreatment composition may range from about 0.1 wt % to about 50 wt % relative to the total weight of the respective composition. A preferred solute content ranges from about 0.1 wt % to about 20 wt % and a more preferred solute content ranges from about 0.2 wt % to about 12 wt % relative to the total weight of the composition. An especially preferred solute content range is about 0.3 wt % to about 11 wt % with contents of about 1 wt %, about 2 wt %, about 3 wt %, about 4 wt %, about 5 wt %, about 6 wt %, about 7 wt % and about 8 wt % being more especially preferred.

Because the solutes of the pretreatment composition are the pretreat molecule, the foregoing solute content also indicates the concentration of pretreat molecule in the pretreatment composition medium with the especially preferred solute content range being the preferred concentration of pretreat molecule in the medium of the pretreatment composition.

The film forming composition includes the first and second components or the unitary component, additives and the pigment or color bodies when the film forming composition is ready for application to keratin fibers. Except for the pigments, these components may be liquids, gels or solids depending upon the individual nature of each component and each constitutes a portion of the total solute content of the film forming composition ready for use. When the film forming composition is maintained separately as the binder and medium, as catalyst and medium and pigment and medium, the especially preferred solute content range given above is the preferred concentration of the first and second components or unitary component of the film forming composition in medium when stored in a separate container. The concentration of pigment in dispersant and medium in a separate container usually has a much higher concentration and in preparation for combination into the film forming composition, several of the different pigments are combined and diluted as described herein to form the coloration factor of the film forming composition. A catalyst, if present, is also in a low solute concentration range as described above when the catalyst is in storage. It is typically combined with the binder in medium just prior to application to keratin fibers.

The ratio of the concentration of first and second components or unitary component binder to PTH alkoxysilane compound (pretreat compound) and other optional additives upon application of the pretreatment composition and film forming composition to keratin fibers is adjusted to provide at least equal alkoxysilyl group and/or other interactive group molar equivalent amounts to assure that the components of pretreatment and film forming compositions will not only condense and/or react with themselves but also with each other. Preferably, the molar equivalent amount of the PTH alkoxysilane compound is larger by from 2% to 20%, preferably 3% to 10%, more preferably from about 5% to about 10% relative to the molar equivalent amount of the film forming components. The concentrations and molar equivalent amounts designed to deliver concentrations of from 2 wt % to 4 wt % of the pretreat compound in the pretreatment composition and the components in the film forming composition and preferably a molar equivalent excess of pretreat compound relative to the binder are calculated and measured into delivery containers so that application of a metered container of the pretreatment composition onto a portion of hair followed by application of a metered container of film forming composition onto the same portion of hair will deliver the desired results of binder and pretreat molecule inter-condensation.

### Plasticizer

If the glass transition temperatures of the coating and preferably color coating and/or the constituents of the film forming and pretreatment compositions are too high for the desired use yet the other properties thereof are appropriate, such as but not limited to color and remanence, one or more plasticizers can be combined with the constituents of the film forming and pretreatment composition embodiments so as to lower the T_{g} of the constituents and provide the appropriate feel and visual properties to the coating and preferably color coating. The plasticizer can be incorporated directly into one or both of the film forming and pretreatment compositions or can be applied to the hair following formation on the keratin fibers of the color composition of the combined film forming and pretreatment compositions but substantially curing the color composition to form the coating and preferably color coating on the keratin fibers. The plasticizer can be chosen from the plasticizers typically used in the field of application. Appropriate selection includes choice of a plasticizer that does not interfere with or compete with the alkoxysilyl condensation or the complementary pair reaction.

The plasticizer or plasticizers can have a molecular mass of less than or equal to 5,000 g/mol, such as less than or equal to 2,000 g/mol, for example less than or equal to 1,000 g/mol, such as less than or equal to 900 g/mol. In at least one embodiment, the plasticizer, for example, has a molecular mass of greater than or equal to 40 g/mol.

Thus, the film forming and pretreatment compositions can also comprise at least one plasticizer. For example, non-limiting mention can be made, alone or as a mixture, of common plasticizers such as: glycols and derivatives thereof, silicones, silicone polyethers, polyesterpolyols; adipic acid esters (such as diisodecyladipate), trimellitic acid esters, sebacic acid esters, azalaeic acid esters; nonlimiting examples of glycol derivatives are diethylene glycol ethyl ether, diethylene glycol methyl ether, diethylene glycol butyl ether or diethylene glycol hexyl ether, ethylene glycol ethyl ether, ethylene glycol butyl ether, or ethylene glycol hexyl ether; polyethylene glycols, polypropylene glycols, polyethylene glycol-polypropylene glycol copolymers, and mixtures thereof, such as high molecular weight polypropylene glycols, for example having a molecular mass ranging from 500 to 15,000, for instance glycol esters; propylene glycol derivatives such as propylene glycol phenyl ether, propylene glycol diacetate, dipropylene glycol ethyl ether, tripropylene glycol methyl ether, diethylene glycol methyl ether, and dipropylene glycol butyl ether. Such compounds are sold by Dow Chemical under the names DOWANOL PPH and DOWANOL DPnB; acid esters, for example esters of carboxylic acids, such as triacids, citrates, phthalates, adipates, carbonates, tartrates, phosphates, and sebacates; esters derived from the reaction of a monocarboxylic acid of formula R₁₁COOH with a diol of formula HOR₁₂OH in which R₁₁ and R₁₂, which can be identical or different, are chosen from a linear, branched or cyclic, saturated, or unsaturated hydrocarbon-based chain containing, for example, from 3 to 15 carbon atoms for example the monoesters resulting from the reaction of isobutyric acid and octanediol such as 2,2,4-trimethyl-1,3-pentanediol, such as the product sold under the reference TEXANOL ESTER ALCOHOL by the company Eastman Chemical; oxyethylenated derivatives, such as oxyethylenated oils, such as plant oils, such as castor oil; mixtures thereof.

Among the esters of tricarboxylic acids mention can be made of the esters of triacids wherein the triacid corresponds to formula

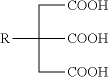

wherein R is a group —H, —OH or —OCOR′ wherein R′ is an alkyl group containing from 1 to 6 carbon atoms. For example, R can be a group —OCOCH₃. The esterifying alcohol for such tricarboxylic acids may be those described above for the monocarboxylic acid esters.

The plasticizer can be present in either or both of the film forming and pretreatment compositions in an amount from about 0.01% to 20%.

### Pigment

The film forming and pretreatment composition embodiments of the present invention make it possible to obtain the preferable colored coatings, without substantially altering the keratin fibers. As used herein, colorant includes the terms “pigment(s) and color body(ies)”. The term pigment generally refers to any particulate colorant or amorphous insoluble color material/body having or containing pigment material that gives keratin fibers color including black and white, such as titanium dioxide that gives only white color to keratin fibers. The pigments are substantially water-insoluble. Color bodies generally refer to partially soluble to soluble color materials such as soluble dyes, Henna, Indigo, anthrocyanin and other similar soluble color compounds. The pigments, to distinguish from dyes presented in molecular form, are also referred to as pigment microparticles or pigment particles. The terms pigment microparticles and pigment particles are synonymous and are used herein interchangeably. The pigments can be organic, inorganic, or a combination of both. The pigments may be in pure form or coated, for example with a polymer or a dispersant.

Selections, multiple kinds and varying forms of the pigment microparticles as described in the following passages can be incorporated in any of the first, second and third components of the multicomponent composition, or can be incorporated in any two of these components or in all three. Preferably, pigment microparticles can be incorporated in either or both of the first and second components. More preferably, pigment particles can be incorporated in the first component.

The at least one pigment that can be used can be chosen from the organic and/or mineral pigments known in the art, such as those described in Kirk-Othmer's Encyclopedia of Chemical Technology and in Ullmann's Encyclopedia of Industrial Chemistry. The pigments comprised in the microparticles comprising at least one pigment will not substantially diffuse or dissolve into keratin fibers. Instead, the pigment comprised in the microparticles comprising at least one pigment will substantially remain separate from but attached to the keratin fibers.

The at least one pigment can be in the form of powder or of pigmentary paste. It can be coated or uncoated. The at least one pigment can be chosen, for example, from mineral pigments, organic pigments, elemental metal and their oxides, and other metal modifications, lakes, pigments with special effects such as nacres or glitter flakes, and mixtures thereof.

### Pigment Shape

The pigment microparticles can have any suitable shape, including substantially spherical. But the pigment microparticles can also be oval, elliptical, tubular, irregular, etc., or even combinations of various shapes. In addition, the pigment microparticles can have two dimensions, length and width/diameter, of similar magnitude. In addition, the pigment microparticles can be micro platelets, i.e. having a thickness that is substantially smaller than the planar dimension. For example, five, ten or even 20 times smaller in thickness than in the planer dimension. In one embodiment with any of the reactive components of the instant invention, the pigments may be surface treated, surface coated or encapsulated.

### Pigment Size

The pigments can be present in the composition in undissolved form. Depending on the shape, the pigments can have a D50[vol] particle diameter of from 0.001 micron to 1 micron.

According to an embodiment, the particle size distribution, either relative to the number or volume of the particles, of the pigment microparticles can be at least bi-modal. A bi-modal particle size distribution has two distinct peaks which are spaced relative from, while tri-modal particle size distribution has three distinct peaks. The term “peak” means a local maximum of the distribution curve. The “distance” between two peaks, expressed relative to the particle size, can be at least 0.05 micron, preferably at least 0.1 micron, such as at least 0.2 micron. Providing an at least bi-modal particle size distribution allows to tailor the optical appearance of the colored hair. For example, the scattering properties varies with the particle size so that particles of different size scatter the light into different directions.

Pigments made from metal and metal like materials which can conduct electricity, and which can absorb light and re-emit the light out of the metal to give the appearance of strong reflectance. While not wishing to be bound by any specific theory, it is believed that the absorbed light will induce alternating electric currents on the metal surface, and that this currents immediately re-emit light out of the metal. Such pigment microparticles can be platelets, e.g., having a thickness that is substantially smaller than the planar dimension. For example, about five, about 10 or even about 400 times smaller in thickness than in the planer. Such platelets can have a planar dimension less than about 30 nm, but with a thickness less than about 10 micron wide. This includes a ratio of 10000 to 30, or 333. Platelets larger in size, such as 50 microns are even available in this thickness of 10 microns, and so the ratios can even go up to 2000.

The pigment microparticles can be a composite formed by two different types of pigment microparticles. Examples include a composite of a 2-dimensional microparticle and at least one micro spherical particle (microsphere), a composite of different micro spherical particles, and a composite of different 2-dimensional particles. Composite particles formed by 2-dimensional microparticles to which micro spherical particles adhere provide an attractive alternative to a pure mixture of 2-dimensional microparticles and micro spherical particles. For example, a metallic 2-dimensional microparticle can carry one or more micro spherical particle such as one or more organic micro spherical particle. The micro spherical particles attached or bonded to the 2-dimensional microparticle can be formed of the same pigment material or can be formed of different pigment material. Composite microparticles formed of 2-dimensional microparticles and micro spherical particles can provide multiple functionality in one particle such as (metallic) reflectance and dielectric scattering, reflectance and absorption.

The pigment microparticles can be both light scattering and absorbing for wavelengths of visible light. While not wishing to bound by any specific theory, it is believed that such pigments can provide a visual effect of lightening the hair. Such pigment microparticles can have a D50[num] value between about 50 nm and about 750 nm, between about 100 nm and about 500 nm or between about 150 nm and about 400 nm. Such materials have a refractive index above about 1.5, above about 1.7 or above about 2.0.

According to an embodiment, different pigment microparticles are combined to provide reflective, transmitting and refractive properties of the hair colored with the color composition described herein. A microparticle combination can be a material composite using at least two different pigment materials to form the pigment microparticles. In addition to, or alternating to, the microparticle combination, a mixture of separate pigment microparticles of different type can be used to bring about the desired reflective, transmitting and refractive properties.

The composite pigments, combination of pigments, and mixtures of pigment microparticles eliminate, or at least significantly reduce, hair penetration and scattering by light and thus eliminate the perception of pigment of natural hair color change.

### Pigment Concentration

The film forming composition for coloring hair fibers according to the present disclosure comprises microparticles comprising at least one pigment. The film forming composition comprises from about 0.01% to about 40%, about 0.05% to about 35%, about 0.1 to about 25%, or about 0.15% and about 20% pigment(s), by weight of the film forming composition.

### Pigment Material

The material of the pigment microparticles can be inorganic or organic. Inorganic-organic mixed pigments are also possible.

According to an embodiment, inorganic pigment(s) may be used. The advantage of inorganic pigment(s) is their excellent resistance to light, weather, and temperature. The inorganic pigment(s) can be of natural origin, and are, for example, derived from material selected from the group consisting of chalk, ochre, umber, green earth, burnt sienna, and graphite. The pigment(s) can preferably be white pigments, such as, for example, titanium dioxide or zinc oxide. The pigment(s) can also be colored pigments, such as, for example, ultramarine or iron oxide red, luster pigments, metal effect pigments, pearlescent pigments, and fluorescent or phosphorescent pigments. The pigment(s) can be selected from the group consisting of metal oxides, hydroxides and oxide hydrates, mixed phase pigments, sulfur-containing silicates, metal sulfides, complex metal cyanides, metal sulfates, chromates and molybdates, alloys, and the metals themselves. The pigment(s) can be selected from the group consisting of titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI 77289), Prussian blue (ferric ferrocyanide, CI 77510), carmine (cochineal), zinc sulfide, barium sulfate, zinc oxide, derivatized titanium dioxide, derivatized zinc sulfide, derivatized zinc oxide, and mixtures thereof. The pigment(s) can be selected from the group consisting of iron oxide, titanium dioxide, mica, borosilicate, and combinations thereof. The pigment(s) can comprise an iron oxide (Fe₂O₃) pigment. The pigment(s) can comprise a combination of mica and titanium dioxide.

The pigment(s) can be pearlescent and colored pigment(s) and can preferably be based on mica which are coated with a metal oxide or a metal oxychloride, such as titanium dioxide or bismuth oxychloride, and optionally further color-imparting substances, such as iron oxides, Prussian blue, ultramarine, and carmine. The color exhibited by a pigment can be adjusted by varying the layer thickness. Such pigments are sold, for example, under the trade names Rona®, Colorona®, Dichrona®, RonaFlair®, Ronastar®, Xirona® and Timiron® all of which are available from Merck, Darmstadt, Germany. For example, Xirona® is a brand for color travel pigments that display color shifting effects depending on the viewing angle and are based on either natural mica, SiO₂ or calcium aluminum borosilicate flakes, coated with varying layers of TiO₂. Pigment(s) from the line KTZ® from Kobo Products, Inc., 3474 So. Clinton Ave., So. Plainfield, USA, are also useful herein, in particular the Surface Treatable KTZ® Pearlescent Pigments from Kobo. Particularly useful are KTZ® FINE WHITE (mica and TiO₂) having a D50 particle diameter of 5 to 25 micron and also KTZ® CELESTIAL LUSTER (mica and TiO₂, 10 to 60 micron) as well as KTZ® CLASSIC WHITE (mica and TiO₂, 10 to 60 micron). Also useful are SynCrystal Sapphire from Eckart Effect Pigments, which is a blue powder comprising platelets of synthetic fluorphlogopite coated with titanium dioxide, ferric ferrocyanide and small amounts of tin oxide. Also useful is SYNCRYSTAL Almond also from Eckart, which is a beige powder with a copper reflection color and is composed of platelets of synthetic fluorphlogopite and coated with titanium dioxide and iron oxides. Also useful is Duocrome® RV 524C from BASF, which provides a two color look via a lustrous red powder with a violet reflection powder due to its composition of mica, titanium dioxide and carmine. The colored pigment(s) can be lightly bright colored pigment(s) and can particularly be white color variations.

The pigment(s) can be organic pigments. The at least one pigment can be an organic pigment. As used herein, the term “organic pigment” means any pigment that satisfies the definition in Ullmann's encyclopedia in the chapter on organic pigments. For instance, the at least one organic pigment can be chosen from nitroso, nitro, azo, xanthene, quinoline, anthraquinone, phthalocyanin, copper phthalocyanin, copper hexadecachlorophthalocyanine, 2-[(2-Methoxy-4-nitrophenyl)azo]-N-(2-methoxyphenyl)-3-oxobutyramide, metal-complex, isoindolinone, isoindoline, quinacridone, perinone, perylene, diketopyrrolopyrrole, thioindigo, dioxazine, triphenylmethane, dimethylquinacridone and quinophthalone compounds, Azo-dyes, Nonionic azo dyes, Anionic Azo dyes, Cationic azo dyes, Complex forming azo dye, aza annulene dyes, aza analogue of diarylmethane dyes, aza annulene dyes, Nitro-dyes and their pigments, Carbonyl dyes and their pigments (for example, Anthrachinon dyes, indigo), Sulfur dyes, Florescence dyes, Anthracene or Insoluble alkali or earth metal acid dyes. Or the pigment can be at least one of uncolored and UV absorbing.

The organic pigment(s) can be selected from the group consisting of natural pigments sepia, gamboge, bone charcoal, Cassel brown, indigo, chlorophyll and other plant pigments. The synthetic organic pigments can be selected from the group consisting of azo pigments, anthraquinoids, indigoids, dioxazine, quinacridone, phthalocyanine, isoindolinone, perylene and perinone, metal complex, alkali blue, diketopyrrolopyrrole pigments, and combinations thereof. A particularly preferred pigment is 7-Bis(1,3-dichloropropan-2-yl)benzo[lmn][3,8]phenanthrolin-1,3,6,8(2H,7H)-tetraon.

The pigment(s) used in the color composition can include at least two different pigments selected from the above pigment group or can include at least three different pigments selected from the above pigment group. According to an embodiment, the pigment(s) used in the color composition can include at least one yellow pigment selected from the yellow pigment group consisting of: a Pigment Yellow 83 (CI 21108), CAS #5567-15-7, Pigment Yellow 155 (C.I. 200310), (CAS: 68516-73-4), Pigment Yellow 180 (C.I. 21290), (CAS: 77804-81-0).

In addition to the at least one yellow pigment, or alternatively, the pigments(s) used in the color composition can include at least one red pigment selected from the red pigment group consisting of: Pigment Red 5 (CI 12490), (CAS #6410-41-9), Pigment Red 112 (CI 12370), (CAS #6535-46-2), Pigment Red 122 (CI 73915), (CAS #980-26-7).

In addition to the at least one yellow pigment and/or the at least one red pigment, or alternatively, the pigments(s) used in the color composition can include at least one green pigment selected from the green pigment group consisting of: Pigment Green 36, (C.I. 74265), (CAS: 14302-13-7).

In addition to the at least one yellow pigment and/or the at least one red pigment and or the at least one green pigment, or alternatively, the pigments(s) used in the color composition can include at least one blue pigment selected from the blue pigment group consisting of: Pigment Blue 16, (CAS: 424827-05-4), Pigment Blue 60 (C.I. 69800), (CAS: 81-77-6), Pigment Blue 66, (C.I. 73000), (CAS: 482-89-3)

In addition to the at least one yellow pigment and/or the at least one red pigment and/or the at least one green pigment, and/or the at least one blue pigment or alternatively, the pigments(s) used in the color composition can include at least one black pigment selected from the black pigment group consisting of: Pigment Black 6 (C.I. 77266), (CAS 1333-86-4), Pigment Black 7 (C.I. 77266), (CAS 1333-86-4). An additional combination can include aluminium flake with a red, blue, green, yellow or any combination thereof.

The pigment(s) can optionally have a surface zeta potential of +15 Mv, preferably ≥±20 Mv, more preferably ≥±25 Mv. The surface zeta potential can be measured with a zetasizer, for example, a Zetasizer 3000 HS. Surface zeta potential measurements are conducted, for example, according to ISO 13099.

For example, the white or colored organic pigments can be chosen from carmine, carbon black, aniline black, melanin, azo yellow, quinacridone, phthalocyanin blue, sorghum red, the blue pigments codified in the Color Index under the references CI 42090, 69800, 69825, 73000, 74100, and 74160, the yellow pigments codified in the Color Index under the references CI 11680, 11710, 15985, 19140, 20040, 21090, 21100, 21108, 47000, 47005 and 77492.

The green pigments codified in the Color Index under the references CI 61565, 61570, 74265, and 74260, the orange pigments codified in the Color Index under the references CI 11725,12075, 15510, 45370, and 71105, the red pigments codified in the Color Index under the references CI 12085, 12120, 12370, 12420, 12490, 14700, 15525, 15580, 15585, 15620, 15630, 15800, 15850, 15865, 15880, 17200, 26100, 45380, 45410, 45430, 58000, 73360, 73915, 75470, and 77491 and the pigments obtained by oxidative polymerization of indole or phenolic derivatives.

Non-limiting examples that can also be mentioned include pigmentary pastes of organic pigments, such as the products sold by the company Hoechst under the names: JAUNE COSMENYL IOG: Pigment Yellow 3 (CI 11710); JAUNE COSMENYL G: Pigment Yellow 1 (CI 11680); ORANGE COSMENYL GR: Pigment Orange 43 (CI 71105); ROUGE COSMENYL R: Pigment Red 4 (CI 12085); CARMINE COSMENYL FB: Pigment Red 5 (CI 12490); VIOLET COSMENYL RL: Pigment Violet 23 (CI 51319); BLEU COSMENYL A2R: Pigment Blue 15.1 (CI 74160); VERT COSMENYL GG: Pigment Green 7 (CI 74260); and NOIR COSMENYL R: Pigment Black 7 (CI 77266).

The at least one pigment in accordance with the present disclosure can also be in the form of at least one composite pigment as described in European Patent Publication No. EP 1 184 426 A2. These composite pigments can be, for example, compounds of particles comprising a mineral core, at least one binder for ensuring the binding of the organic pigments to the core, and at least one organic pigment at least partially covering the core.

The at least one pigment in accordance with the present disclosure can be in the form of small undissolved microparticles, which do not diffuse into the hair color, but deposit on the outer wall of the keratin fiber. Suitable color pigments can be of organic and/or inorganic origin. But the pigments can also be inorganic color pigments, given the excellent light, weather and/or temperature resistance thereof.

Inorganic pigments, whether natural or synthetic in origin, include those produced from chalk, red ocher, umbra, green earth, burnt sienna or graphite, for example. Furthermore, it is possible to use black pigments, such as iron oxide black, color pigments such as ultramarine or iron oxide red, and fluorescent or phosphorescent pigments as inorganic color pigments.

Colored metal oxides, metal hydroxides and metal oxide hydrates, mixed phase pigments, sulfurous silicates, silicates, metal sulfides, complex metal cyanides, metal sulfates, metal chromates and/or metal molybdates are particularly suitable. In particular, preferred color pigments are black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI 77289), iron blue (ferric ferrocyanide, CI 77510) and/or carmine (cochineal).

The at least one pigment can also be colored pearlescent pigments. These are usually mica-based and can be coated with one or more metal oxides from the group consisting of titanium dioxide (CI 77891), black iron oxide (CI 77499), yellow iron oxide (CI 77492), red and brown iron oxide (CI 77491, CI 77499), manganese violet (CI 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, CI 77510).

Mica forms part of the phyllosilicates, including muscovite, phlogopite, paragonite, biotite, lepidolite, and margarite. To produce the pearlescent pigments in combination with metal oxides, the mica, primarily muscovite or phlogopite, is coated with a metal oxide.

The at least one pigment can also be at least one mica-based colored pigment, which is coated with one or more metal oxides from the group consisting of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, Pigment Blue 29), chromium oxide hydrate (CI 77289), chromium oxide (CI 77288) and/or iron blue (ferric ferrocyanide, CI 77510).

The at least one pigment can also be color pigments commercially available, for example, under the trade names Rona®, Colorona®, Dichrona® and Timiron® from Merck, Ariabel® and Unipure® from Sensient, Prestige® from Eckart Cosmetic Colors, and Sunshine® from Sunstar.

The at least one pigment can also be color pigments bearing the trade name Colorona® are, for example: Colorona Copper, Merck, MICA, C1 77491 (IRON OXIDES); Colorona Passion Orange, Merck, Mica, CI 77491 (Iron Oxides), Alumina; Colorona Patina Silver, Merck, MICA, CI 77499 (IRON OXIDES), CI 77891 (TITANIUM DIOXIDE); Colorona RY, Merck, CI 77891 (TITANIUM DIOXIDE), MICA, CI 75470 (CARMINE); Colorona Oriental Beige, Merck, MICA, CI 77891 (TITANIUM DIOXIDE), CI 77491 (IRON OXIDES); Colorona Dark Blue, Merck, MICA, TITANIUM DIOXIDE, FERRIC FERROCYANIDE; Colorona Chameleon, Merck, CI 77491 (IRON OXIDES), MICA; Colorona Aborigine Amber, Merck, MICA, CI 77499 (IRON OXIDES), CI 77891 (TITANIUM DIOXIDE); Colorona Blackstar Blue, Merck, CI 77499 (IRON OXIDES), MICA; Colorona Patagonian Purple, Merck, MICA, CI 77491 (IRON OXIDES), CI 77891 (TITANIUM DIOXIDE), CI 77510 (FERRIC FERROCYANIDE); Colorona Red Brown, Merck, MICA, CI 77491 (IRON OXIDES), CI 77891 (TITANIUM DIOXIDE); Colorona Russet, Merck, CI 77491 (TITANIUM DIOXIDE), MICA, CI 77891 (IRON OXIDES); Colorona Imperial Red, Merck, MICA, TITANIUM DIOXIDE (CI 77891), D&C RED NO. 30 (CI 73360); Colorona Majestic Green, Merck, CI 77891 (TITANIUM DIOXIDE), MICA, CI 77288 (CHROMIUM OXIDE GREENS); Colorona Light Blue, Merck, MICA, TITANIUM DIOXIDE (CI 77891), FERRIC FERROCYANIDE (CI 77510); Colorona Red Gold, Merck, MICA, CI 77891 (TITANIUM DIOXIDE), CI 77491 (IRON); Colorona Gold Plus MP 25, Merck, MICA, TITANIUM DIOXIDE (CI 77891), IRON OXIDES (CI 77491); Colorona Carmine Red, Merck, MICA, TITANIUM DIOXIDE, CARMINE Colorona Blackstar Green, Merck, MICA, CI 77499 (IRON OXIDES); Colorona Bordeaux, Merck, MICA, CI 77491 (IRON OXIDES); Colorona Bronze, Merck, MICA, CI 77491 (IRON OXIDES); Colorona Bronze Fine, Merck, MICA, CI 77491 (IRON OXIDES); Colorona Fine Gold MP 20, Merck, MICA, CI 77891 (TITANIUM DIOXIDE), CI 77491 (IRON OXIDES); Colorona Sienna Fine, Merck, CI 77491 (IRON OXIDES), MICA Colorona Sienna, Merck, MICA, CI 77491 (IRON OXIDES); Colorona Precious Gold, Merck, Mica, CI 77891 (Titanium dioxide), Silica, CI 77491 (Iron oxides), Tin oxide; Colorona Sun Gold Sparkle MP 29, Merck, MICA, TITANIUM DIOXIDE, IRON OXIDES, MICA, CI 77891, CI 77491 (EU); Colorona Mica Black, Merck, CI 77499 (Iron oxides), Mica, CI 77891 (Titanium dioxide) Colorona Bright Gold, Merck, Mica, CI 77891 (Titanium dioxide), CI 77491 (Iron oxides); Colorona Blackstar Gold, Merck, MICA, CI 77499 (IRON OXIDES); color pigments bearing the trade name Unipure® are, for example: Unipure Red LC 381 EM, Sensient C1 77491 (Iron Oxides), Silica; Unipure Black LC 989 EM, Sensient, CI 77499 (Iron Oxides), Silica; Unipure Yellow LC 182 EM, Sensient, CI 77492 (Iron Oxides), Silica.

Among the dyes, non-limiting mention can be made of cochineal carmine. Non-limiting mention can also be made of the dyes known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 1 O (CI 77 002), D&C Green 3 (CI 42 053), and D&C Blue 1 (CI 42 090). A non-limiting example of a lake that can be mentioned is the product known under the following name: D&C Red 7 (CI 15 850:1).

### Color Gamut for Pigment Blends

CIE L*a*b* (CIELAB) is a color space specified by the International Commission on Illumination. It describes all the colors visible to the human eye and serves as a device-independent model to be used as a reference.

The three coordinates of CIELAB represent the lightness of the color (L*=0 yields black and L*=100 indicates diffuse white; specular white may be higher), its position between red/magenta and green (a*, negative values indicate green while positive values indicate magenta) and its position between yellow and blue (b*, negative values indicate blue and positive values indicate yellow).

Since the L*a*b* model is a three-dimensional model, it can be represented properly only in a three-dimensional space. Two-dimensional depictions include chromaticity diagrams: sections of the color solid with a fixed lightness.

Because the red-green and yellow-blue opponent channels are computed as differences of lightness transformations of (putative) cone responses, CIELAB is a chromatic value color space.

In the present invention, the color gamut is determined by preparing samples of the film forming composition without pigment and the adding each pigment to be tested to individual samples of the film forming composition. Samples with pigment are prepared and applied to tress substrates. The samples are cured and then tested for coloration so that the resulting CIELAB lightness or L* value of the colored hair is 60±2. The level of pigment needed will depend on the pigment being tested. For example, hair tresses (Kerling, Natural White special quality) can be prepared as described using samples of a film forming composition applied alone as described in the present invention. A Minolta spectrophotometer CM-2600d can be used to measure the color of the cured and dried hair tresses, five points on both the front and back sides, and the values averaged. The D65 L*a*b values can be calculated. When at least three pigments have each been measured such that their resulting colors reside within the target L* values of 60±2 the color gamut can be calculated. First the lengths of each side of the resulting triangle of each combination of three pigments in the a*b plane are computed using the following expressions. To calculate the distance between pigments 1 and pigment 2 the following equation is used:

$$Side Length {SL}_{12} = {( {( {a}_{pigment 1} - {a}_{pigment 2} )}^{2} + {( {b}_{pigment 1} - {b}_{pigment 2} )}^{2} )}^{0.5} .$$

This is computed for each pair of pigments. Then for a series of three pigments.

The resulting color gamut is calculated using the expression:

$$Color Gamut = {( S ( S - S {L}_{12} ) ( S - S {L}_{13} ) ( S - S {L}_{23} ) )}^{0.5}$$

wherein SL₁₂, SL₁₃, and SL₂₃ are the three lengths of the sides of the triangle within the a*b plane, and S=(SL₁₂+SL₁₃+SL₂₃)/2. Where more than three pigments are used, this calculation can be performed for each combination of the three pigment from the more than three pigments used, and the largest Color Gamut is selected.

The preferable color coating embodiments of the present invention can also have a color gamut of greater than 250, greater than 500, greater than 750, greater than 800, greater than 900, greater than 1100 or even greater than 1250.

### Experiments Performed for Color Gamut

Using the above expression, for each combination of three pigments possible from Color Gamut Tables 1, as illustrated below, the color gamut at a nominal L value of 60 was calculated.

TABLE 1
Color Gamut

| | | | wt % | | | |
|---|---|---|---|---|---|---|
| Pigment | Name | Supplier | level | L | a | b |
| Blue 15 | PV Fast Blue | Clariant | 0.155 | 59.3 | −18.7 | −2.1 |
| | BG-NIP | | | | | |
| Blue 16 | Phthalocyanine | Carbosynth | 0.280 | 59.4 | −17.3 | 1.5 |
| Blue 66 | Indigo 229296 | Aldrich | 0.105 | 60.0 | −3.1 | 6.8 |
| Blue 60 | Paliogen Blau L | BASF | 0.260 | 60.7 | −3.9 | 5.9 |
| | 6482 | | | | | |
| Black 7 | Midnight Black | Geotech | 0.045 | 59.8 | 0.0 | 12.3 |
| Green | Heliogen Green | BASF | 0.509 | 60.1 | −32.8 | 20.2 |
| 36 | K 9362 | | | | | |
| Red 112 | Permanent Red | Clariant | 0.150 | 60.1 | 29.8 | 18.8 |
| | FGR 250 | | | | | |
| Red 122 | Hostaperm Pink | Clariant | 0.140 | 59.5 | 24.9 | 6.1 |
| | E02-EDW VP4034 | | | | | |
| Violet | Ink Jet Magenta | Clariant | 0.200 | 60.6 | 28.1 | 10.1 |
| 19 | E5B 02 M250 | | | | | |
| Red 5 | Permanent Carmine | Clariant | 0.140 | 59.7 | 30.1 | 14.4 |
| | FB01 | | | | | |
| Yellow | Ink Jet Yellow | Clariant | 16.92 | 61.8 | 9.6 | 74.4 |
| 155 | 4GC | | | | | |
| Yellow | Novoperm Yellow | Clariant | 1.059 | 60.0 | 12.5 | 61.8 |
| 83 | HR 70 | | | | | |
| Yellow | Toner Yellow HG | Clariant | 9.16 | 61.4 | 11.2 | 72.8 |
| 180 | | | | | | |

These were formulated within an example formulation described later using an appropriate level of first, second and third compositions.

In an embodiment, a color composition (e.g., a set of a film forming composition and a pretreatment composition applied sequentially, simultaneously or in premixture to keratin fibers) can be applied to the hair in a sequential manner. For example it may be that this first set is applied to the hair which contains pigment microparticles that substantially scatter and/or reflect light such that it produces the visual effect of making the hair look lighter in color, after which a second set can be applied which contains pigment microparticles that substantially absorbs light and provides color to the hair and wherein the combination of the sequential addition of the first and second sets of color compositions provides the final hair color. For example, a first color composition may comprise metallic flakes and the second color composition may contain organic pigment microparticles. It may also be that more than a first and a second color composition are applied to the hair to achieve the desired color result, that three or more color compositions are applied.

### The pH

The film forming and pretreatment composition embodiments in accordance with the present invention may have a pH adjustment attendant with their application to keratin fibers so that the pH upon application may range from about 4 to about 10, preferably about 5 to about 9. The pH is preferably dynamically managed to control the rate of reaction of the reactive constituents of the film forming and pretreatment compositions. Maintaining a slightly basic pH during the mixing and preapplication stages involving these compositions controls the alkoxysilyl condensation under certain circumstances. The condensation may be initiated by reversion to an acidic pH to an appropriate state for reaction.

### Dispersants

It will be apparent to one skilled in the art that careful and selective choice of dispersant can help to maximize performance in terms of maximizing the amount of color produced from an immobilized film, maximizing the remanence or wash fastness, and enabling removal of the color.

The electrostatic, ionic and functional character of the dispersant is chosen to be compatible with and to not interfere with the reactive constituents of the film forming and pretreatment compositions. More preferably, the dispersant is chosen to be compatible with and miscible with the other components of the composition or compositions with and without medium.

The principle of choosing chemically similar dispersant with the binder polymer of the film forming composition can be followed to ensure maximum compatibility.

As well as compatibility as noted above, the other criterion in selecting dispersant(s) is their ability to enable pigment to be dispersed down to the primary particle size, preferably with the minimum amount of input mechanical energy. It will be recognized by someone skilled in the art that the concentration of dispersing agent is also a factor. In general, it is usually required that there is a minimum amount for dispersing activity and that below this, the composition is either not fully dispersed or the dispersant acts as a flocculant.

These two considerations together are used to define preferred materials and their respective concentrations.

It may also be the case, depending on the type of binder polymer used, that the binder itself is also a dispersant. In such cases it is possible that no further dispersing additive may be needed.

Combination of the dispersed pigment mixture with the film forming composition can be made in any manner. This order of combination of the dispersed pigment mixture with the film forming composition delivers the dispersed pigment mixture with the film forming composition layer and on top of the pretreatment layer. While the layers intermix to a slight to moderate to essentially full extent, at least a portion of the dispersed pigment mixture resides over the pretreatment layer. This arrangement of the coating at least in part enables removal of the coating when the “off” techniques described below are practiced.

### Dispersants, Kinds, Properties and Chemistry

Dispersants are amphiphilic or amphiphathic meaning that they are chemical compounds possessing both hydrophilic (water-loving, polar) and lipophilic (fat-loving) properties. Dispersants are surface-active polymers that allow the homogeneous distribution and stabilization of solids, e.g. pigments in a liquid medium, by lowering the interfacial tension between the two components. As a result, agglomerates are broken up into primary particles and protected by a protecting dispersant envelope of a re-agglomeration.

The dispersants can be subdivided on the basis of the stabilization mechanism in
1. dispersants for electrostatic stabilization
   a. Anionic dispersing additives
      i. Polyacrylates
      ii. Polyphosphates
   b. Neutral dispersing additives, e.g, nonionic surfactants
   c. Cationic dispersing additives, e.g., quaternary ammonium organic and/or silicone polymers
2. Dispersants for steric stabilization

### Electrostatic Stabilization

The pigment surface is occupied by an additive carrying an ionic charge. All pigment particles are charged the same. The mutual repulsion by the charge is greater than the attractions of the pigment particles. The electrostatic stabilization has its relevance mostly in water-based paint compositions.
Polyanionic dispersing additives: polycarboxylates (mostly salts of polyacrylic acids), polyphosphates divided into linear polyphosphates and cyclic metaphosphates, polyacrylates
salts of polyacrylic acid, as cations, sodium and ammonium are preferred, these polyacrylates are water-soluble, technical products have molecular weights in the range of 2000 to 20,000 g/mol, optimum is about 8000 g/mol
Sodium and ammonium salts of the homo- or copolymers of acrylic acid, methacrylic acid or maleic acid

### Steric Stabilization

The attractive forces between the pigment particles are effective only over relatively small distances of the particles from each other. The approach of two particles to each other can be prevented by molecules that are firmly anchored to the pigment surface and carry groups that extend from the surface and may reduce the potential for the pigments to contact one another. By sufficiently long chain lengths, agglomeration can be prevented. Also, the substances added to avoid agglomeration and other undesirable pigment particle interactions preferably are chosen to minimize or avoid interaction with the reactive polymers of the color composition.

### Incorporation of Pigment in Dispersant

The pigments described herein can be chosen and/or modified to be similar enough such that a single dispersant can be used. In other instances, where the pigments are different, but compatible, two or more different dispersants can be used. Because of the extreme small size of the pigment microparticles and their affinity, combination of the pigment microparticles and dispersant to form a substantially homogeneous dispersion that can subsequently be modified and/or diluted as desired is to be accomplished before combination with any or all of the film forming composition.

The pigment microparticles can be dispersed and stabilized in the medium by one or more dispersants the properties and kinds of which are described above. The dispersant can either be added to the medium, or to a precursor medium or can form a coating on the microparticles to facilitate dispersion. It is also possible to provide the microparticles with a coating of a dispersant material and additionally provide a further dispersant to the medium, or to a precursor medium, which is used to form the final medium.

The dispersant, either added to the medium or provided as coating, facilitates wetting of the microparticles, dispersing of the microparticles in the medium, and stabilizing of the microparticles in the medium.

The wetting includes replacing of materials, such as air, adsorbed on the surface of the pigment microparticles and inside of agglomerates of the microparticles by the medium. Typically, a complete wetting of the individual microparticles is desired to singularize the particles and to break off agglomerates formed by microparticles adhering to each other.

After wetting, the microparticles can be subjected to de-aggregate and de-agglomerate step, generally referred to as dispersing step. The dispersing step typically includes the impact of mechanical forces such as shear to singularize the microparticles. In addition to shearing to singularize, the microparticles can be broken into even smaller microparticles using, for example, roller mills, high speed mixers, and bead mills. Usual practice involves substantially homogeneous dispersion of the pigments in dispersant through the use of high shear mixing; for example, through use to the appropriate ball mill, ultra high-pressure homogenizer or other composition known by those skilled in the art of pigment dispersion.

During wetting and dispersing, the exposed total surface area of the microparticles increases which is wetted by the dispersant. The amount of the dispersant may be gradually increased during dispersing to account for the increased surface area.

The dispersant also functions as de-flocculation agent keeping the dispersed microparticles in a dispersed state and prevent that they flocculate to form loose aggregates. This stabilization is also needed for long term storage purposes. Different type of stabilization such as electrostatic stabilization and steric stabilization are possible, and the type of dispersant is selected in view of the medium and the material of the microparticles.

The dispersant may be added to a dry powder of the pigment particles when the particles are milled to a desired size. During milling, or any other suitable technique to singularize the pigment particles or to break them into smaller part, the dispersant comes in contact with and adheres to the surface of the microparticles. Freshly generated microparticle surface during milling will be coated by the dispersant so that, after milling, the microparticles with a coating formed by the dispersant are provided.

The coating with the dispersant can also be carried out in a liquid carrier medium to which the dispersant is added. The microparticles can also be milled in the liquid carrier.

Optionally, the pigment microparticles may be coated with small molecules from a pretreatment composition. A portion of the pretreatment composition may be combined with the pre-milled pigment particles according to the procedures described above for wetting and dispersing the microparticles with dispersant. Following the wetting and dispersing procedures, the processed microparticles may be separated from excess pretreatment composition to produce microparticles coated with small molecules. It is believed that during this procedure, the coating of small molecules will begin to condense so that the coating will be at least a partially condensed net of silicone coating the individual microparticles. The coated microparticles may then be wetted and dispersed in dispersant as described above for the wetting and dispersing procedures.

### Additive Components

Additive components for the film forming composition include suspending agents, leveling agents and viscosity control agents. The suspending agents help maintain the pigment particles in dispersed condition and minimize or negate their agglomeration. Suspending agents include fatty acid esters of polyols such as polyethylene glycol and polypropylene glycol. These are similar to plasticizers and function in similar fashion to allow pigment particles to “slip” by each other without retarding or binding interaction. They act as grease in this fashion. Additionally, suspending agents in part participate in promoting the stable dispersion of the pigment particles and avoid settling. The polymers of the film forming composition also participate through their solubilization or interaction with the pigment particles and with the medium. The suspending agents provide another factor for maintaining the stable dispersion. They not only provide the “grease” to facilitate Brownian movement but also in part stabilize through interaction as emulsifiers of the pigment particles in the medium. Optional components also are to be chosen so that they do not interfere or only minimally interfere with the reactive polymer coupling reaction.

Embodiments of the film forming composition embodiments in accordance with the present invention can also optionally contain at least one adjuvant, chosen, for example, from reducing agents, fatty substances, softeners, antifoams, moisturizers, UV-screening agents, mineral colloids, peptizers, solubilizers, fragrances, anionic, cationic, nonionic, or amphoteric surfactants, proteins, vitamins, propellants, oxyethylenated or non-oxyethylenated waxes, paraffins, C₁₀-C₃₀ fatty acids such as stearic acid or lauric acid, and C₁₀-C₃₀ fatty amides such as lauric diethanolamide.

Embodiments of the film forming composition in accordance with the present invention can further optionally contain one or more additives, including, but not limited to, antioxidants (e.g., phenolics, secondary amines, phosphites, thioesters, and combinations thereof), non-reactive diluents (e.g., ethylene glycol, di(ethylene glycol), tetra(ethylene glycol), glycerol, 1,5-pentanediol, ethylene glycol monomethyl ether, ethylene glycol dimethyl ether, triethylene glycol monomethyl ether, 2-ethoxyethanol, solketal, benzonitrile, hexamethylphosphoramide, 2-N-methylpyrrolidinone and N,N-dimethylformamide); dyes, fillers (e.g., silica; carbon black; clay; titanium dioxide; silicates of aluminum, magnesium, calcium, sodium, potassium and mixtures thereof, carbonates of calcium, magnesium and mixtures thereof; oxides of silicon, calcium, zinc, iron, titanium, and aluminum; sulfates of calcium, barium, and lead; alumina trihydrate; magnesium hydroxide and mixtures thereof), plasticizers (e.g., petroleum oils such as ASTM D2226 aromatic oils; paraffinic and naphthenic oils; polyalkylbenzene oils; organic acid monoesters such as alkyl and alkoxyalkyl oleates and stearates; organic acid diesters such as dialkyl, dialkoxyalkyl, and alkyl aryl phthalates, terephthalates, sebacates, adipates, and glutarates; glycol diesters such as tri-, tetra-, and polyethylene glycol dialkanoates; trialkyl trimellitates; trialkyl, trialkoxyalkyl, alkyl diaryl, and triaryl phosphates; chlorinated paraffin oils; coumarone-indene resins; pine tars; vegetable oils such as castor, tall, rapeseed, and soybean oils and esters and epoxidized derivatives thereof, esters of dibasic acids (or their anhydrides) with monohydric alcohols such as o-phthalates, adipates and benzoates; and the like and combinations thereof), processing aids, ultraviolet stabilizers (e.g., a hindered amine, an o-hydroxy-phenylbenzotriazole, a 2-hydroxy-4-alkoxybenzophenone, a salicylate, a cyanoacrylate, a nickel chelate, a benzylidene malonate, oxalanilide, and combinations thereof), and combinations thereof.

An additional additive may be a tactile hair modification agent. These may include, but are not limited to, a softening and/or lubricating and/or anti-static and/or hair alignment and/or anti-frizz benefit and/or impact on the keratin fibers.

Additional additives include filler materials such as but not limited to no chromatic material with a particle size of from about 2 nm to about 500 nm; macromolecular strands or nanoparticles composed of polyolefin such as polyethylene, polypropylene, polybutene, and combinations thereof, clays and mineralite substances such as but not limited to smectites, kaolins, illites, chlorites, attapulgites and intercalated aluminosilicate materials and purified formed thereof and combinations thereof. Additional mineral microparticles may be composed of inorganic metal oxides selected from the group consisting of silica, titanium oxide, zirconium oxide, aluminum oxide, magnesium oxide, boehmite alumina, hydrotalcite. Still other filler material includes but is not limited to carbon nanotubes micrographitic material such as nanofiller of graphite oxide mixed polymer, microbucky balls, clathrates, and crown composites of organic and mineral complexes. Additionally, the filler may be combined, complexed, contain or incorporate a polymer containing one of the members of a complementary reactive pair relating to the first and second components of the reactive polymer composition.

Additives may also include but are not limited to UV filter and UV block substances such as but not limited to avobenzone, bemotrizinol octocrylene, benzophenone-4, ethylhexyl methoxycinnamate, PABA, padimate O, PBSA, cinoxate, dioxybenzone, homosalate, menthyl anthranilate, octyl salicylate, parsol Max, tinosorb S and A2B, Uvinul, amioxate, polyvinylidene fluoride and other similar conjugated organic compounds, radical scavengers, triplet formation inhibitors, metal compounds incorporating chromium, titanium, zinc, nickel, manganese, iron, niobium, silver, gold, aluminum, hafnium, tantalum such as the oxides and similar forms thereof wherein the metal compounds absorb or reflect UV light.

### Topcoat

The topcoat composition is a post dressing composition that may be applied at a later time by the person whose hair has been dressed with the coating and preferably color coatings (hereinafter the user). The topcoat composition may also be applied to the user's hair by a salon professional who has previously dressed the user's hair with coating and preferably color coatings or who is in the process of dressing the user's hair. The topcoat typically contains a readily evaporable medium such as an aqueous alcohol mixture in combination with water repellant compounds, hair setting compounds that may be shampoo and/or water rinse removable, or hair setting compounds that may be slowly removable by cationic shampoo but not by water rinse or ordinary anionic shampoo typically applied as a home shampoo wash of hair.

Water repellant compounds for inclusion in the topcoat composition may include waxes, silicones, organofluoride compounds. Preferred among such repellants is carnauba wax, beeswax, olefinic wax, paraffin. Polyurethanes, polyureas, polyesters, polysilicones and combinations thereof may also constitute constituents of the topcoat composition. Preferably, these polymers have significant numbers of non-reactive functional groups distributed throughout their polymer chains and as pendant groups so that hydrogen bonding, dipolar interaction and ionic interaction with the underlying films on the hair are produced. The presence of such polymers adds water repellency, shine and reasonable buoyancy character to the hair.

Hair setting compounds for inclusion in the topcoat composition may be readily removable with ordinary shampoo washing or may be long lasting in that multiple shampoo washings slowly will remove the hair setting compounds. The hair setting compounds enable retention of a particular set or coiffure under typical environmental conditions, such as rain, humidity and wind. Nevertheless, they may be removed by shampoo washing with commercially available shampoo formulations. The hair setting compounds useful for inclusion in the topcoat composition may be copolymers of an acidic vinyl monomer such as (meth) acrylic acid, a hydrophobic nonionic vinyl monomer such as alkyl (meth)acrylates, and first and second associative monomers such as polyoxyalkyenyl fumaric or similar unsaturated dicarboxylic acids. The compounds may be polyvinylpyrrolidones (PVP), copolymers of PVP and vinyl acetate (VA), acrylate and hydroxyalkyl acrylate copolymers, CARBOPOL (polyacrylic acid), CARBOPOL ETD polymer, xanthan gum, hydrophobically modified cellulose. Still other substances useful as hair setting compounds and as repellant compounds for topcoats are based upon (meth)acrylic copolymers of (meth)acrylic esters of C6 to C20 alkyl groups and (meth)acrylic esters of unsaturated alcohols and hydrophilic monomers such as (meth)acrylic acid. Copolymers of this formulation have unsaturation sites as films applied to the hair. A short UV irradiation of such copolymers as films enables cross linking and conveys wind, rain and shampoo resistance to the topcoat composition. Block copolymers of (meth)acrylic acid, crotonic acid, alkyl (meth)acrylates and minor percentages of olefinic monomers such as styrene provide the holding, low tackiness and high humidity resistance qualities to the topcoat while at the same time enabling readily removal with shampoo washing. Incorporation into the topcoat of a non-tacky pressure sensitive adhesive such as a copolymer of butyl acrylate and methacrylic acid with the percentage of methacrylic acid being minor on the order of 2 to 4 wt % also promotes hold and set. In some instances, a topcoat formulated with (meth)acrylate copolymers that are not readily removable by shampoo washing and display thermoplastic qualities at temperatures about at least 200 C above human body temperature may be useful for reset of hair styles. This version of the topcoat may be warmed with a warm hair dryer and the hair reset to a new style. Cooling the reset hair provides the reset hair style as the thermoplastic polymer retains the shape provided by the reset.

The topcoat composition may contain the polymer compounds as microparticles dispersed in the medium or may be dissolved in solution with the medium. The topcoat may be applied as a liquid composition using a brush, sponge or other similar applicator to coat individual hair strands. Alternatively, the topcoat composition may be incorporated into a spray pump container and applied as an aerosol to the hair. Applied as a spray the topcoat composition preferably is formulated to remain liquid on the hair for a sufficient time to enable gentle brushing to transfer the liquid throughout the hair strands and enable essentially all hair strands to be coated. When hair styling is part of the topcoat process, the hair may be set with mechanical devices or may be set with heat and mechanical manipulation as described above.

### Post Care Composition

The post care composition typically may be applied by the user periodically to preserve the shine, color lastingness and character of the coating and preferably color coating of the hair. The post care composition incorporates ingredients that impart lubrication, feel modifiers, sacrificial semi-fluid films to the hair. Includes are non-ionic surfactants, cationic surfactants such as long chain quaternary ammonium compounds, amosilicone conditioners, fatty acid amide conditioners, fatty alcohol betaines and sultaines, non-penetrating surfactants with a molecular volume larger than about 450 cc per mol. The post care composition may be formulated in a medium such as an aqueous or aqueous alcoholic medium that is capable of volatilization over a short period of time, such as one to five minutes. The post care composition may be applied to keratin fibers as a spray or as a liquid. Also useful as a post care composition is a protective composition that may be applied as a mask to the skin and parts of the user that are not to be treated with the compositions described herein. The protective composition forms a thin film mask on the skin and is readily removable by peeling. Adhesion to the skin is minimal so that peeling does not injure the skin. Compounds in aqueous alcohol solution provide the mask film upon evaporation of the medium. Compounds such as polymers and copolymers of high Mw organic hydroxy acids such as lactic and glycolic acid provide useful peelable masks. The post case composition can be designed to specifically care for the coating on the surface, versus the surface itself. So for example in the context of a coating on the hair surface, rather than use a regular product that is designed to clean and condition the hair surface, the post care composition is tailored to look after and care for the coating upon the hair surface.

### Testing the Flexibility of a Coating

With the coating and preferably color coating prepared on a releasable substrate and isolated as a standalone polymer film it can also be tested for optical density to check that the polymer film does not itself alter the hair appearance of the hair too significantly.

Further the polymer film preferably can be tested to reveal its glass transition point (Tg) as described above so that it is possible to prevent the colored coating from being damaged or cracked and to secure washing and friction remanence.

The coating and preferably color coating can have a surface energy between about 20 and about 50 mN m⁻¹.

Ultimate elongation. The term ultimate elongation refers to the amount of elongation a given material can experience under a specific test method before failure occurs and the material breaks into more than one piece. It is the separation at break divided by the initial separation in the test, multiplied by 100 to give a percentage ultimate elongation.

Young's modulus. Young's modulus, or the Young modulus, is a mechanical property that measures the stiffness of a solid material. It defines the relationship between stress (force per unit area) and strain (proportional deformation) in a material in the linear elasticity regime of a uniaxial deformation. It is the Stress/Strain in this region.

Ultimate compression. The term ultimate compression refers to the amount of compression a given material can experience under a specific test method before failure occurs and the material breaks.

Average repeated elongation before failure. Refers to the number of elongations to a fixed level of elongation repeat cycles that can be performed on a test material before failure occurs and the material breaks into more than one piece

The mechanical properties of the elastomer (Young's Modulus, Ultimate elongation, Ultimate compression, average repeated elongation before failure) are measured in a known manner using a Texture Analyzer TA.XT.Plus (Stable Micro Compositions).

For the Young's Modulus and Ultimate elongation, the elastomer is prepared as a continuous film, for example 10 Mil thick, on a release layer (for example baking paper) using a BYK square applicator or bird type film applicator (for example 5570 Single Bar 6″, 10 mils or 5357 or Square Frame 4″, 5-50 mils). If the elastomer is produced as a diluted composition, those skilled in the art will select the appropriate thickness of the drawdown film to produce a suitable film for testing. The film is left to cure at 25° C. for ˜24 hours or more. The elastomer is removed from the release layer and cut into rectangular sections measuring 30 mm by 10 mm using a scalpel. The thickness is then measured using a calliper to account for any shrinkage or solvent loss during curing. The rectangular film is then attached to the TA instrument using A/MTG Mini Tensile Grips (Stable Micro Systems) within an initial separation of 12 mm. The sample is then elongated at a rate of 0.5 mm s-1 until the elastomer sample breaks. The Young's modulus is defined as the initial slope of the linear portion of the elastic region of the force-elongation curve, which occurs just after the initial force of 5 g is applied. As the initial cross-sectional area is known, the force is converted to MPa to calculate the Youngs modulus. The ultimate elongation is indicated as a percentage, i.e. extension distance at break/initial distance*100. To assess the average repeated elongation before failure, first the ultimate elongation of the sample is measured. The sample and TA instrument are arranged in the same way as to assess the ultimate elongation. The sample is then elongated at a rate of 0.5 mm s-1 to a fixed elongation of 60% of the measured ultimate elongation. The sample then returns to its original state at a rate of 0.5 mm s-1 and the cycle is repeated until the sample breaks, or until a maximum of 2000 repeat cycles.

For the Ultimate compression, the elastomer is prepared as a continuous 3 mm film on a release layer. The film is left to cure at 25° C. for −24 hours or longer. The elastomer is removed from the release layer and a series of cylindrical disks are punched out of the film with a 3.5 mm diameter. The thickness is then measured using a caliper to account for any shrinkage or solvent loss during curing. The rectangular film is then attached to the TA instrument using A/MTG Mini Tensile Grips (Stable Micro Systems) using a compressive cycle. The sample is compressed at a speed of 0.05 mm s-I until the sample breaks. This is observed as a rapid deflection within the stress strain graph during the compressive cycle and is clear to those skilled in the art.

For all of the above mechanical property measurements results given are an average of at least 7 measurements.

### Kits and Container

The film forming composition and the pretreatment composition may be maintained in separate storage compartments or in separate kit form especially if they will react together without special activation. Additionally, the film forming composition complementary pair components and compounds with PTHalkoxysilyl groups may be maintained separately so that they will avoid reaction of the PTH groups with complementary groups, interaction of alkoxysilyl groups and reaction of complementary pair groups. A convenient storage means can be utilized such as plastic squeeze tubes, plastic bottles, glass containers, sachets, multi-compartment containers, tottles, spottles syringes and plunger operated dispensing devices. Unit amounts for combination can be formulated so that the entire contents of a unit of the film forming composition can be combined with the entire contents of the catalyst/promotor for application to the keratin fibers. Alternatively, metered or calibrated dispensing containers with optional brushes and/or sponge pads for providing measured amounts of the components as directed by printed instructions can be provided. With some embodiments, these components can be pre-combined for storage and handling as long as a substantive constituent that would cause in situ linking is maintained in a separate compartment.

Use of the foregoing delivery means enables preparation of an embodiment for practice of the method of the present invention. This embodiment may comprise sequential, simultaneous or premixed application, to keratin fibers, of the pretreatment composition and the film forming composition. Pigment microparticles may be incorporated in the film forming composition. This aspect of application provides an underlayer of pretreatment composition and overlayer of film forming composition on the keratin fibers. Management of the medium removal, temperature of the applied compositions and use of activation agents, if any, will enable transformation to a coating and preferably color coating in which the polymers of these compositions in situ interact to covalently, hydrogen bond, electrostatically, coordinately, ionically, dipolar-wise and entanglement-wise connect as the completed coating and preferably color coating. For the film forming composition the self-reactive or complementary pair reactive groups are chemically reactive so that covalent and/or coordinate bonds are formed between and among these components. Preferably, the components of the film forming composition also combine with PTH alkoxysilane compound(s) of the pretreatment composition so that the components of the film forming composition and the pretreatment composition covalently interact to bind all constituents together. With this aspect, the resulting coating and preferably color coating on keratin fibers provides good remanence against repeated shampooing, rinsing and contact with mild detergents, soap and similar wash substances.

The kit forms for the pretreatment and film forming compositions may also include one or more containers or package units for the materials and/or apparatuses for practice of the Praeparatur and Fundamenta techniques. A package unit for the Praeparatur technique may include one or more containers of various concentrations and kinds of anionic surfactants as well as containers for additives such as pH adjustment and carbonate solutions. An instruction packet may also be included to direct when to use the kinds of anionic surfactant, how to dilute them, how to massage and/or work the surfactant compositions throughout the anagenic hair and how to rinse away and dry the anagenic hair after the Praeparatur treatment. A package unit for the Fundament technique may include one or more containers with various concentrations of PETT formulations as well as the carbonate base additive for preparation of the PETT formulation for use. The Fundamenta package unit may also include a cold plasma pen with associated electronics and attendant wash and rinse compositions. An instruction packet may also be included to direct how to use the PETT and/or cold plasma pen. Additionally, a package unit for acidic oxidation may be included with directions for addition of hydrogen peroxide and adjustment of pH and concentration.

### Application of Praeparatur and Fundamenta Procedures

According to the present invention, one or both of the Praeparatur and Fundamenta procedures may be applied to keratin fibers such as anagen hair. They may be applied separately, applied to different segments of keratin fibers, may be applied sequentially and/or may be applied simultaneously. The Praeparatur procedure typically may be applied first to the anagenic hair and the Fundamenta procedure may be applied as needed.

The Praeparatur procedure typically begins formulation of an aqueous-alcoholic surfactant with the preferred surfactant being an anionic sulfate surfactant. Using high shear mixing procedures and appropriate dilution steps, about 10 to 40 ml of concentrated anionic surfactant mixture of sodium lauryl sulfate and sodium lauryl ether (PEG₁₀) sulfate may be combined with about 150 to 200 ml of distilled water. A mimic swatch prepared as described in the experimental section, may be submersed in the detersive surfactant and briskly agitated with a fine tooth comb for several minutes. If a live salon hair model is the subject of the Praeparatur procedure, they may be asked to place her head over a salon wash basin. The salon operator may then first wet the model's hair with water and then apply the surfactant solution to hair and massage and lather the Praeparatur composition onto the hair and scalp. After a period of time the salon operator may then rinse the product from the hair, and optionally repeat the process again. Depending upon the salon operator's or lab technician's visual inspection and touch of the hair, the salon operator/technician may also use a fine toothed comb or pass a hand held ultrasonic device over segments of the hair doused with detersive solution. The process is continued with optional elevation of the anionic surfactant concentration and optional pH adjustment until the operator/technician's visual inspection and touch of the hair indicates sebum, grime and minerals have been removed to expose bare hair shafts.

The Fundamenta procedure may be applied separate, alone and independent from the Praeparatur procedure or the two may be combined in either order. For a typical combined procedure, the Fundamenta procedure may be applied following the Praeparatur procedure application.

To accomplish the plasma Fundamenta procedure, sections of the salon model's hair or sections of the mimic tress may be exposed to a device producing a cold (ambient temperature) plasma, for example a Revlon PZ2 Plasma Pen. A typical cold plasma generator passes a stream of air, nitrogen or oxygen through a high energy RF or EMF field to produce ions and with air and oxygen, also ozone. The stream of partially ionized gas may be directed toward the hair. The result is a “cold plasma” of partially ionized gas on the keratin fibers. The “cold plasma” may be splayed over and through segments of the Praeparatur treated hair to deep clean the surfaces of the hair strands. The cold plasma is applied at a suitable distance over a period of 1 to 5 minutes, preferably 1 to 3 minutes to provide the desired effect of deep cleansing.

In another Fundamenta procedure, an aqueous solution of at least 10 wt %, preferably at least 20 wt %, more preferably at least 30 wt % polyalkyl ammonium bromide such as of trimethyl cetyl ammonium bromide (PETT) or trimethyl stearyl ammonium bromide (STAB) in either alkali at a pH of about 10 or in thiol at a pH above 7 is applied to the mimic hair tress or to sections of a salon model's hair and massaged throughout the tress or hair sections for a period of from about 5 minutes to 30 minutes, preferably 5 minutes to 10 minutes. This treatment is then rinsed with shampoo at acidic pH (with acetic acid) until the PETT or STAB is removed.

In another Fundamenta procedure, a composition comprising 1.9 to 12% hydrogen peroxide is mixed with a persulfate bleaching composition which can be a powder. The mixed composition is applied to the hair for a period from about 1 minute to 120 minutes, more preferably from 3 to 40 minutes and then rinsed thoroughly from the hair. In an additional alternate Fundamenta procedure, a composition comprising 1.9 to 12% hydrogen peroxide is mixed with a composition contain between 0.1 and 10% of an alkali agent chosen from monoethanolamine or ammonia and ammonium hydroxide. The mixed composition is applied to the hair for a period from about 1 minute to 120 minutes, more preferably from 3 to 40 minutes and then rinsed thoroughly from the hair.

In another Fundamenta procedure, an acidic oxidizer composition is prepared by combining 12% aqueous hydrogen peroxide with an acetic acid solution at pH 3.5-4 to produce a hydrogen peroxide concentration of about 0.5% to about 2.5%. The acidic oxidizer composition is applied to the hair for a period from about 1 minute to 10 minutes, more preferably from 3 to 6 minutes and then rinsed thoroughly from the hair.

In another Fundamenta procedure, a reducing composition, for example such as Wella Creatine (N) Perm Emulsion available from Wella Professionals, is applied to the hair for a period from about 1 minute to 20 minutes, more preferably from 2 to 15 minutes and then rinsed thoroughly from the hair.

Following practice of the Fundamenta procedure alone or with the Praeparatur procedure, the mimic swatch or salon model hair is ready for the Pretreatment step according to the invention.

### Application of Pretreatment Composition

According to the present invention, application of the pretreatment composition to keratin fibers as a pretreatment after application of the Praeparatur and Fundamenta procedures and before application of the film forming composition is at least in part a factor for achievement of the qualities and characteristics of the coating and preferably color coating on keratin fibers. According to this embodiment of the method, the pretreatment is applied on or to at least a portion of the keratin fibers and preferably throughout the keratin fibers.

Pretreatment with the pretreatment composition may be carried out prior to application of the color composition. Pretreatment may be carried out immediately prior to application of the color composition, or at least 1 hour prior to application of the color composition, or at least 24 hours prior to application of the film forming composition, or at least 10 days prior to application of the film forming composition, or at least one month prior to application of the film forming composition. Preferably, pretreatment may be carried out immediately prior to or within a few minutes up to an hour before application of the film forming composition. Typically, the pretreatment composition is at least partially dried with optional heating to at least substantially remove or otherwise eliminate its medium. For example, excess medium form the pretreatment composition on the hair may be removed by contacting the wet coated hair with an absorbent fabric or the wet coated hair may partially dried by heating with a hair drier. Preferably, substantial removal of the medium of the pretreatment composition is accomplished before application of the color composition.

In one embodiment more than one pretreatment composition may be applied to the hair. It may be that two different pretreatment compositions are applied sequentially, to provide a cumulative benefit for the subsequent film forming composition, which is then applied, or it may be that two different pretreatment and optionally two different film forming compositions are applied to substantially different portions of the hair. Such a case may arise when applying to hair which has quite different properties, for example, to sections which have been pre-bleached or color, versus natural hair, or for root versus tip hair. In such cases different pretreatments may be needed to prepare all of the hair for subsequent film forming compositions. While such different pretreatment would be directed to different portions of the hair, it is likely that there would be a least some minor mixing and some areas of the hair would receive both pretreatment compositions. A third case could be where a pre-treatment composition is applied to a section of the hair, for example at the roots, and the second different pretreatment composition is then applied across all of the hair.

### Application of Film Forming Composition

### Following Pretreatment

As described above, the one or more film forming compositions may be applied to the keratin fibers in combination with the foregoing pretreatment with the pretreatment composition. Embodiments of the film forming composition as the first binder component and second binder component which have the complementary pair of binder functional groups may be maintained separately until use. Application of the one or more film forming compositions to pretreated keratin fibers may be preferably accomplished by sequential application to segments of the hair. Once all segments are coated with one or more wet film forming compositions, the one or more film forming compositions may be dried and/or cured to form overlaid coating layers on the keratin fibers. Alternatively, the application and subsequent drying and or curing may be performed section by section across the head. Typically, the rate of condensation of the film forming composition and rate of drying may be pre-adjusted through medium control, pH adjustment if needed, concentration, steric interaction, temperature, and similar factors controlling reaction and/or drying rate so that a premix of the binder components of the film forming composition preferably will not substantially interact before the premix is applied to the keratin fibers. The practice of this step with the pre-treatment embodiment initially introduces the film forming composition on top of the pretreatment layer of small molecules on the keratin fibers. Because the film forming composition is in a medium, penetration, combination, mixing and/or melding of the film forming composition into the pretreatment layer will be accomplished at least in part. The penetration is believed to enable the linking among the binder polymer(s) of the film forming composition, the small molecules of the pretreatment composition and the keratin fibers. Drying and curing of these compositions preferably occur after all compositions have been applied. In this manner, melding among all layers is best achieved.

Application of the one or more film forming compositions to keratin fibers pretreated with the pretreatment composition is preferably carried out after pretreatment. This sequence may be carried out immediately after pretreatment, or at least 1 hour after pretreatment, or at least 24 hours after pretreatment, or at least 10 days after pretreatment, or at least one month after pretreatment.

The sequential, simultaneous or premix application of the film forming composition may be applied to at least a portion of the keratin fibers or may be applied all over the keratin fibers. The portions of the film forming composition may be applied sequentially, simultaneously in a single application over all the keratin fibers or may be applied step-by-step to the keratin fibers. Applying the film forming composition in a step-by-step manner as described above, may help to ensure that the treatable portions of the keratin fibers are saturated with the combined film forming composition and pretreatment composition and may therefore provide a better coverage of the keratin fibers.

### Manipulative Techniques for Application

After each application of the pretreatment composition and one or more film forming compositions have been accomplished, and the wet coated keratin fibers, e.g. treated keratin fibers, optionally rinsed, the treated keratin fibers will begin to cure. If the treated keratin fibers are heated using an elevated temperature, the condensation curing of the alkoxysilyl groups to siloxanyl groups and the complementary pair reaction may be accelerated. The temperature of the keratin fibers can be increased to elevated temperatures above room temperature such as 40° C. or higher, for example using a hair drier. While the keratin fibers are being heated, some form of interdigitated implement can be used to help separate portions of the keratin fibers, and especially separate hair strands from one another. Examples of interdigitated devices include a comb or a brush. The keratin fibers can be heated with a hair drier while simultaneously being combed or brushed until it is dry to the touch. Alternatively, other means can be employed to heat and separate the keratin fibers such as hair simultaneously. For example, using a combination of air movement and vibrations will accomplish distribution of the multicomponent composition throughout the strands of hair.

### Operational Method for Coating Hair

The performance of operational method aspects of the present invention can be applied to keratin fibers to form a coating of the pretreatment and film forming compositions and optional topcoat composition. This aspect of the invention concerns a method for coloring keratin fibers and comprises applying embodiments of one or more pretreatment and film forming compositions for a time sufficient to deposit an effective coating and preferably color coating on the keratin fibers such as each keratin fiber or hair strand. A somewhat to substantially overall distribution of the coating on the length and circumference of each fiber is produced.

To accomplish this aspect, performance of the Praeparatur and Fundamenta procedures to prepare the keratin fibers may be accomplished prior to or overlapping with or simultaneous with the pretreatment application. Depending upon the sequence for the activating and pretreatment steps, embodiments of the pretreatment and film forming compositions may be applied sequentially, overlapping or simultaneously to the keratin fibers according to the sequences described above by brushing, painting, spraying, atomizing, squeezing, printing, rubbing, massaging or in some manner coating the keratin fibers such as hair strands with the embodiments. Following application of a compositional embodiment to the keratin fibers such as hair strands, the composition is set, cured, linked, coordinated and/or otherwise melded together preferably by warming with blown warm air from a hair dryer or similarly treatable to remove the medium, initiate in situ linking of the alkoxysilyl groups and the complementary pair linking as well as hydrogen bonding, molecular entwining and polar interactions among the film forming polymers, the in situ formed silicone network formed from the pretreatment composition and keratin fibers. The setting leaves a substantial to essentially complete overall bonding and binding among these substantive constituents of the coating and preferably color coating on keratin fibers.

The rate or rate of reaction for the film forming composition and pretreatment composition to cure and to bond internally and with each other is the speed at which reactants are converted into products. In the context of the film forming and pretreatment compositions forming adherent colored coatings, the rate refers to the speed at which the covalent and non-covalent bonding occurs. In one embodiment it is preferred that the rate of reaction/drying is not so fast that the resulting elastomer forms before the wetting and spreading on keratinous surface can occur. If the rate of reaction/drying is too fast the resulting elastomer may not then be able to subsequently wet and spread on the hair surface, resulting in an inferior coating of the hair and one that displays less resistance to washing. In contrast, a rate of reaction/drying that is extremely slow will not enable a practical result in typical times for salon coloration treatment. In a preferred embodiment the rate of reaction/drying is slow enough such that the film forming and pretreatment compositions can wet and spread on the keratinous surface, yet also fast enough that a macroscopically continuous film on a keratinous surface is formed as the film bonds/binds covalently/non-covalently. The typical period for accomplishing continuous film formation is preferably less than 48 hours, more preferably in less than 24 hours, even more preferable in less than 12 hours, most preferable in less than 6 hours and especially most preferably in less than 30 minutes following the completion of application and under normal room temperature conditions.

The bonding and binding of the substantive constituents of pretreatment and one or more film forming compositions and the keratin fibers during application provides a coating and preferably color coating that resists removal by washing with dilute mixtures of soap and water or shampoo and water. Color remanence is developed so that washing with dilute aqueous soap solution or dilute aqueous shampoo will not substantially remove the coating, but the coating can be facilely removed by use of a transformation trigger. The properties of the coating include remanence, flexibility, adhesion, abrasion resistance and remanence which are due at least in part to the binding and bonding character of the substantive coating constituents including at least their intermolecular entwining, ionic and electrostatic intermolecular interaction, covalent and/or non-covalent linking, hydrogen bonding, dipole interaction and lipophilic interaction of these substantive constituents.

The pretreatment and film forming compositions and optional topcoat in accordance with the present disclosure can have a viscosity that can be controlled to enable the product to be applied to the hair using either a brush and bowl or a bottle, but with sufficient rheology such that it does not drip and run from the hair onto the face or body.

Alternatively, low viscosity formulations may be applied to the hair via a suitable application device such that it does not drip and run form the hair onto the face and body.

The pretreatment and film forming compositions and optional topcoat can be utilized in concentrated form or in serial dilutions, to provide for a consistent color results substantially along the entire length of the keratin fibers.

The aspect of coloring keratin fibers with a pretreatment and film forming composition and optional topcoat as described above includes a method for this coloring. The method comprises:
(i) applying the above-described pretreatment and film forming compositions to keratin fibers to obtain an effective, deposited coloring amount of the color composition including pigment microparticles and optional additional components;
(ii) setting the pretreatment and film forming compositions by removing or otherwise eliminating the medium (e.g., by drying the composition); and.
(iii) setting the interaction among the reactive constituents of the film forming and pretreatment compositions by initiating the in situ linking among these groups.

During the setting/drying step, color distribution can be facilitated by concurrently moving and/or stroking the hair with an interdigitating device. Interdigitating devices include a comb or brush. The interdigitating device needs to be pulled substantially along the hair strands from root to tip. It can be pulled through at a rate of 0.1 cm s⁻¹ to 50 cm s⁻¹ or at a rate between 0.5 cm s⁻¹ to 20 cm s⁻¹

The pretreatment and film forming compositions and optional topcoat are applied to the keratin fibers in any suitable way including spraying the pretreatment and film forming composition, massaging the keratin fibers by hand, after applying the pretreatment and film forming composition to the hand or by combing, brushing or otherwise applying the pretreatment and film forming composition throughout the keratin fibers.

The methods by which the pretreatment and film forming compositions and optional topcoat composition described herein are applied can be modified, such that the user applies the product in one region of the hair, and then can apply a diluted version in another region of the hair. The dilution formula is specially chosen to be compatible with the colorant formulation and reduces the coloring strength, while maintaining the longevity of the color result. This can effectively be a “blank” formulation, which contains broadly the same materials as the coloring formulation, but with lower or no pigments present. When diluted the ratio of the diluent to colorant can be between about 10:1 and about 1:10, about 8:1 and about 1:2 or about 5:1 and about 1:1.

Alternatively, the amounts of pretreatment and film forming compositions and optional topcoat composition applied can be altered in different regions of the hair, for example half the product is applied in the lengths of the hair, leading to a less colorful result. The difference in amounts applied in one region of the hair versus another can be between about 4:1 and about 1:4 or about 2:1 and about 1:2.

Alternatively, a combination of this approaches may be used to deliver the target color variation.

When the foregoing techniques are not possible to be applied, rather than apply a single hair color, it may be possible to apply two or more hair colors to different regions of the hair. When this is done, the different hair colors preferably provide complimentary colors so as to develop an attractive result. The difference in colors that can be used, based on the end result on hair tresses (as described later—untreated hair tresses) are as follows. As described within
the CIELCh color space on:
Color 1 (LCh) versus Color 2 (LCh)
Color 1 L-15<Color 2 L<Color 1 L+15
0 or Color 1 C-10<Color 2 C<Color 1 C+10
Color 1 h-45<Color 2 h<Color 1 h+45

Those skilled in the art of color measurements will know how to interpret difference in hue angles, h, when they extend from low positive values to those near to 360 degrees due to the periodic circular nature of the hue angle.

The method for use of the pretreatment and film forming compositions and optional topcoat composition in accordance with the present invention can occur during any suitable period. The period of application can be from about 0 to 30 minutes, but in any event a period that is sufficiently long to permit the coating of pigment microparticles to coat and adhere or bind to each separate keratin fiber, substantially along the entire length of each keratin fiber. The resultant is keratin fibers having a color and permanence that is at least equivalent to the color resulting from oxidative hair color, except under much milder conditions.

The pretreatment and film forming compositions described herein can be prepared by the manufacturer as a full shade, e.g., one that is ready to apply to the hair, and then shipped as a discrete unit to the user. The user may need to re-blend the pretreatment and film forming composition prior to application to ensure that the pretreatment and color composition delivers the optimum performance. Such re-blending can require shaking the pretreatment and film forming composition for about 1 to about 120 seconds or from about 3 to about 60 seconds. Re-blending may also be performed by stirring the pretreatment and film forming composition prior to use. This may occur for about 1 to about 120 seconds or from about 3 to about 60 seconds. Although the pretreatment and film forming compositions according to the present invention are designed to provide stable suspensions of the pigment particles, the re-blending to agitate the microparticles and resuspend them in a substantially uniform distribution is desirable.

Multiple compositions comprising different pigments can be blended together prior to application to the keratin fibers. Such blending can be done in a manner so as to apply a plurality of complementary surface colors to the keratin fibers. Typically, a large group of different pigments with small molecule coatings and concentrated in dispersant media are provided as pre-mixes for combining with the film forming composition. The color selection program describe above will determine which selections of the different pigment concentrates will provide the desired color result for the customer's hair. The pre-mixes are metered into the film forming composition at concentrations ready for application to anagenic hair.

The pretreatment and film forming compositions can include multiple layers, involving multiple applications of at least the film forming composition following the pretreatment and film forming compositions. It may be beneficial also to periodically reapply the third component. The techniques for applying multiple layers follow the techniques described above for application of a single pretreatment and color composition.

The coating of pigment microparticles comprising at least one pigment in a coating of the substantive constituents of the pretreatment and film forming compositions can be adhered to the treatable material such as hair utilizing a coating having a total thickness at any given point along the hair fiber of less than about 5 μm, preferably less than about 2 μm as measured using a scanning electron microscope (SEM). To make such measurements, a coated hair sample can be embedded in a suitable resin, and then sectioned root to tip using techniques known to those skilled in the art of scanning electron microscopy. The thickness of the layer on the surface can then be assessed along the line of cuticles over a length of at least 100 μm. The thickness of layer is determined by averaging 10 points evenly spaced over the section of interest.

In the course of application of the pretreatment, film forming compositions and optional topcoat compositions, it is possible to dress portions of hair rather than as one whole uniform area from root to tip and across the regions of the scalp. Several nonlimiting examples help explain a few of the many possible portions of the hair. In a first example the first portion of the hair refers to the hair adjacent to the persons scalp, the so called root hair region which may extend from a few millimeters to several centimeters. In this example the second portion of the hair is not adjacent to the scalp, i.e. the area which is not within the first portion. There may be some overlap between the first and second portions, due to the limitations of physically segregating these two portions on a person's head, but the two portions are different to one another. In a second example, the first portion of the hair refers to the one whole uniform area from root to tip. In this example, the second portion of the hair is then another section of hair from root to tip. Again. these two portions are different to one another, but have a significant area of overlap, with the second portion covering an area which may to some extent overlap with the first portion.

This understanding of the different qualities and attributes of sections of hair on a person's scalp shows that it is appropriate and preferably to apply separately pretreatment and color compositions to sections of hair strands. In addition to varying the concentration of the pigment microparticles and optional coloring agent, different shades and/or colors of pretreatment and film forming compositions can be applied to different sections of a strand of hair or a group of strands of hair. For example, the hair roots, mid sections and tips sometimes or often have different shades of color in their natural condition. This variation can be mimicked, altered or covered through use of differing shades or colors of the pretreatment and film forming compositions. Roots, for example can be covered with a lighter shade and the tips can be covered with a darker shade to produce a two tone variation of the hair. Application to the hair of a first portion of pretreatment and film forming composition followed by stripping the composition from the hair mid sections and ends followed by setting the remaining composition on the hair roots will provide a first hair coating and preferably color coating on the roots. The mid-sections and tips can be dipped or brush applied with a second portion of pretreatment and film forming composition to complete the two color or two tone treatment. The use of multiple pretreatment and film forming compositions to produce multiple coatings on the hair can provide overlapping, sequential or coterminous coatings on the hair according to typical and routine techniques for applying multiple versions of hair color practiced by professional hair salons.

### Post Treatment

An optional post treatment composition can be applied after treating the keratin fibers with the pretreatment and film forming compositions described herein. This can be applied directly after completion of coloring with these compositions. The post treatment can be either single application or multiple application across time. The post treatment can be used to improve one or more of: feel, resistance to shampoo/conditioner/water washing treatments, and shine of the hair. Nonlimiting examples of materials used to improve the feel are those which impart lubricity to the treatable material such as hair strands and/or help the hair strands separate during the drying steps. These materials include, for example silicone conditioners, silicone polyethers, silicone polyglucose, polyisobutene, copolymers of ethylene oxide and propylene oxide, and commonly used cosmetic oils and waxes. Nonlimiting examples of materials used to improve shampoo wash resistance are materials which act as a ‘sacrificial layer’ for example polymeric silicones and their copolymers, silicone resins, cosmetics oils and waxes. Nonlimiting examples of materials used to improve the shine of hair (meaning a decrease of the full width at half maximum parameter of the specular reflection curve as measured by a goniophotometer) are those materials which form a smooth film above the previously applied pigment polymer composite on the hair. In general, any cosmetically known film forming material can be used, but preferred are materials such as polymeric silicones and polycationic materials.

### Removal of Coating and Preferably Color Coating

Coatings and hair colorants made from the pretreatment and film forming composition are very resistant to everyday hair treatments (such as washing with shampoo, conditioner etc) can be removed via use of specifically designed “removal formulations.” These are specific chemical mixtures, described herein, and are designed to work by one or both of two broad mechanisms: cleavage of chemical bonds, either linking groups in the film forming and pretreatment compositions and solvation of components of the colored coating.

First, the mixture can be made to be a solvent for the pigment itself. In this case, the mechanism of removal involves first dissolution of the pigment from the binding matrix, followed by removal from the hair by rinsing with water or some other carrier. In this case it is believed, whilst not being bound by theory, that the chemical nature of the pigment, even when in dissolved form, is such that there is minimal attraction/solubility in the hair matrix itself, thus allowing removal of the color.

Second, the “removal formulation” can be made such that it dissolves, weakens or chemically breaks down the polymer coating holding the pigment on the hair. In this case it is believed, whilst not being bound by theory, that the pigments embedded in the binder matrix are released due to weakening or dissolution of the coating itself and, because the coloring material is a pigment, it has minimal attraction for the hair surface and is too big to penetrate the hair, and in consequence this facilitates removal of the color.

The combination of the above mechanisms will also provide the desired result of removal of the color.

Attacking the functional group bonds of the polymer network of the coating on the treatable material such as hair can have a dramatic impact on the properties of the coating which is adhered to the surface. An agent that cleaves those bonds can act as a trigger agent to divide the polymeric network and enable surfactant and solvent to readily disperse the cleaved coating. Such agents include basic amino alcohols such as dimethylaminoethanol (dimethylethanolamine, DMEA), dimethylaminopropanol, and similar amino alkanol agents such as monoethanolamine, diethanolamine and triethanolamine. These amino alcohols can be formulated in aqueous medium to enable coating removal. Additionally, or alternatively, fatty organic acids such as dodecylbenzene sulfonic acid or oleic acid may be combined with non-aqueous medium such as a volatile hydrocarbon including but not limited to dodecane to trigger removal. These organic acids function as surfactants to lift the coating from the keratin fiber surfaces and to break the functional group bonds which cleaves the polymeric network of the coating. The concentration of the trigger agent in alcoholic medium such as methanol, ethanol or aqueous medium or in non-aqueous medium may range from about 0.1% to about 15% by weight, preferably about 0.5% to about 10% by weight, more preferably about 1% to about 7.5% by weight relative to the total weight of the removal solution.

The polymeric films with in situ produced cross-links may also contain a group such as an ester, amide, urea or urethan group which can function as a cleavable linkage. This linkage is susceptible to hydrolysis and can be cleaved using basic or acid lysis. The cleavage will include a counter-nucleophile which can be water or a small molecular weight monofunctional amine or thiol.

The organic or silicone polymer having chain extensions with siloxane condensation, a strong acid such as dodecyl benzene sulfonic acid (DBSA) or a source of fluoride anion tetrabutylammonium fluoride (TBAF) in appropriate solvent as described in combination with Hansen solubility parameters including δd+δp+δh wherein 8d is from 13 to 25, preferably 15-19 and 8p is from 0 to 15, preferably 0 to 5 and δh is from 0 to 25, preferably 0 to 8.

Additionally, the in chain functional groups such as N-acylurea, urea, urethane, amide and/or ester can be cleaved through use of a small molecular weight monofunctional amine or thiol to attack the functional group and disrupt polymer chains.

Also, if silicone polymeric bridges are present in the silicone polymer or in the organic polymer, an organic acid (such as DBSA) may be used to de-polymerize the chain. TBAF or other organic fluoride such as Olaflur can also be used to de-polymerize the chain. It is also advantageous in all “off” techniques to employ an off reagent also has some surfactant quality.

When the pretreatment and film forming composition is applied to the hair, the multi-application process physically distributes the components to cover all of the hair. The spraying, massaging, combing and/or hand manipulating the pretreatment and film forming compositions produces the full coverage and at the same time leaves thin spots in the otherwise substantially uniform coating. This activity also will aid in the removal process.

Additionally, waxy non-reactive, non-combinable substances having melting points somewhat higher than human body temperature may be incorporated into the pretreatment composition. The concentration of waxy substance may be sufficient to enable heat disruption of the polymer film of the pretreatment layer on the keratin fibers but not enough to prevent the engagement of the polymer film properties of the pretreatment layer. By warming the hair with a hair dryer at a temperature of more than 200 C higher than body temperature, the waxy substance may be melted at least in part so as to disrupt the coating and preferably color coating on the keratin fibers. Combing or brushing can remove disrupted coating and preferably color coating.

Alternatively, an organic solvent soluble polymer such as a cellulose derivative, including but not limited to nitrocellulose, cellulose acetate-butyrate or other solvent soluble polymer may be incorporated into the film forming composition. The amounts and concentrations of the solvent soluble polymer are sufficient to enable the polymer to form separate domains of polymer film within the coating produced from film forming compositions. Contacting such a coating with an organic solvent in aqueous medium will at least in part dissolve the solvent soluble polymer and disrupt the continuous nature of the coating layer. Combing or brushing can remove disrupted coating and preferably color coating.

### Remanence and Treatable Material Inspection

Damage caused to the hair by application of the pretreatment and film forming composition and removal of the resulting coating can be assessed by FT-IR (Fourier Transform Infrared) method, which has been established to be suitable for studying the effects of keratin surface damage. Strassburger, J., J. Soc. Cosmet. Chem., 36, 61-74 (1985); Joy, M. & Lewis, D. M., Int. J. Cosmet. Sci., 13, 249-261 (1991); Signori, V. and Lewis, D. M., Int. J. Cosmet. Sci., 19, 1-13 (1997)). In particular, these authors have shown that the method is suitable for quantifying the amount of cysteic acid. In general, the oxidation of cystine is thought to be a suitable marker by which to monitor the overall oxidation of the keratinous part of the fiber. Net, the measurement of cysteic acid units by FT-IR is commonly used.

Signori and Lewis (D. M., Int. J. Cosmet. Sci., 19, 1-13 (1997)) have shown that FT-IR using a diamond Attenuated Total Internal Reflection (ATR) cell is a sensitive and reproducible way of measuring the cysteic acid content of single fibers and bundles. Hence, the method that can be employed to measure the cysteic acid content of multiple fiber bundles and full hair switches, is based upon the FTIR diamond cell ATR method employed by Signori and Lewis (1997). The detailed description of the method for testing the different damage inhibitors follows thereafter:

A Perkin Elmer Spectrum® 1 Fourier Transform Infrared (FTIR) composition equipped with a diamond Attenuated Total Internal Reflection (ATR) cell may be used to measure the cysteic acid concentration in mammalian or synthetic hair. In this method, hair switches of various sizes and colors can be used. The switches may be platted (˜1 plait per cm) in order to minimize variations in surface area of contact between readings. The Oxidative hair Treatment Protocol described above may be repeated for 5 cycles to mimic the behavior of hair after repeated bleaching cycles. Following this treatment, four readings per switch may be taken (⅓ and ⅔s down the switch on both sides), and an average calculated. Backgrounds may be collected every 4 readings, and an ATR cell pressure of 1 N/m may be employed. The cell may be cleaned with ethanol between each reading, and a contamination check may be performed using the monitor ratio mode of the instrument. As prescribed by Signori &amp; Lewis in 1997, a normalized double derivative analysis routine may be used. The original spectra may be initially converted to absorbance, before being normalized to the 1450 cm⁻¹ band (the characteristic and invariant protein CH₂ stretch). This normalized absorbance may be then twice derivatised using a 13 point averaging. The value of the 1450 cm⁻¹ normalized 2nd derivative of the absorbance at 1040 cm⁻¹ may be taken as the relative concentration of cysteic acid. This figure may be multiplied by −1×10⁻⁴ to recast it into suitable units.

When the compositions of the current invention can be applied to the hair and then removed there can be a non-significant change to the level of damage to the hair, whereas with conventional oxidative colorants there can be a large increase in the measured damage.

The instant disclosure is not limited in scope by the specific compositions and methods described herein, since these embodiments are intended as illustration of several aspects of the disclosure. Any equivalents are intended to be within the scope of this disclosure. Indeed, various modifications in addition to those shown and described herein can be within the grasp of those with ordinary skill in the art. Such modifications are also intended to fall within the scope of the appended statements.

### Color Selection

Also contemplated herein are pretreatment and film forming compositions having a given color area (gamut principle described above) defined by color coordinates (a*, b*) in the color space represented by the L*a*b* color composition, which can be divided into a plurality of color areas. Each of the plurality of colors obtained from the area surrounding a given set of hair fibers is judged to belong to which color area of the colored area of a certain color. The number of colors judged for each color area is counted, and the color of the color area with the largest number of colors is selected as a representative color of the area surrounding a given set of hair fibers. The compositions are capable of delivering colors on hair (test method herein for fade) such that the results colors lie within the range of about 18<L<about 81, about −2<a<about 45, and about −13<b<about 70.

When the color is removed from the keratin fibers the waste water/composition can be treatable to remove the pigments from the waste water effluent composition. This can be achieved by filtration, or through cyclone technology, where the density differences are used to force the pigments to the settle, and the water to pass through.

### Experimental Techniques Mimicing Anagenic Hair

A typical procedure for coloration of anagenic hair may involve application of a permanent oxidative dye formulation or may involve semi-permanent application of a direct dye or may involve temporary coloration that can be removed by a single mild shampoo washing. These three techniques for hair coloration traditionally are applied to anagenic hair without a prior wash of the anagenic hair. The presence of sebum, fatty acids (F layer), natural oils, sweat residue, mineral excretion from skin pores are traditionally regarded as helpful in the practice of these coloration techniques. Of course, if the anagenic hair also contains dirt particles, it is usually combed thoroughly to remove dirt debris but the natural oils, secreted minerals, sebum, fatty acids and the like remain.

It is expected therefore that the remanent results demonstrated by formation of a coating and preferably color coating according to the invention on a treated or untreated tress would also be demonstrated by formation on anagenic hair of a coating and preferably color coating according to the invention. In contrast to this expectation, and as shown by the mimic tress experiments described in priority PCT application PCT/EP2021/057926, titled “Salon Demonstration Regarding Anagenic Hair”, a color coating formulated onto lab tresses displays very strong remanence while the same color coating on the anagenic hair such as hair on the scalp of a live hair model does not display long-lasting remanence. The hue, intensity and shade of the color coating on anagenic hair rapidly decreases with each shampooing and by 3 shampoo washes or less, the color coating is gone, especially for color coating on the root portion of anagenic hair.

In contemplation of these results, it was realized that treated and untreated tresses fundamentally differ in at least one respect from anagenic hair. The treated and untreated tresses, which are the typical universal substrate for keratin fiber experimentation, are not connected to hair follicles and do not receive continuous secretions of sebum, natural oils and fatty acids as well as sweat and mineral secretions from adjacent skin pores. This realization led to experimentation to improve remanence on anagenic hair by converting anagenic hair to hair like that of treated and untreated tresses. These attempts involved initial removal of sebum, natural oils, fatty acid secretions, sweat and mineral secretions by detergent washing as is usually performed on cut hair being prepared for treated tresses. These attempts failed, however. Subsequent body secretions appurtenant to the anagenic hair on the scalp of hair models were found to continue circumvention of the remanence result experienced with treated and untreated tresses.

Continued research has led to a combination of experimental features that have enabled development of a coating and preferably color coating on anagenic hair that displays remanence similar to that displayed by treated and untreated tresses. These aspects include at least the techniques of Praeparatur and Fundamenta in combination with the Pretreatment Composition with PTH alkoxysilane compounds as described above.

These experimental techniques are described in detail in the Experimental Section. Briefly, hair swatches that are not preconditioned or pretreated by bleach or other techniques form the basis for experimental examination of the coating and preferably color coatings described herein. The hair swatches are combined with synthetic sebum, processed through the activating, pretreatment and binding steps. The hair swatches with coating and preferably color coatings are then examined for remanence by repeatedly coating with synthetic sebum and then shampooing. The synthetic sebum recoating and shampooing steps are repeated multiple times to determine the degree of remanence. This experimental technique is detailed in the following examples section titled “full root simulation color remanence test.”

### EXPERIMENTAL SECTION

### Examples

### General

The color compositions described herein within the examples are generally applied to a hair tress. One gram of each of the pretreatment composition and/or film forming composition is applied to each gram of hair tress. The tress is placed on a flat plate or in a bowl and the pretreatment composition and/or film forming composition brushed into the hair to ensure that all of the strands look visibly coated with the composition(s). The hair tress is then dried by heating with a hair dryer while combing until it is dry to the touch and the hairs are individualized.

### Preparation and Application of Film Forming Systems to Color Hair:

The compositions used herein are prepared as described in the following sections prior to starting the applications steps.

### General Description of Treatment and Application of Color Composition Steps:

If required apply at least one Praeparatur composition to the hair tresses and then rinse the hair tresses. Repeat as required.
If required perform a Fundamenta step on the hair tresses.
If required, apply at least one Pre-treatment composition to the hair tresses.
Application of film forming composition to hair tresses followed by curing to produce colored hair tresses.

After the color composition is applied to the hair tress the Full root simulation color remanence test is performed.

### Full Root Simulation Color Remanence Test:

This test was used to determine the color remanence of the color coating under conditions which are designed to mimic a person's anagenic hair and especially a person's root hair. It comprises the follow parts.
Selection and preparation of the hair tress for testing.
The required combination of Praeparatur and/or Fundamenta and/or Pre-treatment and or application of film forming composition.
Remanence procedure and assessment.

### Selection and Preparation of Hair Tresses for Testing.

Light blonde hair tresses are used as these are believed to be a better mimic of real root hair versus using the more processed natural white hair. Prior to application of a color coating to the tresses, a sebum mimic is applied to the tresses as described below to account for the anagenic hair with sebum. The is the typical state of the hair of a consumer where the sebum is excreted from the scalp and wicks along the hair fibers. After converting to the colored coating, the colored tress is washed using a protocol including reapplication of a sebum mimic to imitate the recoating of the hair with sebum originating from the sebaceous glands in-between hair washes. The full root simulation hair tress is termed mimic hair or mimic hair tress throughout this application. A synonym also used in this application to describe mimic hair is “full root simulation hair” or “anagenic hair”

Light blonde hair tresses were purchased (Farbe 9/0 from Kerling International Haarfabrik GmbH, Backnang, Germany) in the form of 10 cm long, 1 cm wide strands. The light blonde hair has in prior testing been shown to be a better mimic of consumers root hair, the hair adjacent to the scalp. Whilst not wishing to be bound to theory, it is thought to be less processed by the supplier prior to preparing hair tresses than the natural white hair tresses. These hair tresses were also used as received. Untreated hair tresses as purchased and as used are not coated with natural or synthetic sebum, but they do have the F layer coating.

The following procedure describes initial sebum application to produce mimic hair. The light blonde hair tresses are treated with synthetic sebum to better simulate root hair found on a person. Sebum measuring 0.1 g of (Hautfett nach BEY, sold by Wfk-Testgewebe GmbH) is applied and worked through the individual hair tress weighing about 1 g. The tress was placed in an over at 400 C for 30 minutes to produce a mimic hair tress.

Steps to apply the color composition (pretreatment and film forming compositions) to the mimic hair tress are performed as described above. The color coating on the mimic hair tress was then allowed to rest at 20° C. and 60-65% RH for 2 days. This temperature was chosen to more closely replicate conditions on a consumers hair before their first hair wash after coloring.

### Remanence Procedure and Assessment.

After these 2 days the following sebum and shampoo sequence was performed upon the mimic hair tress with a color coating prepared as described above.
1. 0.1 g of synthetic sebum was applied to the colored mimic hair tress weighing about 1 g described above. The sebum was rubbed into the tress to distribute it evenly.
2. The tress was placed in the oven at 40° C. for 30 min 3. The hair tress was rinsed for approximately 10 seconds with water (4 L min⁻¹) at approximately 37+/−3° C.
4. 0.1 g “Wella Professional Brilliance Shampoo for fine and normal hair” was applied without dilution to the colored mimic hair tress weighing about 1 g described above.
5. Shampoo was worked into the colored mimic hair tress for about 30 sec with a stroking motion with the fingers.
6. The shampooed colored mimic hair tress was then rinsed with water for approximately 30 seconds.
7. 0.1 g “Wella Professional Brilliance Shampoo for fine and normal hair” was applied without dilution to the colored mimic hair tress weighing about 1 g described above.
8. Shampoo was worked into the colored mimic hair tress for about 30 sec with a stroking motion with the fingers.
9. The shampooed colored mimic hair tress was then rinsed with water for approximately 30 seconds.
10. The rinsed colored mimic hair tress was then dried using a hair dryer while mechanically separating the fibers in the hair tress until it was uniformly dry.

Steps 1-10 described above represent one cycle of the full root simulation color remanence test. These were repeated for a total of 5 cycles to complete the full root simulation color remanence test and resulted in a total of 10 shampoo applications to the hair tress prior to visual assessment. The visual color remanence assessment described below was then performed to assess the color remanence after the full root simulation color remanence test.

Remanence was assessed visually by comparing the washed samples versus a retained tress which had been colored but not washed. Color remanence was graded as either very strong when the color after washing was either unchanged or remaining very intense, strong where the color remained intense but was noticeably less than the starting color, moderate where the color was still visible but obviously less than the starting color, weak where only a low level of color remained and finally no remanence where no color remained visible. To simplify the result tables shown below theses color remanence grades were given the following annotations, very strong=+++, strong=++, moderate=+, weak=0, no remanence −.

### Procedures Used to Produce a Color Coating on Keratin Fibers System.

The pigment was combined with isopropanol and a dispersant and blended using a bead mill to form a pigment paste. This was then used within the following experiments.

TABLE 1.1
Pigment Red 122 Paste composition.

| | wt % |
|---|---|
| Pigment Red 122¹ | 10 |
| Disperbyk 140² | 5 |
| Isopropanol³ | Qs to 100 |

¹Pigment Red 122, Hostaperm Pink E250 obtained from Clariant.
²Diperbyk 140 obtained from BYK-Chemie GmbH.
³Isopropanol obtained from BCD Chemie GmbH, Hamburg, Germany.

### Example 1. Performance of the Color Coating Using an OSSI and a Film Forming Composition Comprising an Organic Polymer Comprising at Least One Alkoxysilyl Group

### Preparation Procedure for Color Coating on Keratin Fibers.

Compositions were applied within 2 hours of making.

### General Coloring Procedure:

To the mimic hair tress described above one or more of the following steps were applied as needed.

### A Praeparatur Procedure Comprising:

1. Mimic hair tress were rinsed for approximately 10 seconds with water (4 L min⁻¹) at approximately 37+/−3° C.
2. 0.1 g “Wella Professional Brilliance Shampoo for fine and normal hair” without dilution was applied to the colored mimic hair tress weighing about 1 g described above.
3. Shampoo was worked into the colored mimic hair tress for about 30 sec with the fingers using a stroking motion.
4. The shampooed colored mimic hair tress was then rinsed with water for approximately 30 seconds.
5. The rinsed colored mimic hair tress was then dried using a hair dryer while mechanically separating the fibers until the tress uniformly dry.
   Multiple Potential Fundamenta Procedures were Selected as Needed from:

### Basic Oxidation Fundamenta Procedure

1. Blondor Multi-Blonde bleach powder available from Wella Professionals was mixed 1 part with 1.5 parts of 12% Welloxon Perfect available from Wella Professionals. The pH of the mixed product was higher than 9, so this was considered as a basic oxidation composition.
2. The tress was treated with a mixture of about 4 g of this mixture applied to each gram of hair.
3. The tress was then incubated in an oven at 45° C. for 30 minutes.
4. The tress was then rinsed in water, 37+−3° C. with a flow rate of 4 L/min for 2 minutes 5. The hair tress was then dried with a standard Hair dryer from Wella.

### Acidic Oxidation Fundamenta Procedure

1. The tress was treated with about 4 g of Wella Creatine Wave (N) Neutralizer Fixierung available from Wella Professionals for each gram of hair. Wella Creatine Wave (N) Neutralizer Fixierung is a hydrogen peroxide based composition with a pH below 4 and a peroxide level of 3%, this was considered as an acidic oxidation composition.
2. The tress was then incubated in an oven at 30° C. for 10 minutes.
3. The tress was then rinsed in water, 37+−3° C. with a flow rate of 4 L/min for 2 minutes
4. The hair tress was then dried with a standard hair dryer from Wella.

### A Plasma Fundamenta Procedure.

1. An atmospheric low temperature plasma pen, Piezobrush® PZ3 (Relyon Plasma, Regensburg, Germany) was used to treat the hair tress.
2. It was held 5 mm from the tress surface and moved slowly up and down along the tress for 3 minutes on each side to perform the Fundamenta step.

### An Alkali Phase Transfer Tenside (APTT) Fundamenta Procedure.

The following solution was prepared, CTAB (C16 alkyl trimethyl ammonium bromide) 0.20%, sodium carbonate, 1.60% and water 98.2%. 50 g of solution was prepared for each tress that was treated in a beaker. This was heated (to a temperature range from about 39° C. to 60° C.). The tress was placed in the alkaline surfactant solution for (lower temperature required more time, 15 min to 30 min) with stirring performed by a magnetic stirrer. Afterwards the tress was removed from the surfactant solution and dried. The following acidic cleaning composition was then prepared. Texapon N70 (70% in Water) 14.29%, Isopropanol 25.00%, Acetic acid 3.00%, Water 57.71%. The following steps were then performed.
1. The treated hair tress was rinsed thoroughly for 2 minutes with water (4 L min⁻¹) at approximately 37+/−3° C.
2. Acidic cleaning composition was applied to hair, using 0.1 g for each gram of hair tress for 60 seconds with the fingers used to distributed composition through the hair tress.
3. The hair swatch was rinsed with water for 60 sec with water (4 L min⁻¹) at approximately 37+/−3° C.
4. Steps 2-3 were repeated two more times.
5. The tress was then blow dried.

### Reduction Fundamenta Procedure

1. The tress was treated with about 4 g of Wella Creatine (N) Perm Emulsion available from Wella Professionals for each gram of hair. Wella Creatine (N) Perm Emulsion is a reductive composition with a pH around 9.
2. The tress was then incubated in an oven at 30° C. for 10 minutes.
3. The tress was then rinsed in water, 37+−3° C. with a flow rate of 4 L/min for 2 minutes 4. The hair tress was then dried with a standard Hair dryer from Wella.

### A Pre-Treatment Process Comprising

1. Hair was treated with the pre-treatment composition described below, one gram of composition per one gram of hair.
2. The composition was left on the hair for 5 min.
3. The hair was then dried using a blow dryer with combing to result in dry hair.

### A Binder Step Process Comprising

1. A freshly prepared film forming coloring composition prepared as described above, 1 gram of composition was applied per 1 gram of hair.
2. Application was accomplished by a slow distribution and spreading on the hair tress, for example, with fingers, brush, comb or other manipulation instrument/tool. The slow distribution was accomplished by application with a syringe or a pipette serially to portions of the hair tress.
3. Excess film forming coloring composition was removed with absorbent tissue material and the resulting colored hair tress was blow dried while combing using a hair dryer to achieve better hair individualization.
4. Treated hair tresses were kept at rest for at room temperature 20° C. at 60-65 00 relative humidity for 2 days.

TABLE 2
Different combinations of Praeparatur and Fundamenta followed by
a pre-treatment and film forming composition done sequentially
or combined.

| | 1A | 1B | 1C | 1D | 1E | 1F | 1G | 1H | 1I | 1J |
|---|---|---|---|---|---|---|---|---|---|---|
| Praeparatur | n/a | X | X | X | X | X | X | X | n/a | X |
| Fundamenta | | | | | | | | | | |
| Basic oxidation | | | X | | | | | | | |
| Plasma | | | | X | | | | | | |
| APTT | | | | | X | | | | | |
| Reductive | | | | | | X | | | | |
| Reductive then acidic oxid | | | | | | | X | | X | X |
| Acidic oxidation | | | | | | | | X | | |
| Pre-treatment | | | | | | | | | | |
| MTMO⁴ | 10.0 | 10.0 | 10.0 | 10.0 | 10.0 | 10.0 | 10.0 | 10.0 | 10.0 | n/a |
| Water | Qs | Qs | Qs | Qs | Qs | Qs | Qs | Qs | Qs | n/a |
| Acetic Acid⁵ | 0.04 | 0.04 | 0.04 | 0.04 | 0.04 | 0.04 | 0.04 | 0.04 | 0.04 | n/a |
| Coloring Composition | | | | | | | | | | |
| Worlee Add VP 2100⁶ | 0.15 | 0.15 | 0.15 | 0.15 | 0.15 | 0.15 | 0.15 | 0.15 | 0.15 | 0.15 |
| WorleePur VPSi 2021⁷ | 6.67 | 6.67 | 6.67 | 6.67 | 6.67 | 6.67 | 6.67 | 6.67 | 6.67 | 6.67 |
| Pigment Red 122 Paste⁸ | 10 | 10 | 10 | 10 | 10 | 10 | 10 | 10 | 10 | 10 |
| MTMO | | | | | | | | | | 1.0 |
| IPA | qs | qs | qs | qs | qs | qs | qs | qs | qs | qs |
| Results | | | | | | | | | | |
| Full root simulation result | − | + | ++ | +++ | +++ | + | +++ | +++ | +++ | +++ |

⁴MTMO 3-mercaptopropyl-trimethoxysilane from Sigma Aldrich
⁵Acetic Acid obtained from VWR
⁶Worlee Add VP 2100, (bis(2-ethylhexyl)phosphate ester) obtained from Worlée-Chemie GmbH.
⁷WorleePur VPSi 2021 is a triethoxysilyl terminated linear polyester of Formula IA with the polyester moiety, the urea and the urethane connector groups as described earlier in the section titled THE SINGLE POLYMER ALKOXYSILANE FILM FORMING COMPOSITION, and was obtained from Worlée-Chemie GmbH.
⁸Pigment paste made according the method described above.

These experiments were performed to show the performance of different color coatings on keratin fibers after the full root simulation color remanence test where the color is subjected to five fresh applications of sebum and a total of 10 shampoo washes. In the control leg, 1A, no Praeparatur or Fundementa steps were used prior to the application of the pre-treatment followed by the coloring composition. This resulted in a no remanence. With the addition of a Preparatur step, 1B, the remanence was increased to moderate. Whilst not wishing to be bound to any particular theory, it's believed that the presence of sebum on the hair surface, such as that found on consumers root hair, acts as something akin to a release layer, and its removal facilitates stronger adhesion of the color coating onto the hair surface. When a basic pH oxidation Fundamenta was added between the Praeparatur and pre-treatment, 1C, the remanence was improved to strong. The use of an alkali cleaning or plasma Fundamenta, 1D-1E provided very strong remanence. The reductive Fundamenta provided moderate performance, IF, which increased to very strong performance when an acidic oxidative Fundamenta was applied after the reduction process, 1G. The use of just the acidic oxidation as a Fundamenta, 1H also provided very strong remanence. 1I removed the Praeparatur step and used the combination of a sequential reductive and acid oxidative Fundamenta and provided very strong performance. Finally, within these series of experiments, 1J used a Praeparatur, and the sequential reductive and acid oxidative Fundamenta. The pre-treatment active was then moved into the coloring composition. This also showed very strong performance. 1J has two advantages versus the other systems. It removes the step of the separate pre-treatment, making the process easier and simpler to perform. It also uses less of the MTMO, and in the system tested significantly reduced the odour profile of the whole process. It has these two advantages, whilst still maintaining a very high level of remanence.

Other factors were also assessed on the hair before the application of the Pre-treatment and the Coloring Composition. This was done to assess the impact of the Fundamenta on the hair itself, which would be masked to some degree after the application of the colored coating onto hair. The level of oxidative damage and feel of the hair. Oxidative damage was assessed using the FT-IR method described above on three hair tresses. High values refer to higher levels of cysteic acid which is associated with more chemical hair damage. Therefore, lower values of FT-IR values are preferred. The feel of the hair was assessed by a panel of 14 experts on a scale of 1 (exceedingly damage) to 5 (feels like natural hair). For the feel rating higher values are preferred.

TABLE 3
Damage assessments of different Fundamenta prior to pre-treatment.

| | 1B | 1C | 1D | 1E | 1F | 1G | 1H |
|---|---|---|---|---|---|---|---|
| Praeparatur | X | X | X | X | X | X | X |
| Fundamenta | | | | | | | |
| Basic oxidation | | X | | | | | |
| Plasma | | | X | | | | |
| APTT | | | | X | | | |
| Reductive | | | | | X | | |
| Reductive then acidic oxid | | | | | | X | |
| Acidic oxidation | | | | | | | X |
| FT-IR Value | 25 | 136 | 25 | 22 | 35 | 50 | 27 |
| Feel Rating | 4.1 | 3.2 | 2.6 | 4.3 | 2.5 | 3.9 | 3.8 |

The FT-IR cysteic acid values showed that the basic oxidation, 1C provided the highest level of oxidative damage. In contrast the combination of reductive followed by acidic oxidation, 1G, provided much lower levels of cysteic acid, and the reductive step alone, 1F produced even less cysteic acid. The remaining Fundamentae, 1D, 1E, 1H, produced no noticeable change in cysteic acid versus 1B. On the feel test, the APTT, 1E, and the sequential application of a reductive then an acidic oxidation, 1G and the acid oxidation only, 1H, Fundamenta provided hair feel comparable to 1B. Further a visual assessment of the color of the tresses was also performed. All tresses appeared to have the same hair lightness as 1B, except the one subjected to a basic oxidation process which was much lighter. While this would be useful to provide colored coatings which are lighter than the original starting hair color, it would not enable the hair to go back to the original color when the colored coating was removed.

### Example 2. Performance of the Color Coating without an OSSI and a Film Forming Composition Comprising an Organic Polymer Comprising at Least One Alkoxysilyl Group

The procedures used within example 1 were reapplied to example 2, where instead of using an OSSI within the pre-treatment, an alternative active was used. These experiments were performed to show the enhanced performance when using an OSSI

TABLE 4
Different combination Praeparatur and different Fundamenta followed by a non-OSSI
pre-treatment and a film forming composition.

| | 2A | 2B | 2C | 2D | 2E | 2F | 2G | 2H |
|---|---|---|---|---|---|---|---|---|
| Praeparatur | n/a | X | X | X | X | X | X | |
| Fundamenta | | | | | | | | |
| Basic oxidation | | | X | | | | | |
| Plasma | | | | X | | | | |
| APTT | | | | | X | | | |
| Reductive | | | | | | X | | |
| Reductive then acidic oxid | | | | | | | X | X |
| Pre-treatment | | | | | | | | |
| MEMO⁹ | 5.0 | 5.0 | 5.0 | 5.0 | 5.0 | 5.0 | 5.0 | 5.0 |
| Water | 5.0 | 5.0 | 5.0 | 5.0 | 5.0 | 5.0 | 5.0 | 5.0 |
| Worlee Add VP 2100 | 0.25 | 0.25 | 0.25 | 0.25 | 0.25 | 0.25 | 0.25 | 0.25 |
| Isopropyl alcohol | qs | qs | qs | qs | qs | qs | qs | qs |
| Coloring Composition | | | | | | | | |
| Worlee Add VP 2100 | 0.15 | 0.15 | 0.15 | 0.15 | 0.15 | 0.15 | 0.15 | 0.15 |
| WorleePur VPSi 2021 | 6.67 | 6.67 | 6.67 | 6.67 | 6.67 | 6.67 | 6.67 | 6.67 |
| Pigment red 122 Paste | 10 | 10 | 10 | 10 | 10 | 10 | 10 | 10 |
| IPA | qs | qs | qs | qs | qs | qs | qs | qs |
| Results | | | | | | | | |
| Full root simulation result | − | 0 | 0 | ++ | ++ | 0 | ++ | + |

⁹MEMO 3-(Methacryloyloxy)propyltrimethoxysilane obtained from Alfa Aesar

These experiments were performed to show the performance of different color coatings on keratin fibers after the full root simulation color remanence test where the color coating was subjected to five fresh applications of sebum and a total of 10 shampoo washes. They contrast the performance obtained with an OSSI within Example 1. Overall, the performance was much weaker for the series in example 2 versus example 1. In the control leg, 2A, with no Praeparatur or Fundementa there was no remanence. With the use of a Praeparatur alone, 2B3, or in combination with a basic oxidation Fundamenta, 2C, the remanence was weak. The performance with the Plasma and alkali cleaning was strong, 2D and 2E. The use of only a reductive Fundamenta after the Praeparatur, 2F resulted in weak performance. Finally, the use of a combination of reductive followed by acidic oxidation Fundementa without a Praeparatur resulted in moderate performance, 2H, which rose to strong remanence when coupled with a Praeparatur step. This shows the enhanced performance of the OSSI.

### Example 3. Performance of the Color Coating Using an OSSI and a Film Forming Composition Comprising a First Component with a First Binder Functional Group and a Second Component with a Second Binder Functional Group and the First and Second Function Groups are Alkenoyloxy and Amine

The procedures used within example 1 were reapplied to example 3, where an alternate film forming composition was tested.

TABLE 5
Different combination Praeparatur and different Fundamenta followed by an OSSI
pre-treatment and an alternate film forming composition.

| | 3A | 3B | 3C | 3D | 3E | 3F | 3G | 3H |
|---|---|---|---|---|---|---|---|---|
| Praeparatur | n/a | X | X | X | X | X | X | |
| Fundamenta | | | | | | | | |
| Basic oxidation | | | X | | | | | |
| Plasma | | | | X | | | | |
| APTT | | | | | X | | | |
| Reductive | | | | | | X | | |
| Reductive then acidic oxid | | | | | | | X | X |
| Pre-treatment | | | | | | | | |
| MTMO | 10.0 | 10.0 | 10.0 | 10.0 | 10.0 | 10.0 | 10.0 | 10.0 |
| Water | Qs | Qs | Qs | Qs | Qs | Qs | Qs | Qs |
| Acetic Acid | 0.04 | 0.04 | 0.04 | 0.04 | 0.04 | 0.04 | 0.04 | 0.04 |
| Coloring Composition | | | | | | | | |
| Silamine 2972¹⁰ | 3.0 | 3.0 | 3.0 | 3.0 | 3.0 | 3.0 | 3.0 | 3.0 |
| Silmer OH ACR Di10¹¹ | 2.0 | 2.0 | 2.0 | 2.0 | 2.0 | 2.0 | 2.0 | 2.0 |
| Pigment red 122 Paste | 10 | 10 | 10 | 10 | 10 | 10 | 10 | 10 |
| IPA | qs | qs | qs | qs | qs | qs | qs | qs |
| Results | | | | | | | | |
| Full root simulation result | − | ++ | + | +++ | +++ | ++ | ++ | ++ |

¹⁰Silamine 2972, a polydimethylsiloxane with pendant/terminal organoamine groups obtained from Siltech Corporation, Canada.
¹¹Silmer OH ACR Di10 a polydimethylsiloxane with pendant/terminal α,β-unsaturated (meth)acryloxy groups obtained from Siltech Corporation, Canada.

In the control leg, 3A, with no Praeparatur or Fundementa, the remanence was no remanence. The use of a Praeparatur and a basic oxidation Fundamenta provided moderate remanence, 3C. The Praeparatur alone, 3B, provided strong performance. The performance with the Plasma and APTT was very strong, 3D and 3E. The use of only a reductive Fundamenta or a combination of reductive followed by acidic oxidation Fundamenta with or without a Praeparatur resulted in strong performance, 3F-3H. These results showed that the OSSI, in combination with Praeparatur and or Fundamenta could provide good color remanence.

### Example 4. Performance of the Color Coating Using an OSSI and a Film Forming Composition Comprising a First Component with a First Binder Functional Group and a Second Component with a Second Binder Functional Group and the First and Second Function Groups Carboxylic Acid and Carbodiimide

The procedures used within example 1 were reapplied to example 4, where an alternative film forming coloring composition was tested.

TABLE 6
Different combination Praeparatur and different Fundamenta followed by an
OSSI pre-treatment and an alternate film forming composition.

| | 4A | 4B | 4C | 4D | 4E | 4F | 4G | 4H |
|---|---|---|---|---|---|---|---|---|
| Praeparatur | n/a | X | X | X | X | X | X | |
| Fundamenta | | | | | | | | |
| Basic oxidation | | | X | | | | | |
| Plasma | | | | X | | | | |
| APTT | | | | | X | | | |
| Reductive | | | | | | X | | |
| Reductive then acidic oxid | | | | | | | X | X |
| Pre-treatment | | | | | | | | |
| MTMO | 10.0 | 10.0 | 10.0 | 10.0 | 10.0 | 10.0 | 10.0 | 10.0 |
| Water | Qs | Qs | Qs | Qs | Qs | Qs | Qs | Qs |
| Acetic Acid | 0.04 | 0.04 | 0.04 | 0.04 | 0.04 | 0.04 | 0.04 | 0.04 |
| Coloring Composition | | | | | | | | |
| Belsil P1101¹² | 10 | 10 | 10 | 10 | 10 | 10 | 10 | 10 |
| Permutex XR-13-554¹³ | 2 | 2 | 2 | 2 | 2 | 2 | 2 | 2 |
| Ammonia¹⁴ | 1.5 | 1.5 | 1.5 | 1.5 | 1.5 | 1.5 | 1.5 | 1.5 |
| Phosphoric acid¹⁵ | 0.1 | 0.1 | 0.1 | 0.1 | 0.1 | 0.1 | 0.1 | 0.1 |
| Pigment red 122 Paste | 10 | 10 | 10 | 10 | 10 | 10 | 10 | 10 |
| IPA | Qs | Qs | Qs | Qs | Qs | Qs | Qs | Qs |
| Results | | | | | | | | |
| Full root simulation result | − | ++ | ++ | +++ | +++ | +++ | +++ | ++ |

¹²Belsil P1101, an organosilicone polymer with pendant/terminal carboxylic acid groups, obtained from Wacker Chemie GmbH
¹³Permutex XR-13-554, an organic polymer with carbodiimide groups obtained from Stahl Holdings B.V.
¹⁴Ammonia obtained as aqueous ammonium hydroxide (25% Ammonia) from Brenntag.
¹⁵Phosphoric Acid added as an 85% w/w aqueous solution from BCD Chemie GmbH.

In the control leg, 4A, with no Praeparatur or Fundementa, the remanence was no remanence. The use of combinations of Prarparatur and Fundamenta provided strong to very strong remanence, 4B-4H. These results showed the performance improvement using an OSSI in combination with Praeparatur and or Fundamenta.

### Example 5. Performance of the Color Coating Using Different OSSI and a Film Forming Composition Comprising an Organic Polymer Comprising at Least One Alkoxysilyl Group

The procedures used within example 1 were reapplied to example 5, where alternate OSSI materials were tested.

TABLE 7
Different OSSI pre-treatment and a film forming composition applied
after a set combination of Praeparatur and Fundamenta.

| | 5A | 5B | 5C | 5D | 5E | 5F | 5G | 5H | 5I |
|---|---|---|---|---|---|---|---|---|---|
| Praeparatur | X | X | X | X | X | X | X | X | X |
| Fundamenta | | | | | | | | | |
| Reductive then acidic oxid | X | X | X | X | X | X | X | X | X |
| Pre-treatment | | | | | | | | | |
| SIP6926.4¹⁶ | | 10 | | | | | | | |
| SIO6704.0¹⁷ | | | 10 | | | | | | |
| SIM6480.0¹⁸ | | | | 10 | | | | | |
| KR-518¹⁹ | | | | | 10 | | | | |
| MTMO | | | | | | 10 | | | |
| X-12-1307MS²⁰ | | | | | | | 10 | | |
| KR-519²¹ | | | | | | | | 10 | |
| X-12-1154²² | | | | | | | | | 10 |
| Ethanol | | Qs | Qs | Qs | Qs | | | Qs | |
| Water | | 5 | 5 | 5 | 5 | Qs | Qs | 5 | |
| Butylacetate²³ | | | | | | | | | Qs |
| Acetic Acid | | 0.08 | 0.08 | 0.08 | 0.08 | 0.08 | 0.08 | 0.08 | |
| Coloring Composition | | | | | | | | | |
| Worlee Add VP 2100 | 0.15 | 0.15 | 0.15 | 0.15 | 0.15 | 0.15 | 0.15 | 0.15 | 0.15 |
| WorleePur VPSi 2021 | 6.67 | 6.67 | 6.67 | 6.67 | 6.67 | 6.67 | 6.67 | 6.67 | 6.67 |
| Pigment red 122 Paste | 10 | 10 | 10 | 10 | 10 | 10 | 10 | 10 | 10 |
| IPA | qs | qs | qs | qs | qs | qs | qs | qs | qs |
| Results | | | | | | | | | |
| Full root simulation result | − | 0 | + | ++ | ++ | +++ | +++ | +++ | +++ |

^{16, 17, 18}were obtained from Gelest, A Group Company of Mitsubishi Chemical.
^{19, 20, 21} and ²²were obtained from Shin Etsu Chemical Co Ltd.
²³Butylacetate was obtained from VWR.

TABLE 8
Details of the materials within the different pre-treatments.

| Material ID/Name | Structure | FEMW | Mw |
|---|---|---|---|
| SIP6926.4 3-(4-Pyridylethyl) Thiopropyl- trimethoxysilane | 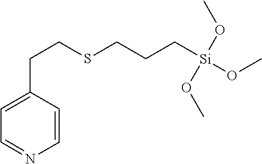 [SMILES: CO[Si](CCCSCCc1ccncc1)(OC)OC] | 301.5 | 301.5 |
| SIO6704.0 S-(Octanoyl) Mercaptopropyl- triethoxysilane | 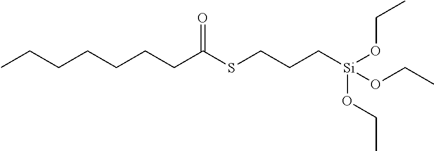 [SMILES: CCCCCCCC(=O)SCCC[Si](OCC)(OCC)OCC] | 364.6 | 364.6 |
| SIM6480.0 11- Mercaptoundecyl- trimethoxysilane | 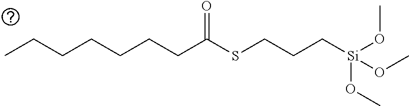 [SMILES: CCCCCCCC(=O)SCCC[Si](OC)(OC)OC] | 308.6 | 308.6 |
| Shin Etsu KR-518 Oligomer of thioalkylalkoxysilane and alkylalkoxy silane | 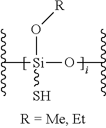 | 800 | 300-3500 Mw |
| Shin Etsu KR-519 Oligomer of thioalkylalkoxysilane and alkylalkoxy silane | 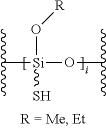 | 450 | 300-3500 Mw |
| Shin Etsu X-12-1154 Organopolymer with pendant alkoxysilyl and alkylthiol groups. | 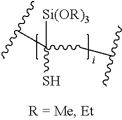 | 240 | 30000 |
| Shin Etsu X-12-1307MS (Trimethoxysilyl) methanethiol | 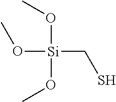 | 168.3 | 168.3 |
| MEMO 3-Mercaptopropyl- trimethoxysilane | 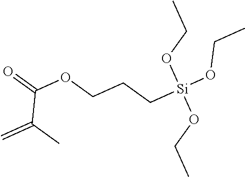 [SMILES: C=C(C)C(=O)OCCC[Si](OCC)(OCC)OCC] | 196.4 | 196.4 |

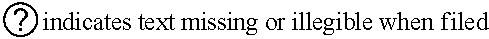

The choice of the solvent used within the pre-treatment was chosen to enable the at least partial solubilization of the silane within the composition. The silanes have a broad range of polarities, and therefore water, ethanol and butylacetate were used. In the control leg, 5A, with no Pre-treatment, the performance was no remanence, no color was observed after the remanence test. The use of the protected thiol, 5B also resulted in weak remanence, but with some color still observed. Whilst not wishing to be bound to any particular theory, it believed that under the conditions tested, the protecting group was not removed sufficiently to provide a strong link to the hair surface, but by changing the conditions the performance could be enhanced. The thioester, 5C, provided moderate remanence. Again, whilst not wishing to be any particular theory, these are believed to produce more free thiol than 5B, and the level could be increased further by optimization of the medium by one skilled in the art. The OSSI used in 5D and 5E gave strong remanence. These both contain free mercapto groups with different spaces or size of the material. 5F is used as reference to the other experimental sections and gave very strong remanence. The OSSI compounds tested within 5G-5I also were able to provide very strong remanence. The different OSSI have very different odour perceptions and one skilled in the art will look to maximise the performance of the thiol with an acceptable odour for users.

### Example 6. Performance of the Color Coating Using Different OSSI and a Film Forming Composition Comprising an Organic Polymer Comprising at Least One Alkoxysilyl Group and Different Ways to Convert to a Colored Coating

The procedures used within example 1 were reapplied to example 6 with the addition of a step to at least partially cure the pre-treatment before the binder step through addition of a catalyst wherein the catalyst comprises an aqueous base, which within these examples was ammonia wherein the catalyst comprises an aqueous base, preferably an aqueous composition of an inorganic or organic nitrogen compound, more preferably ammonia, monoethanolamine or trimethyl amine or triethyl amine
1. After the application of the pre-treatment composition, and the hair drying, 1 g of pre-converter was applied to 1 g of hair and worked through the tress.
2. The tress was left for 5 minutes, and then was dried before moving onto the coloring composition step.

TABLE 8
Different combinations of Praeparatur and/or Fundamenta with
different OSSI pre-treatments, and/or using a pre-converting, before
finally applying the film forming composition.

| | 6A | 6B | 6C | 6D | 6E | 6F | 6G | 6H | 6I |
|---|---|---|---|---|---|---|---|---|---|
| Praeparatur | X | X | X | X | X | X | X | X | X |
| Fundamenta | | | | | | | | | |
| Reductive then acidic oxid | | X | | | X | | | X | |
| Pre-treatment | | | | | | | | | |
| X-12-1307MS | 10 | 10 | 10 | | | | | | |
| KR-519 | | | | 10 | 10 | 10 | | | |
| X-12-1154 | | | | | | | 10 | 10 | 10 |
| Ethanol | | | | Qs | Qs | Qs | | | |
| Water | Qs | Qs | Qs | 5 | 5 | 5 | | | |
| Butylacetate | | | | | | | Qs | Qs | Qs |
| Acetic Acid | 0.08 | 0.08 | 0.08 | 0.08 | 0.08 | 0.08 | | | |
| Pre-Converting step | | | | | | | | | |
| Ammonia | | | 10 | | | 10 | | | 10 |
| Water | | | qs | | | qs | | | qs |
| Coloring Composition | | | | | | | | | |
| Worlee Add VP 2100 | 0.15 | 0.15 | 0.15 | 0.15 | 0.15 | 0.15 | 0.15 | 0.15 | 0.15 |
| WorleePur VPSi 2021 | 6.67 | 6.67 | 6.67 | 6.67 | 6.67 | 6.67 | 6.67 | 6.67 | 6.67 |
| Pigment red 122 Paste | 10 | 10 | 10 | 10 | 10 | 10 | 10 | 10 | 10 |
| IPA | qs | qs | qs | qs | qs | qs | qs | qs | qs |
| Results | | | | | | | | | |
| Full root simulation result | ++ | +++ | +++ | + | +++ | +++ | + | +++ | ++++ |

These results showed that the use of pre-converting step can enhance the remanence of the coating. In these experiments remanence was tested with a Praeparatur, and no Fundamenta, then with a Praeparatur, and a combined Fundamenta of a reductive than acidic oxidation, and finally with a Praeparatur, again with no Fundamenta and then a pre-converting step after the pre-treatment and before the coloring composition. In each of the three OSSI pretreatments tested, X-12-1307MS, 6A-C, KR-519, 6D-F and X-12-1154, 6G-6I, the performance with the pre-converting step on the hair without a Fundamenta is better than without the pre-converting step and the same as the ones using the Fundamenta.

### Example 7. Performance of the Color Coating Using Different OSSI and a Film Forming Composition Comprising an Organic Polymer Comprising at Least One Alkoxysilyl Group with Different Concentration of Reductive Active within the Reduction Processes

The procedures used within example 1 were reapplied to example 7.

TABLE 9
Different Fundamenta tested by concentration of the active reducing
agent and its combination with the acidic oxidation.

| | 7A | 7B | 7C | 7D | 7E | 7F | 7G | 7H |
|---|---|---|---|---|---|---|---|---|
| Praeparatur | X | X | X | X | X | X | X | X |
| Fundamenta | | | | | | | | |
| Reductive Formula | | | | | | | | |
| Thioglycolic acid²⁴ active level | 3 | 9 | 15 | 25 | 3 | 9 | 15 | 25 |
| Ammonia | Use to trim pH to between 9-9.5 | | | | | | | |
| Water | Qs | Qs | Qs | Qs | | | | |
| Acidic oxidation | | | | | X | X | X | X |
| Pre-treatment | | | | | | | | |
| MTMO | 10.0 | 10.0 | 10.0 | 10.0 | 10.0 | 10.0 | 10.0 | 10.0 |
| Water | Qs | Qs | Qs | Qs | Qs | Qs | Qs | Qs |
| Acetic Acid | 0.04 | 0.04 | 0.04 | 0.04 | 0.04 | 0.04 | 0.04 | 0.04 |
| Coloring Composition | | | | | | | | |
| Worlee Add VP 2100 | 0.15 | 0.15 | 0.15 | 0.15 | 0.15 | 0.15 | 0.15 | 0.15 |
| WorleePur VPSi 2021 | 6.67 | 6.67 | 6.67 | 6.67 | 6.67 | 6.67 | 6.67 | 6.67 |
| Pigment Red 122 Paste⁸ | 10 | 10 | 10 | 10 | 10 | 10 | 10 | 10 |
| IPA | qs | qs | qs | qs | qs | qs | qs | qs |
| Results | | | | | | | | |
| Full root simulation result | +++ | +++ | +++ | +++ | +++ | +++ | +++ | +++ |

²⁴Ammonium thioglycolate 70% active obtained from Bruno Bock used a the source for thioglycolic acid.

This series of experiments looked at the level of reducing agent within the Fundamenta. These showed that as the level of reducing agent was increased from 7A to 7D which correlated to an increase from 3 to 25% o of thioglycolic acid, the performance was consistently very strong.

When a sequential acidic oxidation was performed after the reducing treatment the performance from 7E to 7H again the performance was consistently very strong. This shows that a wide range of reductive active may be used within the Praeparatur.

### Example 8. Performance of the Color Coating Using Different OSSI and a Aminoorgano Alkoxysilane and a Film Forming Composition Comprising an Organic Polymer Comprising at Least One Carboxylic Acid Group

The procedures used within example 1 were reapplied to example 8 with two additional processes for making the product which are described below.

### Preparation of a Neutralized PAA Solution (EAA Polymer).

To a beaker the following materials were added. 10 g of Poly(ethylene-co-acrylic acid), a.k.a ethylene-acrylic acid polymer (EAA) from Sigma Aldrich 448672-250G, 90 g of DI water and 0.83 grams of sodium hydroxide granules. The beaker was heated to 92° C. under rapid stirring and a clear to milky emulsion was produced. This was then cooled down to be used in the experimental compositions below.

### Preparation of the Coloring Compositions.

These were prepared using the following method. 0.5 g of the Pigment Red 122 powder was added to speedmixer pot, together with 25 g of the neutralized PAA solution. Mixing beads were also added to assist in the deaggregation of the Pigment Red 122 powder. The speedmixer pot was then mixed at 1550 rpm for 150 seconds. The resulting mixture was strained to removed the mixing beads and the resulting filtered red composition was used as the coloring composition within the experiments.

TABLE 10
Different Fundamenta and the addition of an aminosilane into
the pretreatment composition.

| | 8A | 8B | 8C | 8D | 8E | 8F | 8G | 8H | 8I |
|---|---|---|---|---|---|---|---|---|---|
| Praeparatur | X | X | X | X | X | X | X | X | X |
| Fundamenta | | | | | | | | | |
| Basic oxidation | | X | | | | | | | |
| Plasma | | | X | | | | | | |
| APTT | | | | X | | | | | |
| Reductive | | | | | X | | | | |
| Reductive then acidic oxid | | | | | | X | | | |
| Acidic oxidation | | | | | | | X | | |
| Pre-treatment | | | | | | | | | |
| MTMO | 10 | 10 | 10 | 10 | 10 | 10 | 10 | 70 | 10 |
| SCA²⁵ | | | | | | | | 16.5 | 3.36 |
| Water | Qs | Qs | Qs | Qs | Qs | Qs | Qs | 13.5 | 1.9 |
| HMDS²⁶ | | | | | | | | | Qs |
| Acetic Acid | 0.04 | 0.04 | 0.04 | 0.04 | 0.04 | 0.04 | 0.04 | | |
| Processing Step | | | | | | | | Water | |
| | | | | | | | | Rinse | |
| Coloring Composition | | | | | | | | | |
| Neutralized PAA solution | 25.0 g | 25.0 g | 25.0 g | 25.0 g | 25.0 g | 25.0 g | 25.0 g | 25.0 g | 25.0 g |
| i.e., EAA polymer | | | | | | | | | |
| Pigment Red 122 | 0.5 g | 0.5 g | 0.5 g | 0.5 g | 0.5 g | 0.5 g | 0.5 g | 0.5 g | 0.5 g |
| Results | | | | | | | | | |
| Full root simulation result | 0 | 0 | 0 | 0 | 0 | 0 | 0 | +++ | ++ |

²⁵SCA refers to SIT8398.0, (3-Trimethoxysilylpropyl)Diethylenetriamine from Gelest, A Group Company of Mitsubishi Chemical.
²⁶HMDS refers to hexamethyldisiloxane 98+ % and was obtained from Sigma Aldrich 205389.

Examples 8A-8G used the Praeparatur and Fundamenta combinations used within Examples 1B-1H. In the current examples 8A-8G only weak remanence was observed. Whilst not wishing to be bound to any particular theory, the absence of a charged surface for interactions with the neutralized PAA material is thought to lead to the low remanence. In contrast, example 8H used an additional aminosilane within the pre-treatment. The levels of silanes were high, and a 1 minute rinse was performed after the 5 minute pre-treatment application step, before the hair was dried ready for the coloring composition. The addition of the aminosilane led a very strong remanence of the color result, even in the absence of a Fundamenta. A further example 81 was performed with lower levels of the silanes and no rinsing step before drying the hair ready for the coloring composition. This results in strong remanence.

In addition to the remanence testing, a test was also performed to see if the color coating could be removed from the hair tress. To the color coating hair, 8H, which had not been subjected to any additional sebum or shampoo steps, an off formulation was applied to the hair. This removal composition comprised 10% of Monoethanolamine, 2% 2-n-butoxyethanol, 1% of Carbopol Ultrez 10 and 1.8% of C12-15 Pareth-3 in an aqueous base. 1 g was applied to the 1 g color coated hair tress and worked through the strand with the fingers. After 2 minutes the excess was removed with a paper towel and an additional 1 g of the removal composition was applied and worked into the tress with the fingers. After 2 minutes the excess was again removed with a paper towel, and the tress was then subjected to a shampoo step described above and then dried. The result was that the initial color which was very strong was reduced to weak and was barely noticeable.

### Model Experiment 9

### Demonstration of Disulfide Formation Between Keratin Protein and Thiol Compounds

This model experiment is designed to provide further insight in regard to the reaction of PTH alkoxysilanes (e.g., thiol compounds such as mercaptopropyltrimethoxysilane) with thiol moieties on a keratinous-like surface. The keratinous-like surface consists of a cysteine enriched polymeric surface (cysteine peptide surface). The reaction to be investigated is thought to create disulfide (s-s) bond interactions between the thiol compounds and cysteines. This covalent bonding delivers good remanence of keratin fiber coating formed of the pretreatment, binder and pigment conglomerate as evinced by the following Salon Test Example.

The keratinous-like surface can be prepared according to the procedures described in “Example for Polymeric Peptide foils/Surfaces: Plasma chemical grafting of peptides onto polymeric foils”^{1,2,3}. The keratinous-like surface may also be based upon a Merrifield peptide synthesis which is a well-known technique for synthetic preparation of peptides. The Merrifield technique utilizes a polymeric support to which the C-terminus of the peptide chain is bound. For example, the keratinous-like surface may be prepared as a polyolefin based foil such as a polystyrene formed by polymerization of styrene and a functional olefinic monomer such as 4-hydroxymethyl styrene or hydroxyl protected allyl alcohol. The pendant hydroxy group of the styrene polymer can be combined with the C-terminus of an N-BOC protected amino acid such as glycine to form the polystyrene with pendant glycinyl ester groups. The polymer can be cast as a thin membrane and an N-protected cysteine or N-protected di-cysteine disulfide can be amidated through its carboxyl group to the pendant, bound glycinate ester to form a polystyrene membrane or foil carrying pendant cysteine groups or pendant di-cysteine disulfide groups. ¹ “Plasmachemical grafting of RGD peptides onto polymeric foils to improve the proliferation of cells”, M. Müller, T. Huben and C. Oehr, Meeting abstracts: 4th Congress on Regenerative Biology and Medicine, October 13-15, 2010, Stuttgart, Germany New Rochelle/NY: Liebert, Tissue engineering. Part A, 17(3-4), 562, 2011² “Directed evolution of polypropylene and polystyrene binding peptides”, K. Rubsam, F. Jakob, U. Schwaneberg of Aachen University Aachen Germany, Biotechnology and Bioengineering, 115(2), 321-330, 2017.³ “Merrifield Solid-Phase Peptide Synthesis”, R Merrifield, J. Am. Chem. Soc., 85(14), 2149-2154, 1963.

In a first experiment, the foil or membrane presenting a cysteine (thiol) or di-cysteine disulfide surface may be used as a substrate. When using the membrane with the di-cysteine disulfide surface, a reductive Fundamenta procedure may be applied to cleave the disulfide and produce a membrane carrying pendant bound cysteine presenting free thiol groups. In this example, the membrane may be washed with an appropriate rinsing aid to remove the free cysteine. When using the cysteine (thiol) polymer surface, the reduction Fundamenta procedure is not needed to access free cysteine thiol groups.

In a representative model of the application of the pretreatment composition to the modified keratin fibers, two samples of the previously prepared membrane carrying pendant, bound cysteine presenting accessible thiol groups may be set up. Both samples may be treated with weak acidic oxidizing medium such as 0.5%-1% hydrogen peroxide in mild aqueous acidic acid at pH 4.5-5.5 for a period of about 10 to 30 seconds and then briefly rinsed with water to remove excess oxidizing medium. One membrane sample may be combined with an organic medium carrying mercaptopropyl trimethoxysilane (MTMO). The other membrane sample may be combined with an organic medium carrying a similar trimethoxysilane that is not substituted with a thiol group. Such a silane may be propyltrimethoxysilane (PTMO). Following silane treatment for approximately 5 to 10 minutes, each membrane is repeatedly rinsed with the same organic medium free of any other component to remove excess silane medium. The membrane samples may then be dried with warm air. The medium for this MTMO and PTMO application and rinsing should not include water and especially not water with acetic acid. The presence of an aqueous medium will catalyze the silicone polymer formation from the trimethoxysilane groups. The silicone polymer will coat the membrane so that the subsequent determination of the disulfide linkage will be masked.

The principles underlying the success of the present invention indicate that the MTMO should be bound to the membrane but the PTMO should not. Consequently, the drying step of the membranes should yield the MTMO membrane with a pre-coating of the silane and the PTMO membrane without a pre-coating of silane. A non-thiol containing reducing agent in appropriate non-aqueous medium may be contacted with each membrane sample. An appropriate reducing agent may be sodium borohydride or stannous chloride. The resulting solution produced by combination of the reducing agent and the MTMO membrane should contain either the MTMO or an oligomer of this material. If the methoxysilyl groups of the MTMO condensed during the reduction step, the oligomer would be produced. Depending on its molecular weight, the oligomer will either be soluble in the non-aqueous medium or will appear as a solid. The resulting solution produced by combination of the reducing agent and the PTMO membrane should be devoid of PTMO or an oligomer thereof. The PTMO should have been removed from the membrane during the repeated rinse cycles with the non-aqueous medium.

In an alternative experiment, samples of the MTMO and PTMO membranes may be combined with a coloring composition using the compositions described in foregoing examples 1 and 2, namely the WorleePur VPSi 2021 and the Pigment Red 122 Paste. The coating procedure will follow the procedures outline in examples. The membranes may be repeatedly washed with an aqueous detergent surfactant solution. After about 15 repeats of the shampooing, the MTMO membrane should remain red in color but the PTMO membrane should be the color of the membrane itself, in other words the red color should have been removed. By using a cysteine rich polymer membrane surface, these experiments will enable a specific coordination of increased remanence performance and the covalent attachment of the thiol alkoxysilane to the cysteine surface.

### Salon Test Example

The following test on models with anagenic hair within a test salon further demonstrated the performance of the prototypes described above. Testing was performed in our in-house test salon, with the intent of coloring close to, but not touching the scalp of the model. The protocols applied were similar to those described above.

### Praeparatur.

Prior to any product application the full head of the model was washed with Deep-Cleanser Shampoo from System Professional. The shampoo was applied and massaged into the hair followed by rinsing of the product at the back basin. Then the hair was towel dried and blow dried.

### Fundamenta.

If used the following protocols were followed.

### Reductive.

To a strand of hair, Wella Create Wave reductive formulation was applied and worked into the hair. It was left to work for 10 minutes and then rinsed with water. The hair was then dried with a towel and hair drier.

Reductive then Acidic Oxidation.

The reductive step preceding was used, but rather than dry the hair after rinsing, the Wella Create Wave oxidative neutralisation formulation was worked into the strand. It was left to work for 10 minutes and then rinsed with water. The hair was then dried with a towel and hair drier.

### Pre-Treatment.

The pre-treatment formulation was applied and worked into the target strand with a brush. The product was not applied to the hair directly next to the scalp to prevent skin contact. The pre-treatment was left for five minutes and then blow dried. There was no rinsing step.

### Coloring Composition.

The coloring composition was applied and worked into the target hair strand with a brush to ensure it looked homogenous. The product was not applied to the hair directly next to the scalp to prevent skin contact. The hair strand was then blow dried. Representing the results in images. Images were captured on a standard digital camera, and the following processes were performed to the images to highlight the lasting performance of the red color. This was performed as standard sRGB images cannot be attached to the patent application which would be the easiest way to show the results. The starting sRGB image is processed such that a final image can be presented which shows the CIELAB a* value. a* is the green to red axis within CIELab color space. Green colors have negative a* values and red colors have positive a* values. Such images are greyscale and show red as a light values within the image and non-red areas as dark within the picture. The image is scaled such that an a* of 80 is white and a* of 0 or less is black. To create such an image the following transformations were performed on the starting sRGB image for every pixel. sRGB values of R, G, and B are converted into linear sRGB′ values using F(R) if R/255 >0.04045 is ((R/255+0.055)/1.055)^{2.4} and if R/255≤0.04045 is (R/255)/12.92. This same transformation are applied for R, G and B. The linear sRGB values are then mapped into XYZ tristimulus values with the following transformation:

$$[ \begin{matrix}X \\ Y \\ Z\end{matrix} ] = [ \begin{matrix}0.4124564 & 0.3575761 & 0.1804375 \\ 0.2126729 & 0.7151522 & 0.072175 \\ 0.0193339 & 0.119192 & 0.9503041\end{matrix} ] [ \begin{matrix}linearR \\ linearG \\ linearB\end{matrix} ]$$

These XYZ terms and then used to create the terms f(X/Xₙ), f(Y/Zₙ) and f(Z/Zₙ) with Xₙ=0.950489, Yₙ=1 and Xₙ=1.08884 for D65 Standard Illuminant using the following equations where t=(X/Xₙ), (Y/Zₙ) and (Z/Zₙ); if t >(6/29)³, f(t)=³\sqrt{t}, otherwise f(t)=4/29+t/(3(6/29)²). Finally the a* values are obtained using

$${a}^{*} = 500 ( f ( \frac{X}{{X}_{n}} ) - f ( \frac{Y}{{Y}_{n}} ) ) .$$

Within the resulting a* images the values are plotted on a scale of a*≤0 (black R=G=B=0) to a*=80 (white R=G=B=255).

TABLE 11
Systems tested within the salon.

| | S1 | S2 | S3 | S4 | S5 | S6 |
|---|---|---|---|---|---|---|
| Praeparatur | X | X | X | X | X | X |
| Fundamenta | | | | | | |
| Reductive | | X | | | X | |
| Reductive then acidic oxid | | | X | | | X |
| Pre-treatment | | | | | | |
| MTMO | | | | 10 | 10 | 10 |
| MEMO | 5 | 5 | 5 | | | |
| Ethanol | qs | qs | qs | | | |
| Water | 5 | 5 | 5 | qs | qs | qs |
| Acetic Acid | | | | 0.08 | 0.08 | 0.08 |
| Worlee Add VP 2100 | 0.25 | 0.25 | 0.25 | | | |
| Coloring Composition | | | | | | |
| Worlee Add VP 2100 | 0.15 | 0.15 | 0.15 | 0.15 | 0.15 | 0.15 |
| WorleePur VPSi 2021 | 6.67 | 6.67 | 6.67 | 6.67 | 6.67 | 6.67 |
| Pigment red 122 Paste | 10 | 10 | 10 | 10 | 10 | 10 |
| IPA | qs | qs | qs | qs | qs | qs |

### Salon Results.

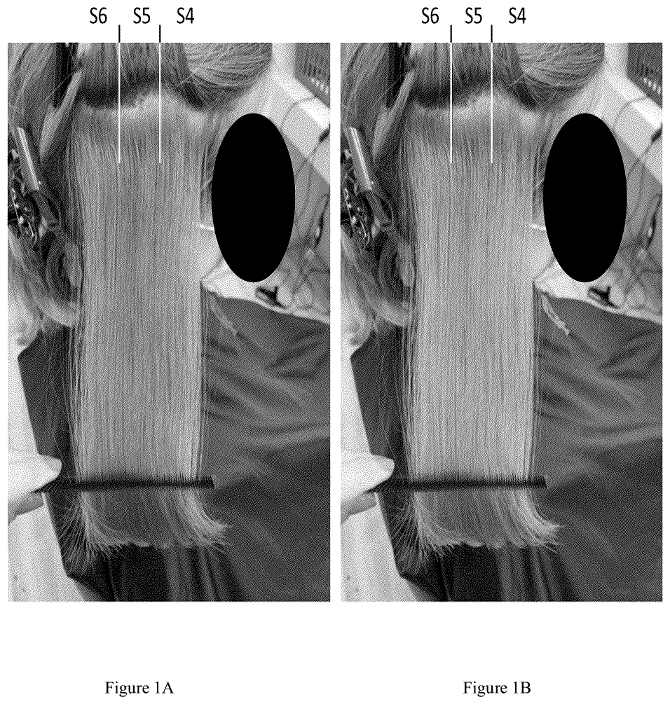

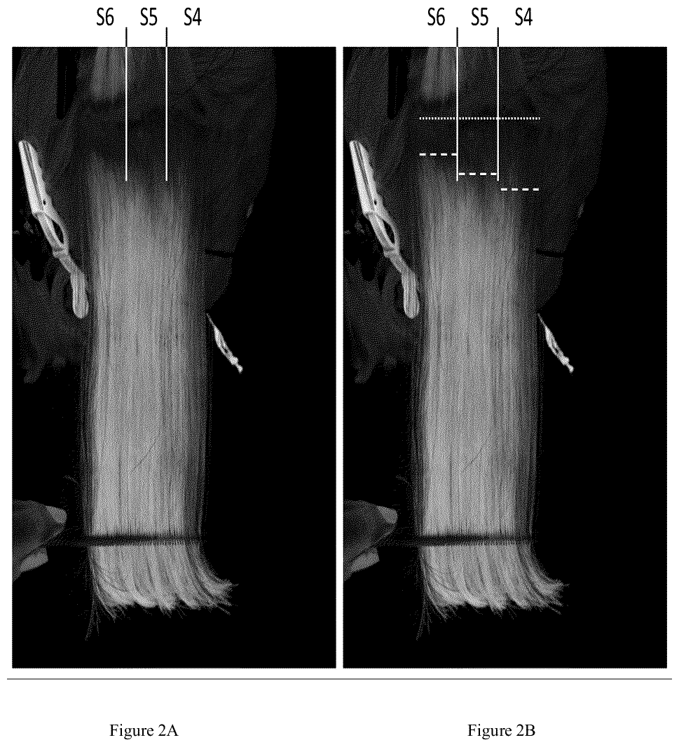

A representative image is shown in FIG. 1A for systems S4-S6. This image was taken after the model had washed their hair at home 15 times, over many days. While results are shown for a single model, they were consistent across a series of models. Note this represents more washing cycles than in the lab testing protocols described earlier to stress test the performance. The left side image, FIG. 1A, is a sRGB color image taken under a d65 illuminate panel, with the different strands S4-S6 annotated onto the image. The red color remanence strands was highly visible and extended towards the hair adjacent to the scalp. The closest red color to the roots was S6 followed by S5 and then S4. Recall, products were not applied all the way to the roots, so for S6, the color was the most highly remanent at the roots. The right sided image, FIG. 1B, shows a typical greyscale image of the same sRGB picture, again with S4-S6 annotated. Such greyscale images can show the main features of the image but were not useful to show where the red color remained on the hair. FIG. 2A shows the a* scale image of the sRGB photo created using the protocol described above. a* is a measure of “red” within the CIELab color space. The a* image plot the a* values from 0 to 80, with 80 being white in the image. The left side image, FIG. 2B, is annotated again with the different strands S4-S6. In this image the light regions on the hair correspond to where the color red remains after the 15 wash cycles. The lightest regions in the image are those that correspond to the reddest regions in the standard sRGB image. Overall, the treated hair strands are light from root to tip indicating a highly remanent red hair color effect after the 15 wash cycles. The red extends close to the root area for S4, even closer to the root for S5 and was closest to the root for S6. This is highlighted in the right hand image, where the approximate location of the end of the color are shown with dashed lines, and the location of the roots are shown with a dotted line.

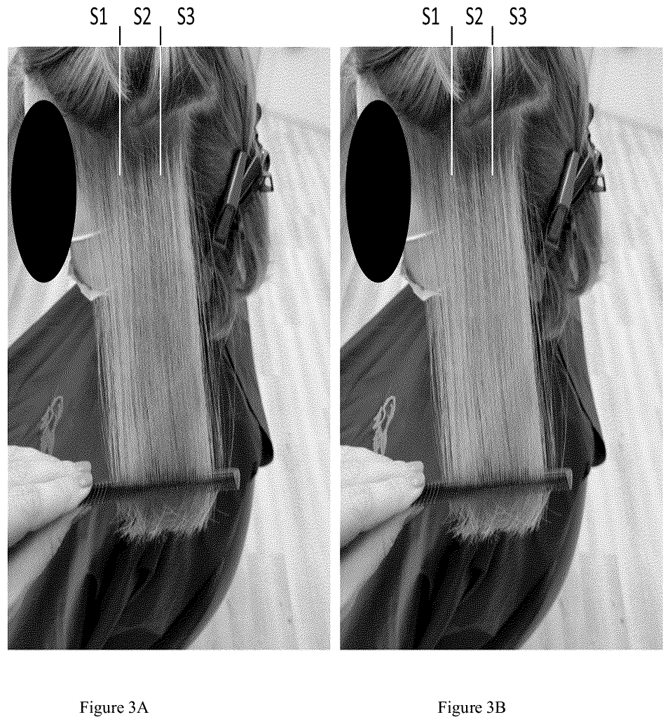

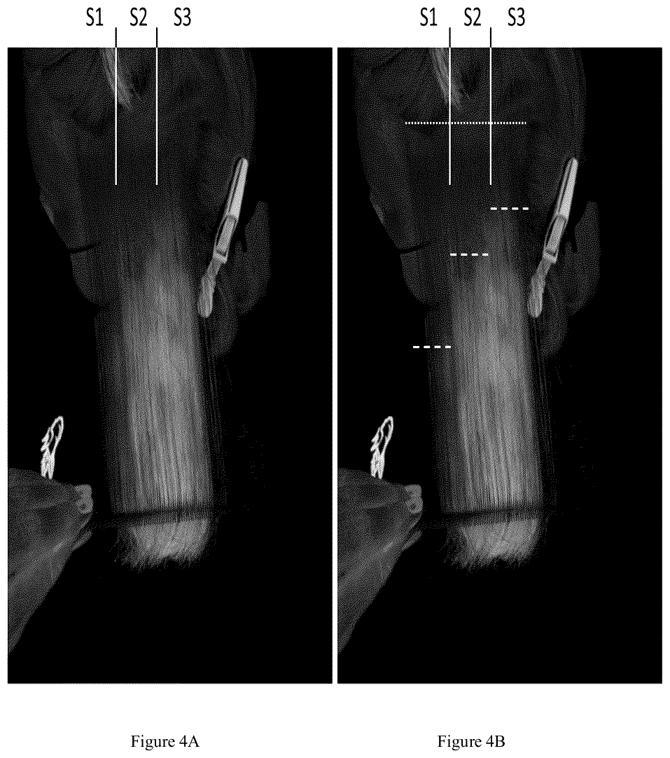

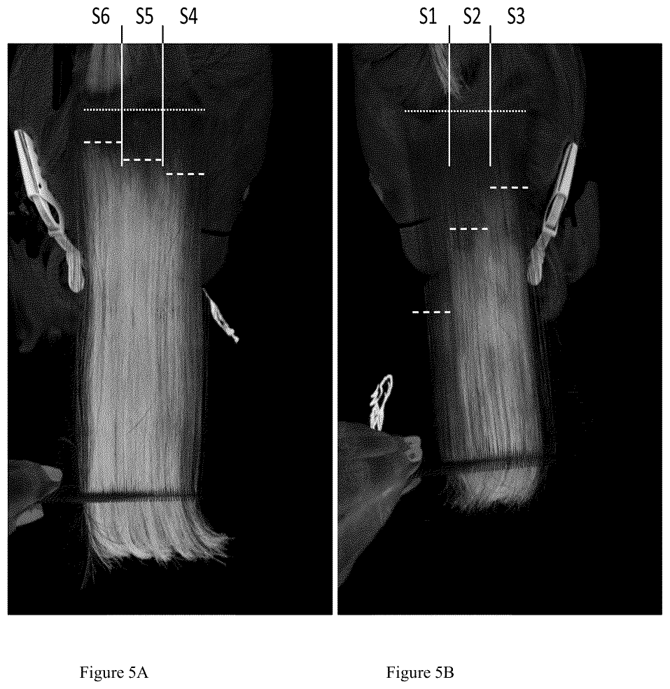

FIGS. 3A and 3B show the other side of the same model also after 15 wash cycles where the systems S1-S3 were applied. In FIG. 3A, the sRGB image, left, the amount of red color was visibly less, both in the lengths and ends and especially near to the roots. In the right hand greyscale image, FIG. 3B, the color information is lost. In FIG. 4A, the a* images, the red becomes noticeable again in the lighter areas of the picture which are the reddest areas. However, it is considerably less than that seen in FIG. 2B. On the right side image, FIG. 4B, the rough location of the color versus the root is drawn in to show the differences between S1, S2 and S3. In S1 the color is a very long way from the root of the model. This is closer to the roots in S2 and closer again in S3. However, the performance of S3 does not meet the level of root performance of the weakest performing from the series S4-S6. This is shown clearly in FIGS. 5A and 5B where the a* images of all test systems are shown together, i.e., FIG. 2B and FIG. 4B. The systems with the OSSI pre-treatment, i.e. thiolorganic alkoxysilane provide a significant increase in color remanence, especially when combined with a Fundamenta.

### STATEMENTS OF EMBODIMENTS OF THE INVENTION

The following statements of embodiments of the invention describe aspects, features and parameters of the methods, compositions and treatments according to the invention. These statements provide further disclosure of these aspects, features and parameters and may serve as claims of the invention.

### Part I, Generic Description Statements

1. A method for producing a coating, preferably a color coating, on keratin fibers comprising:
   an activating step comprising contacting the keratin fibers with either or both of a Praeparatur procedure and a Fundamenta procedure to form modified keratin fibers;
   a pretreatment step comprising applying to the modified keratin fibers a pretreatment composition to form pre-coated keratin fibers;
   a binder step comprising applying to the pre-coated keratin fibers a film forming composition to form a composite film of the film forming composition and pretreatment composition on the keratin fibers and the composite film is capable of converting to the coating;
   conducting the activating and pretreatment steps either simultaneously or sequentially; and,
   optionally and preferably combining at least one colorant with the pretreatment composition and/or the film forming composition;
wherein:
   the Praeparatur procedure comprises a cleaning process;
   the Fundamenta procedure comprises an acidic oxidation process, a basic oxidation process, a plasma process, an alkali phase transfer tenside process, a reduction process or any combination thereof,
   the pretreatment composition comprises a medium, at least a PTH-alkoxysilane compound comprising a PTH-organo-alkoxysilane with at least one PTH group and at least one alkoxysilane group and/or a PTH-organo-multidimethylsiloxane alkoxysilane with at least one PTH group and at least one alkoxysilane group, wherein PTH comprises R³S—, OHC—, H₂C═CR¹⁰—CO₂— or HO—, and R³ comprises hydrogen or a sulfur protecting group; and/or further comprising a disulfide dimer of the PTH alkoxysilane compound wherein PTH is thiol and/or a tetrasulfide dimer of the PTH-alkoxysilane compound wherein PTH is thiol, and optionally further comprising a PTH organic compound and/or an aminoorgano alkoxysilane compound;
   the film forming composition comprises a medium and a binder polymer comprising either a unitary binder polymer with at least one binder functional group or a dual binder polymer comprising a first organic, silicone or organosilicone component and a second organic, silicone or organosilicone component with the first and second components comprising a complementary pair of binder functional groups.
2. A method according to any of the preceding PART I statements wherein the activating step comprising at least one of the processes of the Fundamenta procedure and the pretreatment step comprising applying the pretreatment composition comprising at least a PTH alkoxysilane compound produces a colored coating having a longer lasting color remanence according to the full root simulation color remanence test than the remanence of a colored coating produced without the practice of the activating step comprising at least one of the processes of the Fundamenta procedure and without the practice of the pretreatment step comprising applying a PTH alkoxysilane compound and tested by the full root simulation color remanence test.
3. A method according to statement 2 of PART I wherein the PTH alkoxysilane compound reacts with the hair.
4. A method according to any of the preceding PART I statements wherein the PTH group is a thiol group.
5. A method according to any of the preceding PART I statements wherein the pretreatment composition comprises a PTH alkoxysilane compound comprising at least one of the PTH organo-alkoxysiloxane compound of Formula IIIA, the PTH organo-multi-dimethylsiloxanyl alkoxysilane of Formula IIIB, the disulfide or tetrasulfide dimer of Formula IIIA with PTH as thiol, the disulfide or tetrasulfide dimer of Formula IIIB with PTH as thiol, the cyclic thiol-alkoxysilane compound of Formula IV, and any combination thereof,

(PTH—(CH₂)ₖ—(Y)ₗ)_{d}—(ORG)ₘ-SiR¹₃₋ₙ(OR)ₙ Formula IIIA

PTH—(CH₂)ₖ—(Si(Me)₂O)ₒ—SiR¹₃₋ₙ(OR)ₙ Formula IIIB

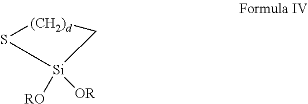

wherein:
   Designator k is an integer of 1 to 20, preferably 1 to 12, more preferably 1 to 6;
   Designator 1 is zero or 1;
   Designator d is an integer of 1, 2 or 3;
   Designator m is zero or an integer of 1 to 6;
   Designator n is an integer of 1 to 3;
   Designator o is an integer of from 1 to 20;
   PTH comprises R³S—, OHC—, H₂C═CR¹⁰—CO₂— or HO—;
   R³ comprises hydrogen, cyano, alkanoyl of 2 to 10 carbons, a phenyl group, a heteroaromatic group, a phenylalkyl group or a heteroaromatic alkyl group in which heteroaromatic group is pyridyl, pyrimidinyl, pyrrolyl or thiophenyl and the alkyl group is a C1-C4 alkyl group, and R¹⁰ may be hydrogen or methyl, such that R³S— may be a thiol group (HS—) or a protected thiol group;
   R¹ comprises C1-C3 alkyl, preferably methyl;
   R comprises a C1-C4 alkyl, preferably C1-C3 alkyl, more preferably methyl or ethyl;
   Y comprises —COO—, —OOC— (carboxyl, oxycarbonyl), ether oxygen, ether thiol, —NMe-NH—, —HNCO—, —CONH—;
   Group ORG comprises:
   (i) a divalent organic group selected from alkyldithioalkyl, alkyldiazoalkyl, alkylurethanylalkyl, alkylureidoalkyl, alkylcarboxylalkyl, alkylamidoalkyl, alkylesteralkyl or alkyl in which each alkyl group independently in each instance is a C1-C20 linear or branched alkyl group, preferably a linear C1-C6 alkyl group, more preferably a linear C1-C3 alkyl group such that ORG connects the left (PTH—(CH₂)ₖ—(Y)ₗ)_{d} section and the right —SiR¹₃₋ₙ(OR²)ₙ section of Formula III; or,
   (ii) a multivalent C1-C20 alkylenyl group of the formula

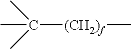

with f as zero or an integer of 1-19 in which the moiety of Formula IIIA comprising (PTH—(CH₂)ₖ—(Y)ₗ)_{d}—(ORG)ₘ becomes Formula A

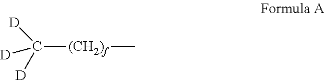

wherein two or three (PTH—(CH₂)ₖ—(Y)ₗ)_{d} sections are connected as D provided that when two D's are (PTH—(CH₂)ₖ—(Y)ₗ)_{d} sections, the third D of Formula A may be hydrogen or C1-C6 alkyl, preferably C1-C3 alkyl, more preferably methyl; and the dangling valence of (CH₂)_{f}— is bound to the right —SiR¹₃₋ₙ(OR²)ₙ section of Formula IIIA.
6. A method according to statement 5 of PART I wherein the pretreatment composition comprises at least a polycondensate of the PTH alkoxysilane of Formula IIIA and/or Formula IIIB with PTH as thiol or protected thiol wherein the PTH alkoxysilane of Formula IIIA and/or Formula IIIB is at least partially polycondensed with itself and/or an alkylalkoxysilane of Formula B wherein R⁸ is a linear or branched alkyl group of 1 to 10 carbons:

R⁸—SiR¹₃₋ₙ(OR)₁ Formula B
to produce a linear or branched oligomeric silicone polycondensate having a silicone chain of a combination of M, D and T groups wherein the polycondensate has pendant alkoxy groups, pendant thiolalkyl groups and/or pendant alkyl groups, and the polycondensate has an M_{w} from 350 to 3500 Da and a functional equivalent M_{w} (FEM_{w}) of the thiol and or protected thiol group from 100 to 900 and a FEM_{w} for the alkoxy groups from 50 to 900.
7. A method according to any of the preceding statements of PART I wherein PTH is thiol (HS—).
8. A method according to statement 5 wherein disulfide dimer comprises two thiolorgano-alkoxysilanes of Formula IIIA or two thiolorgano multidimethylsiloxanyl alkoxysilanes of Formula IIIB joined together at their thiol groups to form a bis[organo-alkoxysilanyl]disulfide or a bis[organo-multidimethylsiloxanyl alkoxysilane]disulfide respectively; and the tetrasulfide dimer comprises two thiolorgano-alkoxysilanes of Formula IIIA or two thiolorgano multidimethylsiloxanyl alkoxysilanes of Formula IIIB joined together at their thiol groups by combination with sulfur as S2 to form a bis[organo-alkoxysilanyl]tetrasulfide or bis[organo-multidimethylsiloxanyl alkoxysilane]tetrasulfide.
9. A method according to any of the preceding statements of PART I wherein PTH is thiol and the thiolorgano alkoxysilane compound and/or the thiolorgano multidimethylsiloxanyl alkoxysilane are present, and the bis[organo-alkoxysilane]disulfide and/or bis[organo-multidimethylsiloxanyl alkoxysilane]disulfide are present and the sulfur functional equivalent weight average molecular weight (FEM_{w}) of the thiol compound is higher than the sulfur FEM_{w} of the disulfide.
10. A method according to any of the preceding statements of PART I reciting the pretreatment composition wherein the PTH organo-alkoxysilane compound comprises Formula OSSI wherein k is an integer of 1 to 20, preferably 1-12, more preferably 1-6, and the multi CH₂ chain may be linear or branched, n is an integer of 1 to 3, R¹ is methyl and R² is methyl or ethyl.

HS—(CH₂)ₖ—SiR¹₃₋ₙ(OR²)ₙ Formula OSSI.
11. A method according to statement 10 wherein Formula OSSI comprises

HS—(CH₂)ₖ—Si(OMe)₃ or HS—(CH₂)ₖ—Si(OEt)₃
wherein k is an integer of 1 to 6, preferably 1-3.
12. A method according to any of the preceding statements of PART I wherein the pretreatment composition also comprises a thiol organic compound of Formula V

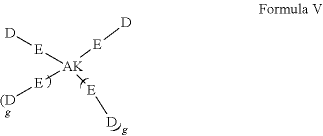

wherein
   D is (PTH—(CH₂)ₖ—(Y)ₗ)_{d} as defined above. Each of the designators g is independently zero or 1. The group E may be a bond or a C1-C6 alkylenyl group; the group Ak is a carbon atom Ak0 or the structures Ak1, Ak2, Ak3, Ak4 depicted as follows wherein the dangling valences of the central carbon of Ak0, Ak1, Ak2 and Ak3 are bonded to E-D and the CH₂ valence is bonded to D; all dangling valences of Ak4 are bound to E-D.

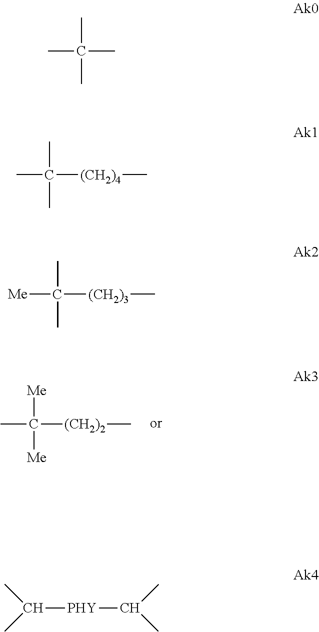

wherein PHY is an oligomer of 2 to 10 units of a C3-C8 α,ω hydroxyalkanoic acid ester having a —O—(CH₂)ₕ—O— at its carboxy terminus and a —(CH₂)ᵢ—O— group at its hydroxyl terminus in which the —O—(CH₂)ₕ—O— and —(CH₂)ᵢ—O— groups are bonded respectively to the CH groups and the designator h is an integer of from 2 to 4 and the designator i is an integer of from 1 to 3.
13. A method according to statement 12 of PART I wherein PTH is thiol (—SH).
14. A method according to any of the preceding statements of PART I wherein the pretreatment composition comprises a combination of PTHorgano-alkoxysilane of Formula IIIA of statement 5 and a PTH organic compound of Formula V of statement 13 in which PTH in both instances is thiol and the thiol FEMw of the thiol organic compound is less than the FEMw of the thiolorgano-alkoxysilane.
15. A method according to any of the preceding statements of PART I wherein the pretreatment composition also comprises the aminoorganoalkoxysilane compound comprising Formula VI:

H₂N—(CH₂)ₘ—(NH—R¹⁴—)ₙ—[ROₜMe₃₋ₜSi—O]_{b}—(—SiMe₂-O)ₚ—[(—SiMe₂₋ᵣ[(CH—₂—)_{m′}—NH₂]ᵣ—O]ₛ—[A]_{c}-[(—SiMe₂-O]ᵤ—(SiMe₃₋ₜORₜ) Formula VI
wherein
   Each instance of R¹⁴ is independently a C1-C6 alkylenyl group;
   R may be methyl or ethyl;
   Designators m and m′ may be an integer of 1 to 3;
   Designators b, r, s, c, may be zero or 1;
   Designator n may be zero or an integer of 1-6, preferably 1-3;
   Designator t is 1 to 3;
   Designators p and u may be zero or an integer of 1 to 12;
   Group A may be a divalent group including dithio, diazo, urethanyl, ureido, carboxyl, amido, ester, or aminoethyloxycarbonyl, or a C1-C20 alkylenyl group connecting the left and right sections of the aminoorgano-alkoxysiloxane compound; or,
   Group A may be a multivalent C1-C20 alkylenyl group connecting two or three left sections and one right section of the aminoorganoalkoxysiloxane compound when a is 2 or 3 and b, p and s are zero; or,
   Group A may be a linear or branched polyethylene imine moiety of from 2 to 2000 ethylene imine units in which case, b, p, s and u are all zero and optionally the group —(SiMe₃-t ORₜ) can be replaced by —NH₂; or,
   Group A may be a terminal group selected from C2-C8 alkylenyl(meth)acrylate or —(CH₂)ₙ—O—CH₂—CHOHCH₂—O₂C(R)═CH₂ wherein R is H or CH₃ and n is an integer of 2 to 8.
16. A method according to statement 15 wherein Formula VI comprises Formula OASI

H₂N—(CH₂)ₘ—(NH—R¹⁴—)ₙ—(SiMe₂O)ₚ-A_{c}(-SiMe₂-O)ᵤSiMe₃₋ₜORₜ Formula OASI
wherein
   m is an integer of 1 to 6;
   n is zero or an integer of 1 to 3;
   p and u are each independently zero or an integer of 1 to 3;
   c is zero or 1;
   t is an integer of 1-3
   A is C1-C6 alkylenyl; and,
   R is methyl or ethyl.
17. A method according to statement 16 wherein Formula OASI is
   H₂N—(CH₂)ₘ—Si(OR)₃ or,
   H₂N—(CH₂)ₘ—(NH—R¹⁴)ₙ—NH—R^{14′}—Si(OR)₃ or,
   H₂N—(CH₂)ₘ—NH—R¹⁴—Si(OR)₃
   wherein m is 2 or 3, n is 1 or 2, each instance of R¹⁴ independently is ethyl or propyl or isobutyl and R^{14′} is propyl, butyl or isobutyl.
18. A method according to statement 17 wherein each instance of R₁₄ is ethyl or propyl and R14′ is propyl or butyl.
19. A method according to any of the preceding statements of PART I wherein the pretreatment step and binder step are conducted simultaneously.
20. A method according to any of the preceding statements of PART I wherein the pretreatment step and binder step are conducted sequentially.
21. A method according to any of the preceding statements of PART I wherein the keratin fibers are anagenic hair, preferably hair on the scalp of a human.
22. A method according to any of the preceding statements of PART I wherein the keratin fibers are anagenic hair which has not been subjected to shampooing, bleaching, and/or reductive process or reductive process followed by acidic oxidative treatment. within at least one day.
23. A method according to any of the preceding statements of PART I wherein the colorant is not present.
24. A method for producing a coating or preferably a color coating on keratin fibers comprising:
   an activating step comprising applying a Praeparatur procedure and a Fundamenta procedure to the keratin fibers to form modified keratin fibers;
   a pretreatment step comprising applying to the modified keratin fibers a pretreatment composition to form pre-coated keratin fibers; and
   a binder step comprising applying to the pre-coated keratin fibers a film forming composition to form a composite film of the film forming composition and pretreatment composition on the keratin fibers and curing the composite film to form the coating;
   conducting the Praeparatur and Fundamenta procedures sequentially or simultaneously;
   conducting the pre-treatment and the binder steps simultaneously or sequentially; and,
   optionally and preferably combining at least one colorant with the pretreatment composition and/or the film forming composition;
wherein:
   the Praeparatur procedure comprises a cleaning process and the Fundamenta procedure comprises an acidic oxidation process or a reduction process or a combination of the reduction process and then the acidic oxidation process;
   the pretreatment composition comprises a medium, at least a PTHorganoalkoxysilane compound comprising the OSSI formula HS—(CH₂)ₖ—SiR¹₃₋ₙ(OR²)ₙ wherein k is an integer of 1 to 20, preferably 1-10, more preferably 1-6, n is an integer of 1 to 3, R¹ is methyl and R² is methyl or ethyl, and optionally an aminoorgano alkoxysiloxane compound comprising the OASI formula H₂N—(CH₂)ₘ—(NH—R¹⁴)ₙ—(SiMe₂O)ₚ-A_{c}-(—SiMe₂-O)ᵤ—SiR³₃₋ₜOR⁴ₜ wherein m is an integer of 1 to 6; n is zero or an integer of 1 to 3; each of p and u independently is zero or an integer of 1 to 3; c is zero or 1; A is C1-C6 alkylenyl; t is an integer of 1-3; R³ is methyl and R⁴ is methyl or ethyl;
   the film forming composition comprises a medium, a first organic, silicone or organosilicone component and a second organic, silicone or organosilicone component wherein the first and second components are the same or different and have binder functional groups.
25. A method according to preceding statement 24 wherein the Praeparatur and Fundamenta procedures are practiced simultaneously and then the Pretreatment and binder steps are practiced simultaneously.
26. A method according to any of the preceding statements of PART I wherein the composite film is converted to the coating on the keratin fibers by application of an external curing method to the composite film.
27. A method according to any of the preceding statements of PART I wherein the pre-treatment composition is at least partially cured before the binder step and the curing method of the coating and preferably the colored coating comprises a procedure selected from drying, heating, and addition of a catalyst or combinations thereof to promote the rate of curing.
28. A method according to statement 23 wherein the catalyst comprises an aqueous base, preferably an aqueous composition of an inorganic or organic nitrogen compound, more preferably ammonia, monoethanolamine or trimethyl amine or triethyl amine.
29. A method according to any of the preceding statements of PART I wherein the curing of the composite coating comprises a procedure selected from drying, heating, heating and drying and addition of a catalyst to promote the rate of curing.
30. A method according to any of the preceding statements of PART I wherein the colorant is present and comprises a pigment or a coated pigment.
31. A method according to any of the preceding statements of PART I wherein the Fundamenta procedure is optionally combined with the pretreatment step, and preferably the Fundamenta procedure is an acidic oxidation process, a basic oxidation process, a reduction process or a combination of a reduction process and then an acidic oxidation process.

### Part II, Activating Steps

All of the PART II statements depend from any and all the foregoing PART I statements.
1. A method according to any of the preceding statements of PART I wherein the activating step includes the Praeparatur procedure and the Fundamenta procedure and the Praeparatur procedure and Fundamenta procedure optionally are combined.
2. A method according to any of the preceding statements of PART I OR PART II wherein the Praeparatur procedure comprises at least a cleaning technique.
3. A method according to any of the preceding statements of PART I OR PART II wherein the process of the Fundamenta procedure is the acidic oxidation process.
4. A method according to any of the preceding statements of PART I OR PART II wherein the process of the Fundamenta procedure is the basic oxidation process.
5. A method according to any of the preceding statements of PART I OR PART II wherein the process of the Fundamenta procedure is the reduction process.
6. A method according to any of the preceding statements of PART I OR PART II wherein the process of the Fundamenta procedure is the Plasma or phase emulsion transfer tenside (PETT) process.
7. A method according to any of the preceding statements of PART I OR PART II wherein the process of the Fundamenta procedure is a combination of the reduction process followed by the acidic oxidation process.
8. A method according to any of the preceding statements of PART I OR PART II wherein the Praeparatur procedure and Fundamenta procedure are conducted sequentially.
9. A method according to any of the PART I or II statements 3, 4, 5, 6, 7 wherein the Praeparatur procedure and Fundamenta procedure are conducted simultaneously.
10. A method according to any of the preceding statements of PART I OR PART II wherein the Praepartur procedure is followed by the reduction process which is followed by the acidic oxidation process.
11. A method according to any of the preceding statements of PART I OR PART II wherein the activating step includes at least one of the processes of the Fundamenta procedure but not the Praeparatur procedure.
12. A method according to statement 11 of PART II wherein the process of the Fundamenta procedure is the acidic oxidation process.
13. A method according to statement 11 of PART II wherein the process of the Fundamenta procedure is the basic oxidation process.
14. A method according to statement 11 of PART II wherein the process of the Fundamenta procedure is the reduction process.
15. A method according to statement 11 of PART II wherein the process of the Fundamenta procedure is the Plasma or PETT process.
16. A method according to statement 11 of PART II wherein the process of the Fundamenta procedure is a combination of the reduction process followed by the acidic oxidation process.
17. A method according to any of statements 12, 13, 14, 15, 16 of PART II, wherein activating step and the pretreatment step are conducted simultaneously.
18. A method according to any of statements 11-16 wherein the activating step and pretreatment step are conducted sequentially.
19. A method according to any of the preceding statements of PART I OR PART II wherein the acidic oxidation process comprises contacting the keratin fibers with an oxidizing agent at a concentration of about 1 percent to about 6 percent in an aqueous or two phase aqueous and liquid hydrocarbon medium at pH 2-5 wherein the concentration is relative to the total weight of the oxidizing agent and medium.
20. A method according to any of the preceding statements of PART I OR PART II, especially statement 19 of PART II, wherein the oxidizing agent is hydrogen peroxide.
21. A method according to any of the preceding statements of PART I OR PART II wherein the thiolalkoxysilane compound(s) and/or disulfide dimers and/or tetrasulfides thereof of the pretreatment step function at least in part as a reducing agent alone so that no separate reduction process of the Fundamenta procedure is practiced or function at least in part as a reducing agent with another reducing agent in the reduction process of the Fundamenta procedure when the Fundamenta procedure is practiced simultaneously or in overlapping sequence with the pretreatment step.
22. A method according to any of the preceding statements of PART I OR PART II wherein the thiolorgano-alkoxysilane compound, thiolorgano-multidimethylsiloxanyl alkoxysilane and/or disulfide dimers and/or tetrasulfides thereof are applied to the keratin fibers simultaneous with the acidic oxidation process.
23. A method according to preceding statements 19-22 of PART II wherein the Praeparatur procedure and the Fundamenta procedure are conducted sequentially.
24. A method according to any of the preceding statements 19-23 of PART II wherein the Fundamenta procedure further comprises a reduction process conducted before the acidic oxidation process.
25. A method according to any of the preceding statements of PART II wherein the keratin fibers are anagenic hair.
26. A method according to any of the preceding statements of PART II wherein the keratin fibers are anagenic hair which has not been subjected to shampooing, bleaching, and/or oxidative permanent dye treatment within at least one day.
27. A method according to any of the preceding statements of PART I OR PART II wherein the Fundamenta procedure comprises a reducing composition comprising a cosmetically acceptable reducing agent and optionally at a pH of neutral to basic.
28. A method according to any of the preceding statements of PART I OR PART II wherein the reducing composition comprises an aqueous or aqueous-organic medium with at least one reducing agent selected from the group consisting of thioglycolic acid, mercaptanes, ammonium thioglycolate, sodium thioglycolate, mercaptoethanol, cysteine, sodium sulfite, glyceryl monothiopropionate, ammonium thiolactate, dithioerythritol, glutathione, dihydrolipoic acid, 1,3-dithiopropanol, thioglycolamide, glyceryl monothioglycolate, sodium bisulfite, sodium hydrogensulfite, sodium thiosulfate, glyceryl thiolactate, ketoglutarate, DTT red, NADH/H+, dihydrolipoic acid, sulfide, disulfite, thiosulfate, sulfite, sodium sulfite, sodium bisulfite, sodium hydrogen sulfite, sodium thiosulfate, a phosphite, an ammonium sulphite or bisulphite, a cysteamine hydrochloride, or a mercapto silane compound, a thioether silane compound, a disulfide silane compound or a cyclothiolsilyl compound comprising a mercapto(C1-C12)alkyl, (C1-C2)alkyl, (C1-C2)alkoxysilane wherein the sum of the alkyl and alkoxy groups is three, a disulfide dimer of the mercaptoalkyl, alkylalkoxy silane, a C1-C10 alkanoyl thioester of the mercaptoalkyl, alkylalkoxy silane, a phenylethyl or pyridinylethyl thioether of the mercaptoalkyl, alkylalkoxy silane, a 1,1-dialkoxy-2-thiol-1-sila cyclopentaalkane or cyclohexaalkane, or any combination of any of the foregoing named reducing agents.
29. A method according to any of the preceding statements of PART I OR PART II wherein the reducing agent is or includes thioglycolic acid, ammonium thioglycolate or glyceryl monothioglycolate.
30. A method according to any of the preceding statements of PART I OR PART II wherein the Praeparatur procedure comprises a cleaning technique comprising application of an aqueous mixture of an anionic, nonionic, amphoteric or zwitterionic surfactant with optional degreasing organic liquid.
31. A method according to any of the preceding statements of PART I OR PART II wherein the keratin fibers are rinsed with water following application of the acidic oxidation process and optionally at least partially drying and before application of the pretreatment step.
32. A method according to any of the preceding statements of PART I OR PART II wherein the Praeparatur procedure further comprises application of an aqueous composition of one or more of keratin softening agents, lipophilic agents, mineral oil, non-ionic surfactants, cationic surfactants amphipathic surfactants, detergent surfactants, alkylpolyethoxylated surfactants, sulfate free surfactants including but not limited to sarcosonates and taurates.
33. A method according to any of the preceding statements of PART I OR PART II wherein the Praeparatur procedure comprises a non-conditioning or substantially non-conditioning anionic surfactant selected from a fatty alkyl sulfate, a fatty alkyl sulfonate, a fatty alkyl carboxylate or any combination thereof, wherein the fatty alkyl group is a linear or branched C8-C24 alkyl group or a polyethoxylated form thereof or a sulfate free surfactant selected from a sarcosinate and/or a taurate at a concentration of from about 2 wt % to about 30 wt % relative to the total weight of the components of the Praeparatur procedure and optional inclusion of agents for adjustment of viscosity and ionicity and optional adjustment of the pH.
34. A method according to any of the preceding statements of PART I OR PART II further comprising agitation of the keratin fibers by mechanical manipulation and/or by ultrasound vibration.
   Part III the Binder with Alkoxysilyl Functional Monogroups

All of the statements of PART III depend upon the any and all of the Statements of PARTS I and II with the proviso that claim 1 of PART III recites the generic description of the film forming Composition of PART III upon which all other statements of PART III also depend. This dependency is specified by the dependency of PART III statement 1 upon any and all of the statements of PARTS I and II while the remaining statements of PART III depend only upon preceding statements of PART III.
1. A method according to any of the preceding PART I and PART II statements wherein the film forming composition comprises a unitary binder polymer which comprises an organic polymer of one or more monomeric units selected from an olefinic carboxylate ester unit, an olefinic carboxamide unit, a carbon-hydrogen olefinic unit, an ester monomeric unit, an amide monomeric unit, a urethane monomeric unit, a urea monomeric unit, and the organic, polymer has at least one pendant and/or terminal binder functional monogroup comprising an alkoxysilyl group.
2. A method according to statement any of the preceding statements of PART III wherein the organic polymer comprises monomeric units of urethane, urea, ester, amine, olefin or any combination thereof, preferably a combination of ester, urethane and optional urea monomeric units.
3. A statement of a method according to any of the preceding statements of PART III wherein the organic polymeric binder comprises at least a compound of Formula I

X₃Si—R¹-Ct-[Poly]_{y}-Ct-R¹—Si—X₃ Formula IA
wherein
X is hydroxy or alkoxy of 1 to 3 carbons;
   R¹ is a C1 to C8 alkylenyl group;
Ct is a connector group of the Formula II —U¹—R²—U²— joining X₃Si—R¹—to Poly, wherein:
U¹ is covalently bonded to R¹ and U² is covalently bonded to Poly;
Each of U¹ and U² independently is a urea or urethane group;
R² is a C2 to C12 alkylenyl group, a C6-C16 alkylcycloalkyl group or a C6-C14 aromatic or alkylaromatic group;
   Poly is a polymer of monomeric units of an organic ester, urethane, urea, amide or polyol or any combination thereof and y designates the number of monomeric units of Poly forming a polymeric backbone wherein y is an integer of from 2 up to about 1 million, preferably up to about 300,000, more preferably up to about 250,000, most preferably up to about 200,000 and Poly is linear or branched, preferably linear; wherein
   The organic ester monomeric unit is formed of a C2-C20 alkane diol or a C6-C10 aromatic diol and a C3 to C10 alkanodioic acid or a C8-C10 aromatic dicarboxylic acid or the unit is formed of a C3-C10 hydroxy alkanoic acid or a C8-C10 aromatic hydroxycarboxylic acid;
   The organic urethane monomeric unit is formed of a C2-C10 alkane diol and an R³-diisocyanate;
   The organic urea monomeric unit is formed of a C2-C10 alkane diamine and an R³-diisocyanate;
   The organic amide monomeric unit is formed of a C2-C10 alkane diamine and a C3 to C10 alkanodioic acid or a C8-C10 aromatic dicarboxylic acid;
   The polyol monomeric unit is a formed of ethylene oxide or propylene oxide;
   R³ is a linear or branched C2 to C12 alkylenyl group, a C6-C16 alkylcycloalkyl group or a C6-C14 aromatic or alkylaromatic group;
Provided that:
   When Poly is an ester monomeric unit, U² is a urethane group and U¹ is a urea group;
   When Poly is a urethane monomeric unit, U² is a urethane group and U¹ is a urea group;
   When Poly is a urea monomeric unit, U² is a urea group and U¹ is a urea group;
   When Poly is an amide monomeric unit, U² and U¹ are both urea groups;
   When Poly is a polyol monomeric unit, U² is a urethane group and U¹ is a urea group; or,
   Alternatively, U¹ may be a urethane group for each of the Polyester, Polyurethane, Polyurea, Polyamide and Polypolyol provisos.
4. A statement of a method according to any of the previous statements of PART III wherein the organic polymer of Formula IA comprises only two alkoxysilyl groups at each of the organic polymer termini or the organic polymer of Formula IA comprises at least three alkoxysilyl groups with two at the organic polymer termini and at least a third pendant along the Poly backbone and wherein the organic polymer comprising at least three alkoxysilyl groups comprises at least one of:
   a Poly ester having at least one ester monomeric unit formed of a C3-C10 triol;
   a Poly urethane having at least one urethane monomeric unit formed of a C3-C10 triol;
   a Poly urea having at least one urea monomeric unit formed of a C3-C10 triamine;
   a Poly amide having at least one amide monomeric unit formed of a C3-C10 triamine; or,
   a Poly polyol having at least one polyol monomeric unit formed of a triol which is glycerin;
   wherein the third hydroxyl of the triol and the third amine group of the triamine is covalently bound to Ct of a pendant alkoxysilyl group of the formula X₃Si—R¹-Ct-; and
   the film forming composition comprises the organic polymer of Formula IA with only two alkoxysilyl groups, or the organic polymer of Formula IA with at least three alkoxysilyl groups or a mixture of both.
5. A statement of a method according to any of the previous statements of PART III wherein the number of pendant alkoxysilyl groups in Poly is in a range of from about 1 to about 1 thousand, preferably 1 about 100, more preferably 1 to about 10, most preferably 1 to about 5.
6. A statement according to any of the preceding statements of PART III wherein the binder comprises only alkoxysilyl groups at each of the binder termini and Poly is linear.
7. A statement of a method according to any of the preceding statements of PART III wherein Poly comprises a mixture of two or more monomeric units selected from the group consisting of ester monomer, urethane monomer, urea monomer, amide monomer and polyol monomer.
8. A statement of a method according to any of the preceding statements of PART III wherein Poly comprises blocks of two or more selected monomeric units.
9. A statement of a method according to any of the preceding statements of PART III wherein Poly comprises a random arrangement of two or more monomeric units.
10. A statement of a method according to any of the preceding statements of PART III wherein the mixture comprises ester and urethane monomers.
11. A statement of a method according to any of the preceding statements of PART III wherein the mixture comprises urethane and polyol monomers.
12. A statement of a method according to any of the preceding statements of PART III wherein the mixture comprises urea and amide monomers.
13. A statement of a method according to any of the preceding statements of PART III wherein the mixture provides hard and soft segments of Poly.
14. A statement of a method according to any of the preceding statements of PART III wherein each of R² and R³ independently is a C4-C6 alkylenyl group, a C7-C14 alkyl cycloalkyl group, or a C13 alkylaromatic group.
15. A statement of a method according to any of the preceding statements of PART III wherein each of R² and R³ independently is isophoronylenyl, toluenylenyl, methylene diphenyl, hexanylenyl, methylene bis(cyclohexylenyl) or naphthalenyl.
16. A statement of a method according to any of the preceding statements of PART III wherein each of R² and R³ independently is isophoronylenyl, hexanylenyl, methylene diphenyl or methylene bis(cyclohexylenyl).
17. A statement of a method according to any of the preceding statements of PART III wherein Poly is at least an ester monomeric unit of ethylene glycol or 1,6-hexanediol and adipic acid, phthalic acid or terphthalic acid and the designator y is from 200 to 250,000.
18. A statement of a method according to any of the preceding statements of PART III wherein Poly is at least a urethan monomeric unit of a 1,6-hexanediol or ethylene glycol and hexane diisocyanate, isophorone diisocyanate, methylene bis (cyclohexylisocyanate), methylene bis(phenylisocyanate), toluene diisocyanate or naphthalene diisocyanate and the designator y is from 200 to 250,000.
19. A statement of a method according to any of the preceding statements of PART III wherein Poly is at least an amide monomeric unit of a 1,6-hexanediamine or 1,2 diaminoethane and adipic acid, phthalic acid or terphthalic acid and the designator y is from 200 to 250,000.
20. A statement of a method according to any of the preceding statements of PART III wherein Poly is at least a polyethylene glycol and the designator y is from 200 to 250,000.
21. A statement of a method according to any of the preceding statements of PART III wherein Formula IA comprises Formula V, which is a preferred formulation of the binder:

(RO)₃Si—(CH₂)—NHCONH—R¹⁰—NHCOO—[—(CH₂)ₑ—O—CO—R²⁰—COO—]_{g}—(CH₂)ₑ—OCONH—R¹⁰—NHCONH—(CH₂)_{c}Si(OR)₃ Formula V
wherein c is an integer of 3 to 6, e is an integer of 2 to 8 and preferably e provides an ethane or butane or hexane diol; R²⁰ is a divalent benzenenyl or is (CH₂)_{f} wherein f is an integer of 4 to 8; and R²⁰ preferably is a residue of terephthalic acid, succinic or adipic acid residues, g is an integer of 10 to 300,000, R¹⁰ is a C4 to C8 alkylenyl group, preferably providing hexylenyl and R is methyl or ethyl.
22. A statement of a method according to any of the preceding statements of PART III wherein the catalyst is present and is a C2-C8 alkyl phosphate ester acid, C2-C8 alkyl sulfate ester acid, a C2-C8 borate ester acid or a mineral acid.
23. A statement of a method according to any of the preceding statements of PART III wherein the binder of the film forming composition has only two triethoxysilyl groups or trimethoxysilyl groups which are positioned at the termini.
24. A statement of a method according to any of the preceding statements of PART III wherein the binder has at least three triethoxysilyl groups or trimethoxysilyl groups.
25. A statement of a method according to any of the preceding statements of PART III wherein the binder of the film forming composition has at least one branch and has trimethoxy or triethoxy silyl groups at its termini including the terminus of the at least one branch.
26. A statement of a method according to any of the preceding statements of PART III wherein the binder of the film forming composition has a weight average molecular weight of about 1KDa to about 500 KDa, preferably about 1 KDa to about 400 KDa, more preferably about 2 KDa to about 300 KDa, most preferably about 2 KDa to about 200 KDa.
27. A statement of a method according to any of the preceding statements of PART III wherein the film forming composition comprises an organic medium without water, preferably for storage, and the film forming composition with organic medium is optionally combined with water prior to application of the film forming composition to keratin fibers.
28. A statement of a method according to any of the preceding statements of PART III wherein the organic medium is a C2-C6 alkanol and an organic liquid ester, preferably isopropanol or isobutanol and butyl acetate, more preferably isopropanol and optionally further comprises water at no more than about 10 wt %, preferably no more than about 5 wt %, more preferably no more than about 2 wt %, most preferably no more than about 1 wt % relative to the total weight of the medium.
29. A statement of a method according to any of the preceding statements of PART III wherein the polymer moiety of the binder of the film forming composition is linear or branched, preferably linear.
   Part IV Binder with Carboxylic Acid Monofunctional Group

All of the statements of PART IV depend upon any and all of the Statements of PARTS I and II with the proviso that claim 1 of PART IV recites the generic description of the film forming Composition of PART IV upon which all other statements of PART IV also depend. This dependency is specified by the dependency of PART IV statement 1 upon any and all of the statements of PARTS I and II while the remaining statements of PART IV depend only upon preceding statements of PART IV.
1. A method according to any of the preceding PART I and PART II statements wherein the film forming composition comprises a unitary binder polymer comprising an organic polymer of one or more monomeric units selected from an olefinic carboxylate ester unit, an olefinic carboxamide unit, a carbon-hydrogen olefinic unit, an ester monomeric unit, an amide monomeric unit, a urethane monomeric unit, a urea monomeric unit, the organic polymer has at least one pendant and/or terminal binder functional monogroup comprising at least one pendant and/or terminal carboxylic acid group; and the first and second components, which are the same, preferably comprise an organic polymer; and optionally the organic polymer comprises at least one pendant organoalkoxysilane group of the formula —(CH₂)ₙ—SiMeₜ₋₃(OR)ₜ wherein n is an integer of 2 to 10, t is an integer of 1 to 3 and the dangling valence of (CH₂) is connected to a carbon of the organic polymer backbone.
2. A method according to any of the preceding statements of PART IV wherein the pretreatment composition comprises the PTH alkoxysilane compound and the aminoorgano alkoxysiloxane compound and the organic polymer with at least one carboxylic acid binder functional monogroup is capable of non-covalent interaction with the pretreatment composition.
3. A method according to any of the preceding statements of PART IV wherein the polymer comprises an organic polymer which comprises repeating units of at least one olefinic acid monomeric unit and at least one non-acid olefinic monomeric unit selected from an olefinic carboxylate ester monomer unit, an olefinic carboxamide monomer unit, a hydrophilic olefinic monomer unit, a lipophilic olefin monomer unit and any combination thereof, wherein:
   the olefinic acid monomeric unit is selected from (meth)acrylic acid, crotonic acid, maleic acid, fumaric acid, itaconic acid, gluconic acid, a C5-C10 ethenoic acid or any combination thereof,
   the olefinic carboxylate ester monomeric unit is selected from a C1-C30 linear or branched alkyl ester of any of the olefinic acid monomeric units or any combination thereof,
   the olefinic carboxamide monomeric unit is selected from an —NH₂, —NR¹H or —NR¹R² amide of any of the olefinic acid monomeric units or any combination thereof wherein R¹ and R² are each independently selected from a C1-C6 linear or branched alkyl;
   the hydrophilic olefinic monomer is a hydroxy alkyl ester of the olefinic carboxylic acid monomeric unit and a linear or branched C2-C24 alkyl diol, or is an aminoalkyl ester of the olefinic carboxylic acid monomeric unit and a linear or branched amino C2-C24 alkyl alcohol or any combination thereof, and
   the lipophilic olefin monomer unit is selected from an olefin compound of the formula

R³HC═CHR⁴
wherein R³ is selected from hydrogen, linear or branched alkyl of one to six carbons, unsubstituted phenyl or phenyl substituted by a linear or branched alkyl of 1 to six carbons, methyl or ethyl carboxylate, carboxamide or hydroxyl, R⁴ is selected from hydrogen, linear or branched alkyl of one to six carbons, unsubstituted phenyl or phenyl substituted by a linear or branched alkyl of 1 to six carbons, methyl or ethyl carboxylate, carboxamide or hydroxyl, or an ethenyl group of the formula —CH═CHR⁵ wherein R⁵ is selected from hydrogen, linear or branched alkyl of one to six carbons, unsubstituted phenyl or phenyl substituted by a linear or branched alkyl of 1 to six carbons, methyl or ethyl carboxylate, carboxamide or hydroxyl.
4. A method according to any of the preceding statements of Part IV wherein the organic polymer comprises repeating units of at least one (meth)acrylate monomer, at least one non-acid olefin monomer and a (meth)acrylic acid monomer, wherein:
   the (meth)acrylate monomer is an ester of (meth)acrylic acid and a C1-C4 alkyl group;
   the non-acid olefin monomer has the formula

HR³C═CHR⁴
wherein each of R³ and R⁴ is independently selected from hydrogen, linear or branched alkyl of one to six carbons, unsubstituted phenyl and phenyl substituted by one or more of a linear or branched alkyl of 1 to six carbons;
   the polymer has an acid value of from about 0.1 to about 200;
   the polymer has a glass transition temperature of from about −60° C. to about 90° C.;
   the polymer has a weight average molecular weight in the range of about 2 KDa to about 10 MDa.
5. A method according to any of the preceding statements of PART IV wherein the organic polymer comprises repeating units of at least one olefinic carboxylic acid monomeric unit and repeating units of a non-acid olefin monomer unit wherein R³ and R⁴ are both hydrogen.
6. A method according to any of the preceding statements of PART IV wherein the olefinic carboxylic acid monomeric unit is acrylic acid.
7. A method according to any of the preceding statements of PART IV wherein the medium is an aqueous or aqueous organic medium and has a basic pH.
8. A method according to any of the preceding statements of PART IV wherein the organic polymer does not have an organoalkoxysilane group.
9. A method according to any of the preceding statements of PART IV wherein the organic polymer has one or more pendant organoalkoxysilane groups.
10. A method according to statement 9 wherein the organic polymer is combined with basic medium immediately prior to application to keratin fibers.
   Part V Binder with Complementary Pair Functional Group Michael Addition

All of the statements of PART V depend upon any and all of the Statements of PARTS I and II with the proviso that claim 1 of PART V recites the generic description of the film forming Composition of PART V upon which all other statements of PART V also depend. This dependency is specified by the dependency of PART I statement 1 upon any and all of the statements of PARTS I and II while the remaining statements of PART V depend only upon preceding statements of PART V.
1. A method according to any of statements of PART I AND PART II wherein:
   the film forming composition comprises a dual binder polymer comprising a first component comprising an organic, silicone or organosilicone polymer having at least one pendant and/or terminal first binder functional group, and a second component comprising a small molecule, pre-polymer or polymer having at least one pendant and/or terminal second binder functional group; and,
   the first and second binder functional groups are a complementary pair selected from the group consisting of (i) alkenoyloxy and amine or (ii) alkenoyloxy and thiol.
2. A method according to any of the preceding statements of PART V wherein the complementary pair of first and second binder functional groups are chemically reactive and capable of forming a covalent connection.
3. A method according to any of the preceding statements of PART V wherein the film forming composition comprises a medium, a first component comprising a silicone polymer having pendant and/or terminal α,β unsaturated alkenoyloxy groups and a second component comprising a silicone polymer having pendant and/or terminal organoamine groups and optional terminal alkoxysilyl groups wherein the first component and second component are each independently linear or branched, preferably linear.
4. A method according to any of the preceding statements of PART V wherein the first component comprises polydimethylsiloxane-type polymer having at least two α,β unsaturated alkenoyloxy groups attached to siloxane units of the polymer.
5. A method according to any of the preceding statements of PART V wherein first component comprises a polydimethylsiloxane-type polymer having at least three α,β unsaturated alkenoyloxy groups attached to siloxane units of the polymer.
6. A method according to any of the preceding statements of PART V wherein the α,β unsaturated alkenoyloxy group comprises the formula R¹R²C═CR³COO—R⁴— wherein each of R¹ and R² independently is a hydrogen or a C1-C6 alkyl group provided that at least one of R¹ and R² is hydrogen; R³ is hydrogen or methyl, and R⁴ is a C1 to C12 alkylenyl group, a C3-C12 cycloalkylalkyl or cycloalkyl group, a C6-C20 arylalkyl group or a C6-C20 aryl group wherein R⁴ is optionally substituted in chain by one or more ether oxygen, thioether sulfur and/or amine groups and/or pendantly by hydroxyl groups and R⁴ is bonded to a silicon of the polydimethyl siloxane-type polymer.
7. A method according to any of the preceding statements of PART V wherein R⁴ is a hydroxy substituted alkylenyl group to provide an α,β unsaturated alkenoyloxy group comprising the formula R¹R²C═CR³COO—CH₂CHOH—CH₂—O—(CH₂)ₙ— wherein n is an integer of from 1 to 6.
8. A method according to any of the preceding statements of PART V wherein the composite film of pretreatment and film forming compositions is dried and cured to form the color coating on the keratin fibers.
9. A method according to any of the preceding statements wherein the first component comprises a silicone polymer of Formula I

X_{z}—SiMe_{3-z}O-(Me₂SiO)ₓ—(Si(—X)MeO)_{y}—SiOMe_{3-z}-X_{z} Formula I
wherein
   each of Me₂SiO and Si(—X)MeO comprise a monomeric siloxane D unit, and X_{z}SiMe_{3-z}O comprises a monomeric siloxane M unit;
   X comprises R¹R²C═CR³COO—R⁴—;
   each of R¹ and R² independently is a hydrogen or a C1-C6 alkyl group, provided that at least one of R¹ and R² is hydrogen;
   R³ is hydrogen or methyl;
   R⁴ is a C1 to C12 alkylenyl group, a C3-C12 cycloalkylalkyl or cycloalkyl group, a C6-C20 arylalkyl group or a C6-C20 aryl group wherein any or all of the groups are optionally substituted in chain by one or more ether oxygen, thioether sulfur and/or amine groups and/or pendantly by hydroxyl groups and R⁴ is attached to silicon of a monomeric D and/or M unit;
   each of the designators x and y independently designates the number of monomeric D siloxane units forming the corresponding linear polymeric silicone backbone, wherein x is an integer of from 1 up to about 100,000 and designator y is zero or an integer of from 1 to 10; designator z for each of the X_{z}SiMe_{3-z}O units is zero or 1 so that the X_{z}SiMe_{3-z}O unit may have a terminal X group or may be an M-type trimethyl siloxy group;
   the sum of x and y is an integer of from about 3 up to about 200,000, preferably up to about 150,000, more preferably up to about 100,000, most preferably up to about 50,000 and especially most preferably up to about 100, with exemplary sums of 10 to 50 and 10 to 20;
   and the multiple monomeric units of Me₂SiO and Si(—X)MeO are randomly distributed in Formula I.
10. A method according to any of the preceding statements of PART V wherein designator y is zero.
11. A method according to any of the preceding statements of PART V wherein designator x is at least 5 and designator y is 1 to 5. 12. A method according to any of the preceding statements of PART V wherein z is zero.
13. A method according to any of the preceding statements of PART V wherein the Si(—X)MeO groups number in a range of from about 1 to about 10, more preferably 1 to about 5, most preferably 1 to 2.
14. A method according to any of the preceding statements of PART V wherein X comprises H₂C═CR³COO—R⁴—.
15. A method according to any of the preceding statements of PART V wherein R⁴— comprises a linear C2-C8 alkylene or —CH₂CHOH—CH₂—O—(CH₂)ₙ— wherein n is an integer of 1 to 6 and the left valence is connected to the carboxyl of H₂C═CR³COO—.
16. A method according to any of the preceding statements of PART V wherein the second component comprises a polydimethylsiloxane-type polymer having one or more pendant and/or terminal organoamine groups attached to siloxane units of the polymer and having one or more terminal alkoxysilyl groups attached to the polymer.
17. A method according to any of the preceding statements of PART V wherein the second component comprises Formula V

M1-(D)_{d}-M2 Formula V
wherein
   M1 and M2 are termini of the second component and may be selected from Me₃SiO units, A-SiMe₂O units and —Si(OR)₃ units wherein R is methyl or ethyl or hydrogen and A is an organoamine or organothiol group of the Formula OA

Y—(R¹⁰—NH)ᵣ—R¹— Formula OA
wherein Y is —NH₂ or —SH; R¹⁰ is a linear or branched C1-C10 alkyl group or a linear or branched C6-C14 alkylaryl group; R¹¹ is a linear or branched C1-C10 alkyl group or a linear or branched C6-C14 alkylaryl group; and designator r is zero or an integer of 1 to 3 and R¹¹ is bonded to silicon when r is other than zero and R¹¹ is bonded to silicon when r is zero and when Y is —SH, r is zero; and
D units form the backbone of the polydimethylsiloxane-type second component with designator d being an integer of from 3 to 20,000 indicating the size of the second component, wherein
the D units are selected from SiMe₂O units (dimethylsiloxane units) and A-SiMeO units; and wherein:
the second component comprises at least one A-SiMeO unit.
18. A method according to any of the preceding statements of PART V wherein the second component of Formula V carries as a first embodiment two terminal A-SiMe₂O units; or as a second embodiment two Me₃SiO units as M1 and M2 and one, two or three A-SiMeO units; or as a third embodiment one terminal Si(OR)₃ unit, one Me₃SiO terminal and one, two or three A-SiMeO units; or as a fourth embodiment two terminal Si(OR)₃ units, and one, two or three A-SiMeO units and for the first through fourth embodiments multiple dimethylsiloxane D units to provide a weight average molecular weight of from about 10 KDa to about 20 KDa.
19. A method according to any of the preceding statements of PART V wherein the second component comprises a linear polydimethylsiloxane-type polymer formed from selections of the following monomeric units

| (Me₃SiO) | (Si(OR)₃ | (A-SiMe₂O) | (SiMe₂O)ₒ | (SiMeO-A)ₚ |
|---|---|---|---|---|
| M-T1 | M-T2 | M-T3 | D-B1 | D-B2 |

wherein
   the SiMe₂O and SiMeO-A groups are D type siloxanyl monomeric units distributed randomly throughout the polydimethylsiloxane-type backbone, and the Me₃SiO, A-SiMe₂O and —Si(OR)₃ groups are M type terminal siloxanyl monomeric units such that any combination of two of the M type monomeric units terminate the second component;
   the designator o primarily indicates the number of corresponding D monomeric units present in the polymer;
   the designator o is an integer of from 2 to 1000;
   the designator p is zero or an integer of from 1 to 10;
   and A is Formula OA.
20. A method according to any of the preceding statements of PART V wherein designator p is zero, designator o is an integer of from 4 to about 500, preferably 200, more preferably up to about 100 and both termini are M-T3 units.
21. A method according to any of the preceding statements of PART V wherein M-T3 is absent, designator p is an integer of from about 2 to about 6, designator o is an integer of from about 4 to about 500, preferably up to 100 and the termini are both M-T2 units.
22. A method according to any of the preceding statements of PART V wherein M-T3 is absent, designator p is 1, designator o is at least 5 and the termini are both M-T1 units.
23. A method according to any of the preceding statements of PART V wherein the second component comprises one M-T1 unit, one M-T2 unit, designator o is about 4 to about 500, preferably up to 100 and designator p is 1, 2 or 3. 24. A method according to any of the preceding statements of PART V wherein the first component is characterized by Formula IV

H₂C═CHCOOCH₂CHOH—CH₂—O—(CH₂)_{c}—SiMe₂O—(SiMe₂O)ₘ—[MeSiO—(—(CH₂)_{c}—O—CH₂—CHOH—CH₂OOC—CH═CH₂)]_{g}-(Me₂SiO)ₚ—OSiMe₂-(CH₂)_{c}—O—CH₂—CHOHCH₂OOCCH═CH₂ Formula IV
wherein c is an integer of 1 to 6, m and p are each independently an integer of from about 2 to about 100, g is zero or an integer of from 1 to about 10, and the dimethylsiloxanyl and acryloyloxyalkylsiloxanyl groups are distributed randomly.
25. A method according to statement 24 of PART V wherein m and p are each independently 5 to 50, g is an integer of from 1 to 5, and c is an integer of 1 to 3.
26. A method according to statement 24 of PART V wherein m and p are each independently 5 to 50, g is zero and c is an integer of 1 to 3.
27. A method according to statement 24 of PART V wherein m and p are each independently 5 to 50, g is an integer of 1, and c is an integer of 1 to 3.
28. A method according to statement 24 of PART V wherein c is 3.
29. A method according to any of the preceding statements of PART V wherein the first component of the film forming composition has a weight average molecular weight of about 0.5 KDa to about 10 KDa, preferably about 0.5 KDa to about 5 KDa, more preferably about 1 KDa to about 5 KDa, most preferably about 1 KDa to about 3 KDa.
30. A method according to any of the preceding statements of PART V wherein the second component of the film forming composition has a weight average molecular weight of about 5KDa to about 50 KDa, preferably about 5 KDa to about 30 KDa, more preferably about 5 KDa to about 20 KDa, most preferably about 8 KDa to about 20 KDa.
31. A method according to any of the preceding statements of PART V wherein the medium is an organic medium, preferably is a C2-C6 alkanol, preferably isopropanol or isobutanol.
32. A method according to any of the preceding statements of PART V wherein the medium is an aqueous-organic medium and the water content is no more than about 10 wt %, preferably no more than about 5 wt %, more preferably no more than about 2 wt %, most preferably no more than about 1 wt % relative to the total weight of the medium.
33. A method according to any of the preceding statements of PART V wherein the concentration of the combination of first component and second component in the film forming composition comprises a weight percent range relative to the total weight of the film forming composition, of from about 2 wt % to about 8 wt %, preferably about 2.5 wt % to about 7 wt %, more preferably about 2.5 wt % to about 6 wt %.
34. A method according to any of the preceding statements of PART V wherein first component and second component in separate media before combining as the film forming composition comprises a weight percent range relative to the total weight of the first component and medium or second component and medium of from about 2 wt % to about 20 wt %, preferably about 2 wt % to about 15 wt %, more preferably about 2 wt % to about 10 wt %.
35. A method according to any of the preceding statements of PART V wherein the pretreatment composition comprises a thiolorganoalkoxy silane and an aminoorganoalkoxy silane.
36. A method according to any of the preceding statements of PART V wherein the pretreatment composition comprises only thiolorganoalkoxy silane.
37. A method according to any of the preceding statements of PART V wherein Y of Formula OA is H₂N— alone.
38. A method according to any of the preceding statements of PART V wherein the film forming composition comprises a linear silicone polymer having at least two pendant and/or terminal α,β unsaturated alkenoyloxy groups and a second component comprising a linear silicone polymer having at least two organoamine groups and at least one alkoxysilyl group.
39. A method according to any of the preceding statements of PART V wherein the film forming polymer comprises a first linear silicone polymer of a backbone of dimethylsiloxane units and having at least two —(CH₂)₃—(O)ₐ—CH₂—CHOH—CH₂—O₂C—CH═CH₂ groups bonded directly to the silicons of at least two of the backbone and/or terminal siloxane units wherein designator a is zero or 1 and wherein the linear silicone polymer has a weight average molecular weight of from about 1KDa to about 3 KDa and the second component comprises linear silicone polymer of a backbone of dimethylsiloxane units with at least two —R⁵—NH—(CH₂)₂—NH₂ groups directly bonded to silicons of the backbone and/or terminal siloxane units wherein R⁵ propyl or isobutyl and wherein the second component polymer has a weight average molecule weight of from about 10 KDa to about 20 KDa.
40. A method according to any of the preceding statements of PART V wherein the second component further comprises at least one trialkoxysilyl-group as a terminal unit.
41. A method according to any of the preceding statements of PART V wherein the first component comprises two —(CH₂)₃—(O)ₐ—CH₂—CHOH—CH₂—O₂C—CH═CH₂ groups with one each at each of the two termini of the first component and optionally one or two pendant —(CH₂)₃—(O)ₐ—CH₂—CHOH—CH₂—O₂C—CH═CH₂ groups on the backbone of the first component wherein designator a is zero or 1; and the second component comprises a terminus with a —R⁵—NH—(CH₂)₂—NH₂ group; a terminus as an alkoxysilane wherein the substituents of the silicone are a combination of methoxy and hydroxy group and at least one or two pendant —R⁵—(CH₂)₃—NH—(CH₂)₂—NH₂ groups.
   Part VI Binder with Complementary Pair Functional Group CDI Addition

All of the statements of PART VI depend upon any and all the Statements of PARTS I and II with the proviso that claim 1 of PART VI recites the generic description of the film forming Composition of PART VI upon which all other statements of PART VI also depend. This dependency is specified by the dependency of PART I statement 1 upon any and all of the statements of PARTS I and II while the remaining statements of PART VI depend only upon preceding statements of PART VI.
1. A method according to any of statements of PART I AND PART II wherein:
   the film forming composition comprises a dual binder polymer comprising a first component comprising an organic, silicone or organosilicone polymer having at least one pendant and/or terminal first binder functional group, and a second component comprising a small molecule, pre-polymer or polymer having at least one pendant and/or terminal second binder functional group; and,
   the first and second binder functional groups are a complementary pair comprising carboxylic acid and carbodiimide.
2. A method according to any of the preceding statements of PART VI wherein the complementary pair of first and second binder functional groups are chemically reactive and capable of forming a covalent connection.
3. A method according to any of the preceding statements of PART VI wherein the first component comprises an olefinic polymer, silicone polymer or olefinic-silicone block copolymer having at least 2 pendant and/or terminal carboxylic acid groups, first component being linear or branched, preferably linear; and
   the second component comprises an alkylenyl, aromatic or alkylenyl aromatic polymer having multiple in chain segments of carbodiimide or a polymer of ester, urethane or urea monomeric residues having pendant and/or terminal alkylenyl single carbodiimide groups, the second component being linear or branched, preferably linear.
4. A method according to any of the preceding statements of PART VI wherein the first component comprises at least at least three carboxylic acid groups.
5. A method according to any of the preceding statements of PART VI wherein the first component comprises an olefinic-silicone block polymer.
6. A method according to any of the preceding statements of PART VI wherein the second component further comprises pendant and/or terminal alkoxysilyl groups.
7. A method according to any of the preceding statements of PART VI wherein the first component comprises an olefinic polymer, a silicone polymer or an olefinic silicone block copolymer having along its backbone two or more pendant and/or terminal carboxylic acid groups, and at least one or more of a pendant group selected from an alkyl alkylenylcarboxylic ester group, an alkyl group, an alkylenyloxycarbonylalkyl group or a hydroxyalkyl group.
8. A method according to any of the preceding statements of PART VI wherein first component is an olefinic or olefinic silicone block copolymer and the pendant and/or terminal carboxylic acid groups are formed from one or more C3-C12 unsaturated mono or dicarboxylic acids, preferably one or more of (meth)acrylic acid, crotonic acid, itaconic acid, maleic acid, fumaric acid, isoprenoic acid, pentenoic acid and/or pentadienoic acid.
9. A method according to any of the preceding statements of PART VI wherein the first component comprises an olefinic, silicone or organosilicone polymer of Formula I

MUE-(MU1)ₓ-(MUX)_{y}-(MU2)_{z}-(MU3)ₐ-(MU3X)_{b}-MUE Formula I
wherein:
   MU1 comprises a hydrophobic olefinic monomeric unit comprising a linear C2-C10 alkene residue, a linear C4-C12 alkadiene residue and/or a C6-C10 aromatic/alkylaromatic vinyl residue,
   MUX comprises an acidic olefinic monomeric unit comprising a linear C3-C10 alkenoic acid residue or a C4-C10 alkadienoic acid residue;
   MU2 comprises a hydrophilic olefinic monomeric unit comprising a vinyl linear C2-C16 alkanoic ester residue, a C1-C14 linear alkyl or hydroxyalkyl linear C2-C14 alkenoic ester residue, a linear C2-C10 alkenoic amide residue or N-C1-C4 alkyl substituted version of the amide residue;
   MU3 comprises a dimethylsiloxane residue;
   MU3X comprises a monomethylsiloxane residue bound to an alkanoic acid of at least 4 carbons with one of the alkyl carbons of the alkanoic acid optionally having a hydroxy group; and
   MUE comprises a single terminal monomeric unit of MU1, MU2, MU3, MUX or MU3X; each of the designators x, y, z, a and b independently designates the number of corresponding monomeric units forming the linear polymeric backbone, wherein each of x, z and a is zero or an integer of from 1 up to about 100,000 and y and b are each zero or an integer of 1 to 100;
   when b is an integer, y may be zero or an integer and when b is zero, y is an integer;
   the sum of x, y, z, a and b is an integer of from about 3 up to about 1 million, preferably up to about 300,000, more preferably up to about 250,000, most preferably up to about 200,000;
   the multiple monomeric units of MU1, MU2 and MU3 are randomly distributed or form blocks in Formula I and the multiple carboxylic acid monomeric units MUX and MU3X are randomly distributed among MU1, MU2 and MU3 units; and,
   the first component being linear or branched, preferably linear.
10. A method according to any of the preceding statements of PART VI wherein designators x and z are each at least 10, designator y is at least 3, designators a and b are both zero and terminal MUE is MUX.
11. A method according to any of the preceding statements of PART VI wherein each of designators x, z and a are 10 to 100, designator y is 1 to 50, designator b is zero, terminal MUE is MUX and the polymer is an organosilicone block copolymer
12. A method according to according to any of the preceding statements of PART VI wherein designators x, y and z are zero, designator a is at least 20, preferably at least 40, designator b is 1 to 50 and terminal MUE is MU3X.
13. A method according to any of the preceding statements of PART VI wherein each of designators x, z and a are 10 to 100, each of designators y and b independently is 1 to 50, terminal MUE is MUX or MU3X and the polymer is an organosilicone block copolymer.
14. A method according to any of the preceding statements of PART VI wherein the monomeric units are randomly distributed.
15. A method according to any of the preceding statements of PART VI wherein the monomeric units form block segments and when the first component is an organic polymer, the MU1 and MU2 units form blocks.
16. A method according to any of the preceding statements of PART VI wherein MU1 is butene, pentene, hexene, styrene, MUX is (meth)acrylic acid, crotonic acid, pentenoic acid, hexenoic acid, fumaric acid, maleic acid, itaconic acid glutaconic acid, citraconic acid or mesaconic acid, MU2 is vinyl acetate, vinyl propanate, vinyl butanate, C1-C3 alkyl or hydroxyalkyl (meth)acrylate, C1-C3 alkyl or hydroxyalkyl crotonate, C1-C3 alkyl or hydroxyalkyl pentanoate, C1-C3 dialkyl or di-(hydroxyalkyl) fumarate, C1-C3 maleate or the corresponding primary amides or C1-C3 alkyl secondary amides and MU3X is MeSi(O)—R¹⁰—COOH wherein R¹⁰ is —(CH₂)ₙ—CHOH—(CH₂)₂—.
17. A method according to any of the preceding statements of PART VI wherein MU1 is hexene or styrene, MUX is (meth)acrylic acid or crotonic acid, MU2 is vinyl acetate, vinyl C8-C12 isoalkanoate, methyl, ethyl or isopropyl (meth)acrylate or the corresponding hydroxymethyl, hydroxyethyl or hydroxyisopropyl analogs, methyl, ethyl or isopropyl crotonate or the corresponding hydroxymethyl, hydroxyethyl or hydroxyisopropyl analogs and MU3X is -MeSi(O)—(CH₂)₃—CHOH—(CH₂)₂—COOH.
18. A method according to any of the preceding statements of PART VI wherein x zero, b is zero, a is at least 10, z is at least 10, y is 1 to 50, MUE is MUX and MU2 and MU3 form a block copolymer with MUX randomly distributed within MU2.
19. A method according to any of the preceding statements of PART VI wherein the first component comprises at least monomeric units of alkyl (meth)acrylate and/or crotonate, and (meth)acrylic acid and/or crotonic acid; and the acid number of the polymer is from about 50 to about 600 preferably about 100 to about 400.
20. A method according to any of the preceding statements of PART VI wherein the first component comprises at least an acid monomer as (meth)acrylic acid and/or crotonic acid at about 0.3% to about 75% by weight, a hydroxyethyl or hydroxypropyl (meth)acrylate and/or crotonate at about 0% to about 20% by weight and a hydrophobic monomer as methyl or ethyl (meth)acrylate and/or crotonate at about 5% to about 20% by weight, wherein all weights are relative to the total weight of the polymer.
21. A method according to any of the preceding statements of PART VI wherein the second component comprises an organic polymer of Formula II, a polymer with in-chain carbodiimide groups or Formula X, a polymer with pendant single carbodiimide groups

Z-(L-N═C═N—)ₚ—Z Formula II

(Poly)_{q}-(K)ₛ-(Poly)ᵣ Formula X
wherein;
   For Formula II, p is an integer of at least 2; and L is an organic second component group comprising saturated aliphatic divalent radical, an aromatic divalent radical or an alkylaromatic divalent radical or a polymer or oligomeric divalent radical with repeating olefinic, carbonate, ester, ether, amide, imine, urethane or urea linkages;
   For Formula X, each Poly is an organic polymer segment of an amide, imine, olefinic, caronate, ester, ether, urethan or urea monomeric residue and preferably the residue is an amide or urea/urethane monomeric residue is based upon a C3 to C6 alkane diamine and a C4-C10 alkane dicarboxylic acid or C4-C10 alkane diisocyanate, or an ester monomeric residue based upon a C3-C6 alkane diol and a C4-C10 alkane dicarboxylic acid and the designators q and r each being an integer of at least 2; K is a pendant carbodiimide group of Formula XI with s being an integer of at least 2

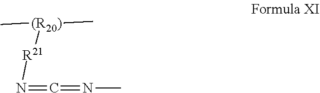

wherein R²⁰ is a C3 to C6 alkylenyl residue, R²¹ is a C3-C6 alkylenyl residue;
   For Formulas II and XI Z is a non-reactive or reactive terminal group of the polycarbodiimide; and, the multiple K's are randomly distributed along the Poly backbone; L or Poly of the second component being linear or branched, preferably linear.
22. A method according to any of the preceding statements of PART VI wherein the second component is Formula II, L is a saturated aliphatic divalent radical selected from linear or branched or cyclic alkylenyl of 2 to 20 carbons, an aromatic divalent radical selected from benzene or diphenyl, or an alkylaromatic divalent radical selected from p-dimethylenylphenyl or methylenyldiphenyl.
23. A method according to any of the preceding statements of PART VI wherein the second component is Formula II, L is a saturated alkylenyl divalent radical of 2 to 6 carbons.
24. A method according to any of the preceding statements of PART VI wherein the second component is Formula II, L is a residue of toluene, diphenylmethane, phenyl, dicyclohexyl methane, methyl-3,5,5,-trimethylcyclohexane, hexane, cyclohexane, norbornane.
25. A method according to any of the preceding statements wherein the second component is Formula II and L is dicyclohexylmethane, methyl-3,5,5-trimethylcyclohexane or hexane.
26. A method according to any of the preceding statements of PART VI wherein the Z is a self-reacting C2-C6 alkylenylalkoxysilyl group.
27. A method according to any of the preceding statements of PART VI wherein the second component is Formula II and Z is a nonreactive group comprising a saturated aliphatic monovalent radical selected from linear or branched or cyclic alkyl of 2 to 20 carbons, an aromatic monovalent radical selected from benzene or diphenyl, or an alkylaromatic monovalent radical selected from p-dimethylenylphenyl or methylenyldiphenyl.
28. A method according to any of the preceding statements of PART VI wherein Z is butane or hexane.
29. A method according to any of the preceding statements of PART VI wherein Z is hexane.
30. A method according to any of the preceding statements wherein the number of carbodiimide groups designated by p is from 2 to 100, preferably from 2 to 50, more preferably from 2 to 10, most preferably 2 to 5.
31. A method according to any of the preceding statements of PART VI wherein the second component is Formula X, Poly is a polyamide or polyurea and the number of pendant carbodiimide K groups designated by s is from 2 to 50, preferably 2 to 10, more preferably 2 to 5.
32. A method according to any of the preceding statements of PART VI wherein the second component is Formula X, Poly is a polyurethane and the number of pendant carbodiimide K groups designated by s is from 2 to 50, preferably 2 to 10, more preferably 2 to 5.
33. A method according to any of the preceding statements of PART VI wherein the second component is Formula X, R²⁰ and R²¹ are each butylenyl or hexylenyl, and Z is butyl or hexyl.
34. A method according to any of the preceding statements of PART VI wherein the first component of the film forming composition has a weight average molecular weight of about 0.5KDa to about 500 KDa, preferably about 0.5 KDa to about 400 KDa, more preferably about 0.5 KDa to about 10 KDa, most preferably about 0.5 KDa to about 3KDa to 5 KDa.
35. A method according to any of the preceding statements of PART VI wherein the second component of the film forming composition has a weight average molecular weight of about 0.5KDa to about 500 KDa, preferably about 0.5 KDa to about 400 KDa, more preferably about 0.5 KDa to about 10 KDa, most preferably about 0.5 KDa to about 3KDa to 5 KDa.
36. A method according to any of the preceding statements of PART VI wherein the medium comprises a hydrocarbon, a silicone or an organic polar liquid, preferable an alcohol and more preferably a C2-C6 alkanol diol or triol, preferably isopropanol, propylene glycol or isobutanol.
37. A method according to any of the preceding statements of PART VI wherein the medium is for application of the film forming composition and comprises an aqueous-organic medium and the water content is no more than about 10 wt %, preferably no more than about 5 wt %, more preferably no more than about 2 wt %, most preferably no more than about 1 wt % relative to the total weight of the medium.
38. A method according to any of the preceding statements of PART VI wherein the medium is for separate storage of first component and second component of the film forming composition and is an anhydrous hydrocarbon, silicone or alcoholic medium.
39. A method according to any of the preceding statements of PART VI wherein the concentration of the first component in the medium of the film forming composition comprises a weight percent range relative to the total weight of the film forming composition, of from about 2 wt % to about 8 wt %, preferably about 3 wt % to about 7 wt %, more preferably about 4 wt % to about 6 wt %.
40. A method according to any of the preceding statements of PART VI wherein second component in the medium of the film forming composition comprises a weight percent range relative to the total weight of the film forming composition, of from about 2 wt % to about 8 wt %, preferably about 3 wt % to about 7 wt %, more preferably about 4 wt % to about 6 wt %.
41. A method according to any of the preceding statements of PART VI wherein
   the first component comprises a polymer having at least three pendant-terminal carboxylic acid groups wherein the polymer is linear and comprises
   an olefinic polymer of one or more monomeric units of (meth)acrylic ester, vinyl C8-C12 isoalkanoate ester, C3-C6 alkene, styrene and/or hydroxyalkyl (meth)acrylate; and one or more monomeric acid units of (meth)acrylic acid maleic acid, crotonic acid, fumaric acid and/or itaconic acid;
   a silicone polymer of a backbone comprising dimethylsiloxane units interspersed with methylsiloxanylalkylcarboxylic acid units and terminated with dimethylsiloxane units; or
   a block copolymer of blocks of the olefin polymer and the silicone polymer; and,
   the second component is linear and comprises multiple segments of a divalent radical coupled together with in-chain carbodiimide groups and terminated with C1-C3 alkylenyltrialkoxysilyl groups wherein the divalent radical is a hexylenyl radical, an isophoronyl radical, a p-dimethylenylphenyl radical or a methylenyl bis(cyclohexanyl) radical and the carbodiimide groups number from 3 to 100.
42. A method according to any of the preceding statements of PART VI wherein the first component comprises the linear olefin-silicone block copolymer of bis-vinyl dimethicone, vinyl C8-C12 isoalkanoate ester, and an olefinic acid monomer selected from crotonic acid, maleic acid and/or (meth)acrylic acid units wherein the first component has at least 3 to 6 carboxylic acid groups per molecule and a weight average molecular weight of from about 1KDa to about 10 KDa and the second component comprises a linear carbodiimide polymer of from about 5 to about 25 carbodiimide groups interconnected with divalent isophoronyl radicals, phenyl-1,4-dimethylenyl radicals, methylenyl bis(cyclohexanyl) radicals and terminated with C3-C6 alkylenyl triethoxysilyl groups wherein the second component has a weight average molecule weight of from about 0.5KDa to about 5 KDa.

### Combination Statements

1. A method according to any of the preceding statements of PARTS I, II, III, IV, V and/or VI wherein the colorant is combined with the film forming composition.
2. A method according to any of the preceding statements of PARTS I, II, III, IV, V and/or VI wherein the modified keratin fibers, the pretreatment composition and the film forming composition are at least partially interconnected and entwined by chemical and/or physical interactions.
3. A method according to any of the preceding statements of PARTS I, II, III, IV, V and/or VI wherein the pretreatment composition is applied to the modified keratin fibers by contacting the fibers with an absorbent material carrying the pretreatment composition to produce pre-coated keratin fibers having thereon the pretreatment composition on the surfaces of the keratin fibers.
4. A statement of a method according to any of the preceding statements of PARTS I, II, III, IV, V and/or VI wherein the absorbent material is a sponge-like solid and sections of the keratin fibers are individually wiped with the absorbent material carrying the pretreatment composition so as to deposit the pretreatment composition on the surfaces of substantially all keratin fibers of each section.
5. A statement of a method according to any of the preceding statements of PARTS I, II, III, IV, V and/or VI wherein the pre-coated keratin fibers are dried.
6. A statement of a method according to any of the preceding statements of PARTS I, II, III, IV, V and/or VI wherein the drying is accomplished at a temperature above ambient so as to cure at least partially the small molecule of the precoated keratin fibers.
7. A statement of a method according to any of the preceding statements of PARTS I, II, III, IV, V and/or VI wherein the primed and/or deep cleaned pre-coated keratin fibers is contacted with an absorbent material carrying the film forming composition to produce a composite film of pretreatment composition and film forming composition on the keratin fibers.
8. A statement of a method according to any of the preceding statements of PARTS I, II, III, IV, V and/or VI wherein the absorbent material is a sponge-like solid and sections of the keratin fibers are individually wiped with the absorbent material carrying the film forming composition so as to deposit the film forming composition on the pre-coated keratin fibers to produce the composite film on the surfaces of substantially all keratin fibers of each section.
9. A statement of a method according to any of the preceding statements of PARTS I, II, III, IV, V and/or VI wherein the composite film on the surfaces of the keratin fibers is dried.
10. A statement of a method according to any of the preceding statements of PARTS I, II, III, IV, V and/or VI wherein the drying is accomplished at a temperature so as to cure at least partially the composite film and produce a color coating on the surfaces of the keratin fibers.
11. A statement of a method according to any of the preceding statements of PARTS I, II, III, IV, V and/or VI wherein the pretreatment composition and the components of the film forming composition in compatible medium are all maintained in separate containers until before use.
12. A statement of a method according to any of the preceding statements of PARTS I, II, III, IV, V and/or VI wherein separate quantities of the first and second components each in a compatible medium are combined to form the film forming composition before application to keratin fibers.
13. A statement of a method according to any of the preceding statements of PARTS I, II, III, IV, V and/or VI wherein a coloration feature of the film forming composition comprises development of a series of premixes of pigments and dispersants and formation of the coloration feature by blending together a selection of premixes to produce a custom color mix of pigment selections and dispersants and combining the custom color mix with the film forming composition wherein the blending together of a selection of premixes is accomplished by correlating a spectrographic coloration analysis of the keratin fibers and applying a computer simulation to the coloration analysis and desired color to identify the selection of premixes for producing the desired color of the keratin fibers; or the coloration feature of the film forming composition comprises a series of film forming compositions in ready-to-use containers each with a premixed pigment combination to provide a selection of colors ready to use for coloration of keratin fibers, especially anagen hair.
14. A method according to any of the preceding statements of PARTS I, II, III, IV, V and/or VI wherein the film forming composition further comprises an additive comprising one or more of a microfibril having a fiber length between 1 nanometer and 10 micrometers; a non-chromatic filler material with a particle size between 2 nm and 500 nm; a polyolefin as macromolecular strands or nanoparticles wherein the nanoparticles are selected from the group consisting of smectites, kaolins, illites, chlorites, attapulgites and mixtures or inorganic metal oxides selected from the group consisting of silica, titanium oxide, zirconium oxide, aluminum oxide, magnesium oxide, boehmite alumina, hydrotalcite; a carbon nanotube; nanofiller of graphite oxide mixed polymer; a graphene additive; one or more UV filters; one or more radical scavengers; one or more triplet formation inhibitors; a metal compound which can absorb or reflect UV light, wherein the metal compounds are selected from chromium, titanium, zinc, nickel, manganese, iron, niobium, silver, gold, aluminum, hafnium, tantalum; polyvinylidene fluoride for UV protection and chemical resistance.
15. A method for removing the color coating from keratin fibers produced according to any of the preceding statements of PARTS I, II, III, IV, V and/or VI comprising contacting the color coating with a removal composition comprising an organic hydrocarbon medium comprising at least dodecyl benzene sulfonic acid and isododecane.
16. A method according to any of the previous removing statements wherein the removal composition is applied to the keratin fibers and thoroughly manipulated throughout the fibers for a period of at least 10 to 15 minutes.
17. A method according to any of the previous removing statements further comprising using an absorbable substrate to wipe the hair to take away the removal composition from the hair after manipulation.
18. A method according to any of the previous removing statements wherein the absorbable substrate is a cotton cloth or one or more paper towels.
19. A method according to any of the previous removing statements further comprising shampooing and rinsing the hair following elimination of the removal composition from the hair.

### Compositions

1. A pretreatment composition comprising a pretreatment composition recited by any of PART I method Statements 1-18.
2. A film forming composition comprising a film forming composition recited by any of PART III, IV, V or VI method statements.

### KIT Statements

B1 A method according to any of the preceding statements of PARTS I, II, III, IV, V and/or VI wherein the compositions for the Praeparatur and Fundamenta procedures, the pretreatment composition and the composition or compositions for film forming composition comprising the unitary binding polymer or the dual binding polymer of first and second components are maintained in separate containers prior to use.
B2 A statement of a kit according to any of the preceding method statements reciting the pretreatment composition and/or the film forming composition wherein the components are maintained in separate containers until use.

### Dispersant

Statement 1, dispersant. The film forming composition of any of the preceding statements of pretreatment compositions and the film forming composition of any of the preceding statements of methods, wherein the film forming composition further comprises: a dispersion of pigment particles and pigment dispersant.

Statement 2, dispersant. The film forming composition of Statement 1 wherein the dispersion of pigment particles and/or color bodies and pigment dispersant is compatible with the film forming composition.

Statement 3, dispersant. The film forming composition of Statement 1 or 2 wherein the dispersion of pigment particles and pigment dispersant is not compatible with the pretreatment composition.

Statement 4, dispersant. The film forming composition of any Statement 1 or 2 wherein the dispersion of pigment particles and/or color bodies and pigment dispersant is compatible with the pretreatment composition.

Statement 6, dispersant. The film forming composition of any of Statements 1-5 wherein the pigment particles and/or color bodies are coated with the pretreat molecule of the pretreatment composition.

### Topcoat

Statement T1, topcoat. A post-color coating composition comprising a medium and a topcoat composite, wherein the topcoat comprises the film forming composition of PART III, IV, V or VI.

Statement T2, topcoat. The post-color coating composition of statement T1 wherein the topcoat composite is one or more of a UV filter, a visible light filter, a radical scavenger, a triplet formation inhibitor, a water repellant, a hair spray formulation for holding a hair style or any combination thereof.

Statement T5, topcoat. The post-color coating composition of Statement T1 wherein the topcoat composite comprises a wear resistant olefinic polymer, a sacrificial non-sticky substance, a polyester, a poly (higher alkyl (meth)acrylate), a shellac, a volatile organic solvent, a fragrance and any combination thereof.

Statement T6. A statement of a method for top coating a color coating on keratin fibers comprising applying a topcoat composite of any of statements T1-T5.

Statement T7. A statement of a method according to statement T6 wherein the topcoat composite is contained in an aerosol container under pressure and is applied as a spray to the keratin fibers with a color coating.

Statement T8. A statement of a method according to statement T7 wherein the topcoat composite is diluted with a C4-C6 perfluoro hydrocarbon and/or carbon dioxide which act as a propellant.

Statement T9. A statement of a method according to any of the preceding claims T7-T9 further comprising drying and curing the topcoat.

### Post Care Compositions

Statement 1, post care. A care composition for caring for a color coating of keratin fibers comprising a medium and a surface care active for overcoating the color coating.

Statement 2, post care. The care composition of Statement 1 wherein the surface care active is a lubricating agent, a sacrificial agent, a feel modifier, an antifade agent or a combination thereof.

Statement 3, post care. The care composition of statement 1, post care, wherein the surface care active is a polymeric or long chain non-polymeric nonionic surfactant or cationic surfactant having a non-penetrating property.

Statement 4, post care. A statement of a method comprising applying a care composition of any of statements 1-3 to a color coating on keratin fibers.

Statement 5. A topcoat-care composition comprising a combination of a care composition of any of statements 1-3 and a topcoat composite of any of statements T1-T5.

Statement 6. A statement of a method comprising applying the topcoat-care composition to a color coating on keratin fibers.

### Miscellaneous Statements

The invention has been described broadly and generically herein. Each of the narrower species and subgeneric groupings falling within the generic disclosure also form part of the invention. This includes the generic description of the invention with a proviso or negative limitation removing any patient matter from the genus, regardless of whether or not the excised material is specifically recited herein. The inventions, examples, results and statement of embodiments described, stated and claimed herein may have attributes and embodiments include, but not limited to, those set forth or described or referenced in this application.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed and as provided by the statements of embodiments. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims and the statements of embodiments.

All patents, publications, scientific articles, web sites and other documents and ministerial references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such patent, publication, scientific article, web site, electronically available information, text book or other referenced material or document.

The written description of this patent application includes all claims, examples and statements of embodiments. All claims and statements of embodiments including all original claims are hereby incorporated by reference in their entirety into the written description portion of the specification and the right is reserved to physically incorporated into the written description or any other portion of the application any and all such claims and statements of embodiments. Thus, for example, under no circumstances may the patent be interpreted as allegedly not providing a written description for a claim on the assertion that the precise wording of the claim is not set forth in haec verba in written description portion of the patent.

While the invention has been described in conjunction with the detailed description thereof, the foregoing description is intended to illustrate and not limit the scope of the invention, which is defined by the scope of the appended claims and the statements of embodiments. Thus, from the foregoing, it will be appreciated that, although specific nonlimiting embodiments of the invention have been described herein for the purpose of illustration, various modifications may be made without deviating from the scope of the invention. Other aspects, advantages, and modifications are within the scope of the following claims and the present invention is not limited except as by the appended claims and the statements of embodiments.

The specific methods and compositions described herein are representative of preferred nonlimiting embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in nonlimiting embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

Unless defined otherwise, all technical and scientific terms used herein have the same meaning as commonly understood by a person of ordinary skill in the art.

What is claimed is:

1. A method for producing a coating, preferably a color coating, on keratin fibers comprising:
an activating step comprising contacting the keratin fibers with either or both of a Praeparatur procedure and a Fundamenta procedure to form modified keratin fibers;
a pretreatment step comprising applying to the modified keratin fibers a pretreatment composition to form pre-coated keratin fibers;
a binder step comprising applying to the pre-coated keratin fibers a film forming composition to form a composite film of the film forming composition and pretreatment composition on the keratin fibers and the composite film is capable of converting to the coating;
conducting the activating and pretreatment steps either simultaneously or sequentially; and,
optionally and preferably combining at least one colorant with the pretreatment composition and/or the film forming composition;
wherein:
the Praeparatur procedure comprises a cleaning process;
the Fundamenta procedure comprises an acidic oxidation process, a basic oxidation process, a plasma process, an alkali phase transfer tenside process, a reduction process or any combination thereof;
the pretreatment composition comprises a medium, at least a PTH-alkoxysilane compound comprising a PTH-organo-alkoxysilane with at least one PTH group and at least one alkoxysilane group and/or a PTH-organo-multidimethylsiloxane alkoxysilane with at least one PTH group and at least one alkoxysilane group, wherein PTH comprises R³S—, OHC—, H₂C═CR¹⁰—CO— or HO—, and R³ comprises hydrogen or a sulfur protecting group; and/or further comprising a disulfide dimer of the PTH alkoxysilane compound wherein PTH is thiol and/or a tetrasulfide dimer of the PTH-alkoxysilane compound wherein PTH is thiol, and optionally further comprising a PTH organic compound and/or an aminoorgano alkoxysilane compound;
the film forming composition comprises a medium and a binder polymer wherein the binder polymer comprises a unitary binder polymer with a binder functional group or a dual binder polymer comprising a first organic, silicone or organosilicone component and a second organic, silicone or organosilicone component wherein the first and second components have complementary binder functional groups.

2. A method according to any of the preceding claims wherein the activating step comprising at least one of the processes of the Fundamenta procedure and the pretreatment step comprising applying the pretreatment composition comprising at least a PTH alkoxysilane compound produces a colored coating having a longer lasting color remanence than the remanence of a colored coating produced without the practice of the activating step comprising at least one of the processes of the Fundamenta procedure and without the practice of the pretreatment step comprising applying a PTH alkoxysilane compound and remanence is determined according to the full root simulation color remanence test.

3. A method according to statement 2 wherein the PTH alkoxysilane compound reacts with the hair.

4. A method according to any of the preceding claims wherein the PTH group is a thiol group.

5. A method according to any of the preceding claims wherein the pretreatment composition comprises a PTH alkoxysilane compound comprising at least one of the PTH organo-alkoxysiloxane compound of Formula IIIA, the PTH organo-multi-dimethylsiloxanyl alkoxysilane of Formula IIIB, the disulfide or tetrasulfide dimer of Formula IIIA with PTH as thiol, the disulfide or tetrasulfide dimer of Formula IIIB with PTH as thiol, the cyclic thiol-alkoxysilane compound of Formula IV, and any combination thereof,
(PTH—(CH₂)ₖ—(Y)ₗ)_{d}—(ORG)ₘ-SiR¹₃₋ₙ(OR)ₙ Formula IIIA
PTH—(CH₂)ₖ—(Si(Me)₂O)ₒ-SiR¹₃₋ₙ(OR)ₙ Formula IIIB 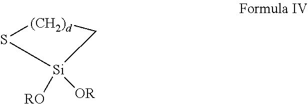
wherein:
Designator k is an integer of 1 to 20, preferably 1 to 12, more preferably 1 to 6;
Designator 1 is zero or 1;
Designator d is an integer of 1, 2 or 3;
Designator m is zero or an integer of 1 to 6;
Designator n is an integer of 1 to 3;
Designator o is an integer of from 1 to 20;
PTH comprises R³S—, OHC—, H₂C═CR¹⁰—CO₂—, HO—;
R³ comprises hydrogen, cyano, alkanoyl of 2 to 10 carbons, a phenyl group, a heteroaromatic group, a phenylalkyl group or a heteroaromatic alkyl group in which heteroaromatic group is pyridyl, pyrimidinyl, pyrrolyl or thiophenyl and the alkyl group is a C1-C4 alkyl group, and R¹⁰ may be hydrogen or methyl, such that R³S— may be a thiol group (HS—) or a protected thiol group;
R comprises a C1-C4 alkyl, preferably C1-C3 alkyl, more preferably methyl or ethyl;
Y comprises —COO—, —OOC— (carboxyl, oxycarbonyl), ether oxygen, ether thiol, —NMe-NH—, —HNCO—, —CONH—;
Group ORG comprises:
(i) a divalent organic group including alkyldithioalkyl, alkyldiazoalkyl, alkylurethanylalkyl, alkylureidoalkyl, alkylcarboxylalkyl, alkylamidoalkyl, alkylesteralkyl or alkyl in which each alkyl group independently in each instance is a C1-C20 linear or branched alkyl group, preferably a linear C1-C6 alkyl group, more preferably a linear C1-C3 alkyl group such that ORG connects the left (PTH—(CH₂)ₖ—(Y)ₗ)_{d} section and the right —SiR¹₃₋ₙ(OR²)ₙ section of Formula III; or,
(ii) a multivalent C1-C20 alkylenyl group of the formula 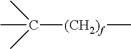
with f as zero or an integer of 1-19 in which the moiety of Formula IIIA comprising (PTH—(CH₂)ₖ—(Y)ₗ)_{d}—(ORG)ₘ becomes Formula A 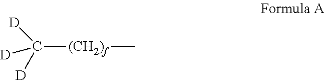
wherein two or three (PTH—(CH₂)ₖ—(Y)ₗ)_{d} sections are connected as D provided that when two D's are (PTH—(CH₂)ₖ—(Y)ₗ)_{d} sections, the third D of Formula A may be hydrogen or C1-C6 alkyl, preferably C1-C3 alkyl, more preferably methyl; and the dangling valence of (CH₂)_{f}— is bound to the right —SiR¹₃₋ₙ(OR²)ₙ section of Formula IIIA.

6. A method according to claim 5 wherein the pretreatment composition comprises at least a polycondensate of the PTH alkoxysilane of Formula IIIA and/or Formula IIIB with PTH as thiol or protected thiol wherein the thiolalkoxysilane of Formula IIIA and/or Formula IIIB is at least partially polycondensed with itself and/or an alkylalkoxysilane of Formula B wherein R⁸ is a linear or branched alkyl group of 1 to 10 carbons:
R⁸—SiR¹₃₋ₙ(OR)ₙ Formula B
to produce a linear or branched oligomeric silicone polycondensate having a silicone chain of a combination of M, D and T groups wherein the polycondensate has pendant alkoxy groups, pendant thiolalkyl groups and/or pendant alkyl groups, and the polycondensate has an M_{w} from 350 to 3500 Da and a functional equivalent M_{w} (FEM_{w}) of the thiol and or protected thiol group from 100 to 900 and a FEM_{W} for the alkoxy groups from 50 to 900.

7. A method according to claim 6 wherein Formula IIIA alone is at least partially polycondensed to form a linear or branched oligomeric silicone polycondensate.

8. A method according to any of the preceding claims wherein PTH is thiol (HS—).

9. A method according to any of the preceding claims reciting the pretreatment composition wherein the PTH organo-alkoxysilane compound comprises Formula OSSI wherein k is an integer of 1 to 20, preferably 1-12, more preferably 1-6, and the multi CH₂ chain may be linear or branched, n is an integer of 1 to 3, R¹ is methyl and R² is methyl or ethyl.
HS—(CH₂)ₖ—SiR¹₃₋ₙ(OR²)ₙ Formula OSSI.

10. A method according to claim 9 wherein Formula OSSI comprises
HS—(CH₂)ₖ—Si(OMe)₃ or HS—(CH₂)ₖ—Si(OEt)₃
wherein k is an integer of 1 to 6, preferably 1-3.

11. A method according to any of the preceding claims wherein the pretreatment composition further comprises a thiol organic compound of Formula V 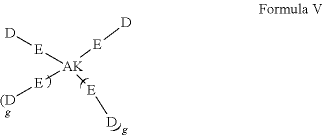
wherein
D is (PTH—(CH₂)ₖ—(Y)ₗ)_{d} as defined above;
each of the designators g is independently zero or 1;
the group E may be a bond or a C1-C6 alkylenyl group;
the group Ak is a carbon atom Ak0 or the structures Ak1, Ak2, Ak3, Ak4 depicted as follows wherein the dangling valences of the central carbon of Ak0, Ak1, Ak2 and Ak3 are bonded to E-D and the CH₂ valence is bonded to D; all dangling valences of Ak4 are bound to E-D: 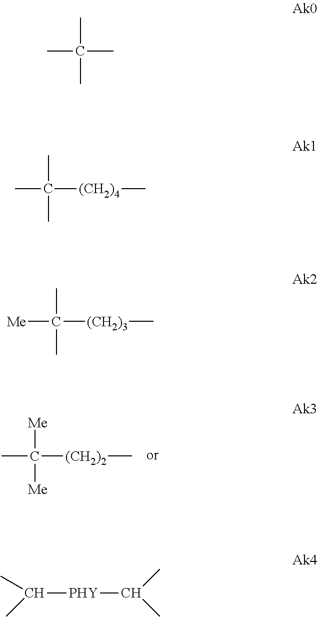
and wherein PHY is an oligomer of 2 to 10 units of a C3-C8 α,ω hydroxyalkanoic acid ester having a —O—(CH₂)ₕ—O— at its carboxy terminus and a —(CH₂)ᵢ—O— group at its hydroxyl terminus in which the —O—(CH₂)ₕ—O— and —(CH₂)ᵢ—O— groups are bonded respectively to the CH groups and the designator h is an integer of from 2 to 4 and the designator i is an integer of from 1 to 3.

12. A method according to claim 11 wherein PTH is thiol (—SH).

13. A method according to any of the preceding claims wherein the pretreatment composition further comprises the aminoorganoalkoxysilane compound comprising Formula VI:
H₂N—(CH₂)ₘ—(NH—R¹⁴—)ₙ—[ROₜMe₃₋ₜSi—O]_{b}—(—SiMe₂-O)ₚ—[(—SiMe₂₋ᵣ[(CH—₂—)_{m′}—NH₂]ᵣ—O]ₛ-[A]-[(—SiMe₂-O]ᵤ—(SiMe₃₋ₜORₜ) Formula VI
wherein
Each instance of R¹⁴ is independently a C1-C6 alkylenyl group;
R may be methyl or ethyl;
Designators m and m′ may be an integer of 1 to 3;
Designators b, r, s, c, may be zero or 1;
Designator n may be zero or an integer of 1-6, preferably 1-3
Designator t may be 1 to 3;
Designators p and u may be zero or an integer of 1 to 12;
Group A may be a divalent group including dithio, diazo, urethanyl, ureido, carboxyl, amido, ester, or aminoethyloxycarbonyl, or a C1-C20 alkylenyl group connecting the left and right sections of the aminoorgano-alkoxysiloxane compound; or,
Group A may be a multivalent C1-C20 alkylenyl group connecting two or three left sections and one right section of the aminoorganoalkoxysiloxane compound when a is 2 or 3 and b, p and s are zero; or,
Group A may be a linear or branched polyethylene imine moiety of from 2 to 2000 ethylene imine units in which case, b, p, s and u are all zero and optionally the group —(SiMe₃₋ₜORₜ) can be replaced by —NH₂; or,
Group A may be a terminal group selected from C2-C8 alkylenyl(meth)acrylate or —(CH₂)ₙ—O—CH₂—CHOHCH₂-02C(R)═CH₂ wherein R is H or CH₃ and n is an integer of 2 to 8.

14. A method according to claim 13 wherein Formula VI comprises Formula OASI
H₂N—(CH₂)ₘ—(NH—R¹⁴—)ₙ—(SiMe₂₀)ₚ-A_{c}(-SiMe₂-O)ᵤ SiMe₃₋ₜORₜ Formula OASI
wherein
m is an integer of 1 to 6;
n is zero or an integer of 1 to 3;
p and u are each independently zero or an integer of 1 to 3;
c is zero or 1;
t is an integer of 1-3, preferably 1 or 2.
A is C1-C6 alkylenyl; and,
R is methyl or ethyl.

15. A method according to claim 14 wherein Formula OASI is
H₂N—(CH₂)ₘ—Si(OR)₃ or H₂N—(CH₂)ₘ—(NH—R¹⁴)ₙ—NH—R^{14′}—Si(OR)₃ or H₂N—(CH₂)ₘ—NH—R¹⁴—Si(OR)₃
wherein m is 2 or 3, n is 1 or 2, each instance of R¹⁴ independently is ethyl or propyl or isobutyl and R^{14′} is propyl, butyl or isobutyl.

16. A method according to any of the preceding claims wherein the pretreatment step and binder step are conducted simultaneously.

17. A method according to any of the preceding claims wherein the pretreatment step and binder step are conducted sequentially.

18. A method according to any of the preceding claims wherein the keratin fibers are anagenic hair, preferably hair on the scalp of a human.

19. A method according to any of the preceding claims wherein the colorant is not present.

20. A method according to any of the preceding claims wherein the curing of the composite coating comprises a procedure selected from drying, heating, heating and drying and addition of a catalyst to promote the rate of curing.

21. A method according to any of the preceding claims wherein the colorant is present and comprises a pigment or a coated pigment.

22. A method according to any of the preceding claims wherein the Fundamenta procedure is optionally combined with the pretreatment step, and preferably the Fundamenta procedure is an acidic oxidation process, a basic oxidation process, a reduction process or a combination of a reduction process and then an acidic oxidation process.

23. A method according to any of the preceding claims wherein the process of the Fundamenta procedure is the reduction process followed by acidic oxidation process.

24. A method according to any of the preceding claims wherein the process of the Fundamenta procedure is the acidic oxidation process.

25. A method according to any of the preceding claims wherein the activating step includes the Praeparatur procedure and the Fundamenta procedure and the Praeparatur procedure and Fundamenta procedure optionally are combined.

26. A method according to any of the preceding claims 1-24 wherein the activating step includes at least one of the processes of the Fundamenta procedure but not the Praeparatur procedure.

27. A method according to any of the preceding claims wherein the film forming composition comprises a unitary binder polymer comprising an organic binder polymer of one or more monomeric units selected from an olefinic carboxylate ester unit, an olefinic carboxamide unit, a carbon-hydrogen olefinic unit, an ester monomeric unit, an amide monomeric unit, a urethane monomeric unit, a urea monomeric unit, and the organic, polymer has at least one pendant and/or terminal binder functional monogroup comprising an alkoxysilyl group.

28. A statement of a method according to claim 27 wherein the organic binder polymer comprises at least a compound of Formula I
X₃Si—R¹-Ct-[Poly]_{y}-Ct-R¹—Si—X₃ Formula IA
wherein
X is hydroxy or alkoxy of 1 to 3 carbons;
R¹ is a C1 to C8 alkylenyl group;
Ct is a connector group of the Formula II —U¹—R²—U²— joining X₃Si—R¹—to Poly, wherein:
U¹ is covalently bonded to R¹ and U² is covalently bonded to Poly;
Each of U¹ and U² independently is a urea or urethane group;
R² is a C2 to C12 alkylenyl group, a C6-C16 alkylcycloalkyl group or a C6-C14 aromatic or alkylaromatic group;
Poly is a polymer of monomeric units of an organic ester, urethane, urea, amide or polyol or any combination thereof and y designates the number of monomeric units of Poly forming a polymeric backbone wherein y is an integer of from 2 up to about 1 million, preferably up to about 300,000, more preferably up to about 250,000, most preferably up to about 200,000 and Poly is linear or branched, preferably linear; wherein
The organic ester monomeric unit is formed of a C2-C20 alkane diol or a C6-C10 aromatic diol and a C3 to C10 alkanodioic acid or a C8-C10 aromatic dicarboxylic acid or the unit is formed of a C3-C10 hydroxy alkanoic acid or a C8-C10 aromatic hydroxycarboxylic acid;
The organic urethane monomeric unit is formed of a C2-C10 alkane diol and an R³-diisocyanate;
The organic urea monomeric unit is formed of a C2-C10 alkane diamine and an R³-diisocyanate;
The organic amide monomeric unit is formed of a C2-C10 alkane diamine and a C3 to C10 alkanodioic acid or a C8-C10 aromatic dicarboxylic acid;
The polyol monomeric unit is a formed of ethylene oxide or propylene oxide;
R³ is a linear or branched C2 to C12 alkylenyl group, a C6-C16 alkylcycloalkyl group or a C6-C14 aromatic or alkylaromatic group;
Provided that:
When Poly is an ester monomeric unit, U² is a urethane group and U¹ is a urea group;
When Poly is a urethane monomeric unit, U² is a urethane group and U¹ is a urea group;
When Poly is a urea monomeric unit, U² is a urea group and U¹ is a urea group;
When Poly is an amide monomeric unit, U² and U¹ are both urea groups;
When Poly is a polyol monomeric unit, U² is a urethane group and U¹ is a urea group; or,
Alternatively, U¹ may be a urethane group for each of the Polyester, Polyurethane, Polyurea, Polyamide and Polypolyol provisos.

29. A method according to any of claims 1-26 wherein the film forming composition comprises a unitary binder polymer which comprises an organic polymer of one or more monomeric units selected from an olefinic carboxylate ester unit, an olefinic carboxamide unit, a carbon-hydrogen olefinic unit, an ester monomeric unit, an amide monomeric unit, a urethane monomeric unit, a urea monomeric unit, the organic polymer has at least one pendant and/or terminal binder functional monogroup comprising at least one pendant and/or terminal carboxylic acid group; and optionally the organic polymer comprises at least one pendant organoalkoxysilane group of the formula —(CH₂)ₙ—SiMeₜ₋₃(OR)ₜ wherein n is an integer of 2 to 10, t is an integer of 1 to 3 and the dangling valence of (CH₂) is connected to a carbon of the organic polymer backbone.

30. A method according to claim 29 wherein the pretreatment composition comprises the PTH alkoxysilane compounds compound and the aminoorgano alkoxysiloxane compound and the organic polymer with at least one carboxylic acid binder functional monogroup is capable of non-covalent interaction with the pretreatment composition.

31. A method according to claim 29 or 30 wherein the binder polymer comprises an organic polymer which comprises repeating units of at least one olefinic acid monomeric unit and at least one non-acid olefinic monomeric unit selected from an olefinic carboxylate ester monomer unit, an olefinic carboxamide monomer unit, a hydrophilic olefinic monomer unit, a lipophilic olefin monomer unit and any combination thereof, wherein:
the olefinic acid monomeric unit is selected from (meth)acrylic acid, crotonic acid, maleic acid, fumaric acid, itaconic acid, gluconic acid, a C5-C10 ethenoic acid or any combination thereof;
the olefinic carboxylate ester monomeric unit is selected from a C1-C30 linear or branched alkyl ester of any of the olefinic acid monomeric units or any combination thereof;
the olefinic carboxamide monomeric unit is selected from an —NH₂, —NR¹H or —NR¹R² amide of any of the olefinic acid monomeric units or any combination thereof wherein R¹ and R² are each independently selected from a C1-C6 linear or branched alkyl;
the hydrophilic olefinic monomer is a hydroxy alkyl ester of the olefinic carboxylic acid monomeric unit and a linear or branched C2-C24 alkyl diol, or is an aminoalkyl ester of the olefinic carboxylic acid monomeric unit and a linear or branched amino C2-C24 alkyl alcohol or any combination thereof; and
the lipophilic olefin monomer unit is selected from an olefin compound of the formula
R³HC═CHR⁴
wherein R³ is selected from hydrogen, linear or branched alkyl of one to six carbons, unsubstituted phenyl or phenyl substituted by a linear or branched alkyl of 1 to six carbons, methyl or ethyl carboxylate, carboxamide or hydroxyl, R⁴ is selected from hydrogen, linear or branched alkyl of one to six carbons, unsubstituted phenyl or phenyl substituted by a linear or branched alkyl of 1 to six carbons, methyl or ethyl carboxylate, carboxamide or hydroxyl, or an ethenyl group of the formula —CH═CHR⁵ wherein R⁵ is selected from hydrogen, linear or branched alkyl of one to six carbons, unsubstituted phenyl or phenyl substituted by a linear or branched alkyl of 1 to six carbons, methyl or ethyl carboxylate, carboxamide or hydroxyl.

32. A method according to any of claims 1-26 wherein:
the film forming composition comprises a dual binder polymer comprising a first component comprising a silicone or organosilicone polymer having at least one pendant and/or terminal first binder functional group, and a second component comprising a small molecule, pre-polymer or polymer having at least one pendant and/or terminal second binder functional group; and,
the first and second binder functional groups are a complementary pair selected from the group consisting of (i) alkenoyloxy and amine or (ii) alkenoyloxy and thiol.

33. A method according to claim 32 wherein the first component comprises a silicone polymer of Formula I
X_{z}—SiMe_{3-z}O-(Me₂SiO)ₓ—(Si(—X)MeO)_{y}—SiOMe_{3-z}-X_{z} Formula I
wherein
each of Me₂SiO and Si(—X)MeO comprise a monomeric siloxane D unit, and X_{z}SiMe_{3-z}O comprises a monomeric siloxane M unit;
X comprises R¹R²C═CR³COO—R⁴—;
each of R¹ and R² independently is a hydrogen or a C1-C6 alkyl group, provided that at least one of R¹ and R² is hydrogen;
R³ is hydrogen or methyl;
R⁴ is a C1 to C12 alkylenyl group, a C3-C12 cycloalkylalkyl or cycloalkyl group, a C6-C20 arylalkyl group or a C6-C20 aryl group wherein any or all of the groups are optionally substituted in chain by one or more ether oxygen, thioether sulfur and/or amine groups and/or pendantly by hydroxyl groups and R⁴ is attached to silicon of a monomeric D and/or M unit;
each of the designators x and y independently designates the number of monomeric D siloxane units forming the corresponding linear polymeric silicone backbone, wherein x is an integer of from 1 up to about 100,000 and designator y is zero or an integer of from 1 to 10;
designator z for each of the X_{z}SiMe_{3-z}O units is zero or 1 so that the X_{z}SiMe_{3-z}O unit may have a terminal X group or may be an M-type trimethyl siloxy group;
the sum of x and y is an integer of from about 3 up to about 200,000, preferably up to about 150,000, more preferably up to about 100,000, most preferably up to about 50,000 and especially most preferably up to about 100, with exemplary sums of 10 to 50 and 10 to 20;
and the multiple monomeric units of Me₂SiO and Si(—X)MeO are randomly distributed in Formula I; and wherein
the second component comprises Formula V
M1-(D)_{d}-M2 Formula V
wherein
M1 and M2 are termini of the second component and may be selected from Me₃SiO units, A-SiMe₂O units and —Si(OR)₃ units wherein R is methyl or ethyl and A is an organoamine or organothiol group of the Formula OA
Y—(R¹⁰—NH)ᵣ—R¹- Formula OA
wherein Y is —NH₂ or —SH; R¹⁰ is a linear or branched C1-C10 alkyl group or a linear or branched C6-C14 alkylaryl group; R¹¹ is a linear or branched C1-C10 alkyl group or a linear or branched C6-C14 alkylaryl group; and designator r is zero or an integer of 1 to 3 and R¹¹ is bonded to silicon when r is other than zero and R¹¹ is bonded to silicon when r is zero and when Y is —SH, r is zero; and
D units form the backbone of the polydimethylsiloxane-type second component with designator d being an integer of from 3 to 20,000 indicating the size of the second component, wherein
the D units are selected from SiMe₂O units (dimethylsiloxane units) and A-SiMeO units; and wherein:
the second component comprises at least one A-SiMeO unit.

34. A method according to any of claims 1-26 wherein:
the film forming composition comprises a dual binder polymer comprising a first component comprising an organic, silicone or organosilicone polymer having at least one pendant and/or terminal first binder functional group and a second component comprising a small molecule, pre-polymer or polymer having at least one pendant and/or terminal second binder functional group; and,
the first and second binder functional groups are a complementary pair comprising carboxylic acid and carbodiimide.

35. A method according to claim 34 wherein the first component comprises an olefinic, silicone or organosilicone polymer of Formula I
MUE-(MU1)ₓ-(MUX)_{y}-(MU2)_{z}-(MU3)ₐ-(MU3X)_{b}-MUE Formula I
wherein
MU1 comprises a hydrophobic olefinic monomeric unit comprising a linear C2-C10 alkene residue, a linear C4-C12 alkadiene residue and/or a C6-C10 aromatic/alkylaromatic vinyl residue,
MUX comprises an acidic olefinic monomeric unit comprising a linear C3-C10 alkenoic acid residue or a C4-C10 alkadienoic acid residue;
MU2 comprises a hydrophilic olefinic monomeric unit comprising a vinyl linear C2-C16 alkanoic ester residue, a C1-C14 linear alkyl or hydroxyalkyl linear C2-C14 alkenoic ester residue, a linear C2-C10 alkenoic amide residue or N-C1-C4 alkyl substituted version of the amide residue;
MU3 comprises a dimethylsiloxane residue;
MU3X comprises a monomethylsiloxane residue bound to an alkanoic acid of at least 4 carbons with one of the alkyl carbons of the alkanoic acid optionally having a hydroxy group; and
MUE comprises a single terminal monomeric unit of MU1, MU2, MU3, MUX or MU3X; each of the designators x, y, z, a and b independently designates the number of corresponding monomeric units forming the linear polymeric backbone, wherein each of x, z and a is zero or an integer of from 1 up to about 100,000 and y and b are each zero or an integer of 1 to 100;
when b is an integer, y may be zero or an integer and when b is zero, y is an integer;
the sum of x, y, z, a and b is an integer of from about 3 up to about 1 million, preferably up to about 300,000, more preferably up to about 250,000, most preferably up to about 200,000;
the multiple monomeric units of MU1, MU2 and MU3 are randomly distributed or form blocks in Formula I and the multiple carboxylic acid monomeric units MUX and MU3X are randomly distributed among MU1, MU2 and MU3 units; and,
the first component being linear or branched, preferably linear; and wherein, the second component comprises an organic polymer of Formula II, a polymer with in-chain carbodiimide groups or Formula X, a polymer with pendant single carbodiimide groups
Z-(L-N═C═N—)ₚ—Z Formula II
(Poly)_{q}-(K)ₛ-(Poly)ᵣ Formula X
wherein;
For Formula II, p is an integer of at least 2; and L is an organic second component group comprising saturated aliphatic divalent radical, an aromatic divalent radical or an alkylaromatic divalent radical or a polymer or oligomeric divalent radical with repeating olefinic, carbonate, ester, ether, amide, imine, urethane or urea linkages;
For Formula X, each Poly is an organic polymer segment of an amide, imine, olefinic, caronate, ester, ether, urethan or urea monomeric residue and preferably the residue is an amide or urea/urethane monomeric residue is based upon a C3 to C6 alkane diamine and a C4-C10 alkane dicarboxylic acid or C4-C10 alkane diisocyanate, or an ester monomeric residue based upon a C3-C6 alkane diol and a C4-C10 alkane dicarboxylic acid and the designators q and r each being an integer of at least 2; K is a pendant carbodiimide group of Formula XI with s being an integer of at least 2 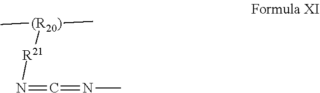
wherein R²⁰ is a C3 to C6 alkylenyl residue, R²¹ is a C3-C6 alkylenyl residue;
For Formulas II and XI Z is a non-reactive or reactive terminal group of the polycarbodiimide; and, the multiple K's are randomly distributed along the Poly backbone; L or Poly of the second component being linear or branched, preferably linear.

36. A method according to any of the preceding claims wherein the colorant is combined with the film forming composition.

37. A method according to any of the preceding claims wherein the colorant is a pigment and/or a coated pigment.

38. A method according to any of the preceding claims wherein the pretreatment composition and the components of the film forming composition in compatible medium are all maintained in separate containers until before use.

39. A method according to any of the preceding claims wherein separate quantities of the first and second components each in a compatible medium are combined to form the film forming composition before application to keratin fibers.
